SEQUENCE LISTING (12 sequences)

SEQ ID NO: 1 - protein, 124 aa, Artificial Sequence
  source: /note=“Description of Artificial Sequence Synthetic polypeptide”
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 2 - protein, 112 aa, Artificial Sequence
  source: /note=“Description of Artificial Sequence Synthetic polypeptide”
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 3 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 107 aa, Artificial Sequence [sequence omitted]

SEQ ID NO: 5 - protein, 5 aa, Artificial Sequence
  source: /note=“Description of Artificial Sequence Synthetic peptide”
SFGMH

SEQ ID NO: 6 - protein, 17 aa, Artificial Sequence
  source: /note=“Description of Artificial Sequence Synthetic peptide”
YISSGSSTIYYGDTVKG

SEQ ID NO: 7 - protein, 15 aa, Artificial Sequence
  source: /note=“Description of Artificial Sequence Synthetic peptide”
EGGYYYGRSYYTMDY

SEQ ID NO: 8 - protein, 16 aa, Artificial Sequence
  source: /note=“Description of Artificial Sequence Synthetic peptide”
RSSQSIVHSNGNTYLE

SEQ ID NO: 9 - protein, 7 aa, Artificial Sequence
  source: /note=“Description of Artificial Sequence Synthetic peptide”
KVSNRFS

SEQ ID NO: 10 - protein, 9 aa, Artificial Sequence
  source: /note=“Description of Artificial Sequence Synthetic peptide”
FQGSHVPPT

SEQ ID NO: 11 - protein, 454 aa, Artificial Sequence
  source: /note=“Description of Artificial Sequence Synthetic polypeptide”
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 219 aa, Artificial Sequence
  source: /note=“Description of Artificial Sequence Synthetic polypeptide”
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2021/0324056 A1
(43) Pub. Date: Oct. 21, 2021

(54) METHODS OF TREATMENT AND PREVENTION OF ALZHEIMER'S DISEASE

(71) Applicant: Eisai R&D Management Co., Ltd., Tokyo (JP)

(72) Inventors: Johan LUTHMAN, Woodcliff Lake, NJ (US); Chad J. SWANSON, Woodcliff Lake, NJ (US); Yong ZHANG, Woodcliff Lake, NJ (US); Shobha DHADDA, Woodcliff Lake, NJ (US); Jinping WANG, Woodcliff Lake, NJ (US); Lynn KRAMER, Woodcliff Lake, NJ (US)

(21) Appl. No.: 17/250,448

(22) PCT Filed: Jul. 23, 2019
(86) PCT No.: PCT/US2019/043067

§ 371 Date: Jan. 22, 2021

Related U.S. Application Data
(60) Provisional application No. 62/874,684, filed on Jul. 16, 2019.
(60) Provisional application No. 62/846,902, filed on May 13, 2019.
(60) Provisional application No. 62/824,162, filed on Mar. 26, 2019.
(60) Provisional application No. 62/749,614, filed on Oct. 23, 2018.
(60) Provisional application No. 62/702,659, filed on Jul. 24, 2018.

(51) Int. Cl. C07K 16/18; A61K 31/542; A61P 25/28

(52) U.S. Cl. CPC C07K 16/18; A61K 2039/505; A61P 25/28; A61K 31/542

(57) ABSTRACT

Provided herein are methods of reducing clinical decline in a subject having early Alzheimer's disease, methods of converting an amyloid positive subject having early Alzheimer's disease to amyloid negative, methods of reducing brain amyloid level in a subject, and methods of preventing Alzheimer's disease, the methods comprising administering a composition comprising a therapeutically effective amount of at least one anti-Aβ protofibril antibody. In some embodiments, the subject is ApoE4-positive. In some embodiments, the at least one anti-Aβ protofibril antibody is BAN2401.

The present application claims the benefit of priority to U.S. Provisional Application No. 62/702,659 filed Jul. 24, 2018; U.S. Provisional Application No. 62/749,614 filed Oct. 23, 2018; U.S. Provisional Application No. 62/824,162 filed Mar. 26, 2019; U.S. Provisional Application No. 62/846,902 filed May 13, 2019; and U.S. Provisional Application No. 62/874,684 filed Jul. 16, 2019; the entire contents of each are incorporated herein by reference.

Alzheimer's disease (AD) is a progressive, neurodegenerative disorder of unknown etiology and the most common form of dementia among older people. In 2006, there were 26.6 million cases of AD in the world (range: 11.4-59.4 million) (Brookmeyer, R., et al., Forecasting the global burden of Alzheimer's Disease. Alzheimer Dement. 2007; 3:186-91), while there were more than 5 million people in the United States reportedly living with AD (Alzheimer's Association. Alzheimer's Association report. 2010 Alzheimer's disease facts and figures. Alzheimer Dement. 2010;6:158-94). By the year 2050, the worldwide prevalence of AD is predicted to grow to 106.8 million (range: 47.2-221.2 million), while in the United States alone the prevalence is estimated to be 11 to 16 million. (Brookmeyer, supra, and 2010 Alzheimer's disease facts and figures, supra).

The disease generally involves a global decline of cognitive function that progresses slowly and leaves end-stage patients bedridden. AD patients typically survive for only 3 to 10 years after symptom onset, although extremes of 2 and 20 years are known. (Hebert, L. E., et al., Alzheimer disease in the U.S. population: prevalence estimates using the 2000 census. Arch Neurol. 2003; 60:1119-1122.) AD is the seventh leading cause of all deaths in the United States and the fifth leading cause of death in Americans older than the age of 65 years, despite the fact that mortality due to AD is greatly underestimated because death certificates rarely attribute the cause of death to AD. (Alzheimer's Association. Alzheimer's Association report. 2010 Alzheimer's disease facts and figures. Alzheimer Dement. 2010; 6:158-94.)

AD represents a significant economic burden across industrialized countries with a substantial impact on healthcare systems and the public purse as well as on patients and their families. In the United States alone, total payments for 2010 were estimated at $172 billion, including $123 billion for Medicare and Medicaid.

To our knowledge, at this time, there is no cure for AD and no way of slowing down the progression of this disease. Current therapeutic agents for treatment of AD include symptomatic therapies such as acetylcholinesterase inhibitors (AChEIs), such as donepezil, and N-methyl-D-aspartate (NMDA)-receptor antagonists, such as memantine. While these agents may improve the symptoms of AD, such as cognitive decline and decline in activities of daily living and behavior, they have not been reported to alter the progression of the disease. Thus, there is an unmet need for methods of treating the progression of and/or preventing AD.

In some embodiments, provided herein is a method of reducing clinical decline in a subject having early Alzheimer's disease comprising administering a composition comprising a therapeutically effective amount of at least one anti-Aβ protofibril antibody. In some embodiments, the subject is an ApoE4 carrier. In some embodiments, the at least one anti-Aβ protofibril antibody is BAN2401.

In some embodiments, the at least one anti-Aβ protofibril antibody, such as BAN2401, prevents Aβ deposition before plaques begin to develop in the brain. In some embodiments, the at least one anti-Aβ protofibril antibody, such as BAN2401, reduces protofibrils and existing plaques in the brain. In some embodiments, the at least one anti-Aβ protofibril antibody, such as BAN2401, prevents Aβ deposition before plaques begin to develop and reduces protofibrils and existing plaques in the brain.

In some embodiments, provided herein is a method of converting a subject having amyloid positive early Alzheimer's disease to a subject having amyloid negative early Alzheimer's disease comprising administering a composition comprising a therapeutically effective amount of at least one anti-Aβ protofibril antibody. In some embodiments, the subject is an ApoE4 carrier. In some embodiments, the at least one anti-Aβ protofibril antibody is BAN2401.

In some embodiments, provided herein is a method of reducing brain amyloid level in a subject having early Alzheimer's disease comprising administering a composition comprising a therapeutically effective amount of at least one anti-Aβ protofibril antibody. In some embodiments, the subject is an ApoE4 carrier. In some embodiments, the at least one anti-Aβ protofibril antibody is BAN2401.

In some embodiments, provided herein is a method of preventing Alzheimer's disease in ApoE4-positive subjects. In some embodiments, said method comprises determining a pre-administration brain amyloid level of a subject and administering a therapeutically effective amount of at least one anti-Aβ protofibril antibody if the brain amyloid level of the subject is above a first predetermined level. In some embodiments, the at least one anti-Aβ protofibril antibody is BAN2401.

### BRIEF DESCRIPTION OF DRAWINGS

FIG. 1 shows the slowing of cognitive decline versus placebo over 18 months, as determined by ADCOMS, for 2.5 mg/kg bi-weekly, 5 mg/kg monthly, 5 mg/kg bi-weekly, 10 mg/kg monthly, and 10 mg/kg bi-weekly doses of BAN2401.

FIG. 2 shows the slowing of cognitive decline versus placebo over 18 months, as determined by ADCOMS, for a dose of 10 mg/kg BAN2401 monthly and a dose of 10 mg/kg BAN2401 bi-weekly.

FIG. 3 shows the slowing of cognitive decline versus placebo over 18 months, as determined by ADCOMS, for a dose of 10 mg/kg BAN2401 monthly and a dose of 10 mg/kg BAN2401 bi-weekly, in subjects having mild cognitive impairment due to Alzheimer's disease dementia - moderate likelihood.

FIG. 4 shows the slowing of cognitive decline versus placebo over 18 months, as determined by ADCOMS, for a dose of 10 mg/kg BAN2401 monthly and a dose of 10 mg/kg BAN2401 bi-weekly, in subjects having mild Alzheimer's disease dementia.

FIG. 5 shows the slowing of cognitive decline versus placebo over 18 months, as determined by ADCOMS, for a dose of 10 mg/kg BAN2401 monthly and a dose of 10 mg/kg BAN2401 bi-weekly, in ApoE4-positive subjects.

FIG. 6 shows the slowing of cognitive decline versus placebo over 18 months, as determined by ADCOMS, for a dose of 10 mg/kg BAN2401 monthly and a dose of 10 mg/kg BAN2401 bi-weekly, in ApoE4-negative subjects.

FIG. 7 shows the slowing of cognitive decline versus placebo over 18 months, as determined by ADAS-cog, for 2.5 mg/kg bi-weekly, 5 mg/kg monthly, 5 mg/kg bi-weekly, 10 mg/kg monthly, and 10 mg/kg bi-weekly doses of BAN2401.

FIG. 8 shows the slowing of cognitive decline versus placebo over 18 months, as determined by ADAS-cog, for a dose of 10 mg/kg BAN2401 monthly and a dose of 10 mg/kg BAN2401 bi-weekly.

FIG. 9 shows the slowing of cognitive decline versus placebo over 18 months, as determined by CDR-SB, for 2.5 mg/kg bi-weekly, 5 mg/kg monthly, 5 mg/kg bi-weekly, 10 mg/kg monthly, and 10 mg/kg bi-weekly doses of BAN2401.

FIG. 10 shows the slowing of cognitive decline versus placebo over 18 months, as determined by CDR-SB, for a dose of 10 mg/kg BAN2401 monthly and a dose of 10 mg/kg BAN2401 bi-weekly.

FIG. 11 shows the reduction of amyloid in the brain, as determined by PET, as a global cortical average versus whole cerebellum reference, over 18 months, for 2.5 mg/kg bi-weekly, 5 mg/kg monthly, 5 mg/kg bi-weekly, 10 mg/kg monthly, and 10 mg/kg bi-weekly doses of BAN2401.

FIG. 12 shows the reduction of amyloid in the brain, as determined by imaging using binding of radiotracers for brain Aβ amyloid and visualized with PET, over 18 months, for 2.5 mg/kg bi-weekly, 5 mg/kg monthly, 5 mg/kg bi-weekly, 10 mg/kg monthly, and 10 mg/kg bi-weekly doses of BAN2401.

FIG. 13 shows the conversion of amyloid positive subjects to amyloid negative subjects, as determined by Visual Read, after 12 and 18 months of treatment for 2.5 mg/kg bi-weekly, 5 mg/kg monthly, 5 mg/kg bi-weekly, 10 mg/kg monthly, and 10 mg/kg bi-weekly doses of BAN2401.

FIG. 14 shows the change in cerebrospinal fluid level of Aα₁₋₄₂ over 18 months for 2.5 mg/kg bi-weekly, 5 mg/kg monthly, 5 mg/kg bi-weekly, 10 mg/kg monthly, and 10 mg/kg bi-weekly doses of BAN2401.

FIG. 15 shows the change in cerebrospinal fluid level of total tau over 18 months for a dose of 10 mg/kg BAN2401 monthly and a dose of 10 mg/kg BAN2401 bi-weekly.

FIG. 16 shows the slowing of cognitive decline versus placebo over 18 months, as determined by ADCOMS, for 2.5 mg/kg bi-weekly, 5 mg/kg monthly, 5 mg/kg bi-weekly, 10 mg/kg monthly, and 10 mg/kg bi-weekly doses of BAN2401, in ApoE4-positive subjects.

FIG. 17 shows the slowing of cognitive decline versus placebo over 18 months, as determined by ADCOMS, for a dose of 10 mg/kg BAN2401 monthly and a dose of 10 mg/kg BAN2401 bi-weekly, in ApoE4-positive subjects having mild cognitive impairment due to Alzheimer's disease—intermediate likelihood.

FIG. 18 shows the slowing of cognitive decline versus placebo over 18 months, as determined by ADCOMS, for a dose of 10 mg/kg BAN2401 monthly and a dose of 10 mg/kg BAN2401 bi-weekly, in ApoE4-positive subjects having mild Alzheimer's disease dementia.

FIG. 19 shows the slowing of cognitive decline versus placebo over 18 months, as determined by ADAS-cog, for 2.5 mg/kg bi-weekly, 5 mg/kg monthly, 5 mg/kg bi-weekly, 10 mg/kg monthly, and 10 mg/kg bi-weekly doses of BAN2401, in ApoE4-positive subjects.

FIG. 20 shows the slowing of cognitive decline versus placebo over 18 months, as determined by ADAS-cog, for 10 mg/kg monthly and 10 mg/kg bi-weekly doses of BAN2401, in ApoE4-positive subjects.

FIG. 21 shows the slowing of cognitive decline versus placebo over 18 months, as determined by ADAS-cog, for a dose of 10 mg/kg BAN2401 monthly and a dose of 10 mg/kg BAN2401 bi-weekly, in ApoE4-positive subjects having mild cognitive impairment due to Alzheimer's disease—intermediate likelihood.

FIG. 22 shows the slowing of cognitive decline versus placebo over 18 months, as determined by ADAS-cog, for a dose of 10 mg/kg BAN2401 monthly and a dose of 10 mg/kg BAN2401 bi-weekly, in ApoE4-positive subjects having mild Alzheimer's disease dementia.

FIG. 23 shows the slowing of cognitive decline versus placebo over 18 months, as determined by CDR-SB, for 2.5 mg/kg bi-weekly, 5 mg/kg monthly, 5 mg/kg bi-weekly, 10 mg/kg monthly, and 10 mg/kg bi-weekly doses of BAN2401, in ApoE4-positive subjects.

FIG. 24 shows the slowing of cognitive decline versus placebo over 18 months, as determined by CDR-SB, for 10 mg/kg monthly and 10 mg/kg bi-weekly doses of BAN2401, in ApoE4-positive subjects.

FIG. 25 shows the slowing of cognitive decline versus placebo over 18 months, as determined by CDR-SB, for a dose of 10 mg/kg BAN2401 monthly and a dose of 10 mg/kg BAN2401 bi-weekly, in ApoE4-positive subjects having mild cognitive impairment due to Alzheimer's disease—intermediate likelihood.

FIG. 26 shows the slowing of cognitive decline versus placebo over 18 months, as determined by CDR-SB, for a dose of 10 mg/kg BAN2401 monthly and a dose of 10 mg/kg BAN2401 bi-weekly, in ApoE4-positive subjects having mild Alzheimer's disease dementia.

FIG. 27 shows the reduction of amyloid in the brain, as determined by visual reads of amyloid PET images, as a global cortical average versus whole cerebellum reference, over 18 months, for 2.5 mg/kg bi-weekly, 5 mg/kg monthly, 5 mg/kg bi-weekly, 10 mg/kg monthly, and 10 mg/kg bi-weekly doses of BAN2401, in ApoE4-positive subjects.

FIG. 28 shows the reduction of amyloid in the brain, as determined by imaging using binding of radiotracers for brain Aβ amyloid and visualized with PET, over 18 months, for 2.5 mg/kg bi-weekly, 5 mg/kg monthly, 5 mg/kg bi-weekly, 10 mg/kg monthly, and 10 mg/kg bi-weekly doses of BAN2401, in ApoE4-positive subjects.

FIG. 29 shows the conversion of amyloid positive, ApoE4-positive subjects to amyloid negative, ApoE4-positive subjects after 12 and 18 months of treatment.

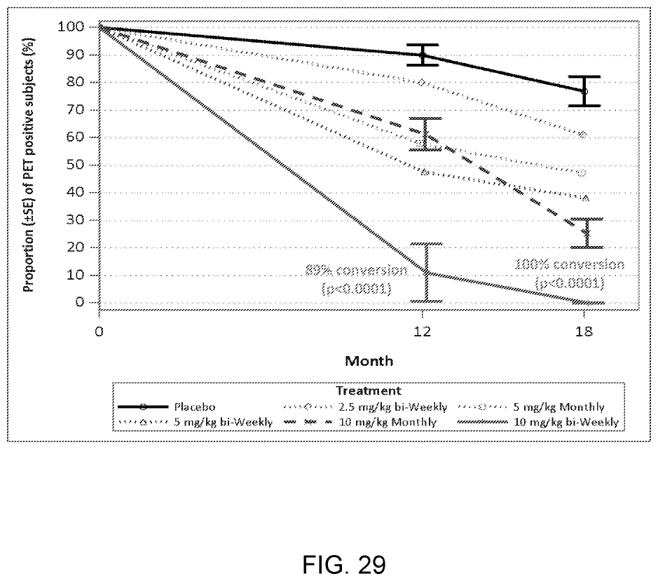

FIG. 30 shows the change in cerebrospinal fluid level of Aβ₁₋₄₂ over 18 months for 2.5 mg/kg bi-weekly, 5 mg/kg monthly, 5 mg/kg bi-weekly, 10 mg/kg monthly, and 10 mg/kg bi-weekly doses of BAN2401 in ApoE4-positive subjects.

FIG. 31 shows the change in cerebrospinal fluid level of total tau over 18 months for a dose of 10 mg/kg BAN2401 monthly and a dose of 10 mg/kg BAN2401 bi-weekly in ApoE4-positive subjects.

FIG. 32 shows the slowing of cognitive decline versus placebo over 18 months, as determined by ADCOMS, for 2.5 mg/kg bi-weekly, 5 mg/kg monthly, 5 mg/kg bi-weekly, 10 mg/kg monthly, and 10 mg/kg bi-weekly doses of BAN2401, in ApoE4-negative subjects.

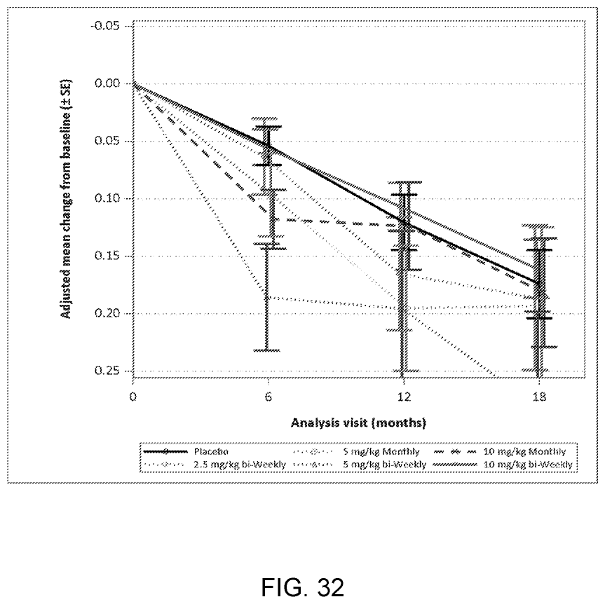

FIG. 33 shows the slowing of cognitive decline versus placebo over 18 months, as determined by ADCOMS, for a dose of 10 mg/kg BAN2401 monthly and a dose of 10 mg/kg BAN2401 bi-weekly, in ApoE4-negative subjects having mild cognitive impairment due to Alzheimer's disease—intermediate likelihood.

FIG. 34 shows the slowing of cognitive decline versus placebo over 18 months, as determined by ADCOMS, for a dose of 10 mg/kg BAN2401 monthly and a dose of 10 mg/kg BAN2401 bi-weekly, in ApoE4-negative subjects having mild Alzheimer's disease dementia.

FIG. 35 shows the slowing of cognitive decline versus placebo over 18 months, as determined by ADAS-cog, for 2.5 mg/kg bi-weekly, 5 mg/kg monthly, 5 mg/kg bi-weekly, 10 mg/kg monthly, and 10 mg/kg bi-weekly doses of BAN2401, in ApoE4-negative subjects.

FIG. 36 shows the slowing of cognitive decline versus placebo over 18 months, as determined by ADAS-cog, for 10 mg/kg monthly and 10 mg/kg bi-weekly doses of BAN2401, in ApoE4-negative subjects.

FIG. 37 shows the slowing of cognitive decline versus placebo over 18 months, as determined by ADAS-cog, for a dose of 10 mg/kg BAN2401 monthly and a dose of 10 mg/kg BAN2401 bi-weekly, in ApoE4-negative subjects having mild cognitive impairment due to Alzheimer's disease—intermediate likelihood.

FIG. 38 shows the slowing of cognitive decline versus placebo over 18 months, as determined by ADAS-cog, for a dose of 10 mg/kg BAN2401 monthly and a dose of 10 mg/kg BAN2401 bi-weekly, in ApoE4-negative subjects having mild Alzheimer's disease dementia.

FIG. 39 shows the slowing of cognitive decline versus placebo over 18 months, as determined by CDR-SB, for 2.5 mg/kg bi-weekly, 5 mg/kg monthly, 5 mg/kg bi-weekly, 10 mg/kg monthly, and 10 mg/kg bi-weekly doses of BAN2401, in ApoE4-negative subjects.

FIG. 40 shows the slowing of cognitive decline versus placebo over 18 months, as determined by CDR-SB, for 10 mg/kg monthly and 10 mg/kg bi-weekly doses of BAN2401, in ApoE4-negative subjects.

FIG. 41 shows the slowing of cognitive decline versus placebo over 18 months, as determined by CDR-SB, for a dose of 10 mg/kg BAN2401 monthly and a dose of 10 mg/kg BAN2401 bi-weekly, in ApoE4-negative subjects having mild cognitive impairment due to Alzheimer's disease—intermediate likelihood.

FIG. 42 shows the slowing of cognitive decline versus placebo over 18 months, as determined by CDR-SB, for a dose of 10 mg/kg BAN2401 monthly and a dose of 10 mg/kg bi-weekly, in ApoE4-negative subjects having mild Alzheimer's disease dementia.

FIG. 43 shows the reduction of amyloid in the brain, as determined by visual reads of amyloid PET images, as a global cortical average versus whole cerebellum reference, over 18 months, for 2.5 mg/kg bi-weekly, 5 mg/kg monthly, 5 mg/kg bi-weekly, 10 mg/kg monthly, and 10 mg/kg bi-weekly doses of BAN2401, in ApoE4-negative subjects.

FIG. 44 shows the reduction of amyloid in the brain, as determined by imaging using binding of radiotracers for brain Aβ amyloid and visualized with PET, over 18 months, for 2.5 mg/kg bi-weekly, 5 mg/kg monthly, 5 mg/kg bi-weekly, 10 mg/kg monthly, and 10 mg/kg bi-weekly doses of BAN2401, in ApoE4-negative subjects.

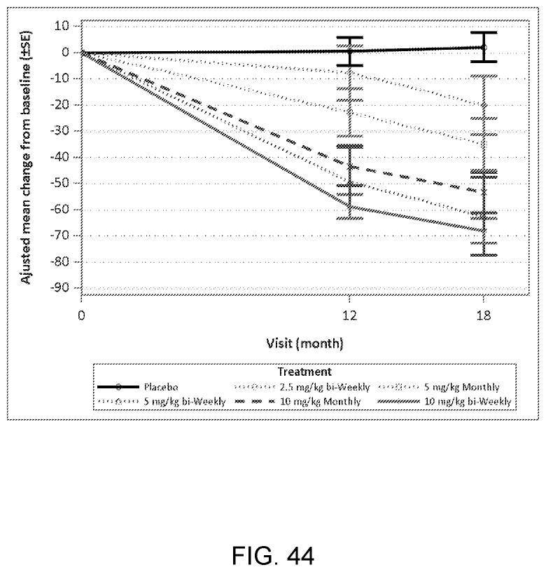

FIG. 45 shows the conversion of amyloid positive, ApoE4-negative subjects to amyloid negative, ApoE4-negative subjects after 12 and 18 months of treatment.

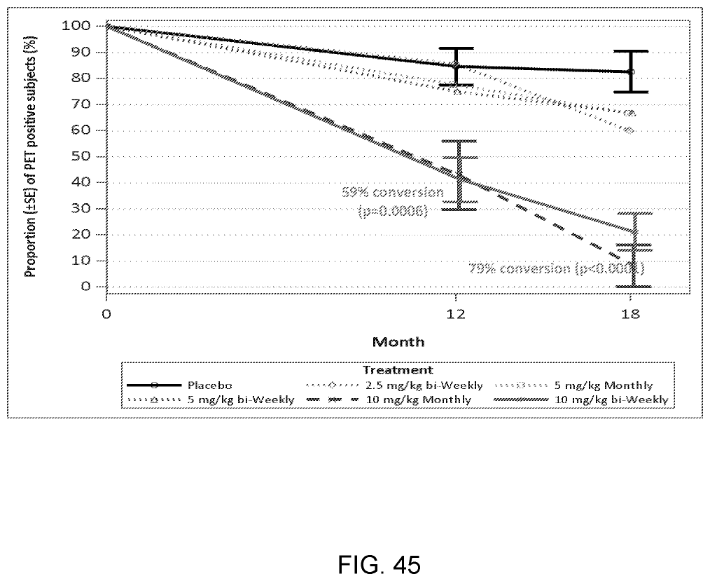

FIG. 46 shows the change in cerebrospinal fluid level of Aβ₁₋₄₂ over 18 months for 2.5 mg/kg bi-weekly, 5 mg/kg monthly, 5 mg/kg bi-weekly, 10 mg/kg monthly, and 10 mg/kg bi-weekly doses of BAN2401 in ApoE4-negative subjects.

FIG. 47 shows the change in cerebrospinal fluid level of total tau over 18 months for a dose of 10 mg/kg monthly and a dose of 10 mg/kg bi-weekly in ApoE4-negative subjects.

FIG. 48 shows the slowing of cognitive decline versus placebo at 18 months, as determined by ADCOMS, ADAS-Cog, or CDR-SB, for a dose of 10 mg/kg BAN2401 bi-weekly, in ApoE4-positive and ApoE4-negative subjects, and for all subjects, regardless of genotype.

FIG. 49 shows the slowing of cognitive decline versus placebo at 18 months, as determined by ADCOMS, ADAS-Cog, or CDR-SB, for a dose of 10 mg/kg BAN2401 bi-weekly, in subjects having mild cognitive impairment due to Alzheimer's disease dementia—moderate likelihood or mild Alzheimer's disease dementia, and for all subjects, regardless of disease type or state.

FIG. 50 shows the slowing of cognitive decline versus placebo at 18 months, as determined by ADCOMS, ADAS-Cog, or CDR-SB, for a dose of 10 mg/kg BAN2401 bi-weekly, in subjects who are concomitantly administered at least one Alzheimer's disease medications other than BAN2401 and subjects who are not concomitantly administered at least one Alzheimer's disease medication other than BAN2401, and for all subjects, regardless of whether the subject is concomitantly administered at least one Alzheimer's disease medication other than BAN2401.

FIG. 51 shows the clearance of brain amyloid versus placebo at 18 months, as determined by PET, for a dose of 10 mg/kg BAN2401 bi-weekly, in the following sub-populations: ApoE4-positive subjects; ApoE4-negative subjects; subjects having mild cognitive impairment due to Alzheimer's disease dementia—moderate likelihood; subjects having mild Alzheimer's disease dementia; subjects who are concomitantly administered at least one Alzheimer's disease medication other than BAN2401; and subjects who are not concomitantly administered at least one Alzheimer's disease medication other than BAN2401.

FIG. 52 shows the slowing of cognitive decline versus placebo at 18 months, as determined by ADCOMS, for a dose of 10 mg/kg BAN2401 bi-weekly, in the following sub-populations: ApoE4-positive subjects; ApoE4-negative subjects; subjects having mild cognitive impairment due to Alzheimer's disease dementia - moderate likelihood; subjects having mild Alzheimer's disease dementia; subjects who are concomitantly administered at least one Alzheimer's disease medication other than BAN2401; and subjects who are not concomitantly administered at least one Alzheimer's disease medication other than BAN2401.

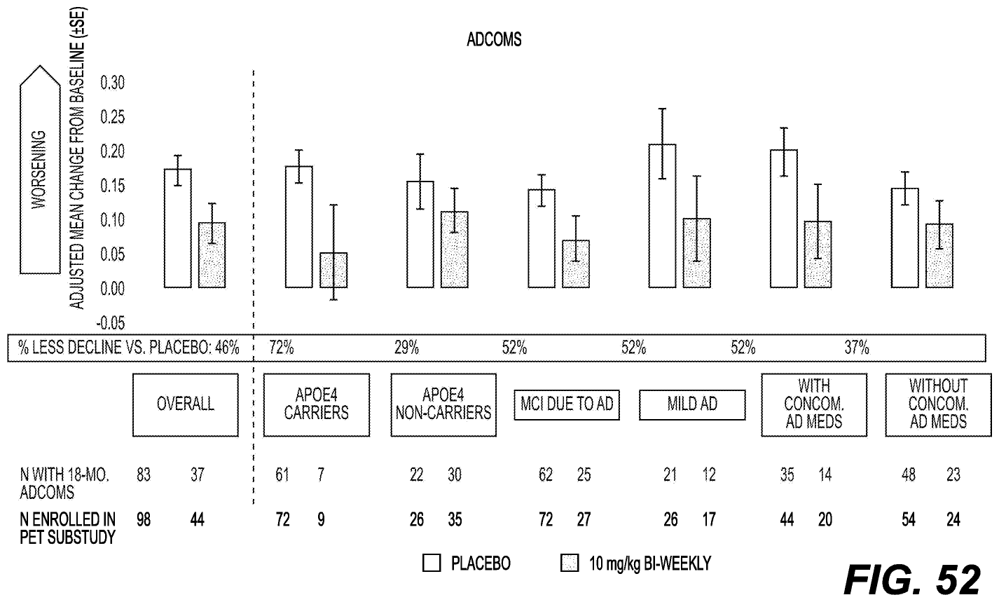

FIG. 53 shows the change in cerebrospinal fluid level of neurogranin at 18 months versus placebo as an average for subjects who received a dose of 10 mg/kg BAN2401 bi-weekly or 10 mg/kg BAN2401 monthly.

FIG. 54 shows the change in cerebrospinal fluid level of phospho-Tau at 18 months versus placebo as an average for subjects who received a dose of 10 mg/kg BAN2401 bi-weekly or 10 mg/kg BAN2401 monthly.

FIG. 55 shows the change in cerebrospinal fluid level of neurofilament light chain at 18 months versus placebo as an average for subjects who received a dose of 10 mg/kg BAN2401 bi-weekly or 10 mg/kg BAN2401 monthly.

FIG. 56 shows the slowing of cognitive decline versus placebo at 18 months, as determined by ADCOMS and PET, fora dose of 10 mg/kg BAN2401 bi-weekly or 10 mg/kg BAN2401 monthly.

FIG. 57 shows a correlation between reduction in amyloid in the brain and an increase in clinical improvement, as determined by ADCOMS, ADAS-Cog, and CDR-SB.

FIG. 58 shows that ApoE4-positive and ApoE4-negative subjects, who were not administered BAN2401, experienced similar rates of disease progression.

FIG. 59 shows the impact of various factors on disease progression, as determined by ADCOMS.

FIG. 60 shows the change in concentration of BAN2401 as a function of time after administration of a dose of 10 mg/kg BAN2401 monthly and 10 mg/kg BAN2401 bi-weekly.

FIG. 61 depicts BAN2401 preferentially binding to larger Aβ protofibrils as compared to monomers, dimers, and oligomers of Aβ peptides.

FIG. 62 shows the proportion of subjects who experienced an ARIA-E event, as a function of maximum plasma concentration of BAN2401.

FIG. 63 shows that administration of BAN2401 significantly reduces amyloid PET values across all doses.

FIG. 64 shows an outline of a study design for the early intervention and prevention of Alzheimer's Disease.

FIG. 65 shows the adjusted mean change (least squares mean, “LSM”) from baseline in cerebrospinal fluid level of phospho-Tau over 18 months versus placebo, for 2.5 mg/kg bi-weekly, 5 mg/kg monthly, 5 mg/kg bi-weekly, 10 mg/kg monthly, and 10 mg/kg bi-weekly doses of BAN2401.

FIG. 66 shows the adjusted mean change (LSM) from baseline in cerebrospinal fluid level of neurogranin over 18 months versus placebo, for 2.5 mg/kg bi-weekly, 5 mg/kg monthly, 5 mg/kg bi-weekly, 10 mg/kg monthly, and 10 mg/kg bi-weekly doses of BAN2401.

FIG. 67 shows the adjusted mean change (LSM) from baseline in cerebrospinal fluid level of neurofilament light chain over 18 months versus placebo, for 2.5 mg/kg bi-weekly, 5 mg/kg monthly, 5 mg/kg bi-weekly, 10 mg/kg monthly, and 10 mg/kg bi-weekly doses of BAN2401.

FIG. 68 shows the adjusted mean change (LSM) from baseline in cerebrospinal fluid level of phospho-Tau over 18 months versus placebo, for the combined 10 mg/kg doses of BAN2401 (10 mg/kg monthly and 10 mg/kg bi-weekly).

FIG. 69 shows the adjusted mean change (LSM) from baseline in cerebrospinal fluid level of phospho-Tau over 18 months versus placebo, for the combined 10 mg/kg doses of BAN2401 (10 mg/kg monthly and 10 mg/kg bi-weekly) in ApoE4-positive subjects.

FIG. 70 shows the adjusted mean change (LSM) from baseline in cerebrospinal fluid level of phospho-Tau over 18 months versus placebo, for the combined 10 mg/kg doses of BAN2401 (10 mg/kg monthly and 10 mg/kg bi-weekly) in ApoE4-negative subjects.

FIG. 71 shows the adjusted mean change (LSM) from baseline in cerebrospinal fluid level of neurogranin over 18 months versus placebo, for the combined 10 mg/kg doses of BAN2401 (10 mg/kg monthly and 10 mg/kg bi-weekly).

FIG. 72 shows the adjusted mean change (LSM) from baseline in cerebrospinal fluid level of neurogranin over 18 months versus placebo, for the combined 10 mg/kg doses of BAN2401 (10 mg/kg monthly and 10 mg/kg bi-weekly) in ApoE4-positive subjects.

FIG. 73 shows the adjusted mean change (LSM) from baseline in cerebrospinal fluid level of neurogranin over 18 months versus placebo, for the combined 10 mg/kg doses of BAN2401 (10 mg/kg monthly and 10 mg/kg bi-weekly) in ApoE4-negative subjects.

FIG. 74 shows the adjusted mean change (LSM) from baseline in cerebrospinal fluid level of neurofilament light chain over 18 months versus placebo, for the combined 10 mg/kg doses of BAN2401 (10 mg/kg monthly and 10 mg/kg bi-weekly).

FIG. 75 shows the adjusted mean change (LSM) from baseline in cerebrospinal fluid level of neurofilament light chain over 18 months versus placebo, for the combined 10 mg/kg doses of BAN2401 (10 mg/kg monthly and 10 mg/kg bi-weekly) in ApoE4-positive subjects.

FIG. 76 shows the adjusted mean change (LSM) from baseline in cerebrospinal fluid level of neurofilament light chain over 18 months versus placebo, for the combined 10 mg/kg doses of BAN2401 (10 mg/kg monthly and 10 mg/kg bi-weekly) in ApoE4-negative subjects.

FIG. 77 shows the correlation between change from baseline of PET standard uptake value ratio (normalized to whole cerebellum mask) and cognitive outcome, as determined by ADCOMS, at 12 and 18 months, for placebo and 2.5 mg/kg bi-weekly, 5 mg/kg monthly, 5 mg/kg bi-weekly, 10 mg/kg monthly, and 10 mg/kg bi-weekly doses of BAN2401.

FIG. 78 shows the correlation between change from baseline of PET standard uptake value ratio (normalized to whole cerebellum mask) and cognitive outcome, as determined by CDR-SB, at 12 and 18 months, for placebo and 2.5 mg/kg bi-weekly, 5 mg/kg monthly, 5 mg/kg bi-weekly, 10 mg/kg monthly, and 10 mg/kg bi-weekly doses of BAN2401.

FIG. 79 shows the correlation between change from baseline of PET standard uptake value ratio (normalized to whole cerebellum mask) and cognitive outcome, as determined by ADAS-Cog, at 12 and 18 months, for placebo and 2.5 mg/kg bi-weekly, 5 mg/kg monthly, 5 mg/kg bi-weekly, 10 mg/kg monthly, and 10 mg/kg bi-weekly doses of BAN2401.

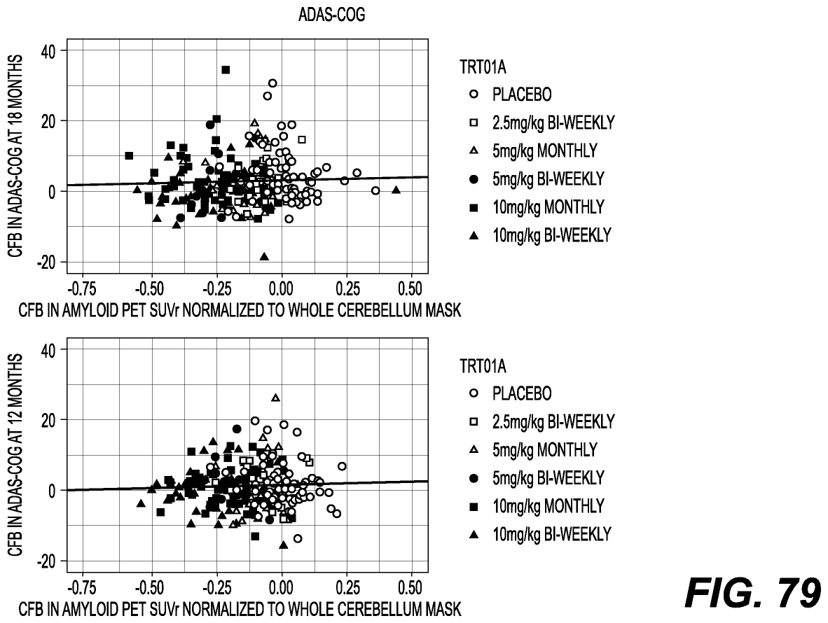

FIG. 80 shows the correlation between change from baseline of PET standard uptake value ratio (normalized to subcortical white matter) and cognitive outcome, as determined by ADCOMS, at 12 and 18 months, for placebo and 2.5 mg/kg bi-weekly, 5 mg/kg monthly, 5 mg/kg bi-weekly, 10 mg/kg monthly, and 10 mg/kg bi-weekly doses of BAN2401.

FIG. 81 shows the correlation between change from baseline of PET standard uptake value ratio (normalized to subcortical white matter) and cognitive outcome, as determined by CDR-SB, at 12 and 18 months, for placebo and 2.5 mg/kg bi-weekly, 5 mg/kg monthly, 5 mg/kg bi-weekly, 10 mg/kg monthly, and 10 mg/kg bi-weekly doses of BAN2401.

FIG. 82 shows the correlation between change from baseline of PET standard uptake value ratio (normalized to subcortical white matter) and cognitive outcome, as determined by ADAS-Cog, at 12 and 18 months, for placebo and 2.5 mg/kg bi-weekly, 5 mg/kg monthly, 5 mg/kg bi-weekly, 10 mg/kg monthly, and 10 mg/kg bi-weekly doses of BAN2401.

### THE “AMYLOID HYPOTHESIS”

The “amyloid hypothesis” proposes that amyloid β (Aβ) peptides play a central role in the pathogenesis of AD. Specifically, it is hypothesized that neurodegeneration in AD may be caused by deposition of Aβ plaques in brain tissue due to an imbalance between Aβ production and Aβ clearance, leading to formation of neurofibrillary tangles containing tau protein. Aβ peptides generally exist in a dynamic continuum of conformational states such that species tend to progress from monomeric Aβ, to soluble Aβ assemblies that include a range of low molecular weight oligomers to higher molecular weight protofibrils, and finally to insoluble fibrils (plaques). A number of immunotherapies have been developed with the intent to reduce the amount of insoluble Aβ fibrils deposited in the brain. However, a simple correlation between the quantity and progressive accumulation of insoluble amyloid plaques and the clinical course of AD has not been determined. While therapeutic strategies continue to focus on removal of insoluble amyloid plaques, an additional approach to therapy may include reducing the toxic Aβ aggregates, such as protofibrils, that may contribute to the neuronal degeneration characteristic of AD. (See, e.g., Dodort, J.-C. and May, P., “Overview on rodent models of Alzheimer's disease.” Curr. Protocols Neurosci. 2005; 9.22-1-9.22-6; Englund, H. et al., “Sensitive ELISA detection of amyloid-β protofibrils in biological samples.” J. Neurochem. 2007; 103:334-45; and Gotz, J. et al., “Transgenic animal models of Alzheimer's disease and related disorders: histopathology, behavior and therapy.” Mol. Psychiat. 2004; 9:664-83.)

It was hypothesized that BAN2401 and other anti-Aβ protofibril antibodies could be used to slow AD progression in subjects at early stages of the disease when amyloid had been deposited in the brain but where the downstream neurodegenerative cascade thought to be triggered by the amyloid deposition was still relatively early in its course (i.e., limited brain tissue loss has been produced and associated clinical deficits are at a minimum). As disclosed herein, using BAN2401 as an exemplary anti-Aβ protofibril antibody, the inventors have discovered a novel method of reducing brain amyloid levels in amyloid positive early AD subjects. Also disclosed herein is a novel method of converting amyloid positive early AD subjects to amyloid negative comprising administering a composition comprising a therapeutically effective amount of at least one anti-Aβ protofibril antibody. A method of reducing clinical decline in amyloid positive early AD subjects comprising administering a composition comprising a therapeutically effective amount of at least one anti-Aβ protofibril antibody is also disclosed herein. As will be discussed, unpredictably beneficial results were achieved when the subjects are ApoE4 positive.

Surprisingly and unpredictably, the inventors also discovered a method of preventing and/or delaying onset of AD in early AD subjects comprising administering a composition comprising a therapeutically effective amount of at least one anti-Aβ protofibril antibody. In some embodiments, the subject is ApoE4 positive. In some embodiments, the at least one anti-Aβ protofibril antibody is BAN2401.

### Definitions

The following are definitions of terms used in the present application.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B”, when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “at least one” means one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-liniting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When a number is recited, either alone or as part of a numerical range, it should be understood that the numerical value can vary above and below the stated value by a variance of 10% of the stated value.

When a range of values is listed herein, it is intended to encompass each value and sub-range within that range. For example, “2.5 mg/kg to 10 mg/kg” is intended to encompass, for example, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 2.5 mg/kg to 3 mg/kg, 2.5 mg/kg to 4.5 mg/kg, 3 mg/kg to 4.5 mg/kg, 4.5 mg/kg to 8 mg/kg, 2.5 mg/kg to 9 mg/kg, and so forth.

“Early AD” or “early Alzheimer's disease,” as used herein, is a continuum of AD severity from mild cognitive impairment due to AD—intermediate likelihood to mild Alzheimer's disease dementia. Subjects with early AD include subjects with mild Alzheimer's disease dementia as defined herein and subject with MCI due to AD-13 intermediate likelihood as defined herein. In some embodiments, subjects with early AD have MMSE of 22 to 30 and CDR global range 0.5 to 1.0.

Subjects with “mild Alzheimer's disease dementia,” as used herein, are subjects meet the NIA-AA core clinical criteria for probable Alzheimer's disease dementia in McKhann, G. M. et al., “The diagnosis of dementia due to Alzheimer's disease: Recommendations from the National Institute on Aging—Alzheimer's Association workgroups on diagnostic guidelines for Alzheimer's disease.” Alzheimer Dement. 2011; 7:263-9. Also included herein are subjects who have a CDR score of 0.5 to 1.0 and a Memory Box score of 0.5 or greater at screening and baseline.

Subjects with “MCI due to AD-13 intermediate likelihood,” as used herein are those identified as such in accordance with the NIA-AA core clinical criteria for mild cognitive impairment due to Alzheimer's disease—intermediate likelihood (see McKhann supra). For example, symptomatic but not demented AD subjects with evidence of brain amyloid pathology making them less heterogeneous and more similar to mild Alzheimer's disease dementia subjects in cognitive and functional decline as measured by the ADCOMS Composite Clinical Score defined herein. Also included are subjects who have a CDR score of 0.5 and a Memory Box score of 0.5 or greater at screening and baseline. Furthermore, subjects who report a history of subjective memory decline with gradual onset and slow progression over the last 1 year before screening, which is corroborated by an informant, are also included herein.

As used herein, “MMSE” refers to the Mini-Mental State Examination, a cognitive instrument commonly used for screening purposes, but also often measured longitudinally in AD clinical trials having a 30-point scale with higher scores indicating less impairment and lower scores indicating more impairment. As used herein, seven items measuring orientation to time and place, registration, recall, attention, language and drawing were assessed. (Folstein, M. F. et al., “Mini-mental state. A practical method for grading the cognitive state of patients for the clinician.” J. Psychiatr. Res. 1975;12:189-98.)

As used herein, “ADAS-cog” refers to Alzheimer's Disease Assessment Scale-Cognitive. The ADAS-cog is a widely used cognitive scale in Alzheimer's disease trials having a structured scale that evaluates memory (word recall, delayed word recall, and word recognition), reasoning (following commands), language (naming, comprehension), orientation, ideational praxis (placing letter in envelope) and constructional praxis (copying geometric designs). (Rosen, W. G. et al., “A new rating scale for Alzheimer's disease.” Am. J. Psychiatry 1984; 141:1356-64.) Ratings of spoken language, language comprehension, word finding difficulty, ability to remember test instructions, maze, and number cancellation were also obtained. The modified version used herein is scored from 0 to 90 points with a score of 0 indicating no impairment, and a score of 90 indicating maximum impairment.

As used herein, “CDR-SB” refers to clinical dementia rating—sum of boxes. The CDR is a clinical scale that describes 5 degrees of impairment in performance on each of 6 categories of function including memory, orientation, judgment and problem solving, community affairs, home and hobbies, and personal care. (Berg, L. et al., “Mild senile dementia of the Alzheimer type: 2. Longitudinal assessment.” Ann. Neurol. 1988; 23:477-84.) The ratings of degree of impairment obtained on each of the 6 categories of function are synthesized into 1 global rating of dementia CDR score (ranging from 0 to 3). A sum of boxes score provides an additional measure of change where each category has a maximum possible score of 3 points and the total score is a sum of the category scores giving a total possible score of 0 to 18 with higher scores indicating more impairment. The global score may be used as a clinical measure of severity of dementia.

As used herein, “ADCOMS” refers to Alzheimer's Disease Composite Score, a composite clinical score based on an analysis of four ADAS-Cog items (delayed word recall, orientation, word recognition, and word finding difficulty), two MMSE items (orientation to time, and drawing), and all six CDR-SB items (personal care, community affairs, home and hobbies, memory, orientation, and judgment and problem solving), as discussed in the Examples and in Wang, J. et al., “ADCOMS: a composite clinical outcome for prodromal Alzheimer's disease trials.” J. Neurol. Neurosurg. Psychiatry. 2016; 87:993-999. ADCOMS was developed to be particularly sensitive to disease progression during early stages of AD, i.e., prodromal and mild AD.

As used herein, “ApoE4-positive” subjects and “ApoE4 carriers” refer to subjects who harbor the ε4 variant of the apolipoprotein gene. The ε4 variant is one of several major alleles of the apolipoprotein gene. The gene is generally responsible for metabolism of fats. It has been found that carriers of the apolipoprotein ε4 show significantly greater rates of amyloid retention when compared to non-carriers. (Drzezga, A. et al, “Effect of APOE genotype on amyloid plaque load and gray matter volume in Alzheimer disease.” Neurology. 2009; 72:1487-94.) In some embodiments, the subject is a heterozygous carrier of the apolipoprotein E ε4 gene allele. In some embodiments, the subject is a homozygous carrier of the apolipoprotein E ε4 gene allele.

As used herein, whether an early AD subject is “amyloid-positive” or “amyloid-negative” is determined based on whether or not the patient has a positive amyloid load as indicated by longitudinal PET assessment of an amyloid imaging agent uptake into the brain and/or a CSF assessment of the presence of amyloid pathology using assessments of markers such as Aβ₁₋₄₂ (soluble CSF biomarker analysis). In some embodiments, a qualitative visual read of PET scans will be used to determine amyloid positive and amyloid negative by categorizing subjects as having either “normal” or “abnormal” uptake on the basis of the PET image pattern. Readers will have been trained and certified to recognize brain PET images with abnormal or normal patterns of uptake, or the detection of amyloid is done through a semi-quantitative or quantitative approach.

As used herein, the term “treat” refers to obtaining beneficial or desired results including, but not limited to, therapeutic benefit, by which is meant eradication or amelioration of the underlying condition being treated or of one or more of the physiological symptoms associated therewith.

As used herein, the term “prevent” refers to obtaining beneficial or desired results including, but not limited to, prophylactic benefit. For prophylactic benefit, the composition may be administered to a subject at risk of developing Alzheimer's disease, to a subject having one or more preclinical symptoms but not clinical symptoms of Alzheimer's disease, or to a subject reporting one or more of the physiological symptoms of Alzheimer's disease, even though a clinical diagnosis of having Alzheimer's has not been made. As used herein “prevention” may further include therapeutic benefit, by which is meant eradication or amelioration of the underlying condition being treated or of one or more of the physiological symptoms associated therewith.

Pre-AD biomarker levels that may suggest the development of Alzheimer's disease include, but are not limited to, brain amyloid level, cerebrospinal fluid level of Aβ₁₋₄₂, cerebrospinal fluid level of total tau, cerebrospinal fluid level of neurogranin, and cerebrospinal fluid level of neurofilament light chain. For example, it has been found that subjects treated with elenbecestat (E2609), a BACE inhibitor, who had amyloid baseline PET standard uptake value ratios (SUVr values) of 1.4 to 1.9, exhibited the greatest slowing of cognitive decline while on treatment. See Lynch, S. Y. et al. “Elenbecestat, a BACE inhibitor: results from a Phase 2 study in subjects with mild cognitive impairment and mild-to-moderate dementia due to Alzheimer's disease.” Poster P4-389, Alzheimer's Association International Conference, Jul. 22-26, 2018, Chicago, Ill., USA. Similarly, it has been found that subjects having a baseline florbetapir amyloid PET SUVr levels below 1.2 do not exhibit enough cognitive decline to be detectable, whereas subjects having SUVr levels above 1.6 appear to correlate with a plateau effect in which amyloid level has reached a saturation level and treatment does not result in a change of cognitive measures. See Dhadda, S. et al., “Baseline florbetapir amyloid PET standard update value ratio (SUVr) can predict clinical progression in prodromal Alzheimer's disease (pAD).” Poster P4-291, Alzheimer's Association International Conference, Jul. 22-26, 2018, Chicago, Ill., USA.

As used herein, the terms “serious adverse event” or “SAE” means an event that (1) results in death; (2) is life-threatening; (3) requires inpatient hospitalization or prolongation of existing hospitalization; (4) results in persistent or significant disability/incapacity; and/or (5) is a congenital anomaly/birth defect, which is observed after administration of a composition described herein.

The severity of a serious adverse event may be assessed based on a uniform scale used in the art. For example, the seriousness of a subject's serious adverse event may be evaluated according to the National Cancer Institute's “Common Terminology Criteria for Adverse Events” or “CTCAE.” The descriptions for the various CTCAE adverse event grades are set forth below:
Grade 1: mild; asymptomatic or mild symptoms; clinical or diagnostic observations only; intervention not indicated.
Grade 2: moderate; minimal, local or noninvasive intervention indicated; limiting age-appropriate instrumental activities of daily living.
Grade 3: severe or medically significant but not immediately life-threatening; hospitalization or prolongation of hospitalization indicated; disabling; limiting self-care activities of daily living.
Grade 4: life-threatening consequences; urgent intervention indicated.
Grade 5: death related to the adverse event.

As used herein, the term “elenbecestat” refers to the compound N-[3-((4aS,5R,7aS)-2-am ino-5-methyl-4a,5,7,7a-tetrahydro-4H-fluro[3,4-d][1,3]thiazin-7a-yl)-4-fluorophenyl]-5-difluoromethylpyrazine-2-carboxamide or a pharmaceutically acceptable salt thereof. Elenbecestat is a Beta-site Amyloid Precursor Protein Cleaving Enzyme 1 (BACE1) inhibitor (see, e.g., U.S. Pat. Nos. 8,158,620 and 8,426,584) and is also known as E2609.

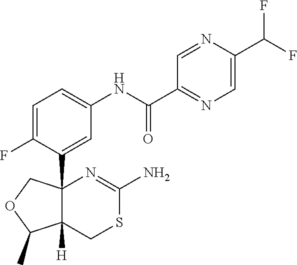 [SMILES: C[C@H]1OC[C@]2(c3cc(NC(=O)c4cnc(C(F)F)cn4)ccc3F)N=C(N)SC[C@H]12]

At least one anti-Aβ protofibril antibody

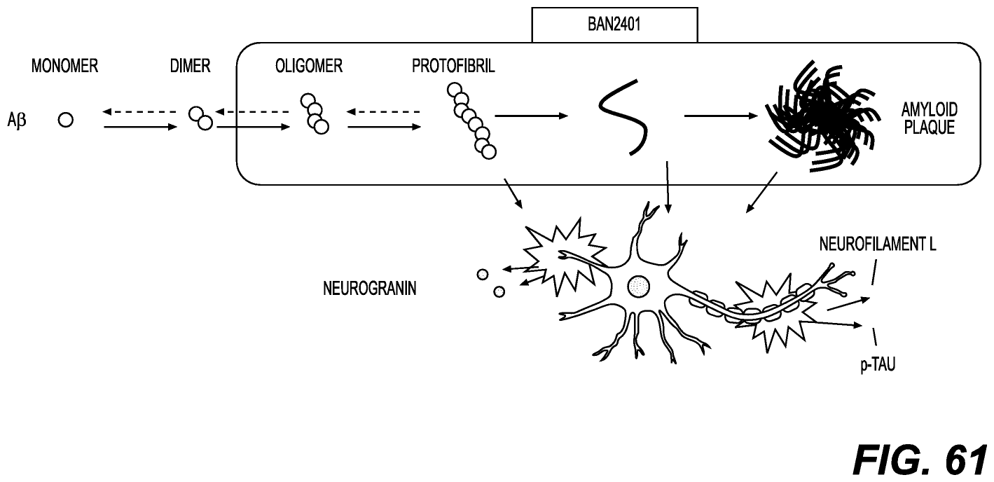

As used herein, the at least one anti-Aβ protofibril antibody is chosen from monoclonal antibodies (mAbs) that preferentially bind to large soluble amyloid β (Aβ) oligomers and/or aggregates (also termed protofibrils) as compared to, e.g., Aβ monomers. See, e.g., FIG. 61. As used herein, the term “preferentially bind(s)” refers to an antibody that binds to Aβ oligomers and/or protofibrils with at least 10-fold, at least 20-fold, at least 30-fold, at least 40-fold, at least 50-fold, at least 60-fold, at least 70-fold, at least 80-fold, at least 90-fold, at least 100-fold, at least 125-fold, at least 150-fold, at least 175-fold, at least 200-fold, at least 225-fold, at least 250-fold, at least 275-fold, at least 300-fold, at least 325 fold, at least 350-fold, at least 375-fold, at least 400-fold, at least 425-fold, at least 450-fold, at least 475-fold, at least 500-fold, at least 550-fold, at least 600-fold, at least 650-fold, at least 700-fold, at least 750-fold, at least 800-fold, at least 850-fold, at least 900-fold, at least 950-fold, at least 1000-fold, or at least 1050 fold greater potency than its potency for binding other forms of Aβ peptides (e.g., fibrils and monomers, respectively). The potency for binding the various forms of Aβ can be determined by a method well-known in the art, e.g., an ELISA assay and surface plasmon resonance (SPR).

In some embodiments, selectivity of the at least one anti-Aβ protofibril antibody is measured by an ELISA assay. In some embodiments, preferential binding of the at least one anti-Aβ protofibril antibody is measured by surface plasmon resonance.

In some embodiments, preferential binding of BAN2401 is measured by an ELISA assay. In some embodiments, selectivity of BAN2401 is measured by surface plasmon resonance.

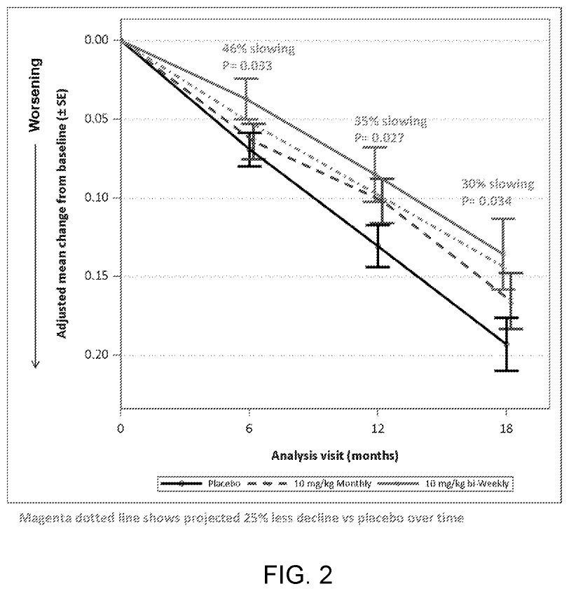

For example, it has been found that BAN2401 binds to Aβ protofibrils with 200-1000-fold greater potency than to Aβ monomers and that BAN2401 binds to Aβ protofibrils with greater than 40-fold potency than to Aβ fibrils. International Patent Application Publication No. WO 2007/108756 A1 at p. 13 and FIG. 2; see also Lord, A. et al., “An amyloid-β protofibril-selective antibody prevents amyloid formation in a mouse model of Alzheimer's disease.” 2009; 26: 425-34; and Swanson, C. J. et al. “Pharmacology of BAN2401: A Monoclonal Antibody Selective for Aβ Protofibrils.” Poster P4-286, Alzheimer's Association International Conference, Jul. 13-18, 2013. This difference in potency was determined by a sandwich ELISA assay, where BAN2401 was coated on the wells of a well plate and different Aβ forms were added to the wells in increasing concentrations. Id. Measurement of bound Aβ forms was made by adding biotinylated mAb158 and HRP-labelled Streptavidine, and color development was measured according to the manufacturer's procedure. Similarly, it has been found that BAN2401 has a weak affinity for the N-terminal part of the Aβ peptide and Aβ monomers, and no binding to the C-terminal fragment of Aβ was observed. Id. Affinity was determined by using a competitive ELISA assay, where an ELISA plate was coated with human Aβ protofibrils and BAN2401 was subsequently incubated with increasing amounts of different Aβ forms. Id. The incubation mix was added to the microtiter plate wells and free antibody was allowed to bind to immobilized protofibrils in the well, and the bound BAN2401 antibody was measured by a second antibody. Id.

In some embodiments, the at least one anti-Aβ protofibril antibody comprises three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

The assignment of amino acids to each domain is, generally, in accordance with the definitions of SEQUENCES OF PROTEINS OF IMMUNOLOGICAL INTEREST (Kabat et al., 5th ed., U.S. Department of Health and Human Services, NIH Publication No. 91-3242, 1991, hereafter referred to as “Kabat report”).

In some embodiments, the at least one anti-Aβ protofibril antibody comprises a human constant region. In some embodiments, the human constant region of the at least one anti-Aβ protofibril antibody comprises a heavy chain constant region chosen from IgG1, IgG2, IgG3, IgG4, IgM, IgA, IgE, and any allelic variation thereof as disclosed in the Kabat report. Any one or more of such sequences may be used in the present disclosure. In some embodiments, the heavy chain constant region is chosen from IgG1 and allelic variations thereof. The amino acid sequence of human IgG1 constant region is known in the art and set out in SEQ ID NO: 3.

In some embodiments, the human constant region of the at least one anti-Aβ antibody comprises a light chain constant region chosen from K-A-chain constant regions and any allelic variation thereof as discussed in the Kabat report. Any one or more of such sequences may be used in the present disclosure. In some embodiments, the light chain constant region is chosen from K and allelic variations thereof. The amino acid sequence of human K chain constant region is known in the art and set out in SEQ ID NO: 4.

In some embodiments, the at least one anti-Aβ protofibril antibody comprises human heavy and light chain variable region frameworks. In some embodiments, the at least one anti-Aβ protofibril antibody comprises a heavy chain variable region comprising an amino acid sequence of SEQ ID NO: 1, and a light chain variable region comprising an amino acid sequence of SEQ ID NO: 2. In some embodiments, the at least one anti-Aβ protofibril antibody comprises a human IgG1 heavy chain constant region, and a human Ig kappa light chain constant region. In some embodiments, the at least one anti-Aβ protofibril antibody comprises a heavy chain constant region comprising an amino acid sequence of SEQ ID NO: 3, and a light chain constant region comprising an amino acid sequence of SEQ ID NO: 4.

In some embodiments, the at least one anti-Aβ protofibril antibody is BAN2401. BAN2401 is a humanized IgG1 monoclonal version of mAb158, which is a murine monoclonal antibody raised to target protofibrils and disclosed in WO 2007/108756 and Journal of Alzheimer's Disease 43: 575-588 (2015). BAN2401 is at least one anti-Aβ protofibril antibody, demonstrating low affinity for Aβ monomer while binding with high selectivity to soluble Aβ aggregate species. For example, BAN2401 has been reported demonstrates an approximately 1000-fold and 5-fold to 10-fold higher selectivity for soluble Aβ protofibrils than for Aβ monomers or Aβ-insoluble fibrils, respectively.

BAN2401 comprises (a) a heavy chain variable domain comprising the amino acid sequence of SEQ ID NO:1 and (b) a light chain variable domain comprising the amino acid sequence of SEQ ID NO:2. The full length sequence of BAN2401 is set forth in SEQ ID NO: 11 and is described in WO 2007/108756 and in Journal of Alzheimer's Disease 43:575-588 (2015).

Other non-limiting examples of suitable antibodies for use as the at least one anti-Aβ protofibril antibody in the present disclosure include those disclosed in WO 2002/003911, WO 2005/123775, WO 2007/108756, WO 2011/001366, WO 2011/104696, and WO 2016/005466.

Therapeutically Effective Amount of at Least One anti-Aβ Protofibril Antibody

The methods of the present disclosure comprise administering to a subject a composition comprising a therapeutically effective amount of at least one anti-Aβ protofibril antibody. As used herein, the term a “therapeutically effective amount” refers to an amount of a compound or pharmaceutical composition sufficient to product a desired therapeutic effect.

One of ordinary skill in the art will understand that the therapeutically effective amount of the at least one anti-Aβ protofibril antibody administered to a subject may depend upon a number of factors including pharmacodynamic characteristics, route of administration, frequency of treatment, and health, age, and weight of the subject to be treated and, with the information disclosed herein, will be able to determine the appropriate amount for each subject.

In some embodiments, the therapeutically effective amount is a dose chosen to improve efficacy and/or maintain efficacy and improve at least one of safety and tolerability. In some embodiments, the therapeutically effective amount is chosen to lower at least one side effect and simultaneously improve efficacy and/or maintain efficacy.

In some embodiments, 0.5 mg/kg to 45 mg/kg, 0.5 mg/kg to 40 mg/kg, 0.5 mg/kg to 35 mg/kg, 0.5 mg/kg to 30 mg/kg, 0.5 mg/kg to 25 mg/kg, 0.5 mg/kg to 20 mg/kg, 0.5 mg/kg to 15 mg/kg, 0.5 mg/kg to 10 mg/kg, 0.5 mg/kg to 5 mg/kg, or 0.5 mg/kg to 2.5 mg/kg of at least one anti-Aβ protofibril antibody is administered to the subject relative to body weight of the subject.

In some embodiments, 2.5 mg/kg to 45 mg/kg, 2.5 mg/kg to 40 mg/kg, 2.5 mg/kg to 35 mg/kg, 2.5 mg/kg to 30 mg/kg, 2.5 mg/kg to 25 mg/kg, 2.5 mg/kg to 20 mg/kg, 2.5 mg/kg to 15 mg/kg, 2.5 mg/kg to 10 mg/kg, or 2.5 mg/kg to 5 mg/kg of at least one anti-Aβ protofibril antibody is administered to the subject relative to body weight of the subject.

In some embodiments, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, or 5 mg/kg to 10 mg/kg, of at least one anti-Aβ protofibril antibody is administered to the subject relative to body weight of the subject.

In some embodiments, 7.5 mg/kg to 45 mg/kg, 7.5 mg/kg to 40 mg/kg, 7.5 mg/kg to 35 mg/kg, 7.5 mg/kg to 30 mg/kg, 7.5 mg/kg to 25 mg/kg, 7.5 mg/kg to 20 mg/kg, 7.5 mg/kg to 15 mg/kg, or 7.5 mg/kg to 10 mg/kg of at least one anti-Aβ protofibril antibody is administered to the subject relative to body weight of the subject.

In some embodiments, from 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg of at least one anti-Aβ protofibril antibody is administered to the subject relative to body weight of the subject. In some embodiments, up to 20 mg/kg, 19 mg/kg, 18 mg/kg, 17 mg/kg, 16 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, or 0.5 mg/kg of at least one anti-Aβ protofibril antibody is administered to the subject relative to body weight of the subject.

In some embodiments, 0.5 mg/kg of at least one anti-Aβ protofibril antibody is administered to the subject relative to body weight of the subject. In some embodiments, 1 mg/kg of at least one anti-Aβ protofibril antibody is administered to the subject relative to body weight of the subject. In some embodiments, 2 mg/kg of at least one anti-Aβ protofibril antibody is administered to the subject relative to body weight of the subject. In some embodiments, 2.5 mg/kg of at least one anti-Aβ protofibril antibody is administered to the subject relative to body weight of the subject. In some embodiments, 3 mg/kg of at least one anti-Aβ protofibril antibody is administered to the subject relative to body weight of the subject. In some embodiments, 4 mg/kg of at least one anti-Aβ protofibril antibody is administered to the subject relative to body weight of the subject. In some embodiments, 5 mg/kg of at least one anti-Aβ protofibril antibody is administered to the subject relative to body weight of the subject. In some embodiments, 6 mg/kg of at least one anti-Aβ protofibril antibody is administered to the subject relative to body weight of the subject. In some embodiments, 7 mg/kg of at least one anti-Aβ protofibril antibody is administered to the subject relative to body weight of the subject. In some embodiments, 7.5 mg/kg of at least one anti-Aβ protofibril antibody is administered to the subject relative to body weight of the subject. In some embodiments, 8 mg/kg of at least one anti-Aβ protofibril antibody is administered to the subject relative to body weight of the subject. In some embodiments, 9 mg/kg of at least one anti-Aβ protofibril antibody is administered to the subject relative to body weight of the subject. In some embodiments, 10 mg/kg of at least one anti-Aβ protofibril antibody is administered to the subject relative to body weight of the subject. In some embodiments, 11 mg/kg of at least one anti-Aβ protofibril antibody is administered to the subject relative to body weight of the subject. In some embodiments, 12 mg/kg of at least one anti-Aβ protofibril antibody is administered to the subject relative to body weight of the subject. In some embodiments, 12.5 mg/kg of at least one anti-Aβ protofibril antibody is administered to the subject relative to body weight of the subject. In some embodiments, 13 mg/kg of at least one anti-Aβ protofibril antibody is administered to the subject relative to body weight of the subject. In some embodiments, 14 mg/kg of at least one anti-Aβ protofibril antibody is administered to the subject relative to body weight of the subject. In some embodiments, 15 mg/kg of at least one anti-Aβ protofibril antibody is administered to the subject relative to body weight of the subject. In some embodiments, 16, 17, 18, 19, or 20 mg/kg of at least one anti-Aβ protofibril antibody is administered to the subject relative to body weight of the subject. In some embodiments, 21, 22, 23, 24, or 25 mg/kg of at least one anti-Aβ protofibril antibody is administered to the subject relative to body weight of the subject.

In some embodiments, 27.5 mg/kg, 30 mg/kg, 32.5 mg/kg, 35 mg/kg, 37.5 mg/kg, 40 mg/kg, 42.5 mg/kg, 45 mg/kg, 47.5 mg/kg, or 50 mg/kg of at least one anti-Aβ protofibril antibody is administered to the subject relative to body weight of the subject.

As mentioned, in some embodiments, the at least one anti-Aβ protofibril antibody is BAN2401. Accordingly, in some embodiments, 0.5 mg/kg to 45 mg/kg, 0.5 mg/kg to 40 mg/kg, 0.5 mg/kg to 35 mg/kg, 0.5 mg/kg to 30 mg/kg, 0.5 mg/kg to 25 mg/kg, 0.5 mg/kg to 20 mg/kg, 0.5 mg/kg to 15 mg/kg, 0.5 mg/kg to 10 mg/kg, 0.5 mg/kg to 5 mg/kg, or 0.5 mg/kg to 2.5 mg/kg of BAN2401 is administered to the subject relative to body weight of the subject.

In some embodiments, 2.5 mg/kg to 45 mg/kg, 2.5 mg/kg to 40 mg/kg, 2.5 mg/kg to 35 mg/kg, 2.5 mg/kg to 30 mg/kg, 2.5 mg/kg to 25 mg/kg, 2.5 mg/kg to 20 mg/kg, 2.5 mg/kg to 15 mg/kg, 2.5 mg/kg to 10 mg/kg, or 2.5 mg/kg to 5 mg/kg of BAN2401 is administered to the subject relative to body weight of the subject.

In some embodiments, 5 mg/kg to 45 mg/kg, 5 mg/kg to 40 mg/kg, 5 mg/kg to 35 mg/kg, 5 mg/kg to 30 mg/kg, 5 mg/kg to 25 mg/kg, 5 mg/kg to 20 mg/kg, 5 mg/kg to 15 mg/kg, or 5 mg/kg to 10 mg/kg of BAN2401 is administered to the subject relative to body weight of the subject.

In some embodiments, 7.5 mg/kg to 45 mg/kg, 7.5 mg/kg to 40 mg/kg, 7.5 mg/kg to 35 mg/kg, 7.5 mg/kg to 30 mg/kg, 7.5 mg/kg to 25 mg/kg, 7.5 mg/kg to 20 mg/kg, 7.5 mg/kg to 15 mg/kg, or 7.5 mg/kg to 10 mg/kg of BAN2401 is administered to the subject relative to body weight of the subject.

In some embodiments, from 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg of BAN2401 is administered to the subject relative to body weight of the subject. In some embodiments, up to 20 mg/kg, 19 mg/kg, 18 mg/kg, 17 mg/kg, 16 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, or 0.5 mg/kg of BAN2401 is administered to the subject relative to body weight of the subject.

In some embodiments, 0.5 mg/kg of BAN2401 is administered to the subject relative to body weight of the subject. In some embodiments, 1 mg/kg of BAN2401 is administered to the subject relative to body weight of the subject. In some embodiments, 2 mg/kg of BAN2401 is administered to the subject relative to body weight of the subject. In some embodiments, 2.5 mg/kg of BAN2401 is administered to the subject relative to body weight of the subject. In some embodiments, 3 mg/kg of BAN2401 is administered to the subject relative to body weight of the subject. In some embodiments, 4 mg/kg of BAN2401 is administered to the subject relative to body weight of the subject. In some embodiments, 5 mg/kg of BAN2401 is administered to the subject relative to body weight of the subject. In some embodiments, 6 mg/kg of BAN2401 is administered to the subject relative to body weight of the subject. In some embodiments, 7 mg/kg of BAN2401 is administered to the subject relative to body weight of the subject. In some embodiments, 7.5 mg/kg of BAN2401 is administered to the subject relative to body weight of the subject. In some embodiments, 8 mg/kg of BAN2401 is administered to the subject relative to body weight of the subject. In some embodiments, 9 mg/kg of BAN2401 is administered to the subject relative to body weight of the subject. In some embodiments, 10 mg/kg of BAN2401 is administered to the subject relative to body weight of the subject. In some embodiments, 11 mg/kg of BAN2401 is administered to the subject relative to body weight of the subject. In some embodiments, 12 mg/kg of BAN2401 is administered to the subject relative to body weight of the subject. In some embodiments, 12.5 mg/kg of BAN2401 is administered to the subject relative to body weight of the subject. In some embodiments, 13 mg/kg of BAN2401 is administered to the subject relative to body weight of the subject. In some embodiments, 14 mg/kg of BAN2401 is administered to the subject relative to body weight of the subject. In some embodiments, 15 mg/kg of BAN2401 is administered to the subject relative to body weight of the subject. In some embodiments, 16, 17, 18, 19, or 20 mg/kg of BAN2401 is administered to the subject relative to body weight of the subject. In some embodiments, 21, 22, 23, 24, or 25 mg/kg of BAN2401 is administered to the subject relative to body weight of the subject. In some embodiments, 27.5 mg/kg, 30 mg/kg, 32.5 mg/kg, 35 mg/kg, 37.5 mg/kg, 40 mg/kg, 42.5 mg/kg, 45 mg/kg, 47.5 mg/kg, or 50 mg/kg of BAN2401 is administered to the subject relative to body weight of the subject.

In some embodiments, 2.5 mg/kg to 10 mg/kg of BAN2401 is administered to the subject relative to body weight of the subject. In some embodiments, 5 mg/kg to 10 mg/kg of BAN2401 is administered to the subject relative to body weight of the subject. In some embodiments, 2.5 mg/kg of BAN2401 is administered to the subject relative to body weight of the subject. In some embodiments, 5 mg/kg of BAN2401 is administered to the subject relative to body weight of the subject. In some embodiments, 7.5 mg/kg of BAN2401 is administered to the subject relative to body weight of the subject. In some embodiments, 10 mg/kg of BAN2401 is administered to the subject relative to body weight of the subject.

Dosing Regimens for at Least One anti-Aβ Protofibril Antibody

The methods of the present disclosure comprise administering to a subject a composition comprising a therapeutically effective amount of at least one anti-Aβ protofibril antibody. One of ordinary skill in the art will understand that any of the therapeutically effective amounts of the at least one anti-Aβ protofibril antibody disclosed above may be administered one or more times according to one or more dosing regimens. One of ordinary skill in the art will be able to determine, depending upon a number of factors including pharmacodynamic characteristics, route of administration, dose, and health, age, and weight of the subject to be treated and, with the information disclosed herein, the appropriate dosing regimen(s) for each subject.

In some embodiments, a composition comprising at least one anti-Aβ protofibril antibody is administered every day, every other day, every third day, once every week, once every two weeks (“biweekly”), once every four weeks (“four-week interval”), once every month, once every six weeks, once every eight weeks, once every two months, once every ten weeks, once every twelve weeks, once every three months, once every fourteen weeks, once every sixteen weeks, once every four months, once every eighteen weeks, once every twenty weeks, once every five months, once every 22 weeks, once every 24 weeks, once every six months, once every eight months, once every seven months, once every eight months, once every nine months, once every ten months, once every eleven months, once every twelve months, once every thirteen months, once every thirteen months, once every fourteen months, once every fifteen months, once every sixteen months, once every seventeen months, or once every eighteen months. In some embodiments, a composition comprising at least one anti-Aβ protofibril antibody is administered every day, every other day, every third day, once every week, once every two weeks (“biweekly”), once every four weeks (“four-week interval”), or once every month. In some embodiments, a composition comprising a therapeutically effective amount of at least one anti-Aβ protofibril antibody is administered once every two weeks or once every four weeks. In some embodiments, a composition comprising a therapeutically effective amount of at least one anti-Aβ protofibril antibody is administered once every two weeks. In some embodiments, a composition comprising a therapeutically effective amount of at least one anti-Aβ protofibril antibody is administered once every four weeks.

In some embodiments, a composition comprising a therapeutically effective amount of at least one anti-Aβ protofibril antibody is administered once every week. In some embodiments, a composition comprising a therapeutically effective amount of at least one anti-Aβ protofibril antibody is administered once every two weeks. In some embodiments, a composition comprising a therapeutically effective amount of at least one anti-Aβ protofibril antibody is administered once every three weeks. In some embodiments, a composition comprising a therapeutically effective amount of at least one anti-Aβ protofibril antibody is administered once every four weeks. In some embodiments, a composition comprising a therapeutically effective amount of at least one anti-Aβ protofibril antibody is administered once every month.

In some embodiments, a composition comprising a therapeutically effective amount of BAN2401 is administered once every week. In some embodiments, a composition comprising a therapeutically effective amount of BAN2401 is administered once every two weeks. In some embodiments, a composition comprising a therapeutically effective amount of BAN2401 is administered once every three weeks. In some embodiments, a composition comprising a therapeutically effective amount of BAN2401 is administered once every four weeks. In some embodiments, a composition comprising a therapeutically effective amount of BAN2401 is administered once every month.

In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every week. In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every two weeks. In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every three weeks. In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every four weeks. In some embodiments, a composition comprising 2.5 mg/kg, 5 mg/kg, 7.5 mg/kg, or 10 mg/kg of at least one anti-Aβ protofibril antibody relative to body weight of the subject is administered to the subject once every month.

In some embodiments, a composition comprising 10 mg/kg of at least one anti-Aβ protofibril antibody relative to body weight of the subject is administered to the subject once every two weeks. In some embodiments, a composition comprising 10 mg/kg of at least one anti-Aβ protofibril antibody relative to body weight of the subject is administered to the subject once every month.

In some embodiments, a composition comprising 2.5 mg/kg, 5 mg/kg, 7.5 mg/kg, or 10 mg/kg of BAN2401 relative to body weight of the subject is administered to the subject once every week. In some embodiments, a composition comprising 2.5 mg/kg, 5 mg/kg, 7.5 mg/kg, or 10 mg/kg of BAN2401 relative to body weight of the subject is administered to the subject once every two weeks. In some embodiments, a composition comprising 2.5 mg/kg, 5 mg/kg, 7.5 mg/kg, or 10 mg/kg of BAN2401 relative to body weight of the subject is administered to the subject once every three weeks. In some embodiments, a composition comprising 2.5 mg/kg, 5 mg/kg, 7.5 mg/kg, or 10 mg/kg of BAN2401 relative to body weight of the subject is administered to the subject once every four weeks. In some embodiments, a composition comprising 2.5 mg/kg, 5 mg/kg, 7.5 mg/kg, or 10 mg/kg of BAN2401 relative to body weight of the subject is administered to the subject once every month.

In some embodiments, a composition comprising 10 mg/kg of BAN2401 relative to body weight of the subject is administered to the subject once every two weeks. In some embodiments, a composition comprising 10 mg/kg of BAN2401 relative to body weight of the subject is administered to the subject once every month.

Composition Comprising at Least One anti-Aβ Protofibril Antibody

In some embodiments, the at least one anti-Aβ protofibril antibody is comprised in a composition. In some embodiments, the composition consists of at least one anti-Aβ protofibril antibody. In some embodiments, the composition comprises at least one anti-Aβ protofibril antibody and further comprises at least one additional component. The at least one additional component can be chosen from suitable physiologically acceptable excipients for human and/or veterinary use.

The compositions of the present disclosure may be in the form of a tablet, pill, capsule, solution, and/or any other suitable form deemed appropriate by one of ordinary skill in the art. The route of administration of the compositions of the present disclosure may be any suitable route, including intravenous, subcutaneous, oral, and nasal. In some embodiments, the composition is formulated as a sterile, non-pyrogenic liquid for intravenous administration. In some embodiments, the composition is a saline solution.

In some embodiments, the at least one additional component in the composition is chosen from buffers. In some embodiments, the at least one additional component in the composition is chosen from emulsifiers. In some embodiments, the at least one additional component in the composition is chosen from sodium citrate, sodium chloride, and polysorbate 80. In some embodiments, the sodium citrate may be present at a concentration ranging from 1 mM to 150 mM. In some embodiments, the sodium citrate may be present at a concentration of 25 mM. In some embodiments, the sodium chloride may be present at a concentration ranging from 25 mM to 250 mM. In some embodiments, the sodium citrate may be present at a concentration of 125 mM. In some embodiments, the polysorbate 80 may be present at a concentration ranging from 0.001% (w/v) to 2% (w/v). In some embodiments, the polysorbate 80 may be present at a concentration of 0.02% (w/v).

In some embodiments, the composition is a liquid dosage form comprising at least one anti-Aβ protofibril antibody, such as BAN2401, and further comprising, for instance, sodium citrate, sodium chloride, and polysorbate 80. In some embodiments, the composition comprises 10 mg/mL at least one anti-Aβ protofibril antibody, such as BAN2401, 25 mM sodium citrate, 125 mM sodium chloride, and 0.2% (w/v) polysorbate 80, and has a pH 5.7.

### Therapeutic Effect

### Reduction of Clinical Decline

Provided herein is a method of reducing clinical decline in a subject having early Alzheimer's disease comprising administering to said subject a composition comprising a therapeutically effective amount of at least one anti-Aβ protofibril antibody disclosed herein. In some embodiments, the subject having early Alzheimer's disease has been diagnosed as having mild cognitive impairment due to Alzheimer's disease—intermediate likelihood and/or has been diagnosed as having mild Alzheimer's disease dementia. In some embodiments, the subject having early Alzheimer's disease is ApoE4-positive.

Any of the anti-Aβ protofibril antibodies, therapeutically acceptable amounts thereof, dosing regimens therefor, and compositions comprising the same that are disclosed herein may be used in the method of reducing clinical decline in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month. In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, or at least 46% relative to placebo as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 35% relative to placebo as determined by ADCOMS. In some embodiments, the clinical decline is reduced by 20% to 30% relative to placebo as determined by ADCOMS. In some embodiments, the clinical decline is reduced by 27% to 35% relative to placebo as determined by ADCOMS. In some embodiments, the clinical decline is reduced by at least 20% relative to placebo as determined by ADCOMS. In some embodiments, the clinical decline is reduced by at least 35% relative to placebo as determined by ADCOMS. In some embodiments, the clinical decline is reduced by at least 20% as determined by ADCOMS. In some embodiments, the clinical decline is reduced by at least 30% as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 45% relative to placebo as determined by ADCOMS after 6 months of administration of the composition comprising a therapeutically effective amount of at least one anti-Aβ protofibril antibody. In some embodiments, the clinical decline is reduced by at least 35% relative to placebo as determined by ADCOMS after 12 months of administration of the composition comprising a therapeutically effective amount of at least one anti-Aβ protofibril antibody. In some embodiments, the clinical decline is reduced by at least 30% relative to placebo as determined by ADCOMS after 18 months of administration of the composition comprising a therapeutically effective amount of at least one anti-Aβ protofibril antibody. In some embodiments, the clinical decline is reduced by at least 46% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, or at least 52% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 33% relative to placebo as determined by ADCOMS, wherein the subject has been diagnosed as having mild cognitive impairment due to Alzheimer's disease—intermediate likelihood. In some embodiments, the clinical decline is reduced by at least 20%, such as by at 25% or at least 28%, relative to placebo as determined by ADCOMS, wherein the subject has been diagnosed as having mild cognitive impairment due to Alzheimer's disease—intermediate likelihood. In some embodiments, the clinical decline is reduced by at least 25%, such as by at least 30% or at least 33%, relative to placebo as determined by ADCOMS, wherein the subject has been diagnosed as having mild cognitive impairment due to Alzheimer's disease—intermediate likelihood. In some embodiments, the clinical decline is reduced by at least 25%, such as by at least 30%, at least 35%, at least 40%, at least 45%, at least 50%, or at least 52% relative to placebo as determined by ADCOMS, wherein the subject has been diagnosed as having mild cognitive impairment due to Alzheimer's disease—intermediate likelihood. In some embodiments, the clinical decline is reduced by at least 52% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 30% relative to placebo as determined by ADCOMS after 6 months of administration of the composition comprising a therapeutically effective amount of at least one anti-Aβ protofibril antibody. In some embodiments, the clinical decline in the subject diagnosed as having mild cognitive impairment due to Alzheimer's disease—intermediate likelihood is reduced by at least 25% relative to placebo as determined by ADCOMS after 12 months of administration of the composition comprising a therapeutically effective amount of at least one anti-Aβ protofibril antibody. In some embodiments, the clinical decline in the subject diagnosed as having mild cognitive impairment due to Alzheimer's disease—intermediate likelihood is reduced by at least 30% relative to placebo as determined by ADCOMS after 18 months of administration of the composition comprising a therapeutically effective amount of at least one anti-Aβ protofibril antibody. In some embodiments, the clinical decline in the subject diagnosed as having mild cognitive impairment due to Alzheimer's disease—intermediate likelihood is reduced by at least 52% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, or at least 33% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 38% relative to placebo as determined by ADCOMS, wherein the subject has been diagnosed as having mild cognitive impairment due to Alzheimer's disease—intermediate likelihood. In some embodiments, the clinical decline is reduced by at least 20%, such as by at 25%, at least 28%, or at least 33%, relative to placebo as determined by ADCOMS, wherein the subject has been diagnosed as having mild cognitive impairment due to Alzheimer's disease—intermediate likelihood. In some embodiments, the clinical decline is reduced by at least 25%, such as by at least 30% or at least 33%, relative to placebo as determined by ADCOMS, wherein the subject has been diagnosed as having mild cognitive impairment due to Alzheimer's disease—intermediate likelihood. In some embodiments, the clinical decline is reduced by at least 33% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 33% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41% at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, or at least 78% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 80% relative to placebo as determined by ADCOMS, wherein the subject has been diagnosed as having Alzheimer's disease dementia. In some embodiments, the clinical decline is reduced by 35% to 78% relative to placebo as determined by ADCOMS, wherein the subject has been diagnosed as having Alzheimer's disease dementia. In some embodiments, the clinical decline is reduced by at least 35% relative to placebo as determined by ADCOMS, wherein the subject has been diagnosed as having mild Alzheimer's disease dementia. In some embodiments, the clinical decline is reduced by at least 50%, such as by at least 52% or at least 53% relative to placebo as determined by ADCOMS, wherein the subject has been diagnosed as having mild Alzheimer's disease dementia. In some embodiments, the clinical decline is reduced by at least 70%, such as by at least 75% or at least 78%, relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 70% relative to placebo as determined by ADCOMS after 6 months of administration of the composition comprising a therapeutically effective amount of at least one anti-Aβ protofibril antibody. In some embodiments, the clinical decline in the subject diagnosed as having mild Alzheimer's disease dementia is reduced by at least 50% relative to placebo as determined by ADCOMS after 12 months of administration of the composition comprising a therapeutically effective amount of at least one anti-Aβ protofibril antibody. In some embodiments, the clinical decline in the subject diagnosed as having mild Alzheimer's disease dementia is reduced by at least 30% relative to placebo as determined by ADCOMS after 18 months of administration of the composition comprising a therapeutically effective amount of at least one anti-Aβ protofibril antibody. In some embodiments, the clinical decline in the subject diagnosed as having mild Alzheimer's disease dementia is reduced by at least 52% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, or at least 35% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 38% relative to placebo as determined by ADCOMS, wherein the subject has been diagnosed as having mild Alzheimer's disease dementia. In some embodiments, the clinical decline is reduced by at least 20%, such as by at 25%, at least 28%, or at least 35%, relative to placebo as determined by ADCOMS, wherein the subject has been diagnosed as having mild Alzheimer's disease dementia. In some embodiments, the clinical decline is reduced by at least 25%, such as by at least 30% or at least 35%, relative to placebo as determined by ADCOMS, wherein the subject has been diagnosed as having mild Alzheimer's disease dementia. In some embodiments, the clinical decline is reduced by at least 35% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 35% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 75%, at least 80%, at least 85%, at least 90%, at least 95%, at least 100%, at least 110%, at least 120%, at least 130%, at least 140%, or at least 150% relative to placebo as determined by ADAS-cog. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 150% relative to placebo as determined by ADAS-cog. In some embodiments, the clinical decline is reduced by 45% to 145% relative to placebo as determined by ADAS-cog. In some embodiments, the clinical decline is reduced by 45% to 55% relative to placebo as determined by ADAS-cog. In some embodiments, the clinical decline is reduced by at least 30% relative to placebo as determined by ADAS-cog. In some embodiments, the clinical decline is reduced by at least 35% relative to placebo as determined by ADAS-cog. In some embodiments, the clinical decline is reduced by at least 40% as determined by ADAS-cog. In some embodiments, the clinical decline is reduced by at least 45% as determined by ADAS-cog. In some embodiments, the clinical decline is reduced by at least 47% as determined by ADAS-cog. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 100%, such as at least 120% or at least 140%, relative to placebo as determined by ADAS-cog after 6 months of administration of the composition comprising a therapeutically effective amount of at least one anti-Aβ protofibril antibody. In some embodiments, the clinical decline is reduced by at least 40%, such as at least 45%, relative to placebo as determined by ADAS-cog after 12 months of administration of the composition comprising a therapeutically effective amount of at least one anti-Aβ protofibril antibody. In some embodiments, the clinical decline is reduced by at least 40%, such as at least 45%, relative to placebo as determined by ADAS-cog after 18 months of administration of the composition comprising a therapeutically effective amount of at least one anti-Aβ protofibril antibody. In some embodiments, the clinical decline is reduced by at least 47%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 56%, at least 47%, at least 48%, at least 49%, at least 50%, at least 51%, at least 52%, at least 53%, at least 54%, at least 55%, at least 56%, at least 57%, or at least 58% relative to placebo as determined by ADAS-cog, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject has been diagnosed as having mild cognitive impairment due to Alzheimer's disease—intermediate likelihood. In some embodiments, the clinical decline is reduced by at least 50%, such as by at 52%, at least 55%, or at least 58%, relative to placebo as determined by ADAS-cog, wherein the subject has been diagnosed as having mild cognitive impairment due to Alzheimer's disease—intermediate likelihood. In some embodiments, the clinical decline is reduced by at least 58% relative to placebo as determined by ADAS-cog, wherein the subject has been diagnosed as having mild cognitive impairment due to Alzheimer's disease-13 intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 58% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, or at least 41% relative to placebo as determined by ADAS-cog, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 30% to 50% relative to placebo as determined by ADAS-cog, wherein the subject has been diagnosed as having mild Alzheimer's disease dementia. In some embodiments, the clinical decline is reduced by at least 35%, such as by at 38%, at least 40%, or at least 41%, relative to placebo as determined by ADAS-cog, wherein the subject has been diagnosed as having mild Alzheimer's disease dementia. In some embodiments, the clinical decline is reduced by at least 41% relative to placebo as determined by ADAS-cog, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 41% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40% relative to placebo as determined by CDR-SB. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 60% relative to placebo as determined by CDR-SB. In some embodiments, the clinical decline is reduced by 25% to 60% relative to placebo as determined by CDR-SB. In some embodiments, the clinical decline is reduced by 25% to 50% relative to placebo as determined by CDR-SB. In some embodiments, the clinical decline is reduced by at least 20% relative to placebo as determined by CDR-SB. In some embodiments, the clinical decline is reduced by at least 30% relative to placebo as determined by CDR-SB. In some embodiments, the clinical decline is reduced by at least 25%, such as at least 26% or at least 28%, as determined by CDR-SB. In some embodiments, the clinical decline is reduced by at least 30%, such as at least 35% or at least 38%, as determined by CDR-SB. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 30%, such as at least 35% or at least 40%, relative to placebo as determined by CDR-SB after 6 months of administration of the composition comprising a therapeutically effective amount of at least one anti-Aβ protofibril antibody. In some embodiments, the clinical decline is reduced by at least 30%, such as at least 35% or at least 45%, relative to placebo as determined by CDR-SB after 12 months of administration of the composition comprising a therapeutically effective amount of at least one anti-Aβ protofibril antibody. In some embodiments, the clinical decline is reduced by at least 20%, such as at least 25%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, or at least 14% relative to placebo as determined by CDR-SB, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 10% to 20% relative to placebo as determined by CDR-SB, wherein the subject has been diagnosed as having mild cognitive impairment due to Alzheimer's disease—intermediate likelihood. In some embodiments, the clinical decline is reduced by at least 5%, such as by at 10%, at least 12%, or at least 14%, relative to placebo as determined by CDR-SB, wherein the subject has been diagnosed as having mild cognitive impairment due to Alzheimer's disease—intermediate likelihood. In some embodiments, the clinical decline is reduced by at least 14% relative to placebo as determined by CDR-SB, wherein the subject has been diagnosed as having mild cognitive impairment due to Alzheimer's disease-13 intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 14% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, or at least 51% relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 60% relative to placebo as determined by CDR-SB, wherein the subject has been diagnosed as having mild Alzheimer's disease dementia. In some embodiments, the clinical decline is reduced by at least 45%, such as by at 48%, at least 50%, or at least 51%, relative to placebo as determined by CDR-SB, wherein the subject has been diagnosed as having mild Alzheimer's disease dementia. In some embodiments, the clinical decline is reduced by at least 51% relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 51% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of at least one anti-Aβ protofibril antibody. In some embodiments, the reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the reduction in clinical decline is determined after 1 month of administration of the composition comprising a therapeutically effective amount of at least one anti-Aβ protofibril antibody. In some embodiments, the reduction in clinical decline is determined after 6 months of administration of the composition comprising a therapeutically effective amount of at least one anti-Aβ protofibril antibody. In some embodiments, the reduction in clinical decline is determined after 12 months of administration of the composition comprising a therapeutically effective amount of at least one anti-Aβ protofibril antibody. In some embodiments, the reduction in clinical decline is determined after 18 months of administration of the composition comprising a therapeutically effective amount of at least one anti-Aβ protofibril antibody. In some embodiments, the reduction in clinical decline is determined after 60 months of administration of the composition comprising a therapeutically effective amount of at least one anti-Aβ protofibril antibody. In some embodiments, the reduction in clinical decline is determined after 63 months of administration of the composition comprising a therapeutically effective amount of at least one anti-Aβ protofibril antibody.

In some embodiments, the reduction in clinical decline is determined after administration of a composition comprising a therapeutically effective amount of BAN2401.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of BAN2401. In some embodiments, the reduction in clinical decline is determined after 1 month, 6 months, 12 months, 18 months, 60 months, and/or 63 months of administration of the composition comprising a therapeutically effective amount of BAN2401. In some embodiments, the reduction in clinical decline is determined after 1 month of administration of the composition comprising a therapeutically effective amount of BAN2401. In some embodiments, the reduction in clinical decline is determined after 6 months of administration of the composition comprising a therapeutically effective amount of BAN2401. In some embodiments, the reduction in clinical decline is determined after 12 months of administration of the composition comprising a therapeutically effective amount of BAN2401. In some embodiments, the reduction in clinical decline is determined after 18 months of administration of the composition comprising a therapeutically effective amount of BAN2401. In some embodiments, the reduction in clinical decline is determined after 60 months of administration of the composition comprising a therapeutically effective amount of BAN2401. In some embodiments, the reduction in clinical decline is determined after 63 months of administration of the composition comprising a therapeutically effective amount of BAN2401.

In some embodiments, the subject is ApoE4-positive.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, or at least 74% relative to placebo as determined by ADCOMS, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 60% to 80%, such as by 63% to 74%, relative to placebo as determined by ADCOMS, wherein the subject is ApoE4-positive. In some embodiments, the clinical decline is reduced by at least 60%, such as at least 63%, relative to placebo as determined by ADCOMS, wherein the subject is ApoE4-positive. In some embodiments, the clinical decline is reduced by at least 65%, such as at least 67%, relative to placebo as determined by ADCOMS, wherein the subject is ApoE4-positive. In some embodiments, the clinical decline is reduced by at least 70%, such as at least 74%, relative to placebo as determined by ADCOMS, wherein the subject is ApoE4-positive.

In some embodiments, the clinical decline in the ApoE4-positive subject is reduced by at least 70% relative to placebo as determined by ADCOMS after 6 months of administration of the composition comprising a therapeutically effective amount of at least one anti-Aβ protofibril antibody. In some embodiments, the clinical decline in the ApoE4-positive subject is reduced by at least 60% relative to placebo as determined by ADCOMS after 12 months of administration of the composition comprising a therapeutically effective amount of at least one anti-Aβ protofibril antibody. In some embodiments, the clinical decline in the ApoE4-positive subject is reduced by at least 50%, such as at least 55% or at least 60%, relative to placebo as determined by ADCOMS after 18 months of administration of the composition comprising a therapeutically effective amount of at least one anti-Aβ protofibril antibody. In some embodiments, the clinical decline in the ApoE4-positive subject is reduced by at least 63%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 290%, at least 295%, at least 300%, at least 305%, at least 310%, at least 315%, at least 320%, at least 325%, at least 330%, or at least 331% relative to placebo as determined by ADAS-cog, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced 70% to 400%, such as 80% to 350%, relative to placebo as determined by ADAS-cog, wherein the subject is ApoE4-positive. In some embodiments, the clinical decline is reduced by at least 70%, such as at least 75% or at least 80%, relative to placebo as determined by ADAS-cog, wherein the subject is ApoE4-positive. In some embodiments, the clinical decline is reduced by at least 80%, such as at least 90% or at least 100%, relative to placebo as determined by ADAS-cog, wherein the subject is ApoE4-positive. In some embodiments, the clinical decline is reduced by at least 300%, such as at least 330%, relative to placebo as determined by ADAS-cog, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject is reduced by at least 300% relative to placebo as determined by ADAS-cog after 6 months of administration of the composition comprising a therapeutically effective amount of at least one anti-Aβ protofibril antibody. In some embodiments, the clinical decline in the ApoE4-positive subject is reduced by at least 80%, such as at least 90% or at least 100%, relative to placebo as determined by ADAS-cog after 12 months of administration of the composition comprising a therapeutically effective amount of at least one anti-Aβ protofibril antibody. In some embodiments, the clinical decline in the ApoE4-positive subject is reduced by at least 70%, such as at least 75%, at least 80%, or at least 84%, relative to placebo as determined by ADAS-cog after 18 months of administration of the composition comprising a therapeutically effective amount of at least one anti-Aβ protofibril antibody. In some embodiments, the clinical decline in the ApoE4-positive subject is reduced by at least 84%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, or at least 87% relative to placebo as determined by CDR-SB, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 35% to 150%, such as 40% to 100% or 45% to 90%, relative to placebo as determined by CDR-SB, wherein the subject is ApoE4-positive. In some embodiments, the clinical decline is reduced by at least 35%, such as at least 40% or at least 45%, relative to placebo as determined by CDR-SB, wherein the subject is ApoE4-positive. In some embodiments, the clinical decline is reduced by at least 50%, such as at least 55% or at least 60%, relative to placebo as determined by CDR-SB, wherein the subject is ApoE4-positive. In some embodiments, the clinical decline is reduced by at least 70%, such as at least 80% or at least 85%, relative to placebo as determined by CDR-SB, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject is reduced by at least 35%, such as at least 40% or at least 45%, relative to placebo as determined by CDR-SB after 6 months of administration of the composition comprising a therapeutically effective amount of at least one anti-Aβ protofibril antibody. In some embodiments, the clinical decline in the ApoE4-positive subject is reduced by at least 70%, such as at least 75% or at least 80%, relative to placebo as determined by CDR-SB after 12 months of administration of the composition comprising a therapeutically effective amount of at least one anti-Aβ protofibril antibody. In some embodiments, the clinical decline in the ApoE4-positive subject is reduced by at least 50%, such as at least 55% or at least 60%, relative to placebo as determined by CDR-SB after 18 months of administration of the composition comprising a therapeutically effective amount of at least one anti-Aβ protofibril antibody. In some embodiments, the clinical decline in the ApoE4-positive subject is reduced by at least 60%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, or at least 59% relative to placebo as determined by ADCOMS, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 30% to 70%, such as 38% to 59%, relative to placebo as determined by ADCOMS, wherein the subject is ApoE4-positive, and wherein the subject has been diagnosed as having mild cognitive impairment due to Alzheimer's disease—intermediate likelihood. In some embodiments, the clinical decline is reduced by at least 30%, such as at least 35% or at least 38%, relative to placebo as determined by ADCOMS, wherein the subject is ApoE4-positive, and wherein the subject has been diagnosed as having mild cognitive impairment due to Alzheimer's disease—intermediate likelihood. In some embodiments, the clinical decline is reduced by at least 45%, such as at least 50% or at least 53%, relative to placebo as determined by ADCOMS, wherein the subject is ApoE4-positive, and wherein the subject has been diagnosed as having mild cognitive impairment due to Alzheimer's disease—intermediate likelihood. In some embodiments, the clinical decline is reduced by at least 50%, such as at least 55% or at least 59%, relative to placebo as determined by ADCOMS, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease—intermediate likelihood is reduced by at least 50%, such as at least 55%, relative to placebo as determined by ADCOMS after 6 months of administration of the composition comprising a therapeutically effective amount of at least one anti-Aβ protofibril antibody. In some embodiments, the clinical decline in the ApoE4-positive subject diagnosed as mild cognitive impairment due to Alzheimer's disease—intermediate likelihood is reduced by at least 30%, such as at least 35%, relative to placebo as determined by ADCOMS after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease—intermediate likelihood is reduced by at least 45%, such as at least 50% or at least 55%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, or at least 211% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild Alzheimer's disease dementia is reduced by at least 100%, such as at least 110%, relative to placebo as determined by ADCOMS after 6 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 100%, such as at least 110%, relative to placebo as determined by ADCOMS after 12 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 65%, such as at least 70% or at least 75%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, or at least 211% relative to placebo as determined by ADAS-Cog, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 300%, such as 45% to 250% or 50% to 250%, relative to placebo as determined by ADAS-Cog, wherein the subject is ApoE4-positive, and wherein the subject has been diagnosed as having mild cognitive impairment due to Alzheimer's disease—intermediate likelihood. In some embodiments, the clinical decline is reduced by at least 40%, such as at least 45% or at least 50%, relative to placebo as determined by ADAS-Cog, wherein the subject is ApoE4-positive, and wherein the subject has been diagnosed as having mild cognitive impairment due to Alzheimer's disease—intermediate likelihood. In some embodiments, the clinical decline is reduced by at least 60, such as at least 70%, at least 75%, or at least 80%, relative to placebo as determined by ADAS-Cog, wherein the subject is ApoE4-positive, and wherein the subject has been diagnosed as having mild cognitive impairment due to Alzheimer's disease—intermediate likelihood. In some embodiments, the clinical decline is reduced by at least 100%, such as at least 150% or at least 200% relative to placebo as determined by ADAS-Cog, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease—intermediate likelihood is reduced by at least 100%, such as at least 150% or at least 200%, relative to placebo as determined by ADAS-cog after 6 months of administration of the composition comprising a therapeutically effective amount of at least one anti-Aβ protofibril antibody. In some embodiments, the clinical decline in the ApoE4-positive subject diagnosed as mild cognitive impairment due to Alzheimer's disease—intermediate likelihood is reduced by at least 40%, such as at least 45% or at least 50%, relative to placebo as determined by ADAS-cog after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease-13 intermediate likelihood is reduced by at least 50%, such as at least 60%, at least 70%, or at least 75%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45% relative to placebo as determined by CDR-SB, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 90%, such as 25% to 80% or 30% to 75%, relative to placebo as determined by CDR-SB, wherein the subject is ApoE4-positive, and wherein the subject has been diagnosed as having mild cognitive impairment due to Alzheimer's disease—intermediate likelihood. In some embodiments, the clinical decline is reduced by at least 25%, such as at least 30%, relative to placebo as determined by CDR-SB, wherein the subject is ApoE4-positive, and wherein the subject has been diagnosed as having mild cognitive impairment due to Alzheimer's disease—intermediate likelihood. In some embodiments, the clinical decline is reduced by at least 30%, such as at least 35% or 40%, relative to placebo as determined by CDR-SB, wherein the subject is ApoE4-positive, and wherein the subject has been diagnosed as having mild cognitive impairment due to Alzheimer's disease—intermediate likelihood. In some embodiments, the clinical decline is reduced by at least 35%, such as at least 40% or 45%, relative to placebo as determined by CDR-SB, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease—intermediate likelihood is reduced by at least 35%, such as at least 40% or at least 45%, relative to placebo as determined by CDR-SB composition comprising a therapeutically effective amount of at least one anti-Aβ protofibril antibody. In some embodiments, the clinical decline in the ApoE4-positive subject diagnosed as mild cognitive impairment due to Alzheimer's disease—intermediate likelihood is reduced by at least 20%, such as at least 25% or at least 30%, relative to placebo as determined by CDR-SB after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease—intermediate likelihood is reduced by at least 35%, such as at least 40%, relative to placebo as determined by CDR-SB after 18 months of administration of the composition comprising a therapeutically effective amount of at least one anti-protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, or at least 119% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 76% to 119% relative to placebo as determined by ADCOMS, wherein the subject is ApoE4-positive, and wherein the subject has been diagnosed as having mild Alzheimer's disease dementia. In some embodiments, the clinical decline is reduced by 76% relative to placebo as determined by ADCOMS, wherein the subject is ApoE4-positive, and wherein the subject has been diagnosed as having mild Alzheimer's disease dementia. In some embodiments, the clinical decline is reduced by 113% relative to placebo as determined by ADCOMS, wherein the subject is ApoE4-positive, and wherein the subject has been diagnosed as having mild Alzheimer's disease dementia. In some embodiments, the clinical decline is reduced by 119% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, at least 119%, at least 120%, at least 121%, at least 122%, at least 123%, at least 124%, at least 125%, at least 126%, at least 127%, at least 128%, at least 129%, at least 130%, at least 131%, at least 132%, at least 133%, at least 134%, at least 135%, at least 136%, at least 137%, at least 138%, at least 139%, at least 140%, at least 141%, at least 142%, at least 143%, at least 144%, at least 145%, at least 146%, at least 147%, at least 148%, at least 149%, at least 150%, at least 151%, at least 152%, at least 153%, at least 154%, at least 155%, at least 156%, at least 157%, at least 158%, at least 159%, at least 160%, at least 161%, at least 162%, at least 163%, at least 164%, at least 165%, at least 166%, at least 167%, at least 168%, at least 169%, at least 170%, at least 171%, at least 172%, at least 173%, at least 174%, at least 175%, at least 176%, at least 177%, at least 178%, at least 179%, at least 180%, at least 190%, at least 200%, at least 210%, at least 220%, at least 230%, at least 240%, at least 250%, at least 275%, at least 300%, at least 325%, at least 350%, at least 375%, at least 400%, at least 425%, at least 450%, at least 475%, at least 500%, at least 550%, at least 600%, at least 650%, at least 700%, at least 750%, at least 800%, at least 850%, at least 900%, at least 950%, at least 1000%, at least 1001%, at least 1002%, at least 1003%, at least 1004%, at least 1005%, at least 1006%, at least 1007%, at least 1008%, at least 1009%, at least 1010%, at least 1011%, at least 1012%, at least 1013%, at least 1014%, at least 1015%, at least 1016%, at least 1017%, at least 1018%, at least 1019%, at least 1020%, at least 1021%, at least 1022%, or at least 1023% relative to placebo as determined by ADAS-Cog, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 58% to 1023% relative to placebo as determined by ADAS-Cog, wherein the subject is ApoE4-positive, and wherein the subject has been diagnosed as having mild Alzheimer's disease dementia. In some embodiments, the clinical decline is reduced by 58% relative to placebo as determined by ADAS-Cog, wherein the subject is ApoE4-positive, and wherein the subject has been diagnosed as having mild Alzheimer's disease dementia. In some embodiments, the clinical decline is reduced by 171% relative to placebo as determined by ADAS-Cog, wherein the subject is ApoE4-positive, positive, and wherein the subject has been diagnosed as having mild Alzheimer's disease dementia. In some embodiments, the clinical decline is reduced by 1023% relative to placebo as determined by ADAS-Cog, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, at least 119%, at least 120%, at least 121%, at least 122%, at least 123%, at least 124%, at least 125%, at least 126%, at least 127%, at least 128%, at least 129%, at least 130%, at least 131%, at least 132%, at least 133%, at least 134%, at least 135%, at least 136%, at least 137%, at least 138%, at least 139%, at least 140%, at least 141%, at least 142%, at least 143%, at least 144%, at least 145%, at least 146%, at least 147%, at least 148%, at least 149%, at least 150%, at least 151%, at least 152%, at least 153%, at least 154%, at least 155%, at least 156%, at least 157%, at least 158%, at least 159%, at least 160%, at least 161%, at least 162%, at least 163%, at least 164%, at least 165%, at least 166%, at least 167%, at least 168%, at least 169%, at least 170%, at least 171%, at least 172%, at least 173%, or at least 174% relative to placebo as determined by CDR-SB, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 70% to 200%, such as 75% to 180% or 82% to 174%, relative to placebo as determined by CDR-SB, wherein the subject is ApoE4-positive, and wherein the subject has been diagnosed as having mild Alzheimer's disease dementia. In some embodiments, the clinical decline is reduced by at least 70%, such as at least 80% relative to placebo as determined by CDR-SB, wherein the subject is ApoE4-positive, and wherein the subject has been diagnosed as having mild Alzheimer's disease dementia. In some embodiments, the clinical decline is reduced by at least 75%, such as at least 80% or at least 85%, relative to placebo as determined by CDR-SB, wherein the subject is ApoE4-positive, and wherein the subject has been diagnosed as having mild Alzheimer's disease dementia. In some embodiments, the clinical decline is reduced by at least 150%, such as at least 160% or 170%, relative to placebo as determined by CDR-SB, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild Alzheimer's disease dementia is reduced by at least 70%, such as at least 75%, at least 80%, or at least 85%, relative to placebo as determined by CDR-SB composition comprising a therapeutically effective amount of at least one anti-Aβ protofibril antibody. In some embodiments, the clinical decline in the ApoE4-positive subject diagnosed as having mild Alzheimer's disease dementia is reduced by at least 130%, such as at least 140%, at least 150%, at least 160%, or at least 170%, relative to placebo as determined by CDR-SB after 12 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 65%, such as at least 70%, at least 75%, or at least 80%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the subject is ApoE4-negative.

In some embodiments, the clinical decline is reduced by at least 1%, at least 2%, at least 3%, at least 4%, at least 5%, at least 6%, at least 7%, at least 8%, at least 9%, at least 10%, at least 11%, or at least 12% relative to placebo as determined by ADCOMS, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 5% to 15% relative to placebo as determined by ADCOMS, wherein the subject is ApoE4-negative. In some embodiments, the clinical decline is reduced by at least 5%, such as at least 7%, relative to placebo as determined by ADCOMS, wherein the subject is ApoE4-negative. In some embodiments, the clinical decline is reduced by at least 10%, such as at least 12%, relative to placebo as determined by ADCOMS, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-negative subject is reduced by at least −2% relative to placebo as determined by ADCOMS after 6 months of administration of the composition comprising a therapeutically effective amount of at least one anti-Aβ protofibril antibody. In some embodiments, the clinical decline in the ApoE4-negative subject is reduced by at least 10% relative to placebo as determined by ADCOMS after 12 months of administration of the composition comprising a therapeutically effective amount of at least one anti-Aβ protofibril antibody. In some embodiments, the clinical decline in the ApoE4-negative subject is reduced by at least 5%, such as at least 7%, relative to placebo as determined by ADCOMS after 18 months of administration of the composition comprising a therapeutically effective amount of at least one anti-Aβ protofibril antibody. In some embodiments, the clinical decline in the ApoE4-negative subject is reduced by at least 7%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, or at least 72% relative to placebo as determined by ADAS-cog, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 80% relative to placebo as determined by ADAS-cog, wherein the subject is ApoE4-negative. In some embodiments, the clinical decline is reduced by at least 35%, such as at least 40% or at least 43%, relative to placebo as determined by ADAS-cog, wherein the subject is ApoE4-negative. In some embodiments, the clinical decline is reduced by at least 40%, such as at least 45% or at least 46%, relative to placebo as determined by ADAS-cog, wherein the subject is ApoE4-negative. In some embodiments, the clinical decline is reduced by at least 65%, such as at least 70% or at least 72%, relative to placebo as determined by ADAS-cog, wherein the subject is ApoE4-negative. In some embodiments, the clinical decline is reduced by at least 43%, relative to placebo as determined by ADAS-cog, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is increased by 7%, 6%, 5%, 5%, 3%, 2%, or 1% relative to placebo as determined by CDR-SB, wherein the subject is ApoE4-negative. In some embodiments, the clinical decline is reduced by at least 1%, at least 2%, or at least 3% relative to placebo as determined by CDR-SB, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 3% relative to placebo as determined by CDR-SB, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody. In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, or at least 26% relative to placebo as determined by ADCOMS, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 15% to 26% relative to placebo as determined by ADCOMS, wherein the subject is ApoE4-negative, and wherein the subject has been diagnosed as having mild cognitive impairment due to Alzheimer's disease—intermediate likelihood. In some embodiments, the clinical decline is reduced by 15% relative to placebo as determined by ADCOMS, wherein the subject is ApoE4-negative, and wherein the subject has been diagnosed as having mild cognitive impairment due to Alzheimer's disease—intermediate likelihood. In some embodiments, the clinical decline is reduced by 26% relative to placebo as determined by ADCOMS, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, or at least 166% relative to placebo as determined by ADAS-Cog, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 200%, such as 60% to 180% or 65% to 170%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the clinical decline is reduced by at least 50%, such as at least 55% or at least 65%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the clinical decline is reduced by at least 70%, such as at least 75% or at least 80%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the clinical decline is reduced by at least 150%, such as at least 160%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-negative subject is reduced by at least 150%, such as at least 160%, relative to placebo as determined by ADAS-Cog after 6 months of administration of the composition comprising a therapeutically effective amount of at least one anti-Aβ protofibril antibody. In some embodiments, the clinical decline in the ApoE4-negative subject is reduced by at least 70%, such as at least 75% or at least 80%, relative to placebo as determined by ADAS-Cog after 12 months of administration of the composition comprising a therapeutically effective amount of at least one anti-Aβ protofibril antibody. In some embodiments, the clinical decline in the ApoE4-negative subject is reduced by at least 50%, such as at least 60% or at least 65%, relative to placebo as determined by ADAS-Cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, or at least 5% relative to placebo as determined by CDR-SB, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 5% relative to placebo as determined by CDR-SB, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12% relative to placebo as determined by CDR-SB, wherein the subject is ApoE4-negative, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 10%, such as at least 12%, relative to placebo as determined by CDR-SB, wherein the subject is ApoE4-negative, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

Conversion of a Subject from Amyloid Positive to Amyloid Negative

Also provided herein is a method of converting an amyloid-positive subject to an amyloid-negative subject. In some embodiments, said method comprises administering to said subject a composition comprising at least one anti-Aβ protofibril antibody disclosed herein. In some embodiments, said subject having early Alzheimer's disease has been diagnosed as having mild cognitive impairment due to Alzheimer's disease—intermediate likelihood and/or has been diagnosed as having mild Alzheimer's disease dementia.

Any of the anti-Aβ protofibril antibodies, therapeutically acceptable amounts thereof, dosing regimens therefor, and compositions comprising the same that are disclosed herein may be used in the method of converting an amyloid-positive subject to an amyloid-negative subject. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month.

In some embodiments, administration of the composition results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, or at least 81% of the subjects being converted from amyloid positive to amyloid negative, as determined by visual reads of amyloid PET images.

In some embodiments, administration of the composition results in a conversion of 50% to 100%, such as 60% to 90%, of subjects from amyloid positive to amyloid negative, as determined by visual reads of amyloid PET images. In some embodiments, administration of the composition results in at least 55%, such as at least 60% or at least 65%, of the subjects being amyloid negative, as determined by visual reads of amyloid PET images, after 12 months of administration of the composition comprising a therapeutically effective amount of at least one anti-Aβ protofibril antibody. In some embodiments, administration of the composition results in at least 70%, such as at least 75% or at least 80%, of the subjects being amyloid negative, as determined by visual reads of amyloid PET images, after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, administration of the composition results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images, wherein the subjects are ApoE4-positive.

In some embodiments, administration of the composition results in 75% to 100%, such as 80% to 100% or 85% to 100% of the subjects being amyloid negative, as determined by visual reads of amyloid PET images, wherein the subjects are ApoE4-positive. In some embodiments, administration of the composition results in at least 75%, such as at least 80% or at least 85%, of the subjects being amyloid negative, as determined by visual reads of amyloid PET images, wherein the subjects are ApoE4-positive. In some embodiments, administration of the composition results in 100% of the subjects being amyloid negative, as determined by visual reads of amyloid PET images, wherein the subjects are ApoE4-positive.

In some embodiments, at least 75%, such as at least 80% or at least 85%, of the ApoE4-positive subjects are amyloid negative, as determined by visual reads of amyloid PET images, after 12 months of administration of the composition comprising a therapeutically effective amount of at least one anti-Aβ protofibril antibody. In some embodiments, at least 75%, such as at least 80%, at least 85%, at least 90%, or at least 95%, of the ApoE4-positive subjects are amyloid negative, as determined by visual reads of amyloid PET images, after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, administration of the composition results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, or at least 79% of the subjects being amyloid negative, as determined by visual reads of amyloid PET images, wherein the subjects are ApoE4-negative.

In some embodiments, administration of the composition results in 50% to 100%, such as 55% to 90%, of the subjects being amyloid negative, as determined by visual reads of amyloid PET images, wherein the subjects are ApoE4-negative. In some embodiments, administration of the composition results in at least 50% of the subjects being amyloid negative, as determined by visual reads of amyloid PET images, wherein the subjects are ApoE4-negative. In some embodiments, administration of the composition results in at least 70% of the subjects being amyloid negative, as determined by visual reads of amyloid PET images, wherein the subjects are ApoE4-negative.

In some embodiments, the clinical decline is reduced by 35% to 50% relative to placebo as determined by ADCOMS, wherein the subject is not concomitantly administered at least one Alzheimer's disease medication other than BAN2401. In some embodiments, the clinical decline is reduced by at least 35%, such as by at 38%, at least 40%, or at least 41%, relative to placebo as determined by ADCOMS, wherein the subject is not concomitantly administered at least one Alzheimer's disease medication other than BAN2401. In some embodiments, the clinical decline is reduced by at least 41%, relative to placebo as determined by ADCOMS, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody other than BAN2401.

In some embodiments, the clinical decline in the subject who is not concomitantly administered at least one Alzheimer's disease medication is reduced by at least 41% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, or at least 59% relative to placebo as determined by ADAS-cog, wherein the subject is not concomitantly administered at least one Alzheimer's disease medication. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject is not concomitantly administered at least one Alzheimer's disease medication. In some embodiments, the clinical decline is reduced by at least 50%, such as by at 55%, at least 57%, or at least 59%, relative to placebo as determined by ADAS-cog, wherein the subject is not concomitantly administered at least one Alzheimer's disease medication. In some embodiments, the clinical decline is reduced by at least 59% relative to placebo as determined by ADAS-cog, wherein the subject is not concomitantly administered at least one Alzheimer's disease medication. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject who is not concomitantly administered at least one Alzheimer's disease medication is reduced by at least 59% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45% relative to placebo as determined by CDR-SB, wherein the subject is not concomitantly administered at least one Alzheimer's disease medication. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject is not concomitantly administered at least one Alzheimer's disease medication. In some embodiments, the clinical decline is reduced by at least 35%, such as by at 40%, at least 42%, or at least 45%, relative to placebo as determined by CDR-SB, wherein the subject is not concomitantly administered at least one Alzheimer's disease medication. In some embodiments, the clinical decline is reduced by at least 45% relative to placebo as determined by CDR-SB, wherein the subject is not concomitantly administered at least one Alzheimer's disease medication. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject who is not concomitantly administered at least one Alzheimer's disease medication is reduced by at least 45% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

### Concomitant Administration of At Least One Anti-An Protofibril Antibody and At Least One Alzheimer's Disease Medication Other Than BAN2401

In some embodiments, provided herein is a method of reducing clinical decline in a subject having early Alzheimer's disease comprising concomitantly administering a therapeutically effective amount of at least one anti-Aβ protofibril antibody and a therapeutically effective amount of at least one Alzheimer's disease medication other than BAN2401. In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, or at least 23% relative to placebo as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 15% to 30% relative to placebo as determined by ADCOMS. In some embodiments, the clinical decline is reduced by at least 15%, such as by at 20%, at least 21%, or at least 23%, relative to placebo as determined by ADCOMS. In some embodiments, the clinical decline is reduced by at least 23% relative to placebo as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject who is concomitantly administered a therapeutically effective amount of at least one anti-Aβ protofibril antibody and a therapeutically effective amount of at least one Alzheimer's disease medication other than BAN2401 is reduced by at least 23% relative to placebo as determined by ADCOMS after 18 months of administration of the therapeutically effective amount of at least one anti-Aβ protofibril antibody. In some embodiments, 10 mg/kg of at least one anti-Aβ protofibril antibody is administered once every two weeks or once every month. In some embodiments, the at least one anti-Aβ protofibril antibody is BAN2401. In some embodiments, the at least one Alzheimer's disease medication is chosen from elenbecestat, donepezil, galantamine, memantine, and rivastigmine. In some embodiments, the at least one Alzheimer's disease medication is a combination of donepezil and memantine.

In some embodiments, donepezil may be administered at its approved dose. In some embodiments, galantamine may be administered at its approved dose. In some embodiments, memantine may be administered at its approved dose. In some embodiments, rivastigmine may be administered at its approved dose.

In some embodiments, elenbecestat may be administered at a dose ranging from 5 mg/ day to 100 mg/day, 10 mg/day to 75 mg/day, 5 mg/day to 50 mg/day, or 15 mg/day to 50 mg/day. In some embodiments, elenbecestat may be administered at a dose ranging from about 5 mg/ day to about 100 mg/day, about 10 mg/day to about 75 mg/day, about 5 mg/day to about 50 mg/day, or about 15 mg/day to about 50 mg/day. In some embodiments, elenbecestat may be administered at a dose of 5 mg/day, 10 mg/day, 15 mg/day, 20 mg/day, 25 mg/day, 30 mg/day, or 50 mg/day dosage. In some embodiments, elenbecestat may be administered at a dose of 5 mg/day. In some embodiments, elenbecestat may be administered at a dose of 15 mg/day. In some embodiments, elenbecestat may be administered at a dose of 50 mg/day.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, or at least 41% relative to placebo as determined by ADCOMS. In some embodiments, the above-recited reduction in clinical decline is determined after 1 month, 6 months, 12 months, 18 months, 60 months, and/or 63 months of administration of the therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, or at least 39% relative to placebo as determined by ADAS-cog, wherein the subject is concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 30% to 50% relative to placebo as determined by ADAS-cog, wherein the subject is concomitantly administered at least one Alzheimer's disease medication other than BAN2401. In some embodiments, the clinical decline is reduced by at least 30%, such as by at least 35%, at least 37%, or at least 39%, relative to placebo as determined by ADAS-cog, wherein the subject is concomitantly administered at least one Alzheimer's disease medication other than BAN2401. In some embodiments, the clinical decline is reduced by at least 39% relative to placebo as determined by ADAS-cog, wherein the subject is concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject who is concomitantly administered at least one Alzheimer's disease medication is reduced by at least 39% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401. In some embodiments, the at least one Alzheimer's disease medication is chosen from elenbecestat, donepezil, galantamine, memantine, and rivastigmine. In some embodiments, the at least one Alzheimer's disease medication is a combination of donepezil and memantine.

In some embodiments, donepezil may be administered at its approved dose. In some embodiments, galantamine may be administered at its approved dose. In some embodiments, memantine may be administered at its approved dose. In some embodiments, rivastigmine may be administered at its approved dose.

In some embodiments, elenbecestat may be administered at a dose ranging from 5 mg/ day to 100 mg/day, 10 mg/day to 75 mg/day, 5 mg/day to 50 mg/day, or 15 mg/day to 50 mg/day. In some embodiments, elenbecestat may be administered at a dose ranging from about 5 mg/ day to about 100 mg/day, about 10 mg/day to about 75 mg/day, about 5 mg/day to about 50 mg/day, or about 15 mg/day to about 50 mg/day. In some embodiments, elenbecestat may be administered at a dose of 5 mg/day, 10 mg/day, 15 mg/day, 20 mg/day, 25 mg/day, 30 mg/day, or 50 mg/day dosage. In some embodiments, elenbecestat may be administered at a dose of 5 mg/day. In some embodiments, elenbecestat may be administered at a dose of 15 mg/day. In some embodiments, elenbecestat may be administered at a dose of 50 mg/day.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% relative to placebo as determined by CDR-SB, wherein the subject is concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 10% to 30% relative to placebo as determined by CDR-SB, wherein the subject is concomitantly administered at least one Alzheimer's disease medication other than BAN2401. In some embodiments, the clinical decline is reduced by at least 10%, such as by at least 15%, at least 17%, or at least 20%, relative to placebo as determined by CDR-SB, wherein the subject is concomitantly administered at least one Alzheimer's disease medication other than BAN2401. In some embodiments, the clinical decline is reduced by at least 20% relative to placebo as determined by CDR-SB, wherein the subject is concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject who is concomitantly administered at least one Alzheimer's disease medication is reduced by at least 20% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401. In some embodiments, the at least one Alzheimer's disease medication is chosen from elenbecestat, donepezil, galantamine, memantine, and rivastigmine. In some embodiments, the at least one Alzheimer's disease medication is a combination of donepezil and memantine.

In some embodiments, donepezil may be administered at its approved dose. In some embodiments, galantamine may be administered at its approved dose. In some embodiments, memantine may be administered at its approved dose. In some embodiments, rivastigmine may be administered at its approved dose.

In some embodiments, elenbecestat may be administered at a dose ranging from 5 mg/ day to 100 mg/day, 10 mg/day to 75 mg/day, 5 mg/day to 50 mg/day, or 15 mg/day to 50 mg/day. In some embodiments, elenbecestat may be administered at a dose ranging from about 5 mg/ day to about 100 mg/day, about 10 mg/day to about 75 mg/day, about 5 mg/day to about 50 mg/day, or about 15 mg/day to about 50 mg/day. In some embodiments, elenbecestat may be administered at a dose of 5 mg/day, 10 mg/day, 15 mg/day, 20 mg/day, 25 mg/day, 30 mg/day, or 50 mg/day dosage. In some embodiments, elenbecestat may be administered at a dose of 5 mg/day. In some embodiments, elenbecestat may be administered at a dose of 15 mg/day. In some embodiments, elenbecestat may be administered at a dose of 50 mg/day.

### Reduction of Brain Amyloid Level

Also provided herein is a method of reducing brain amyloid level in a subject in need thereof comprising administering a composition comprising a therapeutically effective amount of at least one anti-Aβ protofibril antibody disclosed herein.

In some embodiments, the subject has early Alzheimer's disease. In some embodiments, the subject has Alzheimer's disease, Down's Syndrome, chronic traumatic encephalopathy, cerebral amyloid angiopathy, Lewy Body Dementia, or another brain disease or conditions with Aβ peptide-containing soluble and/or insoluble Aβ aggregates.

One of ordinary skill in the art will understand that, in addition to subjects having Alzheimer's disease, Aβ plaque deposits are present in the brains of subjects having other neurodegenerative diseases and conditions and thus that the methods disclosed herein will be beneficial for subjects having such neurodegenerative diseases and/or conditions. Such diseases and conditions are known to include, for example, Down's Syndrome, chronic traumatic encephalopathy, cerebral amyloid angiopathy, and Lewy Body Dementia. (See, e.g., Catafau et al., “Amyloid PET imaging: applications beyond Alzheimer's disease,” Clin. Transl. Imaging 3(1): 39-55 (2015); and Banerjee, G. et al., “The increasing impact of cerebral amyloid angiopathy: essential new insights for clinical practice,” J. Neurol. Neurosurg. Psychiatry 88: 982-994 (2017).)

In some embodiments, the subject having early Alzheimer's disease has been diagnosed as having mild cognitive impairment due to Alzheimer's disease—intermediate likelihood and/or has been diagnosed as having mild Alzheimer's disease dementia. In some embodiments, the subject having early Alzheimer's disease is ApoE4-positive.

Any of the anti-Aβ protofibril antibodies, therapeutically acceptable amounts thereof, dosing regimens therefor, and compositions comprising the same that are disclosed herein may be used in the method of reducing brain amyloid level in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month.

In some embodiments, the at least one anti-Aβ protofibril antibody is BAN2401.

In some embodiments, said method results in a reduced brain amyloid level after administration relative to the brain amyloid level prior to said administration. In some embodiments, the brain amyloid level is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the brain amyloid level prior to said administration. In some embodiments, the above-recited reductions in brain amyloid levels are determined by visual reads of amyloid PET images and are expressed as PET standard uptake value ratios (SUVr values).

Amyloid positron emission tomography (PET) imaging can be used to confirm the presence of amyloid pathology in the brain of early AD subjects in the screening phase of the study and/or to evaluate the effects of the at least one anti-AB antibody on amyloid levels in the brain, both by whole brain analysis (e.g., the average of 5-6 cortical regions) and brain region analysis. In some embodiments, the adjusted mean change from baseline in a subject's PET SUVr value is reduced by at least −0.10, at least −0.15, at least −0.20, at least −0.25, at least −0.30, at least −0.35, at least −0.40, at least −0.45, at least −0.50, at least−0.55, at least −0.60, at least −0.65, at least −0.70, at least −0.75, at least −0.80, at least −0.85, at least −0.90, or at least −0.95 relative to baseline. In some embodiments, the adjusted mean change from baseline in a subject's PET SUVr value is reduced by -0.20 to -0.30.

In some embodiments, comparing global cortical average versus whole cerebellum reference, the adjusted mean change from baseline in a subject's PET SUVr value is reduced by at least −0.20, such as at least −0.25, after 12 months of administration of the composition comprising a therapeutically effective amount of at least one anti-Aβ protofibril antibody. In some embodiments, the adjusted mean change from baseline in a subject's PET SUVr value is reduced by at least −0.25, such as at least −0.30, after 18 months of administration of the composition comprising a therapeutically effective amount of at least one anti-Aβ protofibril antibody.

In some embodiments, the reduction of amyloid in the brain is determined by imaging using binding of radiotracers for brain Aβ amyloid and visualized with PET. In some embodiments, the reduction in the adjusted mean change from baseline is at least −50, such as at least −55 or at least −59 centiloid after 12 months of administration of the composition comprising a therapeutically effective amount of at least one anti-Aβ protofibril antibody. In some embodiments, the reduction in the adjusted mean change from baseline is at least −60, such as at least −65 or at least −70 centiloid after 18 months of administration of the composition comprising a therapeutically effective amount of at least one anti-Aβ protofibril antibody.

In some embodiments, said method results in a reduced cerebrospinal fluid Aβ₁₋₄₂ level relative to the cerebrospinal fluid Aβ₁₋₄₂ level prior to said administration. In some embodiments, said method results in a reduction of cerebrospinal fluid Aβ₁₋₄₂ level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Aβ₁₋₄₂ level prior to said administration.

In some embodiments, administration of the composition results in a brain amyloid level reduction of −0.20 to −0.45, such as from −0.25 to −0.35 as determined by visual reads of amyloid PET images, wherein the subject is ApoE4-positive. In some embodiments, administration of the composition results in a brain amyloid level reduction of at least −0.25, as determined by visual reads of amyloid PET images, wherein the subject is ApoE4-positive. In some embodiments, administration of the composition results in a brain amyloid level reduction of at least 0.30, as determined by visual reads of amyloid PET images, wherein the subject is ApoE4-positive.

In some embodiments, administration of the composition results in a brain amyloid level reduction of at least −0.01, at least −0.02, at least −0.03, at least −0.04, at least −0.05, at least −0.06, at least −0.07, at least −0.08, at least −0.09, at least −0.10, at least −0.11, at least −0.12, at least −0.13, at least −0.14, at least −0.15, at least −0.16, at least −0.17, at least −0.18, at least −0.19, at least −0.20, at least −0.21, at least −0.22, at least −0.23, at least −0.24, at least −0.25, at least −0.26, at least −0.27, at least −0.28, or at least −0.29 relative to placebo, as determined by visual reads of amyloid PET images, wherein the subject is ApoE4-negative.

In some embodiments, administration of the composition results in a brain amyloid level reduction of −0.10 to −0.40, as determined by visual reads of amyloid PET images, wherein the subject is ApoE4-negative. In some embodiments, administration of the composition results in a brain amyloid level reduction of at least −0.20, as determined by visual reads of amyloid PET images, wherein the subject is ApoE4-negative. In some embodiments, administration of the composition results in a brain amyloid level reduction of at least −0.25, as determined by visual reads of amyloid PET images, wherein the subject is ApoE4-negative.

### Biomarker Changes

### Cerebrospinal Fluid Level of Neurogranin

In some embodiments, administration of a composition comprising a therapeutically effective amount of at least one anti-Aβ protofibril antibody disclosed herein to a subject results in a reduction in cerebrospinal fluid level of neurogranin in the subject. In some embodiments, the administration to a subject of a composition comprising a therapeutically effective amount of at least one anti-protofibril antibody disclosed herein results in a reduction of at least 1%, at least 2%, at least 3%, at least 4%, at least 5%, at least 6%, at least 7%, at least 8%, at least 9%, or at least 10%, relative to baseline, in cerebrospinal fluid level of neurogranin.

In some embodiments, administration to a subject of a composition comprising a therapeutically effective amount of at least one anti-Aβ protofibril antibody disclosed herein results in a reduction in cerebrospinal fluid level of neurogranin after 18 months of administration of the composition comprising a therapeutically effective amount of at least one anti-Aβ protofibril antibody. In some embodiments, administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, or at least 10%, relative to baseline, cerebrospinal fluid level of neurogranin after 18 months of administration of the composition.

In some embodiments, administration to a subject of a composition comprising a therapeutically effective amount of at least one anti-Aβ protofibril antibody disclosed herein results in a reduction of at least about 25 pg/mL, at least about 30 pg/mL, at least about 35 pg/mL, at least about 40 pg/mL, at least about 45 pg/mL, at least about 50 pg/mL, at least about 55 pg/mL, at least about 60 pg/mL, or at least about 65 pg/mL, relative to baseline, cerebrospinal fluid level of neurogranin. In some embodiments, administration to a subject of a composition comprising a therapeutically effective amount of at least one anti-Aβ protofibril antibody disclosed herein results in a reduction of at least about 65 pg/mL, relative to baseline, cerebrospinal fluid level of neurogranin.

In some embodiments, administration to a subject of a composition comprising a therapeutically effective amount of at least one anti-Aβ protofibril antibody disclosed herein results in a reduction of at least about 25 pg/mL, at least about 30 pg/mL, at least about 35 pg/mL, at least about 40 pg/mL, at least about 45 pg/mL, at least about 50 pg/mL, at least about 55 pg/mL, at least about 60 pg/mL, or at least about 65 pg/mL, relative to baseline, of cerebrospinal fluid level of neurogranin after 18 months of administration of the composition comprising a therapeutically effective amount of at least one anti-Aβ protofibril antibody. In some embodiments, administering to a subject a composition comprising a therapeutically effective amount of at least one anti-Aβ protofibril antibody disclosed herein results in a reduction of at least 65 pg/mL, relative to baseline, of cerebrospinal fluid level of neurogranin after 18 months of administration of the composition.

In some embodiments, the at least one anti-Aβ protofibril antibody is BAN2401.

In some embodiments, the therapeutically effective amount of at least one anti-Aβ protofibril antibody is 10 mg/kg. In some embodiments, the composition comprising a therapeutically effective amount of at least one anti-Aβ protofibril antibody disclosed herein is administered bi-weekly or monthly. In some embodiments, a composition comprising 10 mg/kg of BAN2401 is administered bi-weekly. In some embodiments, a composition comprising 10 mg/kg of BAN2401 is administered monthly.

### Cerebrospinal Fluid Level of Neurofilament Light Chain

In some embodiments, administration to a subject of a composition comprising a therapeutically effective amount of at least one anti-Aβ protofibril antibody disclosed herein results in a reduction, relative to placebo, in cerebrospinal fluid level of neurofilament light chain. In some embodiments, administration to a subject of a composition comprising a therapeutically effective amount of at least one anti-Aβ protofibril antibody disclosed herein results in a reduction of at least 5%, at least 10%, at least 15%, at least 20%, at least 25%, at least 30%, at least 35%, at least 40%, at least 45%, or at least 50%, relative to placebo, in cerebrospinal fluid level of neurofilament light chain.

In some embodiments, administration to a subject of a composition comprising a therapeutically effective amount of at least one anti-Aβ protofibril antibody disclosed herein results in a reduction, relative to placebo, in cerebrospinal fluid level of neurofilament light chain after 18 months of administration of the composition. In some embodiments, administration to a subject of a composition comprising a therapeutically effective amount of at least one anti-Aβ protofibril antibody disclosed herein results in a reduction, relative to placebo, of at least 5%, at least 10%, at least 15%, at least 20%, at least 25%, at least 30%, at least 35%, at least 40%, at least 45%, or at least 50%, relative to baseline, in cerebrospinal fluid level of neurofilament light chain after 18 months of administration of the composition.

In some embodiments, administration to a subject of a composition comprising a therapeutically effective amount of at least one anti-Aβ protofibril antibody disclosed herein results in production of more than about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, relative to baseline, of cerebrospinal fluid level of neurofilament light chain. In some embodiments, administration to a subject of a composition comprising a therapeutically effective amount of at least one anti-Aβ protofibril antibody disclosed herein results in production of no more than about 75 pg/mL, relative to baseline, of cerebrospinal fluid level of neurofilament light chain.

In some embodiments, administration to a subject of a composition comprising a therapeutically effective amount of at least one anti-Aβ protofibril antibody disclosed herein results in production of more than about 35 pg/mL, about 40 pg/mL, about 45 pg/mL, about 50 pg/mL, about 55 pg/mL, about 60 pg/mL, about 65 pg/mL, about 70 pg/mL, about 75 pg/mL, relative to baseline, of cerebrospinal fluid level of neurofilament light chain after 18 months of administration of the composition. In some embodiments, administration to a subject of a composition comprising a therapeutically effective amount of at least one anti-Aβ protofibril antibody disclosed herein results in production of no more than about 75 pg/mL, relative to baseline, of cerebrospinal fluid level of neurofilament light chain after 18 months of administration of the composition.

In some embodiments, the at least one anti-Aβ protofibril antibody is BAN2401.

In some embodiments, the therapeutically effective amount of at least one anti-Aβ protofibril antibody disclosed herein is 10 mg/kg. In some embodiments, a composition comprising a therapeutically effective amount of at least one anti-Aβ protofibril antibody disclosed herein is administered bi-weekly or monthly. In some embodiments, a composition comprising 10 mg/kg of BAN2401 is administered bi-weekly. In some embodiments, a composition comprising 10 mg/kg of BAN2401 is administered monthly.

### Cerebrospinal Fluid Level of Phospho-Tau

In some embodiments, administration to a subject of a composition comprising a therapeutically effective amount of at least one anti-Aβ protofibril antibody disclosed herein results in a reduction in cerebrospinal fluid level of phospho-Tau. In some embodiments, administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, at least 10%, at least 11%, at least 12%, or at least 13% relative to baseline, of cerebrospinal fluid level of phospho-Tau.

In some embodiments, administration to a subject of a composition comprising a therapeutically effective amount of at least one anti-Aβ protofibril antibody disclosed herein results in a reduction in cerebrospinal fluid level of phospho-Tau after 18 months of administration of the composition. In some embodiments, administration to a subject of a composition comprising a therapeutically effective amount of at least one anti-Aβ protofibril antibody disclosed herein results in a reduction of at least 1%, at least 2%, at least 3%, at least 4%, at least 5%, at least 6%, at least 7%, at least 8%, at least 9%, at least 10%, at least 11%, at least 12%, or at least 13%, relative to baseline, of cerebrospinal fluid level of phospho-Tau after 18 months of administration of the composition comprising a therapeutically effective amount of at least one anti-Aβ protofibril antibody.

In some embodiments, administration to a subject of a composition comprising a therapeutically effective amount of at least one anti-Aβ protofibril antibody disclosed herein results in a reduction of at least about 65 pg/mL, at least about 70 pg/mL, at least about 75 pg/mL, at least about 80 pg/mL, at least about 85 pg/mL, at least about 90 pg/mL, or at least about 95 pg/mL, relative to baseline, of cerebrospinal fluid level of phospho-Tau. In some embodiments, administration to a subject of a composition comprising a therapeutically effective amount of at least one anti-Aβ protofibril antibody disclosed herein results in a reduction of at least about 95 pg/mL, relative to baseline, of cerebrospinal fluid level of phospho-Tau.

In some embodiments, administration to a subject of a composition comprising a therapeutically effective amount of at least one anti-Aβ protofibril antibody disclosed herein results in a reduction of at least about 65 pg/mL, at least about 70 pg/mL, at least about 75 pg/mL, at least about 80 pg/mL, at least about 85 pg/mL, at least about 90 pg/mL, or at least about 95 pg/mL, relative to baseline, of cerebrospinal fluid level of phospho-Tau after 18 months of administration of the composition comprising a therapeutically effective amount of at least one anti-Aβ protofibril antibody. In some embodiments, administration to a subject of a composition comprising a therapeutically effective amount of at least one anti-Aβ protofibril antibody disclosed herein results in a reduction of at least 95 pg/mL, relative to baseline, of cerebrospinal fluid level of phospho-Tau after 18 months of administration of the composition comprising a therapeutically effective amount of at least one anti-Aβ protofibril antibody.

In some embodiments, the at least one anti-Aβ protofibril antibody is BAN2401.

In some embodiments, the therapeutically effective amount of at least one anti-Aβ protofibril antibody is 10 mg/kg. In some embodiments, the composition comprising a therapeutically effective amount of at least one anti-Aβ protofibril antibody disclosed herein is administered bi-weekly or monthly. In some embodiments, a composition comprising 10 mg/kg of BAN2401 is administered bi-weekly. In some embodiments, a composition comprising 10 mg/kg of BAN2401 is administered monthly.

### Methods of Treating Alzheimer's Disease

### Reduction in Clinical Decline

Provided herein is a method of treating a subject having early Alzheimer's disease comprising administering to said subject a composition comprising a therapeutically effective amount of at least one anti-Aβ protofibril antibody disclosed herein, wherein clinical decline of the subject is reduced by at least 35% relative to placebo as determined by ADCOMS after 6 months of administration of the composition, by at least 30% relative to placebo as determined by ADCOMS after 12 months of administration of the composition, and/or by at least 25% relative to placebo as determined by ADCOMS after 18 months of administration of the composition. In some embodiments, the subject having early Alzheimer's disease has been diagnosed as having mild cognitive impairment due to Alzheimer's disease—intermediate likelihood and/or has been diagnosed as having mild Alzheimer's disease dementia. In some embodiments, the subject having early Alzheimer's disease is ApoE4-positive.

Any of the anti-Aβ protofibril antibodies, therapeutically acceptable amounts thereof, dosing regimens therefor, and compositions comprising the same that are disclosed herein may be used in the method of reducing clinical decline in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month. In some embodiments, the clinical decline is reduced by at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, or at least 46% relative to placebo as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 35% relative to placebo as determined by ADCOMS. In some embodiments, the clinical decline is reduced by 20% to 30% relative to placebo as determined by ADCOMS. In some embodiments, the clinical decline is reduced by 27% to 35% relative to placebo as determined by ADCOMS. In some embodiments, the clinical decline is reduced by at least 20% relative to placebo as determined by ADCOMS. In some embodiments, the clinical decline is reduced by at least 35% relative to placebo as determined by ADCOMS. In some embodiments, the clinical decline is reduced by at least 20% as determined by ADCOMS. In some embodiments, the clinical decline is reduced by at least 30% as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 45% relative to placebo as determined by ADCOMS after 6 months of administration of the composition comprising a therapeutically effective amount of at least one anti-Aβ protofibril antibody. In some embodiments, the clinical decline is reduced by at least 35% relative to placebo as determined by ADCOMS after 12 months of administration of the composition comprising a therapeutically effective amount of at least one anti-Aβ protofibril antibody. In some embodiments, the clinical decline is reduced by at least 30% relative to placebo as determined by ADCOMS after 18 months of administration of the composition comprising a therapeutically effective amount of at least one anti-Aβ protofibril antibody. In some embodiments, the clinical decline is reduced by at least 46% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, or at least 52% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 33% relative to placebo as determined by ADCOMS, wherein the subject has been diagnosed as having mild cognitive impairment due to Alzheimer's disease—intermediate likelihood. In some embodiments, the clinical decline is reduced by at least 20%, such as by at 25% or at least 28%, relative to placebo as determined by ADCOMS, wherein the subject has been diagnosed as having mild cognitive impairment due to Alzheimer's disease—intermediate likelihood. In some embodiments, the clinical decline is reduced by at least 25%, such as by at least 30% or at least 33%, relative to placebo as determined by ADCOMS, wherein the subject has been diagnosed as having mild cognitive impairment due to Alzheimer's disease—intermediate likelihood. In some embodiments, the clinical decline is reduced by at least 25%, such as by at least 30%, at least 35%, at least 40%, at least 45%, at least 50%, or at least 52% relative to placebo as determined by ADCOMS, wherein the subject has been diagnosed as having mild cognitive impairment due to Alzheimer's disease—intermediate likelihood. In some embodiments, the clinical decline is reduced by at least 52% relative to placebo as determined by ADCOMS, wherein the subject has been diagnosed as having mild cognitive impairment due to Alzheimer's disease-13 intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 30% relative to placebo as determined by ADCOMS after 6 months of administration of the composition comprising a therapeutically effective amount of at least one anti-Aβ protofibril antibody. In some embodiments, the clinical decline in the subject diagnosed as having mild cognitive impairment due to Alzheimer's disease—intermediate likelihood is reduced by at least 25% relative to placebo as determined by ADCOMS after 12 months of administration of the composition comprising a therapeutically effective amount of at least one anti-Aβ protofibril antibody. In some embodiments, the clinical decline in the subject diagnosed as having mild cognitive impairment due to Alzheimer's disease—intermediate likelihood is reduced by at least 30% relative to placebo as determined by ADCOMS after 18 months of administration of the composition comprising a therapeutically effective amount of at least one anti-Aβ protofibril antibody. In some embodiments, the clinical decline in the subject diagnosed as having mild cognitive impairment due to Alzheimer's disease—intermediate likelihood is reduced by at least 52% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, or at least 33% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 38% relative to placebo as determined by ADCOMS, wherein the subject has been diagnosed as having mild cognitive impairment due to Alzheimer's disease—intermediate likelihood. In some embodiments, the clinical decline is reduced by at least 20%, such as by at 25%, at least 28%, or at least 33%, relative to placebo as determined by ADCOMS, wherein the subject has been diagnosed as having mild cognitive impairment due to Alzheimer's disease—intermediate likelihood. In some embodiments, the clinical decline is reduced by at least 25%, such as by at least 30% or at least 33%, relative to placebo as determined by ADCOMS, wherein the subject has been diagnosed as having mild cognitive impairment due to Alzheimer's disease—intermediate likelihood. In some embodiments, the clinical decline is reduced by at least 33% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 33% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, or at least 78% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 80% relative to placebo as determined by ADCOMS, wherein the subject has been diagnosed as having Alzheimer's disease dementia. In some embodiments, the clinical decline is reduced by 35% to 78% relative to placebo as determined by ADCOMS, wherein the subject has been diagnosed as having Alzheimer's disease dementia. In some embodiments, the clinical decline is reduced by at least 35% relative to placebo as determined by ADCOMS, wherein the subject has been diagnosed as having mild Alzheimer's disease dementia. In some embodiments, the clinical decline is reduced by at least 50%, such as by at least 52% or at least 53% relative to placebo as determined by ADCOMS, wherein the subject has been diagnosed as having mild Alzheimer's disease dementia. In some embodiments, the clinical decline is reduced by at least 70%, such as by at least 75% or at least 78%, relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 70% relative to placebo as determined by ADCOMS after 6 months of administration of the composition comprising a therapeutically effective amount of at least one anti-Aβ protofibril antibody. In some embodiments, the clinical decline in the subject diagnosed as having mild Alzheimer's disease dementia is reduced by at least 50% relative to placebo as determined by ADCOMS after 12 months of administration of the composition comprising a therapeutically effective amount of at least one anti-Aβ protofibril antibody. In some embodiments, the clinical decline in the subject diagnosed as having mild Alzheimer's disease dementia is reduced by at least 30% relative to placebo as determined by ADCOMS after 18 months of administration of the composition comprising a therapeutically effective amount of at least one anti-Aβ protofibril antibody. In some embodiments, the clinical decline in the subject diagnosed as having mild Alzheimer's disease dementia is reduced by at least 52% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, or at least 35% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 38% relative to placebo as determined by ADCOMS, wherein the subject has been diagnosed as having mild Alzheimer's disease dementia. In some embodiments, the clinical decline is reduced by at least 20%, such as by at 25%, at least 28%, or at least 35%, relative to placebo as determined by ADCOMS, wherein the subject has been diagnosed as having mild Alzheimer's disease dementia. In some embodiments, the clinical decline is reduced by at least 25%, such as by at least 30% or at least 35%, relative to placebo as determined by ADCOMS, wherein the subject has been diagnosed as having mild Alzheimer's disease dementia. In some embodiments, the clinical decline is reduced by at least 35% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 35% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 75%, at least 80%, at least 85%, at least 90%, at least 95%, at least 100%, at least 110%, at least 120%, at least 130%, at least 140%, or at least 150% relative to placebo as determined by ADAS-cog. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 150% relative to placebo as determined by ADAS-cog. In some embodiments, the clinical decline is reduced by 45% to 145% relative to placebo as determined by ADAS-cog. In some embodiments, the clinical decline is reduced by 45% to 55% relative to placebo as determined by ADAS-cog. In some embodiments, the clinical decline is reduced by at least 30% relative to placebo as determined by ADAS-cog. In some embodiments, the clinical decline is reduced by at least 35% relative to placebo as determined by ADAS-cog. In some embodiments, the clinical decline is reduced by at least 40% as determined by ADAS-cog. In some embodiments, the clinical decline is reduced by at least 45% as determined by ADAS-cog. In some embodiments, the clinical decline is reduced by at least 47% as determined by ADAS-cog. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 100%, such as at least 120% or at least 140%, relative to placebo as determined by ADAS-cog after 6 months of administration of the composition comprising a therapeutically effective amount of at least one anti-Aβ protofibril antibody. In some embodiments, the clinical decline is reduced by at least 40%, such as at least 45%, relative to placebo as determined by ADAS-cog after 12 months of administration of the composition comprising a therapeutically effective amount of at least one anti-Aβ protofibril antibody. In some embodiments, the clinical decline is reduced by at least 40%, such as at least 45%, relative to placebo as determined by ADAS-cog after 18 months of administration of the composition comprising a therapeutically effective amount of at least one anti-Aβ protofibril antibody. In some embodiments, the clinical decline is reduced by at least 47%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 56%, at least 47%, at least 48%, at least 49%, at least 50%, at least 51%, at least 52%, at least 53%, at least 54%, at least 55%, at least 56%, at least 57%, or at least 58% relative to placebo as determined by ADAS-cog, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject has been diagnosed as having mild cognitive impairment due to Alzheimer's disease—intermediate likelihood. In some embodiments, the clinical decline is reduced by at least 50%, such as by at 52%, at least 55%, or at least 58%, relative to placebo as determined by ADAS-cog, wherein the subject has been diagnosed as having mild cognitive impairment due to Alzheimer's disease—intermediate likelihood. In some embodiments, the clinical decline is reduced by at least 58% relative to placebo as determined by ADAS-cog, wherein the subject has been diagnosed as having mild cognitive impairment due to Alzheimer's disease-13 intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 58% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, or at least 41% relative to placebo as determined by ADAS-cog, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 30% to 50% relative to placebo as determined by ADAS-cog, wherein the subject has been diagnosed as having mild Alzheimer's disease dementia. In some embodiments, the clinical decline is reduced by at least 35%, such as by at 38%, at least 40%, or at least 41%, relative to placebo as determined by ADAS-cog, wherein the subject has been diagnosed as having mild Alzheimer's disease dementia. In some embodiments, the clinical decline is reduced by at least 41%, relative to placebo as determined by ADAS-cog, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 41% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40% relative to placebo as determined by CDR-SB. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 60% relative to placebo as determined by CDR-SB. In some embodiments, the clinical decline is reduced by 25% to 60% relative to placebo as determined by CDR-SB. In some embodiments, the clinical decline is reduced by 25% to 50% relative to placebo as determined by CDR-SB. In some embodiments, the clinical decline is reduced by at least 20% relative to placebo as determined by CDR-SB. In some embodiments, the clinical decline is reduced by at least 30% relative to placebo as determined by CDR-SB. In some embodiments, the clinical decline is reduced by at least 25%, such as at least 26% or at least 28%, as determined by CDR-SB. In some embodiments, the clinical decline is reduced by at least 30%, such as at least 35% or at least 38%, as determined by CDR-SB. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 30%, such as at least 35% or at least 40%, relative to placebo as determined by CDR-SB after 6 months of administration of the composition comprising a therapeutically effective amount of at least one anti-Aβ protofibril antibody. In some embodiments, the clinical decline is reduced by at least 30%, such as at least 35% or at least 45%, relative to placebo as determined by CDR-SB after 12 months of administration of the composition comprising a therapeutically effective amount of at least one anti-Aβ protofibril antibody. In some embodiments, the clinical decline is reduced by at least 20%, such as at least 25%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, or at least 14% relative to placebo as determined by CDR-SB, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 10% to 20% relative to placebo as determined by CDR-SB, wherein the subject has been diagnosed as having mild cognitive impairment due to Alzheimer's disease—intermediate likelihood. In some embodiments, the clinical decline is reduced by at least 5%, such as by at 10%, at least 12%, or at least 14%, relative to placebo as determined by CDR-SB, wherein the subject has been diagnosed as having mild cognitive impairment due to Alzheimer's disease—intermediate likelihood. In some embodiments, the clinical decline is reduced by at least 14% relative to placebo as determined by CDR-SB, wherein the subject has been diagnosed as having mild cognitive impairment due to Alzheimer's disease-13 intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 14% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, or at least 51% relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 60% relative to placebo as determined by CDR-SB, wherein the subject has been diagnosed as having mild Alzheimer's disease dementia. In some embodiments, the clinical decline is reduced by at least 45%, such as by at 48%, at least 50%, or at least 51%, relative to placebo as determined by CDR-SB, wherein the subject has been diagnosed as having mild Alzheimer's disease dementia. In some embodiments, the clinical decline is reduced by at least 51% relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 51% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of at least one anti-Aβ protofibril antibody. In some embodiments, the reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the reduction in clinical decline is determined after 1 month of administration of the composition comprising a therapeutically effective amount of at least one anti-Aβ protofibril antibody. In some embodiments, the reduction in clinical decline is determined after 6 months of administration of the composition comprising a therapeutically effective amount of at least one anti-Aβ protofibril antibody. In some embodiments, the reduction in clinical decline is determined after 12 months of administration of the composition comprising a therapeutically effective amount of at least one anti-Aβ protofibril antibody. In some embodiments, the reduction in clinical decline is determined after 18 months of administration of the composition comprising a therapeutically effective amount of at least one anti-Aβ protofibril antibody. In some embodiments, the reduction in clinical decline is determined after 60 months of administration of the composition comprising a therapeutically effective amount of at least one anti-Aβ protofibril antibody. In some embodiments, the reduction in clinical decline is determined after 63 months of administration of the composition comprising a therapeutically effective amount of at least one anti-Aβ protofibril antibody.

In some embodiments, the reduction in clinical decline is determined after administration of a composition comprising a therapeutically effective amount of BAN2401.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of BAN2401. In some embodiments, the reduction in clinical decline is determined after 1 month, 6 months, 12 months, 18 months, 60 months, and/or 63 months of administration of the composition comprising a therapeutically effective amount of BAN2401. In some embodiments, the reduction in clinical decline is determined after 1 month of administration of the composition comprising a therapeutically effective amount of BAN2401. In some embodiments, the reduction in clinical decline is determined after 6 months of administration of the composition comprising a therapeutically effective amount of BAN2401. In some embodiments, the reduction in clinical decline is determined after 12 months of administration of the composition comprising a therapeutically effective amount of BAN2401. In some embodiments, the reduction in clinical decline is determined after 18 months of administration of the composition comprising a therapeutically effective amount of BAN2401. In some embodiments, the reduction in clinical decline is determined after 60 months of administration of the composition comprising a therapeutically effective amount of BAN2401. In some embodiments, the reduction in clinical decline is determined after 63 months of administration of the composition comprising a therapeutically effective amount of BAN2401.

In some embodiments, the subject is ApoE4-positive.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, or at least 74% relative to placebo as determined by ADCOMS, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 60% to 80%, such as by 63% to 74%, relative to placebo as determined by ADCOMS, wherein the subject is ApoE4-positive. In some embodiments, the clinical decline is reduced by at least 60%, such as at least 63%, relative to placebo as determined by ADCOMS, wherein the subject is ApoE4-positive. In some embodiments, the clinical decline is reduced by at least 65%, such as at least 67%, relative to placebo as determined by ADCOMS, wherein the subject is ApoE4-positive. In some embodiments, the clinical decline is reduced by at least 70%, such as at least 74%, relative to placebo as determined by ADCOMS, wherein the subject is ApoE4-positive.

In some embodiments, the clinical decline in the ApoE4-positive subject is reduced by at least 70% relative to placebo as determined by ADCOMS after 6 months of administration of the composition comprising a therapeutically effective amount of at least one anti-Aβ protofibril antibody. In some embodiments, the clinical decline in the ApoE4-positive subject is reduced by at least 60% relative to placebo as determined by ADCOMS after 12 months of administration of the composition comprising a therapeutically effective amount of at least one anti-Aβ protofibril antibody. In some embodiments, the clinical decline in the ApoE4-positive subject is reduced by at least 50%, such as at least 55% or at least 60%, relative to placebo as determined by ADCOMS after 18 months of administration of the composition comprising a therapeutically effective amount of at least one anti-Aβ protofibril antibody. In some embodiments, the clinical decline in the ApoE4-positive subject is reduced by at least 63%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 290%, at least 295%, at least 300%, at least 305%, at least 310%, at least 315%, at least 320%, at least 325%, at least 330%, or at least 331% relative to placebo as determined by ADAS-cog, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced 70% to 400%, such as 80% to 350%, relative to placebo as determined by ADAS-cog, wherein the subject is ApoE4-positive. In some embodiments, the clinical decline is reduced by at least 70%, such as at least 75% or at least 80%, relative to placebo as determined by ADAS-cog, wherein the subject is ApoE4-positive. In some embodiments, the clinical decline is reduced by at least 80%, such as at least 90% or at least 100%, relative to placebo as determined by ADAS-cog, wherein the subject is ApoE4-positive. In some embodiments, the clinical decline is reduced by at least 300%, such as at least 330%, relative to placebo as determined by ADAS-cog, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject is reduced by at least 300% relative to placebo as determined by ADAS-cog after 6 months of administration of the composition comprising a therapeutically effective amount of at least one anti-Aβ protofibril antibody. In some embodiments, the clinical decline in the ApoE4-positive subject is reduced by at least 80%, such as at least 90% or at least 100%, relative to placebo as determined by ADAS-cog after 12 months of administration of the composition comprising a therapeutically effective amount of at least one anti-Aβ protofibril antibody. In some embodiments, the clinical decline in the ApoE4-positive subject is reduced by at least 70%, such as at least 75%, at least 80%, or at least 84%, relative to placebo as determined by ADAS-cog after 18 months of administration of the composition comprising a therapeutically effective amount of at least one anti-Aβ protofibril antibody. In some embodiments, the clinical decline in the ApoE4-positive subject is reduced by at least 84%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, or at least 87% relative to placebo as determined by CDR-SB, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 35% to 150%, such as 40% to 100% or 45% to 90%, relative to placebo as determined by CDR-SB, wherein the subject is ApoE4-positive. In some embodiments, the clinical decline is reduced by at least 35%, such as at least 40% or at least 45%, relative to placebo as determined by CDR-SB, wherein the subject is ApoE4-positive. In some embodiments, the clinical decline is reduced by at least 50%, such as at least 55% or at least 60%, relative to placebo as determined by CDR-SB, wherein the subject is ApoE4-positive. In some embodiments, the clinical decline is reduced by at least 70%, such as at least 80% or at least 85%, relative to placebo as determined by CDR-SB, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject is reduced by at least 35%, such as at least 40% or at least 45%, relative to placebo as determined by CDR-SB after 6 months of administration of the composition comprising a therapeutically effective amount of at least one anti-Aβ protofibril antibody. In some embodiments, the clinical decline in the ApoE4-positive subject is reduced by at least 70%, such as at least 75% or at least 80%, relative to placebo as determined by CDR-SB after 12 months of administration of the composition comprising a therapeutically effective amount of at least one anti-Aβ protofibril antibody. In some embodiments, the clinical decline in the ApoE4-positive subject is reduced by at least 50%, such as at least 55% or at least 60%, relative to placebo as determined by CDR-SB after 18 months of administration of the composition comprising a therapeutically effective amount of at least one anti-Aβ protofibril antibody. In some embodiments, the clinical decline in the ApoE4-positive subject is reduced by at least 60%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, or at least 59% relative to placebo as determined by ADCOMS, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 30% to 70%, such as 38% to 59%, relative to placebo as determined by ADCOMS, wherein the subject is ApoE4-positive, and wherein the subject has been diagnosed as having mild cognitive impairment due to Alzheimer's disease—intermediate likelihood. In some embodiments, the clinical decline is reduced by at least 30%, such as at least 35% or at least 38%, relative to placebo as determined by ADCOMS, wherein the subject is ApoE4-positive, and wherein the subject has been diagnosed as having mild cognitive impairment due to Alzheimer's disease—intermediate likelihood. In some embodiments, the clinical decline is reduced by at least 45%, such as at least 50% or at least 53%, relative to placebo as determined by ADCOMS, wherein the subject is ApoE4-positive, and wherein the subject has been diagnosed as having mild cognitive impairment due to Alzheimer's disease—intermediate likelihood. In some embodiments, the clinical decline is reduced by at least 50%, such as at least 55% or at least 59%, relative to placebo as determined by ADCOMS, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease—intermediate likelihood is reduced by at least 50%, such as at least 55%, relative to placebo as determined by ADCOMS after 6 months of administration of the composition comprising a therapeutically effective amount of at least one anti-Aβ protofibril antibody. In some embodiments, the clinical decline in the ApoE4-positive subject diagnosed as mild cognitive impairment due to Alzheimer's disease—intermediate likelihood is reduced by at least 30%, such as at least 35%, relative to placebo as determined by ADCOMS after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease—intermediate likelihood is reduced by at least 45%, such as at least 50% or at least 55%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, or at least 211% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild Alzheimer's disease dementia is reduced by at least 100%, such as at least 110%, relative to placebo as determined by ADCOMS after 6 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 100%, such as at least 110%, relative to placebo as determined by ADCOMS after 12 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 65%, such as at least 70% or at least 75%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, or at least 211% relative to placebo as determined by ADAS-Cog, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 300%, such as 45% to 250% or 50% to 250%, relative to placebo as determined by ADAS-Cog, wherein the subject is ApoE4-positive, and wherein the subject has been diagnosed as having mild cognitive impairment due to Alzheimer's disease—intermediate likelihood. In some embodiments, the clinical decline is reduced by at least 40%, such as at least 45% or at least 50%, relative to placebo as determined by ADAS-Cog, wherein the subject is ApoE4-positive, and wherein the subject has been diagnosed as having mild cognitive impairment due to Alzheimer's disease—intermediate likelihood. In some embodiments, the clinical decline is reduced by at least 60, such as at least 70%, at least 75%, or at least 80%, relative to placebo as determined by ADAS-Cog, wherein the subject is ApoE4-positive, and wherein the subject has been diagnosed as having mild cognitive impairment due to Alzheimer's disease—intermediate likelihood. In some embodiments, the clinical decline is reduced by at least 100%, such as at least 150% or at least 200% relative to placebo as determined by ADAS-Cog, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease—intermediate likelihood is reduced by at least 100%, such as at least 150% or at least 200%, relative to placebo as determined by ADAS-cog after 6 months of administration of the composition comprising a therapeutically effective amount of at least one anti-Aβ protofibril antibody. In some embodiments, the clinical decline in the ApoE4-positive subject diagnosed as mild cognitive impairment due to Alzheimer's disease—intermediate likelihood is reduced by at least 40%, such as at least 45% or at least 50%, relative to placebo as determined by ADAS-cog after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease-13 intermediate likelihood is reduced by at least 50%, such as at least 60%, at least 70%, or at least 75%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45% relative to placebo as determined by CDR-SB, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 90%, such as 25% to 80% or 30% to 75%, relative to placebo as determined by CDR-SB, wherein the subject is ApoE4-positive, and wherein the subject has been diagnosed as having mild cognitive impairment due to Alzheimer's disease—intermediate likelihood. In some embodiments, the clinical decline is reduced by at least 25%, such as at least 30%, relative to placebo as determined by CDR-SB, wherein the subject is ApoE4-positive, and wherein the subject has been diagnosed as having mild cognitive impairment due to Alzheimer's disease—intermediate likelihood. In some embodiments, the clinical decline is reduced by at least 30%, such as at least 35% or 40%, relative to placebo as determined by CDR-SB, wherein the subject is ApoE4-positive, and wherein the subject has been diagnosed as having mild cognitive impairment due to Alzheimer's disease—intermediate likelihood. In some embodiments, the clinical decline is reduced by at least 35%, such as at least 40% or 45%, relative to placebo as determined by CDR-SB, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease—intermediate likelihood is reduced by at least 35%, such as at least 40% or at least 45%, relative to placebo as determined by CDR-SB composition comprising a therapeutically effective amount of at least one anti-Aβ protofibril antibody. In some embodiments, the clinical decline in the ApoE4-positive subject diagnosed as mild cognitive impairment due to Alzheimer's disease—intermediate likelihood is reduced by at least 20%, such as at least 25% or at least 30%, relative to placebo as determined by CDR-SB after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease—intermediate likelihood is reduced by at least 35%, such as at least 40%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, or at least 119% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 76% to 119% relative to placebo as determined by ADCOMS, wherein the subject is ApoE4-positive, and wherein the subject has been diagnosed as having mild Alzheimer's disease dementia. In some embodiments, the clinical decline is reduced by 76% relative to placebo as determined by ADCOMS, wherein the subject is ApoE4-positive, and wherein the subject has been diagnosed as having mild Alzheimer's disease dementia. In some embodiments, the clinical decline is reduced by 113% relative to placebo as determined by ADCOMS, wherein the subject is ApoE4-positive, and wherein the subject has been diagnosed as having mild Alzheimer's disease dementia. In some embodiments, the clinical decline is reduced by 119% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, at least 119%, at least 120%, at least 121%, at least 122%, at least 123%, at least 124%, at least 125%, at least 126%, at least 127%, at least 128%, at least 129%, at least 130%, at least 131%, at least 132%, at least 133%, at least 134%, at least 135%, at least 136%, at least 137%, at least 138%, at least 139%, at least 140%, at least 141%, at least 142%, at least 143%, at least 144%, at least 145%, at least 146%, at least 147%, at least 148%, at least 149%, at least 150%, at least 151%, at least 152%, at least 153%, at least 154%, at least 155%, at least 156%, at least 157%, at least 158%, at least 159%, at least 160%, at least 161%, at least 162%, at least 163%, at least 164%, at least 165%, at least 166%, at least 167%, at least 168%, at least 169%, at least 170%, at least 171%, at least 172%, at least 173%, at least 174%, at least 175%, at least 176%, at least 177%, at least 178%, at least 179%, at least 180%, at least 190%, at least 200%, at least 210%, at least 220%, at least 230%, at least 240%, at least 250%, at least 275%, at least 300%, at least 325%, at least 350%, at least 375%, at least 400%, at least 425%, at least 450%, at least 475%, at least 500%, at least 550%, at least 600%, at least 650%, at least 700%, at least 750%, at least 800%, at least 850%, at least 900%, at least 950%, at least 1000%, at least 1001%, at least 1002%, at least 1003%, at least 1004%, at least 1005%, at least 1006%, at least 1007%, at least 1008%, at least 1009%, at least 1010%, at least 1011%, at least 1012%, at least 1013%, at least 1014%, at least 1015%, at least 1016%, at least 1017%, at least 1018%, at least 1019%, at least 1020%, at least 1021%, at least 1022%, or at least 1023% relative to placebo as determined by ADAS-Cog, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 58% to 1023% relative to placebo as determined by ADAS-Cog, wherein the subject is ApoE4-positive, and wherein the subject has been diagnosed as having mild Alzheimer's disease dementia. In some embodiments, the clinical decline is reduced by 58% relative to placebo as determined by ADAS-Cog, wherein the subject is ApoE4-positive, and wherein the subject has been diagnosed as having mild Alzheimer's disease dementia. In some embodiments, the clinical decline is reduced by 171% relative to placebo as determined by ADAS-Cog, wherein the subject is ApoE4-positive, and wherein the subject has been diagnosed as having mild Alzheimer's disease dementia. In some embodiments, the clinical decline is reduced by 1023% relative to placebo as determined by ADAS-Cog, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, at least 119%, at least 120%, at least 121%, at least 122%, at least 123%, at least 124%, at least 125%, at least 126%, at least 127%, at least 128%, at least 129%, at least 130%, at least 131%, at least 132%, at least 133%, at least 134%, at least 135%, at least 136%, at least 137%, at least 138%, at least 139%, at least 140%, at least 141%, at least 142%, at least 143%, at least 144%, at least 145%, at least 146%, at least 147%, at least 148%, at least 149%, at least 150%, at least 151%, at least 152%, at least 153%, at least 154%, at least 155%, at least 156%, at least 157%, at least 158%, at least 159%, at least 160%, at least 161%, at least 162%, at least 163%, at least 164%, at least 165%, at least 166%, at least 167%, at least 168%, at least 169%, at least 170%, at least 171%, at least 172%, at least 173%, or at least 174% relative to placebo as determined by CDR-SB, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 70% to 200%, such as 75% to 180% or 82% to 174%, relative to placebo as determined by CDR-SB, wherein the subject is ApoE4-positive, and wherein the subject has been diagnosed as having mild Alzheimer's disease dementia. In some embodiments, the clinical decline is reduced by at least 70%, such as at least 80% relative to placebo as determined by CDR-SB, wherein the subject is ApoE4-positive, and wherein the subject has been diagnosed as having mild Alzheimer's disease dementia. In some embodiments, the clinical decline is reduced by at least 75%, such as at least 80% or at least 85%, relative to placebo as determined by CDR-SB, wherein the subject is ApoE4-positive, and wherein the subject has been diagnosed as having mild Alzheimer's disease dementia. In some embodiments, the clinical decline is reduced by at least 150%, such as at least 160% or 170%, relative to placebo as determined by CDR-SB, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild Alzheimer's disease dementia is reduced by at least 70%, such as at least 75%, at least 80%, or at least 85%, relative to placebo as determined by CDR-SB composition comprising a therapeutically effective amount of at least one anti-Aβ protofibril antibody. In some embodiments, the clinical decline in the ApoE4-positive subject diagnosed as having mild Alzheimer's disease dementia is reduced by at least 130%, such as at least 140%, at least 150%, at least 160%, or at least 170%, relative to placebo as determined by CDR-SB after 12 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 65%, such as at least 70%, at least 75%, or at least 80%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the subject is ApoE4-negative.

In some embodiments, the clinical decline is reduced by at least 1%, at least 2%, at least 3%, at least 4%, at least 5%, at least 6%, at least 7%, at least 8%, at least 9%, at least 10%, at least 11%, or at least 12% relative to placebo as determined by ADCOMS, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 5% to 15% relative to placebo as determined by ADCOMS, wherein the subject is ApoE4-negative. In some embodiments, the clinical decline is reduced by at least 5%, such as at least 7%, relative to placebo as determined by ADCOMS, wherein the subject is ApoE4-negative. In some embodiments, the clinical decline is reduced by at least 10%, such as at least 12%, relative to placebo as determined by ADCOMS, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-negative subject is reduced by at least −2% relative to placebo as determined by ADCOMS after 6 months of administration of the composition comprising a therapeutically effective amount of at least one anti-Aβ protofibril antibody. In some embodiments, the clinical decline in the ApoE4-negative subject is reduced by at least 10% relative to placebo as determined by ADCOMS after 12 months of administration of the composition comprising a therapeutically effective amount of at least one anti-Aβ protofibril antibody. In some embodiments, the clinical decline in the ApoE4-negative subject is reduced by at least 5%, such as at least 7%, relative to placebo as determined by ADCOMS after 18 months of administration of the composition comprising a therapeutically effective amount of at least one anti-Aβ protofibril antibody. In some embodiments, the clinical decline in the ApoE4-negative subject is reduced by at least 7%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, or at least 72% relative to placebo as determined by ADAS-cog, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 80% relative to placebo as determined by ADAS-cog, wherein the subject is ApoE4-negative. In some embodiments, the clinical decline is reduced by at least 35%, such as at least 40% or at least 43%, relative to placebo as determined by ADAS-cog, wherein the subject is ApoE4-negative. In some embodiments, the clinical decline is reduced by at least 40%, such as at least 45% or at least 46%, relative to placebo as determined by ADAS-cog, wherein the subject is ApoE4-negative. In some embodiments, the clinical decline is reduced by at least 65%, such as at least 70% or at least 72%, relative to placebo as determined by ADAS-cog, wherein the subject is ApoE4-negative. In some embodiments, the clinical decline is reduced by at least 43%, relative to placebo as determined by ADAS-cog, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is increased by 7%, 6%, 5%, 5%, 3%, 2%, or 1% relative to placebo as determined by CDR-SB, wherein the subject is ApoE4-negative. In some embodiments, the clinical decline is reduced by at least 1%, at least 2%, or at least 3% relative to placebo as determined by CDR-SB, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 3% relative to placebo as determined by CDR-SB, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, or at least 166% relative to placebo as determined by ADAS-Cog, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 200%, such as 60% to 180% or 65% to 170%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the clinical decline is reduced by at least 50%, such as at least 55% or at least 65%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the clinical decline is reduced by at least 70%, such as at least 75% or at least 80%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the clinical decline is reduced by at least 150%, such as at least 160%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-negative subject is reduced by at least 150%, such as at least 160%, relative to placebo as determined by ADAS-Cog after 6 months of administration of the composition comprising a therapeutically effective amount of at least one anti-Aβ protofibril antibody. In some embodiments, the clinical decline in the ApoE4-negative subject is reduced by at least 70%, such as at least 75% or at least 80%, relative to placebo as determined by ADAS-Cog after 12 months of administration of the composition comprising a therapeutically effective amount of at least one anti-Aβ protofibril antibody. In some embodiments, the clinical decline in the ApoE4-negative subject is reduced by at least 50%, such as at least 60% or at least 65%, relative to placebo as determined by ADAS-Cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, or at least 5% relative to placebo as determined by CDR-SB, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 5% relative to placebo as determined by CDR-SB, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12% relative to placebo as determined by CDR-SB, wherein the subject is ApoE4-negative, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 10%, such as at least 12%, relative to placebo as determined by CDR-SB, wherein the subject is ApoE4-negative, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 35% to 50% relative to placebo as determined by ADCOMS, wherein the subject is not concomitantly administered at least one Alzheimer's disease medication other than BAN2401. In some embodiments, the clinical decline is reduced by at least 35%, such as by at 38%, at least 40%, or at least 41%, relative to placebo as determined by ADCOMS, wherein the subject is not concomitantly administered at least one Alzheimer's disease medication other than BAN2401. In some embodiments, the clinical decline is reduced by at least 41%, relative to placebo as determined by ADCOMS, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody other than BAN2401.

In some embodiments, the clinical decline in the subject who is not concomitantly administered at least one Alzheimer's disease medication other than BAN2401 is reduced by at least 41% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, or at least 59% relative to placebo as determined by ADAS-cog, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject is not concomitantly administered at least one Alzheimer's disease medication other than BAN2401. In some embodiments, the clinical decline is reduced by at least 50%, such as by at 55%, at least 57%, or at least 59%, relative to placebo as determined by ADAS-cog, wherein the subject is not concomitantly administered at least one Alzheimer's disease medication other than BAN2401. In some embodiments, the clinical decline is reduced by at least 59% relative to placebo as determined by ADAS-cog, wherein the subject is not concomitantly administered at least one Alzheimer's disease medication. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject who is not concomitantly administered at least one Alzheimer's disease medication other than BAN2401 is reduced by at least 59% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45% relative to placebo as determined by CDR-SB, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject is not concomitantly administered at least one Alzheimer's disease medication other than BAN2401. In some embodiments, the clinical decline is reduced by at least 35%, such as by at 40%, at least 42%, or at least 45%, relative to placebo as determined by CDR-SB, wherein the subject is not concomitantly administered at least one Alzheimer's disease medication other than BAN2401. In some embodiments, the clinical decline is reduced by at least 45% relative to placebo as determined by CDR-SB, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject who is not concomitantly administered at least one Alzheimer's disease medication other than BAN2401 is reduced by at least 45% relative to placebo as determined by CDR-SB after 18 months of administration of the composition comprising a therapeutically effective amount of at least one anti-Aβ protofibril antibody. In some embodiments, the composition comprises 10 mg/kg of at least one anti-β protofibril antibody and is administered once every two weeks or once every month. In some embodiments, the at least one anti-Aβ protofibril antibody is BAN2401.

Treating A Subject having Early Alzheimer's Disease Resulting in Reduction of Severity of Symptom Relative to Severity Prior to Treatment

Provided herein is a method of treating a subject having early Alzheimer's disease comprising administering to said subject a composition comprising a therapeutically effective amount of at least one anti-Aβ protofibril antibody disclosed herein, wherein the severity of at least one symptom associated with Alzheimer's disease is reduced by at least 10%, 20%, 30%, 40%, 50%, 60%, 70%, 80%, 90%, or 95% relative to the severity of the same symptom in the same subject prior to treatment. In some embodiments, the subject having early Alzheimer's disease has been diagnosed as having mild cognitive impairment due to Alzheimer's disease—intermediate likelihood and/or has been diagnosed as having mild Alzheimer's disease dementia. In some embodiments, the subject having early Alzheimer's disease is ApoE4-positive.

In some embodiments, the severity of at least one symptom associated with Alzheimer's disease is reduced by at least 1%. In some embodiments, the severity of at least one symptom associated with Alzheimer's disease is reduced by at least 10%. In some embodiments, the severity of at least one symptom associated with Alzheimer's disease is reduced by at least 20%. In some embodiments, the severity of at least one symptom associated with Alzheimer's disease is reduced by at least 30%. In some embodiments, the severity of at least one symptom associated with Alzheimer's disease is reduced by at least 40%. In some embodiments, the severity of at least one symptom associated with Alzheimer's disease is reduced by at least 50%. In some embodiments, the severity of at least one symptom associated with Alzheimer's disease is reduced by at least 60%. In some embodiments, the severity of at least one symptom associated with Alzheimer's disease is reduced by at least 70%. In some embodiments, the severity of at least one symptom associated with Alzheimer's disease is reduced by at least 80%. In some embodiments, the severity of at least one symptom associated with Alzheimer's disease is reduced by at least 90%. In some embodiments, the severity of at least one symptom associated with Alzheimer's disease is reduced by at least 95%.

In some embodiments, the above-recited reduction in severity is determined after 1 month, 6 months, 12 months, 18 months, 60 months, and/or 63 months of administration of the composition comprising a therapeutically effective amount of at least one anti-Aβ protofibril antibody.

In some embodiments, the severity of the at least one symptom associated with Alzheimer's disease is determined by ADCOMS, PET, MMSE, CDR-SB, and/or ADAS-Cog.

In some embodiments, the at least one symptom associated with Alzheimer's disease is chosen from clinical decline and brain amyloid level.

Any of the anti-Aβ protofibril antibodies, therapeutically acceptable amounts thereof, dosing regimens therefor, and compositions comprising the same that are disclosed herein may be used in the method of treating a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month.

In some embodiments, the at least one symptom associated with Alzheimer's disease is clinical decline.

In some embodiments, the at least one symptom associated with Alzheimer's disease is brain amyloid level.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of BAN2401. In some embodiments, the reduction in clinical decline is determined after 1 month, 6 months, 12 months, 18 months, 60 months, and/or 63 months of administration of the composition comprising a therapeutically effective amount of BAN2401. In some embodiments, the reduction in clinical decline is determined after 1 month of administration of the composition comprising a therapeutically effective amount of BAN2401. In some embodiments, the reduction in clinical decline is determined after 6 months of administration of the composition comprising a therapeutically effective amount of BAN2401. In some embodiments, the reduction in clinical decline is determined after 12 months of administration of the composition comprising a therapeutically effective amount of BAN2401. In some embodiments, the reduction in clinical decline is determined after 18 months of administration of the composition comprising a therapeutically effective amount of BAN2401. In some embodiments, the reduction in clinical decline is determined after 60 months of administration of the composition comprising a therapeutically effective amount of BAN2401. In some embodiments, the reduction in clinical decline is determined after 63 months of administration of the composition comprising a therapeutically effective amount of BAN2401.

In some embodiments, the subject is ApoE4-positive. In some embodiments, the subject is ApoE4-negative.

Treating a Subject having Early Alzheimer's Disease Resulting in Reduction of Severity of Symptom Relative to Severity of Symptom in Placebo-treated Subject

Provided herein is a method of treating a subject having early Alzheimer's disease comprising administering to said subject a composition comprising a therapeutically effective amount of at least one anti-Aβ protofibril antibody disclosed herein, wherein the severity of at least one symptom associated with Alzheimer's disease is reduced by at least 10%, 20%, 30%, 40%, 50%, 60%, 70%, 80%, 90%, or 95% relative to the severity of the same symptom in subjects that received placebo. In some embodiments, the subject having early Alzheimer's disease has been diagnosed as having mild cognitive impairment due to Alzheimer's disease—intermediate likelihood and/or has been diagnosed as having mild Alzheimer's disease dementia. In some embodiments, the subject having early Alzheimer's disease is ApoE4-positive.

In some embodiments, the severity of at least one symptom associated with Alzheimer's disease is reduced by at least 1%. In some embodiments, the severity of at least one symptom associated with Alzheimer's disease is reduced by at least 10%. In some embodiments, the severity of at least one symptom associated with Alzheimer's disease is reduced by at least 20%. In some embodiments, the severity of at least one symptom associated with Alzheimer's disease is reduced by at least 30%. In some embodiments, the severity of at least one symptom associated with Alzheimer's disease is reduced by at least 40%. In some embodiments, the severity of at least one symptom associated with Alzheimer's disease is reduced by at least 50%. In some embodiments, the severity of at least one symptom associated with Alzheimer's disease is reduced by at least 60%. In some embodiments, the severity of at least one symptom associated with Alzheimer's disease is reduced by at least 70%. In some embodiments, the severity of at least one symptom associated with Alzheimer's disease is reduced by at least 80%. In some embodiments, the severity of at least one symptom associated with Alzheimer's disease is reduced by at least 90%. In some embodiments, the severity of at least one symptom associated with Alzheimer's disease is reduced by at least 95%.

In some embodiments, the above-recited reduction in severity is determined after 1 month, 6 months, 12 months, 18 months, 60 months, and/or 63 months of administration of the composition comprising a therapeutically effective amount of at least one anti-Aβ protofibril antibody.

In some embodiments, the severity of the at least one symptom associated with Alzheimer's disease is determined by ADCOMS, PET, MMSE, CDR-SB, and/or ADAS-Cog.

In some embodiments, the at least one symptom associated with Alzheimer's disease is chosen from clinical decline and brain amyloid level.

Any of the anti-Aβ protofibril antibodies, therapeutically acceptable amounts thereof, dosing regimens therefor, and compositions comprising the same that are disclosed herein may be used in the method of treating a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month.

In some embodiments, the at least one symptom associated with Alzheimer's disease is clinical decline.

In some embodiments, the at least one symptom associated with Alzheimer's disease is brain amyloid level.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, or at least 46% relative to placebo as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 35% relative to placebo as determined by ADCOMS. In some embodiments, the clinical decline is reduced by 20% to 30% relative to placebo as determined by ADCOMS. In some embodiments, the clinical decline is reduced by 27% to 35% relative to placebo as determined by ADCOMS. In some embodiments, the clinical decline is reduced by at least 20% relative to placebo as determined by ADCOMS. In some embodiments, the clinical decline is reduced by at least 35% relative to placebo as determined by ADCOMS. In some embodiments, the clinical decline is reduced by at least 20% as determined by ADCOMS. In some embodiments, the clinical decline is reduced by at least 30% as determined by ADCOMS. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 45% relative to placebo as determined by ADCOMS after 6 months of administration of the composition comprising a therapeutically effective amount of at least one anti-protofibril antibody. In some embodiments, the clinical decline is reduced by at least 35% relative to placebo as determined by ADCOMS after 12 months of administration of the composition comprising a therapeutically effective amount of at least one anti-Aβ protofibril antibody. In some embodiments, the clinical decline is reduced by at least 30% relative to placebo as determined by ADCOMS after 18 months of administration of the composition comprising a therapeutically effective amount of at least one anti-Aβ protofibril antibody. In some embodiments, the clinical decline is reduced by at least 46% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, or at least 52% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 33% relative to placebo as determined by ADCOMS, wherein the subject has been diagnosed as having mild cognitive impairment due to Alzheimer's disease—intermediate likelihood. In some embodiments, the clinical decline is reduced by at least 20%, such as by at 25% or at least 28%, relative to placebo as determined by ADCOMS, wherein the subject has been diagnosed as having mild cognitive impairment due to Alzheimer's disease—intermediate likelihood. In some embodiments, the clinical decline is reduced by at least 25%, such as by at least 30% or at least 33%, relative to placebo as determined by ADCOMS, wherein the subject has been diagnosed as having mild cognitive impairment due to Alzheimer's disease—intermediate likelihood. In some embodiments, the clinical decline is reduced by at least 25%, such as by at least 30%, at least 35%, at least 40%, at least 45%, at least 50%, or at least 52% relative to placebo as determined by ADCOMS, wherein the subject has been diagnosed as having mild cognitive impairment due to Alzheimer's disease—intermediate likelihood. In some embodiments, the clinical decline is reduced by at least 52% relative to placebo as determined by ADCOMS, wherein the subject has been diagnosed as having mild cognitive impairment due to Alzheimer's disease-13 intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 30% relative to placebo as determined by ADCOMS after 6 months of administration of the composition comprising a therapeutically effective amount of at least one anti-Aβ protofibril antibody. In some embodiments, the clinical decline in the subject diagnosed as having mild cognitive impairment due to Alzheimer's disease—intermediate likelihood is reduced by at least 25% relative to placebo as determined by ADCOMS after 12 months of administration of the composition comprising a therapeutically effective amount of at least one anti-Aβ protofibril antibody. In some embodiments, the clinical decline in the subject diagnosed as having mild cognitive impairment due to Alzheimer's disease—intermediate likelihood is reduced by at least 30% relative to placebo as determined by ADCOMS after 18 months of administration of the composition comprising a therapeutically effective amount of at least one anti-Aβ protofibril antibody. In some embodiments, the clinical decline in the subject diagnosed as having mild cognitive impairment due to Alzheimer's disease—intermediate likelihood is reduced by at least 52% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, or at least 33% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 38% relative to placebo as determined by ADCOMS, wherein the subject has been diagnosed as having mild cognitive impairment due to Alzheimer's disease—intermediate likelihood. In some embodiments, the clinical decline is reduced by at least 20%, such as by at 25%, at least 28%, or at least 33%, relative to placebo as determined by ADCOMS, wherein the subject has been diagnosed as having mild cognitive impairment due to Alzheimer's disease—intermediate likelihood. In some embodiments, the clinical decline is reduced by at least 25%, such as by at least 30% or at least 33%, relative to placebo as determined by ADCOMS, wherein the subject has been diagnosed as having mild cognitive impairment due to Alzheimer's disease—intermediate likelihood. In some embodiments, the clinical decline is reduced by at least 33% relative to placebo as determined by ADCOMS, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 33% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, or at least 78% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 80% relative to placebo as determined by ADCOMS, wherein the subject has been diagnosed as having Alzheimer's disease dementia. In some embodiments, the clinical decline is reduced by 35% to 78% relative to placebo as determined by ADCOMS, wherein the subject has been diagnosed as having Alzheimer's disease dementia. In some embodiments, the clinical decline is reduced by at least 35% relative to placebo as determined by ADCOMS, wherein the subject has been diagnosed as having mild Alzheimer's disease dementia. In some embodiments, the clinical decline is reduced by at least 50%, such as by at least 52% or at least 53% relative to placebo as determined by ADCOMS, wherein the subject has been diagnosed as having mild Alzheimer's disease dementia. In some embodiments, the clinical decline is reduced by at least 70%, such as by at least 75% or at least 78%, relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 70% relative to placebo as determined by ADCOMS after 6 months of administration of the composition comprising a therapeutically effective amount of at least one anti-Aβ protofibril antibody. In some embodiments, the clinical decline in the subject diagnosed as having mild Alzheimer's disease dementia is reduced by at least 50% relative to placebo as determined by ADCOMS after 12 months of administration of the composition comprising a therapeutically effective amount of at least one anti-Aβ protofibril antibody. In some embodiments, the clinical decline in the subject diagnosed as having mild Alzheimer's disease dementia is reduced by at least 30% relative to placebo as determined by ADCOMS after 18 months of administration of the composition comprising a therapeutically effective amount of at least one anti-Aβ protofibril antibody. In some embodiments, the clinical decline in the subject diagnosed as having mild Alzheimer's disease dementia is reduced by at least 52% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, or at least 35% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 28% to 38% relative to placebo as determined by ADCOMS, wherein the subject has been diagnosed as having mild Alzheimer's disease dementia. In some embodiments, the clinical decline is reduced by at least 20%, such as by at 25%, at least 28%, or at least 35%, relative to placebo as determined by ADCOMS, wherein the subject has been diagnosed as having mild Alzheimer's disease dementia. In some embodiments, the clinical decline is reduced by at least 25%, such as by at least 30% or at least 35%, relative to placebo as determined by ADCOMS, wherein the subject has been diagnosed as having mild Alzheimer's disease dementia. In some embodiments, the clinical decline is reduced by at least 35% relative to placebo as determined by ADCOMS, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 35% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 75%, at least 80%, at least 85%, at least 90%, at least 95%, at least 100%, at least 110%, at least 120%, at least 130%, at least 140%, or at least 150% relative to placebo as determined by ADAS-cog. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 150% relative to placebo as determined by ADAS-cog. In some embodiments, the clinical decline is reduced by 45% to 145% relative to placebo as determined by ADAS-cog. In some embodiments, the clinical decline is reduced by 45% to 55% relative to placebo as determined by ADAS-cog. In some embodiments, the clinical decline is reduced by at least 30% relative to placebo as determined by ADAS-cog. In some embodiments, the clinical decline is reduced by at least 35% relative to placebo as determined by ADAS-cog. In some embodiments, the clinical decline is reduced by at least 40% as determined by ADAS-cog. In some embodiments, the clinical decline is reduced by at least 45% as determined by ADAS-cog. In some embodiments, the clinical decline is reduced by at least 47% as determined by ADAS-cog. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 100%, such as at least 120% or at least 140%, relative to placebo as determined by ADAS-cog after 6 months of administration of the composition comprising a therapeutically effective amount of at least one anti-Aβ protofibril antibody. In some embodiments, the clinical decline is reduced by at least 40%, such as at least 45%, relative to placebo as determined by ADAS-cog after 12 months of administration of the composition comprising a therapeutically effective amount of at least one anti-Aβ protofibril antibody. In some embodiments, the clinical decline is reduced by at least 40%, such as at least 45%, relative to placebo as determined by ADAS-cog after 18 months of administration of the composition comprising a therapeutically effective amount of at least one anti-Aβ protofibril antibody. In some embodiments, the clinical decline is reduced by at least 47%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 56%, at least 47%, at least 48%, at least 49%, at least 50%, at least 51%, at least 52%, at least 53%, at least 54%, at least 55%, at least 56%, at least 57%, or at least 58% relative to placebo as determined by ADAS-cog, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject has been diagnosed as having mild cognitive impairment due to Alzheimer's disease—intermediate likelihood. In some embodiments, the clinical decline is reduced by at least 50%, such as by at 52%, at least 55%, or at least 58%, relative to placebo as determined by ADAS-cog, wherein the subject has been diagnosed as having mild cognitive impairment due to Alzheimer's disease—intermediate likelihood. In some embodiments, the clinical decline is reduced by at least 58% relative to placebo as determined by ADAS-cog, wherein the subject has been diagnosed as having mild cognitive impairment due to Alzheimer's disease-13 intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 58% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, or at least 41% relative to placebo as determined by ADAS-cog, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 30% to 50% relative to placebo as determined by ADAS-cog, wherein the subject has been diagnosed as having mild Alzheimer's disease dementia. In some embodiments, the clinical decline is reduced by at least 35%, such as by at 38%, at least 40%, or at least 41%, relative to placebo as determined by ADAS-cog, wherein the subject has been diagnosed as having mild Alzheimer's disease dementia. In some embodiments, the clinical decline is reduced by at least 41% relative to placebo as determined by ADAS-cog, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 41% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40% relative to placebo as determined by CDR-SB. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 60% relative to placebo as determined by CDR-SB. In some embodiments, the clinical decline is reduced by 25% to 60% relative to placebo as determined by CDR-SB. In some embodiments, the clinical decline is reduced by 25% to 50% relative to placebo as determined by CDR-SB. In some embodiments, the clinical decline is reduced by at least 20% relative to placebo as determined by CDR-SB. In some embodiments, the clinical decline is reduced by at least 30% relative to placebo as determined by CDR-SB. In some embodiments, the clinical decline is reduced by at least 25%, such as at least 26% or at least 28%, as determined by CDR-SB. In some embodiments, the clinical decline is reduced by at least 30%, such as at least 35% or at least 38%, as determined by CDR-SB. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 30%, such as at least 35% or at least 40%, relative to placebo as determined by CDR-SB after 6 months of administration of the composition comprising a therapeutically effective amount of at least one anti-Aβ protofibril antibody. In some embodiments, the clinical decline is reduced by at least 30%, such as at least 35% or at least 45%, relative to placebo as determined by CDR-SB after 12 months of administration of the composition comprising a therapeutically effective amount of at least one anti-Aβ protofibril antibody. In some embodiments, the clinical decline is reduced by at least 20%, such as at least 25%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, or at least 14% relative to placebo as determined by CDR-SB, wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 10% to 20% relative to placebo as determined by CDR-SB, wherein the subject has been diagnosed as having mild cognitive impairment due to Alzheimer's disease—intermediate likelihood. In some embodiments, the clinical decline is reduced by at least 5%, such as by at 10%, at least 12%, or at least 14%, relative to placebo as determined by CDR-SB, wherein the subject has been diagnosed as having mild cognitive impairment due to Alzheimer's disease—intermediate likelihood. In some embodiments, the clinical decline is reduced by at least 14% relative to placebo as determined by CDR-SB, wherein the subject has been diagnosed as having mild cognitive impairment due to Alzheimer's disease-13 intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild cognitive impairment due to Alzheimer's disease-13 intermediate likelihood is reduced by at least 14% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, or at least 51% relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 60% relative to placebo as determined by CDR-SB, wherein the subject has been diagnosed as having mild Alzheimer's disease dementia. In some embodiments, the clinical decline is reduced by at least 45%, such as by at 48%, at least 50%, or at least 51%, relative to placebo as determined by CDR-SB, wherein the subject has been diagnosed as having mild Alzheimer's disease dementia. In some embodiments, the clinical decline is reduced by at least 51%, relative to placebo as determined by CDR-SB, wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject diagnosed as having mild Alzheimer's disease dementia is reduced by at least 51% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of at least one anti-Aβ protofibril antibody. In some embodiments, the reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the reduction in clinical decline is determined after 1 month of administration of the composition comprising a therapeutically effective amount of at least one anti-Aβ protofibril antibody. In some embodiments, the reduction in clinical decline is determined after 6 months of administration of the composition comprising a therapeutically effective amount of at least one anti-Aβ protofibril antibody. In some embodiments, the reduction in clinical decline is determined after 12 months of administration of the composition comprising a therapeutically effective amount of at least one anti-Aβ protofibril antibody. In some embodiments, the reduction in clinical decline is determined after 18 months of administration of the composition comprising a therapeutically effective amount of at least one anti-Aβ protofibril antibody. In some embodiments, the reduction in clinical decline is determined after 60 months of administration of the composition comprising a therapeutically effective amount of at least one anti-Aβ protofibril antibody. In some embodiments, the reduction in clinical decline is determined after 63 months of administration of the composition comprising a therapeutically effective amount of at least one anti-Aβ protofibril antibody.

In some embodiments, the reduction in clinical decline is determined after administration of a composition comprising a therapeutically effective amount of BAN2401.

In some embodiments, the reduction in clinical decline is determined after 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 30 months, 36 months, 42 months, 48 months, 54 months, 60 months, 63 months, 66 months, and/or 72 months of administration of the composition comprising a therapeutically effective amount of BAN2401. In some embodiments, the reduction in clinical decline is determined after 1 month, 6 months, 12 months, 18 months, 60 months, and/or 63 months of administration of the composition comprising a therapeutically effective amount of BAN2401. In some embodiments, the reduction in clinical decline is determined after 1 month of administration of the composition comprising a therapeutically effective amount of BAN2401. In some embodiments, the reduction in clinical decline is determined after 6 months of administration of the composition comprising a therapeutically effective amount of BAN2401. In some embodiments, the reduction in clinical decline is determined after 12 months of administration of the composition comprising a therapeutically effective amount of BAN2401. In some embodiments, the reduction in clinical decline is determined after 18 months of administration of the composition comprising a therapeutically effective amount of BAN2401. In some embodiments, the reduction in clinical decline is determined after 60 months of administration of the composition comprising a therapeutically effective amount of BAN2401. In some embodiments, the reduction in clinical decline is determined after 63 months of administration of the composition comprising a therapeutically effective amount of BAN2401.

In some embodiments, the subject is ApoE4-positive.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, or at least 74% relative to placebo as determined by ADCOMS, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 60% to 80%, such as by 63% to 74%, relative to placebo as determined by ADCOMS, wherein the subject is ApoE4-positive. In some embodiments, the clinical decline is reduced by at least 60%, such as at least 63%, relative to placebo as determined by ADCOMS, wherein the subject is ApoE4-positive. In some embodiments, the clinical decline is reduced by at least 65%, such as at least 67%, relative to placebo as determined by ADCOMS, wherein the subject is ApoE4-positive. In some embodiments, the clinical decline is reduced by at least 70%, such as at least 74%, relative to placebo as determined by ADCOMS, wherein the subject is ApoE4-positive.

In some embodiments, the clinical decline in the ApoE4-positive subject is reduced by at least 70% relative to placebo as determined by ADCOMS after 6 months of administration of the composition comprising a therapeutically effective amount of at least one anti-Aβ protofibril antibody. In some embodiments, the clinical decline in the ApoE4-positive subject is reduced by at least 60% relative to placebo as determined by ADCOMS after 12 months of administration of the composition comprising a therapeutically effective amount of at least one anti-Aβ protofibril antibody. In some embodiments, the clinical decline in the ApoE4-positive subject is reduced by at least 50%, such as at least 55% or at least 60%, relative to placebo as determined by ADCOMS after 18 months of administration of the composition comprising a therapeutically effective amount of at least one anti-Aβ protofibril antibody. In some embodiments, the clinical decline in the ApoE4-positive subject is reduced by at least 63%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 290%, at least 295%, at least 300%, at least 305%, at least 310%, at least 315%, at least 320%, at least 325%, at least 330%, or at least 331% relative to placebo as determined by ADAS-cog, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced 70% to 400%, such as 80% to 350%, relative to placebo as determined by ADAS-cog, wherein the subject is ApoE4-positive. In some embodiments, the clinical decline is reduced by at least 70%, such as at least 75%, at least 80%, or at least 84%, relative to placebo as determined by ADAS-cog, wherein the subject is ApoE4-positive. In some embodiments, the clinical decline in the ApoE4-positive subject is reduced by at least 84%, relative to placebo as determined by ADAS-cog after 18 months of administration of the composition comprising a therapeutically effective amount of at least one anti-Aβ protofibril antibody. In some embodiments, the clinical decline is reduced by at least 80%, such as at least 90% or at least 100%, relative to placebo as determined by ADAS-cog, wherein the subject is ApoE4-positive. In some embodiments, the clinical decline is reduced by at least 300%, such as at least 330%, relative to placebo as determined by ADAS-cog, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject is reduced by at least 300% relative to placebo as determined by ADAS-cog after 6 months of administration of the composition comprising a therapeutically effective amount of at least one anti-Aβ protofibril antibody. In some embodiments, the clinical decline in the ApoE4-positive subject is reduced by at least 80%, such as at least 90% or at least 100%, relative to placebo as determined by ADAS-cog after 12 months of administration of the composition comprising a therapeutically effective amount of at least one anti-Aβ protofibril antibody. In some embodiments, the clinical decline in the ApoE4-positive subject is reduced by at least 70%, such as at least 75%, at least 80%, or at least 84%, relative to placebo as determined by ADAS-cog after 18 months of administration of the composition comprising a therapeutically effective amount of at least one anti-Aβ protofibril antibody. In some embodiments, the clinical decline in the ApoE4-positive subject is reduced by at least 84%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, or at least 87% relative to placebo as determined by CDR-SB, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 35% to 150%, such as 40% to 100% or 45% to 90%, relative to placebo as determined by CDR-SB, wherein the subject is ApoE4-positive. In some embodiments, the clinical decline is reduced by at least 35%, such as at least 40% or at least 45%, relative to placebo as determined by CDR-SB, wherein the subject is ApoE4-positive. In some embodiments, the clinical decline is reduced by at least 50%, such as at least 55% or at least 60%, relative to placebo as determined by CDR-SB, wherein the subject is ApoE4-positive. In some embodiments, the clinical decline is reduced by at least 70%, such as at least 80% or at least 85%, relative to placebo as determined by CDR-SB, wherein the subject is ApoE4-positive. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject is reduced by at least 35%, such as at least 40% or at least 45%, relative to placebo as determined by CDR-SB after 6 months of administration of the composition comprising a therapeutically effective amount of at least one anti-Aβ protofibril antibody. In some embodiments, the clinical decline in the ApoE4-positive subject is reduced by at least 70%, such as at least 75% or at least 80%, relative to placebo as determined by CDR-SB after 12 months of administration of the composition comprising a therapeutically effective amount of at least one anti-Aβ protofibril antibody. In some embodiments, the clinical decline in the ApoE4-positive subject is reduced by at least 50%, such as at least 55% or at least 60%, relative to placebo as determined by CDR-SB after 18 months of administration of the composition comprising a therapeutically effective amount of at least one anti-protofibril antibody. In some embodiments, the clinical decline in the ApoE4-positive subject is reduced by at least 60%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, or at least 59% relative to placebo as determined by ADCOMS, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 30% to 70%, such as 38% to 59%, relative to placebo as determined by ADCOMS, wherein the subject is ApoE4-positive, and wherein the subject has been diagnosed as having mild cognitive impairment due to Alzheimer's disease—intermediate likelihood. In some embodiments, the clinical decline is reduced by at least 30%, such as at least 35% or at least 38%, relative to placebo as determined by ADCOMS, wherein the subject is ApoE4-positive, and wherein the subject has been diagnosed as having mild cognitive impairment due to Alzheimer's disease—intermediate likelihood. In some embodiments, the clinical decline is reduced by at least 45%, such as at least 50% or at least 53%, relative to placebo as determined by ADCOMS, wherein the subject is ApoE4-positive, and wherein the subject has been diagnosed as having mild cognitive impairment due to Alzheimer's disease—intermediate likelihood. In some embodiments, the clinical decline is reduced by at least 50%, such as at least 55% or at least 59%, relative to placebo as determined by ADCOMS, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease—intermediate likelihood is reduced by at least 50%, such as at least 55%, relative to placebo as determined by ADCOMS after 6 months of administration of the composition comprising a therapeutically effective amount of at least one anti-Aβ protofibril antibody. In some embodiments, the clinical decline in the ApoE4-positive subject diagnosed as mild cognitive impairment due to Alzheimer's disease—intermediate likelihood is reduced by at least 30%, such as at least 35%, relative to placebo as determined by ADCOMS after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease—intermediate likelihood is reduced by at least 45%, such as at least 50% or at least 55%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, or at least 211% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild Alzheimer's disease dementia is reduced by at least 100%, such as at least 110%, relative to placebo as determined by ADCOMS after 6 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 100%, such as at least 110%, relative to placebo as determined by ADCOMS after 12 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 65%, such as at least 70% or at least 75%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, or at least 211% relative to placebo as determined by ADAS-Cog, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 300%, such as 45% to 250% or 50% to 250%, relative to placebo as determined by ADAS-Cog, wherein the subject is ApoE4-positive, and wherein the subject has been diagnosed as having mild cognitive impairment due to Alzheimer's disease—intermediate likelihood. In some embodiments, the clinical decline is reduced by at least 40%, such as at least 45% or at least 50%, relative to placebo as determined by ADAS-Cog, wherein the subject is ApoE4-positive, and wherein the subject has been diagnosed as having mild cognitive impairment due to Alzheimer's disease—intermediate likelihood. In some embodiments, the clinical decline is reduced by at least 60, such as at least 70%, at least 75%, or at least 80%, relative to placebo as determined by ADAS-Cog, wherein the subject is ApoE4-positive, and wherein the subject has been diagnosed as having mild cognitive impairment due to Alzheimer's disease—intermediate likelihood. In some embodiments, the clinical decline is reduced by at least 100%, such as at least 150% or at least 200% relative to placebo as determined by ADAS-Cog, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease—intermediate likelihood is reduced by at least 100%, such as at least 150% or at least 200%, relative to placebo as determined by ADAS-cog after 6 months of administration of the composition comprising a therapeutically effective amount of at least one anti-Aβ protofibril antibody. In some embodiments, the clinical decline in the ApoE4-positive subject diagnosed as mild cognitive impairment due to Alzheimer's disease—intermediate likelihood is reduced by at least 40%, such as at least 45% or at least 50%, relative to placebo as determined by ADAS-cog after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease—intermediate likelihood is reduced by at least 50%, such as at least 60%, at least 70%, or at least 75%, relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45% relative to placebo as determined by CDR-SB, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 20% to 90%, such as 25% to 80% or 30% to 75%, relative to placebo as determined by CDR-SB, wherein the subject is ApoE4-positive, and wherein the subject has been diagnosed as having mild cognitive impairment due to Alzheimer's disease—intermediate likelihood. In some embodiments, the clinical decline is reduced by at least 25%, such as at least 30%, relative to placebo as determined by CDR-SB, wherein the subject is ApoE4-positive, and wherein the subject has been diagnosed as having mild cognitive impairment due to Alzheimer's disease—intermediate likelihood. In some embodiments, the clinical decline is reduced by at least 30%, such as at least 35% or 40%, relative to placebo as determined by CDR-SB, wherein the subject is ApoE4-positive, and wherein the subject has been diagnosed as having mild cognitive impairment due to Alzheimer's disease—intermediate likelihood. In some embodiments, the clinical decline is reduced by at least 35%, such as at least 40% or 45%, relative to placebo as determined by CDR-SB, wherein the subject is ApoE4-positive, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild cognitive impairment due to Alzheimer's disease—intermediate likelihood is reduced by at least 35%, such as at least 40% or at least 45%, relative to placebo as determined by CDR-SB composition comprising a therapeutically effective amount of at least one anti-Aβ protofibril antibody. In some embodiments, the clinical decline in the ApoE4-positive subject diagnosed as mild cognitive impairment due to Alzheimer's disease—intermediate likelihood is reduced by at least 20%, such as at least 25% or at least 30%, relative to placebo as determined by CDR-SB after 12 months of administration of the composition comprising a therapeutically effective amount of at least one anti-Aβ protofibril antibody. In some embodiments, the clinical decline in the ApoE4-positive subject diagnosed as having mild cognitive impairment due to Alzheimer's disease—intermediate likelihood is reduced by at least 35%, such as at least 40%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, or at least 119% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 76% to 119% relative to placebo as determined by ADCOMS, wherein the subject is ApoE4-positive, and wherein the subject has been diagnosed as having mild Alzheimer's disease dementia. In some embodiments, the clinical decline is reduced by 76% relative to placebo as determined by ADCOMS, wherein the subject is ApoE4-positive, and wherein the subject has been diagnosed as having mild Alzheimer's disease dementia. In some embodiments, the clinical decline is reduced by 113% relative to placebo as determined by ADCOMS, wherein the subject is ApoE4-positive, and wherein the subject has been diagnosed as having mild Alzheimer's disease dementia. In some embodiments, the clinical decline is reduced by 119% relative to placebo as determined by ADCOMS, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, at least 119%, at least 120%, at least 121%, at least 122%, at least 123%, at least 124%, at least 125%, at least 126%, at least 127%, at least 128%, at least 129%, at least 130%, at least 131%, at least 132%, at least 133%, at least 134%, at least 135%, at least 136%, at least 137%, at least 138%, at least 139%, at least 140%, at least 141%, at least 142%, at least 143%, at least 144%, at least 145%, at least 146%, at least 147%, at least 148%, at least 149%, at least 150%, at least 151%, at least 152%, at least 153%, at least 154%, at least 155%, at least 156%, at least 157%, at least 158%, at least 159%, at least 160%, at least 161%, at least 162%, at least 163%, at least 164%, at least 165%, at least 166%, at least 167%, at least 168%, at least 169%, at least 170%, at least 171%, at least 172%, at least 173%, at least 174%, at least 175%, at least 176%, at least 177%, at least 178%, at least 179%, at least 180%, at least 190%, at least 200%, at least 210%, at least 220%, at least 230%, at least 240%, at least 250%, at least 275%, at least 300%, at least 325%, at least 350%, at least 375%, at least 400%, at least 425%, at least 450%, at least 475%, at least 500%, at least 550%, at least 600%, at least 650%, at least 700%, at least 750%, at least 800%, at least 850%, at least 900%, at least 950%, at least 1000%, at least 1001%, at least 1002%, at least 1003%, at least 1004%, at least 1005%, at least 1006%, at least 1007%, at least 1008%, at least 1009%, at least 1010%, at least 1011%, at least 1012%, at least 1013%, at least 1014%, at least 1015%, at least 1016%, at least 1017%, at least 1018%, at least 1019%, at least 1020%, at least 1021%, at least 1022%, or at least 1023% relative to placebo as determined by ADAS-Cog, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 58% to 1023% relative to placebo as determined by ADAS-Cog, wherein the subject is ApoE4-positive, and wherein the subject has been diagnosed as having mild Alzheimer's disease dementia. In some embodiments, the clinical decline is reduced by 58% relative to placebo as determined by ADAS-Cog, wherein the subject is ApoE4-positive, and wherein the subject has been diagnosed as having mild Alzheimer's disease dementia. In some embodiments, the clinical decline is reduced by 171 relative to placebo as determined by ADAS-Cog, wherein the subject is ApoE4-positive, and wherein the subject has been diagnosed as having mild Alzheimer's disease dementia. In some embodiments, the clinical decline is reduced by 1023% relative to placebo as determined by ADAS-Cog, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 111%, at least 112%, at least 113%, at least 114%, at least 115%, at least 116%, at least 117%, at least 118%, at least 119%, at least 120%, at least 121%, at least 122%, at least 123%, at least 124%, at least 125%, at least 126%, at least 127%, at least 128%, at least 129%, at least 130%, at least 131%, at least 132%, at least 133%, at least 134%, at least 135%, at least 136%, at least 137%, at least 138%, at least 139%, at least 140%, at least 141%, at least 142%, at least 143%, at least 144%, at least 145%, at least 146%, at least 147%, at least 148%, at least 149%, at least 150%, at least 151%, at least 152%, at least 153%, at least 154%, at least 155%, at least 156%, at least 157%, at least 158%, at least 159%, at least 160%, at least 161%, at least 162%, at least 163%, at least 164%, at least 165%, at least 166%, at least 167%, at least 168%, at least 169%, at least 170%, at least 171%, at least 172%, at least 173%, or at least 174% relative to placebo as determined by CDR-SB, wherein the subject is ApoE4positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 70% to 200%, such as 75% to 180% or 82% to 174%, relative to placebo as determined by CDR-SB, wherein the subject is ApoE4-positive, and wherein the subject has been diagnosed as having mild Alzheimer's disease dementia. In some embodiments, the clinical decline is reduced by at least 70%, such as at least 80% relative to placebo as determined by CDR-SB, wherein the subject is ApoE4-positive, and wherein the subject has been diagnosed as having mild Alzheimer's disease dementia. In some embodiments, the clinical decline is reduced by at least 75%, such as at least 80% or at least 85%, relative to placebo as determined by CDR-SB, wherein the subject is ApoE4-positive, and wherein the subject has been diagnosed as having mild Alzheimer's disease dementia. In some embodiments, the clinical decline is reduced by at least 150%, such as at least 160% or 170%, relative to placebo as determined by CDR-SB, wherein the subject is ApoE4-positive, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-positive subject diagnosed as having mild Alzheimer's disease dementia is reduced by at least 70%, such as at least 75%, at least 80%, or at least 85%, relative to placebo as determined by CDR-SB composition comprising a therapeutically effective amount of at least one anti-Aβ protofibril antibody. In some embodiments, the clinical decline in the ApoE4-positive subject diagnosed as having mild Alzheimer's disease dementia is reduced by at least 130%, such as at least 140%, at least 150%, at least 160%, or at least 170%, relative to placebo as determined by CDR-SB after 12 months of administration of the composition comprising a therapeutically effective amount of at least one anti-Aβ protofibril antibody. In some embodiments, the clinical decline in the ApoE4-positive subject diagnosed as having mild Alzheimer's disease dementia is reduced by at least 65%, such as at least 70%, at least 75%, or at least 80%, relative to placebo as determined by CDR-SB after 18 months of administration of the composition comprising a therapeutically effective amount of at least one anti-Aβ protofibril antibody. In some embodiments, the composition comprises 10 mg/kg of at least one anti-β protofibril antibody and is administered once every two weeks or once every month. In some embodiments, the at least one anti-Aβ protofibril antibody is BAN2401.

In some embodiments, the subject is ApoE4-negative.

In some embodiments, the clinical decline is reduced by at least 1%, at least 2%, at least 3%, at least 4%, at least 5%, at least 6%, at least 7%, at least 8%, at least 9%, at least 10%, at least 11%, or at least 12% relative to placebo as determined by ADCOMS, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 5% to 15% relative to placebo as determined by ADCOMS, wherein the subject is ApoE4-negative. In some embodiments, the clinical decline is reduced by at least 5%, such as at least 7%, relative to placebo as determined by ADCOMS, wherein the subject is ApoE4-negative. In some embodiments, the clinical decline is reduced by at least 10%, such as at least 12%, relative to placebo as determined by ADCOMS, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-negative subject is reduced by at least −2% relative to placebo as determined by ADCOMS after 6 months of administration of the composition comprising a therapeutically effective amount of at least one anti-Aβ protofibril antibody. In some embodiments, the clinical decline in the ApoE4-negative subject is reduced by at least 10% relative to placebo as determined by ADCOMS after 12 months of administration of the composition comprising a therapeutically effective amount of at least one anti-Aβ protofibril antibody. In some embodiments, the clinical decline in the ApoE4-negative subject is reduced by at least 5%, such as at least 7%, relative to placebo as determined by ADCOMS after 18 months of administration of the composition comprising a therapeutically effective amount of at least one anti-Aβ protofibril antibody. In some embodiments, the clinical decline in the ApoE4-negative subject is reduced by at least 7%, relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, or at least 72% relative to placebo as determined by ADAS-cog, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 40% to 80% relative to placebo as determined by ADAS-cog, wherein the subject is ApoE4-negative. In some embodiments, the clinical decline is reduced by at least 35%, such as at least 40% or at least 43%, relative to placebo as determined by ADAS-cog, wherein the subject is ApoE4-negative. In some embodiments, the clinical decline is reduced by at least 40%, such as at least 45% or at least 46%, relative to placebo as determined by ADAS-cog, wherein the subject is ApoE4-negative. In some embodiments, the clinical decline is reduced by at least 65%, such as at least 70% or at least 72%, relative to placebo as determined by ADAS-cog, wherein the subject is ApoE4-negative. In some embodiments, the clinical decline is reduced by at least 43%, relative to placebo as determined by ADAS-cog, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is increased by 7%, 6%, 5%, 5%, 3%, 2%, or 1% relative to placebo as determined by CDR-SB, wherein the subject is ApoE4-negative. In some embodiments, the clinical decline is reduced by at least 1%, at least 2%, or at least 3% relative to placebo as determined by CDR-SB, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 3% relative to placebo as determined by CDR-SB, wherein the subject is ApoE4-negative. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody. In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, or at least 26% relative to placebo as determined by ADCOMS, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 15% to 26% relative to placebo as determined by ADCOMS, wherein the subject is ApoE4-negative, and wherein the subject has been diagnosed as having mild cognitive impairment due to Alzheimer's disease—intermediate likelihood. In some embodiments, the clinical decline is reduced by 15% relative to placebo as determined by ADCOMS, wherein the subject is ApoE4-negative, and wherein the subject has been diagnosed as having mild cognitive impairment due to Alzheimer's disease—intermediate likelihood. In some embodiments, the clinical decline is reduced by 26% relative to placebo as determined by ADCOMS, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 100%, at least 101%, at least 102%, at least 103%, at least 104%, at least 105%, at least 106%, at least 107%, at least 108%, at least 109%, at least 110%, at least 115%, at least 120%, at least 125%, at least 130%, at least 135%, at least 140%, at least 145%, at least 150%, at least 155%, at least 160%, at least 165%, or at least 166% relative to placebo as determined by ADAS-Cog, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 200%, such as 60% to 180% or 65% to 170%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the clinical decline is reduced by at least 50%, such as at least 55% or at least 65%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the clinical decline is reduced by at least 70%, such as at least 75% or at least 80%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the clinical decline is reduced by at least 150%, such as at least 160%, relative to placebo as determined by ADAS-Cog, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the ApoE4-negative subject is reduced by at least 150%, such as at least 160%, relative to placebo as determined by ADAS-Cog after 6 months of administration of the composition comprising a therapeutically effective amount of at least one anti-Aβ protofibril antibody. In some embodiments, the clinical decline in the ApoE4-negative subject is reduced by at least 70%, such as at least 75% or at least 80%, relative to placebo as determined by ADAS-Cog after 12 months of administration of the composition comprising a therapeutically effective amount of at least one anti-β protofibril antibody. In some embodiments, the clinical decline in the ApoE4-negative subject is reduced by at least 50%, such as at least 60% or at least 65%, relative to placebo as determined by ADAS-Cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, or at least 5% relative to placebo as determined by CDR-SB, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 5% relative to placebo as determined by CDR-SB, wherein the subject is ApoE4-negative, and wherein the subject has been diagnosed as having mild cognitive impairment due to Alzheimer's disease—intermediate likelihood. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12% relative to placebo as determined by CDR-SB, wherein the subject is ApoE4-negative, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by at least 10%, such as at least 12%, relative to placebo as determined by CDR-SB, wherein the subject is ApoE4-negative, and wherein the subject has been diagnosed as having mild Alzheimer's disease dementia. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 35% to 50% relative to placebo as determined by ADCOMS, wherein the subject is not concomitantly administered at least one Alzheimer's disease medication other than BAN2401. In some embodiments, the clinical decline is reduced by at least 35%, such as by at 38%, at least 40%, or at least 41%, relative to placebo as determined by ADCOMS, wherein the subject is not concomitantly administered at least one Alzheimer's disease medication other than BAN2401. In some embodiments, the clinical decline is reduced by at least 41%, relative to placebo as determined by ADCOMS, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody other than BAN2401.

In some embodiments, the clinical decline in the subject who is not concomitantly administered at least one Alzheimer's disease medication is reduced by at least 41% relative to placebo as determined by ADCOMS after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, or at least 59% relative to placebo as determined by ADAS-cog, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject is not concomitantly administered at least one Alzheimer's disease medication other than BAN2401. In some embodiments, the clinical decline is reduced by at least 50%, such as by at 55%, at least 57%, or at least 59%, relative to placebo as determined by ADAS-cog, wherein the subject is not concomitantly administered at least one Alzheimer's disease medication other than BAN2401. In some embodiments, the clinical decline is reduced by at least 59% relative to placebo as determined by ADAS-cog, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject who is not concomitantly administered at least one Alzheimer's disease medication other than BAN2401 is reduced by at least 59% relative to placebo as determined by ADAS-cog after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the clinical decline is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, or at least 45% relative to placebo as determined by CDR-SB, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline is reduced by 50% to 70% relative to placebo as determined by ADAS-cog, wherein the subject is not concomitantly administered at least one Alzheimer's disease medication other than BAN2401. In some embodiments, the clinical decline is reduced by at least 35%, such as by at 40%, at least 42%, or at least 45%, relative to placebo as determined by CDR-SB, wherein the subject is not concomitantly administered at least one Alzheimer's disease medication other than BAN2401. In some embodiments, the clinical decline is reduced by at least 45% relative to placebo as determined by CDR-SB, wherein the subject is not concomitantly administered at least one Alzheimer's disease medication other than BAN2401. In some embodiments, the above-recited reduction in clinical decline is determined after 1 month, 6 months, 12 months, 18 months, 60 months, and/or 63 months of administration of the composition comprising a therapeutically effective amount of at least one anti-Aβ protofibril antibody.

In some embodiments, the clinical decline in the subject who is not concomitantly administered at least one Alzheimer's disease medication other than BAN2401 is reduced by at least 45% relative to placebo as determined by CDR-SB after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

Prevention and/or Delay of Onset of Alzheimer's Disease

Also provided herein is a method of preventing and/or delaying onset of Alzheimer's disease in ApoE4-positive subjects. In some embodiments, said method comprises determining the brain amyloid level of a subject and then, if the brain amyloid level of the subject is above a first predetermined level, administering a composition comprising a therapeutically effective amount of at least one anti-Aβ protofibril antibody.

In some embodiments, the method further comprises measuring the post-administration brain amyloid level of the subject.

In some embodiments, the method further comprises determining a cerebrospinal fluid level of Aβ₁₋₄₂ and/or cerebrospinal fluid total tau level.

In some embodiments, the method further comprises determining a cerebrospinal fluid level of neurogranin.

In some embodiments, the method further comprises determining a cerebrospinal fluid level of neurofilament light chain.

In some embodiments, said method further comprises administering the composition if the post-administration brain amyloid level is above a second predetermined level.

In some embodiments, said method further comprises monitoring the brain amyloid level of the subject after administration until the brain amyloid level of the subject is below a first predetermined level.

In some embodiments, said method further comprises administering the composition if the post-administration cerebrospinal fluid level of Aβ₁₋₄₂ and/or cerebrospinal fluid total tau level is above a predetermined level.

In some embodiments, said method further comprises administering the composition if the post-administration cerebrospinal fluid level of neurogranin is above a predetermined level.

In some embodiments, said method further comprises administering the composition if the post-administration neurofilament light chain is above a predetermined level.

In some embodiments, said method further comprises administering at least one additional therapeutic agent. In some embodiments, the at least one additional therapeutic agent is chosen from BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof. In some embodiments, the at least one additional therapeutic agent is a BACE inhibitor. In some embodiments, the BACE inhibitor is chosen from CNP520, BI-1181181, LY2886721, LY3202626, PF-06751979, RG7129, atabecestat, elenbecestat, lanabecestat, and verubecestat. In some embodiments, the BACE inhibitor is elenbecestat.

Any of the anti-Aβ protofibril antibodies, therapeutically acceptable amounts thereof, dosing regimens therefor, and compositions comprising the same that are disclosed herein may be used in the method of reducing brain amyloid level in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month.

In some embodiments, the at least one anti-Aβ protofibril antibody is BAN2041.

In some embodiments, administration of the composition comprising at least one anti-Aβ protofibril antibody results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images.

In some embodiments, administration of the composition comprising at least one anti-Aβ protofibril antibody results in 75% to 100%, such as 80% to 100% or 85% to 100% of the subjects being amyloid negative, as determined by visual reads of amyloid PET images. In some embodiments, administration of the composition comprising at least one anti-Aβ protofibril antibody results in at least 75%, such as at least 80% or at least 85%, of the subjects being amyloid negative, as determined by visual reads of amyloid PET images. In some embodiments, administration of the composition comprising at least one anti-Aβ protofibril antibody results in 100% of the subjects being amyloid negative, as determined by visual reads of amyloid PET images.

In some embodiments, at least 75%, such as at least 80% or at least 85%, of the subjects are amyloid negative, as determined by visual reads of amyloid PET images, after 12 months of administration of the composition comprising a therapeutically effective amount of at least one anti-Aβ protofibril antibody. In some embodiments, at least 75%, such as at least 80%, at least 85%, at least 90%, or at least 95%, of the subjects are amyloid negative, as determined by visual reads of amyloid PET images, after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the method results in a reduced brain amyloid level after administration relative to the brain amyloid level prior to the administration. In some embodiments, the brain amyloid level is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the brain amyloid level prior to said administration.

In some embodiments, the above-recited reductions in brain amyloid levels are determined by visual reads of amyloid PET images and are expressed as PET standard uptake value ratios (SUVr values).

In some embodiments, the adjusted mean change in a subject's PET SUVr value is reduced by at least −0.10, at least −0.15, at least −0.20, at least −0.25, at least −0.30, at least −0.35, at least −0.40, at least −0.45, at least −0.50, at least −0.55, at least −0.60, at least −0.65, at least −0.70, at least −0.75, at least −0.80, at least −0.85, at least −0.90, or at least −0.95 relative to the brain amyloid level prior to administration of the composition comprising at least one anti-Aβ protofibril antibody. In some embodiments, the adjusted mean change in a subject's PET SUVr value from the brain amyloid level prior to the administration of the composition comprising at least one anti-Aβ protofibril antibody is reduced by −0.20 to −0.30.

In some embodiments, comparing global cortical average versus whole cerebellum reference, the adjusted mean change from the brain amyloid level prior to the administration of the composition comprising at least one anti-Aβ protofibril antibody in a subject's PET SUVr value is reduced by at least −0.20, such as at least −0.25, after 12 months of administration of the composition comprising a therapeutically effective amount of at least one anti-Aβ protofibril antibody. In some embodiments, the adjusted mean change in a subject's PET SUVr value is reduced by at least −0.25, such as at least −0.30, relative to the subject's PET SUVr value prior to the administration, after 18 months of administration of the composition comprising a therapeutically effective amount of at least one anti-Aβ protofibril antibody.

In some embodiments, the reduction of amyloid in the brain is determined by imaging using binding of radiotracers for brain Aβ amyloid and visualized with PET. In some embodiments, the reduction in the adjusted mean change from the subject's level prior to the administration of the composition comprising a therapeutically effective amount of at least one anti-Aβ protofibril antibody is at least −50, such as at least −55 or at least −59 centiloid after 12 months of administration of the composition comprising a therapeutically effective amount of at least one anti-Aβ protofibril antibody. In some embodiments, the reduction in the adjusted mean change from the subject's level prior to the administration of the composition comprising a therapeutically effective amount of at least one anti-Aβ protofibril antibody is at least −60, such as at least −65 or at least −70 centiloid after 18 months of administration of the composition comprising a therapeutically effective amount of at least one anti-Aβ protofibril antibody.

In some embodiments, the method results in a reduced cerebrospinal fluid Aβ₁₋₄₂ level relative to the cerebrospinal fluid Aβ₁₋₄₂ level prior to the administration. In some embodiments, the method results in a reduction of cerebrospinal fluid Aβ₁₋₄₂ level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Aβ₁₋₄₂ level prior to the administration.

In some embodiments, administration of the composition comprising at least one anti-Aβ protofibril antibody results in a brain amyloid level reduction of −0.20 to −0.45, such as from −0.25 to −0.35 as determined by visual reads of amyloid PET images, relative to the brain amyloid level prior to administration of the composition comprising at least one anti-Aβ protofibril antibody. In some embodiments, administration of the composition comprising at least one anti-Aβ protofibril antibody results in a brain amyloid level reduction of at least −0.25, as determined by visual reads of amyloid PET images, relative to the brain amyloid level prior to administration of the composition comprising at least one anti-Aβ protofibril antibody. In some embodiments, administration of the composition comprising at least one anti-Aβ protofibril antibody results in a brain amyloid level reduction of at least 0.30, as determined by visual reads of amyloid PET images, relative to the brain amyloid level prior to administration of the composition comprising at least one anti-Aβ protofibril antibody.

Also provided herein is another method of preventing and/or delaying onset Alzheimer's disease in ApoE4-positive subjects. In some embodiments, said method comprises determining the brain amyloid level of a subject and then, if the brain amyloid level of the subject is above a first predetermined level, administering a composition comprising a therapeutically effective amount of at least one anti-Aβ protofibril antibody and a composition comprising a therapeutically effective amount of at least one therapeutic agent chosen from BACE inhibitors, gamma secretase inhibitors, gamma secretase modulators, Aβ peptide generation inhibitors other than said at least one anti-Aβ protofibril antibody, and agents that lower the levels of Aβ peptide other than said at least one anti-Aβ protofibril antibody.

In some embodiments, the method further comprises measuring the post-administration brain amyloid level of the subject.

In some embodiments, the method further comprises determining a cerebrospinal fluid level of Aβ₁₋₄₂ and/or cerebrospinal fluid total tau level.

In some embodiments, the method further comprises determining a cerebrospinal fluid level of neurogranin.

In some embodiments, the method further comprises determining a cerebrospinal fluid level of neurofilament light chain.

In some embodiments, said method further comprises administering a composition comprising a therapeutically effective amount of at least one anti-Aβ protofibril antibody and a composition comprising a therapeutically effective amount of at least one therapeutic agent chosen from BACE inhibitors, gamma secretase inhibitors, gamma secretase modulators, Aβ peptide generation inhibitors other than said at least one anti-Aβ protofibril antibody, and agents that lower the levels of Aβ peptide other than said at least one anti-Aβ protofibril antibody if the post-administration brain amyloid level is above a second predetermined level.

In some embodiments, said method further comprises administering the composition if the post-administration cerebrospinal fluid level of Aβ₁₋₄₂ and/or cerebrospinal fluid total tau level is above a predetermined level.

In some embodiments, said method further comprises administering the composition if the post-administration cerebrospinal fluid level of neurogranin is above a predetermined level.

In some embodiments, said method further comprises administering the composition if the post-administration cerebrospinal fluid level of neurofilament light chain is above a predetermined level.

In some embodiments, said method further comprises monitoring the brain amyloid level of the subject after administration until the brain amyloid level of the subject is below a first predetermined level.

In some embodiments, said method further comprises administering at least one additional therapeutic agent. In some embodiments, the at least one additional therapeutic agent is chosen from BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof.

In some embodiments, the at least one additional therapeutic agent is a BACE inhibitor. In some embodiments, the BACE inhibitor is elenbecestat.

Any of the anti-Aβ protofibril antibodies, therapeutically acceptable amounts thereof, dosing regimens therefor, and compositions comprising the same that are disclosed herein may be used in the method of reducing brain amyloid level in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month.

In some embodiments, the at least one anti-Aβ protofibril antibody is BAN2041.

In some embodiments, the at least one therapeutic agent is a BACE inhibitor. In some embodiments, the BACE inhibitor is elenbecestat.

In some embodiments, administration of the composition comprising at least one anti-Aβ protofibril antibody and the composition comprising a therapeutically effective amount of at least one therapeutic agent results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images.

In some embodiments, administration of the composition comprising at least one anti-Aβ protofibril antibody and the composition comprising a therapeutically effective amount of at least one therapeutic agent results in 75% to 100%, such as 80% to 100% or 85% to 100% of the subjects being amyloid negative, as determined by visual reads of amyloid PET images. In some embodiments, administration of the composition comprising at least one anti-Aβ protofibril antibody and the composition comprising a therapeutically effective amount of at least one therapeutic agent results in at least 75%, such as at least 80% or at least 85%, of the subjects being amyloid negative, as determined by visual reads of amyloid PET images. In some embodiments, administration of the composition comprising at least one anti-Aβ protofibril antibody and the composition comprising a therapeutically effective amount of at least one therapeutic agent results in 100% of the subjects being amyloid negative, as determined by visual reads of amyloid PET images.

In some embodiments, at least 75%, such as at least 80% or at least 85%, of the subjects are amyloid negative, as determined by visual reads of amyloid PET images, after 12 months of administration of the composition comprising a therapeutically effective amount of at least one anti-Aβ protofibril antibody and the composition comprising a therapeutically effective amount of at least one therapeutic agent. In some embodiments, at least 75%, such as at least 80%, at least 85%, at least 90%, or at least 95%, of the subjects are amyloid negative, as determined by visual reads of amyloid PET images, after 18 months of administration of the composition comprising a therapeutically effective amount of at least one anti-Aβ protofibril antibody and the composition comprising a therapeutically effective amount of at least one therapeutic agent. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the method results in a reduced brain amyloid level after administration relative to the brain amyloid level prior to the administration. In some embodiments, the brain amyloid level is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the brain amyloid level prior to said administration. In some embodiments, the above-recited reductions in brain amyloid levels are determined by visual reads of amyloid PET images and are expressed as PET standard uptake value ratios (SUVr values).

In some embodiments, the adjusted mean change in a subject's PET SUVr value is reduced by at least −0.10, at least −0.15, at least −0.20, at least −0.25, at least −0.30, at least −0.35, at least −0.40, at least −0.45, at least −0.50, at least −0.55, at least −0.60, at least −0.65, at least −0.70, at least −0.75, at least −0.80, at least −0.85, at least −0.90, or at least −0.95 relative to the brain amyloid level prior to administration of the composition comprising at least one anti-Aβ protofibril antibody and the composition comprising a therapeutically effective amount of at least one therapeutic agent. In some embodiments, the adjusted mean change in a subject's PET SUVr value from the brain amyloid level prior to the administration of the composition comprising at least one anti-Aβ protofibril antibody and the composition comprising a therapeutically effective amount of at least one therapeutic agent is reduced by −0.20 to −0.30.

In some embodiments, comparing global cortical average versus whole cerebellum reference, the adjusted mean change from the brain amyloid level prior to the administration of the composition comprising at least one anti-Aβ protofibril antibody and the composition comprising a therapeutically effective amount of at least one therapeutic agent in a subject's PET SUVr value is reduced by at least −0.20, such as at least −0.25, after 12 months of administration of the composition comprising a therapeutically effective amount of at least one anti-Aβ protofibril antibody and the composition comprising a therapeutically effective amount of at least one therapeutic agent. In some embodiments, the adjusted mean change in a subject's PET SUVr value is reduced by at least −0.25, such as at least −0.30, relative to the subject's PET SUVr value prior to the administration, after 18 months of administration of the composition comprising a therapeutically effective amount of at least one anti-Aβ protofibril antibody and the composition comprising a therapeutically effective amount of at least one therapeutic agent.

In some embodiments, the reduction of amyloid in the brain is determined by imaging using binding of radiotracers for brain Aβ amyloid and visualized with PET. In some embodiments, the reduction in the adjusted mean change from the subject's level prior to the administration of the composition comprising a therapeutically effective amount of at least one anti-Aβ protofibril antibody and the composition comprising a therapeutically effective amount of at least one therapeutic agent is at least −50, such as at least −55 or at least −59 centiloid after 12 months of administration of the composition comprising a therapeutically effective amount of at least one anti-Aβ protofibril antibody and the composition comprising a therapeutically effective amount of at least one therapeutic agent. In some embodiments, the reduction in the adjusted mean change from the subject's level prior to the administration of the composition comprising a therapeutically effective amount of at least one anti-Aβ protofibril antibody is at least −60, such as at least −65 or at least −70 centiloid after 18 months of administration of the composition comprising a therapeutically effective amount of at least one anti-Aβ protofibril antibody and the composition comprising a therapeutically effective amount of at least one therapeutic agent.

In some embodiments, the method results in a reduced cerebrospinal fluid Aβ₁₋₄₂ level relative to the cerebrospinal fluid Aβ₁₋₄₂ level prior to the administration. In some embodiments, the method results in a reduction of cerebrospinal fluid Aβ₁₋₄₂ level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Aβ₁₋₄₂ level prior to the administration.

In some embodiments, administration of the composition comprising at least one anti-Aβ protofibril antibody and the composition comprising a therapeutically effective amount of at least one therapeutic agent results in a brain amyloid level reduction of −0.20 to −0.45, such as from −0.25 to −0.35 as determined by visual reads of amyloid PET images, relative to the brain amyloid level prior to administration of the composition comprising at least one anti-Aβ protofibril antibody and the composition comprising a therapeutically effective amount of at least one therapeutic agent. In some embodiments, administration of the composition comprising at least one anti-Aβ protofibril antibody and the composition comprising a therapeutically effective amount of at least one therapeutic agent results in a brain amyloid level reduction of at least −0.25, as determined by visual reads of amyloid PET images, relative to the brain amyloid level prior to administration of the composition comprising at least one anti-Aβ protofibril antibody. In some embodiments, administration of the composition comprising at least one anti-Aβ protofibril antibody and the composition comprising a therapeutically effective amount of at least one therapeutic agent results in a brain amyloid level reduction of at least 0.30, as determined by visual reads of amyloid PET images, relative to the brain amyloid level prior to administration of the composition comprising at least one anti-Aβ protofibril antibody and the composition comprising a therapeutically effective amount of at least one therapeutic agent.

Prevention and/or Delay of Onset of Alzheimer's Disease

Also provided herein is a method of preventing and/or delaying onset Alzheimer's disease in a subject. In some embodiments, the subject is ApoE4-positive. In some embodiments, the subject is ApoE4-negative. In some embodiments, the method comprises determining the brain amyloid level of a subject and then, if the brain amyloid level of the subject is above a first predetermined level, administering a composition comprising a therapeutically effective amount of at least one anti-Aβ protofibril antibody.

In some embodiments, the method further comprises measuring the post-administration brain amyloid level of the subject.

In some embodiments, the method further comprises determining a cerebrospinal fluid level of Aβ₁₋₄₂ and/or cerebrospinal fluid total tau level.

In some embodiments, the method further comprises determining a cerebrospinal fluid level of neurogranin.

In some embodiments, the method further comprises determining a cerebrospinal fluid level of neurofilament light chain.

In some embodiments, said method further comprises administering the composition if the post-administration brain amyloid level is above a second predetermined level.

In some embodiments, said method further comprises monitoring the brain amyloid level of the subject after administration until the brain amyloid level of the subject is below a first predetermined level.

In some embodiments, said method further comprises administering the composition if the post-administration cerebrospinal fluid level of Aβ₁₋₄₂ and/or cerebrospinal fluid total tau level is above a predetermined level.

In some embodiments, said method further comprises administering the composition if the post-administration cerebrospinal fluid level of neurogranin is above a predetermined level.

In some embodiments, said method further comprises administering the composition if the post-administration neurofilament light chain is above a predetermined level.

In some embodiments, said method further comprises administering at least one additional therapeutic agent. In some embodiments, the at least one additional therapeutic agent is chosen from BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof. In some embodiments, the at least one additional therapeutic agent is a BACE inhibitor. In some embodiments, the BACE inhibitor is chosen from CNP520, BI-1181181, LY2886721, LY3202626, PF-06751979, RG7129, atabecestat, elenbecestat, lanabecestat, and verubecestat. In some embodiments, the BACE inhibitor is elenbecestat.

Any of the anti-Aβ protofibril antibodies, therapeutically acceptable amounts thereof, dosing regimens therefor, and compositions comprising the same that are disclosed herein may be used in the method of reducing brain amyloid level in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month.

In some embodiments, the at least one anti-Aβ protofibril antibody is BAN2041.

In some embodiments, administration of the composition comprising at least one anti-Aβ protofibril antibody results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images.

In some embodiments, administration of the composition comprising at least one anti-Aβ protofibril antibody results in 75% to 100%, such as 80% to 100% or 85% to 100% of the subjects being amyloid negative, as determined by visual reads of amyloid PET images. In some embodiments, administration of the composition comprising at least one anti-Aβ protofibril antibody results in at least 75%, such as at least 80% or at least 85%, of the subjects being amyloid negative, as determined by visual reads of amyloid PET images. In some embodiments, administration of the composition comprising at least one anti-Aβ protofibril antibody results in 100% of the subjects being amyloid negative, as determined by visual reads of amyloid PET images.

In some embodiments, at least 75%, such as at least 80% or at least 85%, of the subjects are amyloid negative, as determined by visual reads of amyloid PET images, after 12 months of administration of the composition comprising a therapeutically effective amount of at least one anti-Aβ protofibril antibody. In some embodiments, at least 75%, such as at least 80%, at least 85%, at least 90%, or at least 95%, of the subjects are amyloid negative, as determined by visual reads of amyloid PET images, after 18 months of administration of the composition comprising a therapeutically effective amount of at least one anti-Aβ protofibril antibody. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the method results in a reduced brain amyloid level after administration relative to the brain amyloid level prior to the administration. In some embodiments, the brain amyloid level is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the brain amyloid level prior to said administration. In some embodiments, the above-recited reductions in brain amyloid levels are determined by visual reads of amyloid PET images and are expressed as PET standard uptake value ratios (SUVr values).

In some embodiments, the adjusted mean change in a subject's PET SUVr value is reduced by at least −0.10, at least −0.15, at least −0.20, at least −0.25, at least −0.30, at least −0.35, at least −0.40, at least −0.45, at least −0.50, at least −0.55, at least −0.60, at least −0.65, at least −0.70, at least −0.75, at least −0.80, at least −0.85, at least −0.90, or at least −0.95 relative to the brain amyloid level prior to administration of the composition comprising at least one anti-Aβ protofibril antibody. In some embodiments, the adjusted mean change in a subject's PET SUVr value from the brain amyloid level prior to the administration of the composition comprising at least one anti-Aβ protofibril antibody is reduced by −0.20 to −0.30.

In some embodiments, comparing global cortical average versus whole cerebellum reference, the adjusted mean change from the brain amyloid level prior to the administration of the composition comprising at least one anti-Aβ protofibril antibody in a subject's PET SUVr value is reduced by at least −0.20, such as at least −0.25, after 12 months of administration of the composition comprising a therapeutically effective amount of at least one anti-Aβ protofibril antibody. In some embodiments, the adjusted mean change in a subject's PET SUVr value is reduced by at least −0.25, such as at least −0.30, relative to the subject's PET SUVr value prior to the administration, after 18 months of administration of the composition comprising a therapeutically effective amount of at least one anti-Aβ protofibril antibody.

In some embodiments, the reduction of amyloid in the brain is determined by imaging using binding of radiotracers for brain Aβ amyloid and visualized with PET. In some embodiments, the reduction in the adjusted mean change from the subject's level prior to the administration of the composition comprising a therapeutically effective amount of at least one anti-Aβ protofibril antibody is at least −50, such as at least −55 or at least −59 centiloid after 12 months of administration of the composition comprising a therapeutically effective amount of at least one anti-Aβ protofibril antibody. In some embodiments, the reduction in the adjusted mean change from the subject's level prior to the administration of the composition comprising a therapeutically effective amount of at least one anti-Aβ protofibril antibody is at least −60, such as at least −65 or at least −70 centiloid after 18 months of administration of the composition comprising a therapeutically effective amount of at least one anti-Aβ protofibril antibody.

In some embodiments, the method results in a reduced cerebrospinal fluid Aβ₁₋₄₂ level relative to the cerebrospinal fluid Aβ₁₋₄₂ level prior to the administration. In some embodiments, the method results in a reduction of cerebrospinal fluid Aβ₁₋₄₂ level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Aβ₁₋₄₂ level prior to the administration.

In some embodiments, administration of the composition comprising at least one anti-Aβ protofibril antibody results in a brain amyloid level reduction of −0.20 to −0.45, such as from −0.25 to −0.35 as determined by visual reads of amyloid PET images, relative to the brain amyloid level prior to administration of the composition comprising at least one anti-Aβ protofibril antibody. In some embodiments, administration of the composition comprising at least one anti-Aβ protofibril antibody results in a brain amyloid level reduction of at least −0.25, as determined by visual reads of amyloid PET images, relative to the brain amyloid level prior to administration of the composition comprising at least one anti-Aβ protofibril antibody. In some embodiments, administration of the composition comprising at least one anti-Aβ protofibril antibody results in a brain amyloid level reduction of at least 0.30, as determined by visual reads of amyloid PET images, relative to the brain amyloid level prior to administration of the composition comprising at least one anti-Aβ protofibril antibody.

Also provided herein is another method of preventing and/or delaying onset Alzheimer's disease in subjects. In some embodiments, the subject is ApoE4-positive. In some embodiments, the subject is ApoE4-negative. In some embodiments, said method comprises determining the brain amyloid level of a subject and then, if the brain amyloid level of the subject is above a first predetermined level, administering a composition comprising a therapeutically effective amount of at least one anti-Aβ protofibril antibody and a composition comprising a therapeutically effective amount of at least one therapeutic agent chosen from BACE inhibitors, gamma secretase inhibitors, gamma secretase modulators, Aβ peptide generation inhibitors other than said at least one anti-Aβ protofibril antibody, and agents that lower the levels of Aβ peptide other than said at least one anti-Aβ protofibril antibody.

In some embodiments, the method further comprises measuring the post-administration brain amyloid level of the subject.

In some embodiments, the method further comprises determining a cerebrospinal fluid level of Aβ₁₋₄₂ and/or cerebrospinal fluid total tau level.

In some embodiments, the method further comprises determining a cerebrospinal fluid level of neurogranin.

In some embodiments, the method further comprises determining a cerebrospinal fluid level of neurofilament light chain.

In some embodiments, said method further comprises administering a composition comprising a therapeutically effective amount of at least one anti-Aβ protofibril antibody and a composition comprising a therapeutically effective amount of at least one therapeutic agent chosen from BACE inhibitors, gamma secretase inhibitors, gamma secretase modulators, Aβ peptide generation inhibitors other than said at least one anti-Aβ protofibril antibody, and agents that lower the levels of Aβ peptide other than said at least one anti-Aβ protofibril antibody if the post-administration brain amyloid level is above a second predetermined level.

In some embodiments, said method further comprises administering the composition if the post-administration cerebrospinal fluid level of Aβ₁₋₄₂ and/or cerebrospinal fluid total tau level is above a predetermined level.

In some embodiments, said method further comprises administering the composition if the post-administration cerebrospinal fluid level of neurogranin is above a predetermined level.

In some embodiments, said method further comprises administering the composition if the post-administration cerebrospinal fluid level of neurofilament light chain is above a predetermined level.

In some embodiments, said method further comprises monitoring the brain amyloid level of the subject after administration until the brain amyloid level of the subject is below a first predetermined level.

In some embodiments, said method further comprises administering at least one additional therapeutic agent. In some embodiments, the at least one additional therapeutic agent is chosen from BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof.

In some embodiments, the at least one additional therapeutic agent is a BACE inhibitor. In some embodiments, the BACE inhibitor is elenbecestat.

Any of the anti-Aβ protofibril antibodies, therapeutically acceptable amounts thereof, dosing regimens therefor, and compositions comprising the same that are disclosed herein may be used in the method of reducing brain amyloid level in a subject having early Alzheimer's disease. For example, in some embodiments, a composition comprising 2.5 mg/kg, 5 mg/kg, 7.5 mg/kg, or 10 mg/kg of at least one anti-Aβ protofibril antibody such as BAN2401 relative to body weight of the subject is administered to the subject once every week, once every two weeks, once every three weeks, once every four weeks, or once every month.

In some embodiments, the at least one anti-Aβ protofibril antibody is BAN2041.

In some embodiments, the at least one therapeutic agent is a BACE inhibitor. In some embodiments, the BACE inhibitor is elenbecestat.

In some embodiments, administration of the composition comprising at least one anti-Aβ protofibril antibody and the composition comprising a therapeutically effective amount of at least one therapeutic agent results in at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% of the subjects being amyloid negative, as determined by visual reads of amyloid PET images.

In some embodiments, administration of the composition comprising at least one anti-Aβ protofibril antibody and the composition comprising a therapeutically effective amount of at least one therapeutic agent results in 75% to 100%, such as 80% to 100% or 85% to 100% of the subjects being amyloid negative, as determined by visual reads of amyloid PET images. In some embodiments, administration of the composition comprising at least one anti-Aβ protofibril antibody and the composition comprising a therapeutically effective amount of at least one therapeutic agent results in at least 75%, such as at least 80% or at least 85%, of the subjects being amyloid negative, as determined by visual reads of amyloid PET images. In some embodiments, administration of the composition comprising at least one anti-Aβ protofibril antibody and the composition comprising a therapeutically effective amount of at least one therapeutic agent results in 100% of the subjects being amyloid negative, as determined by visual reads of amyloid PET images.

In some embodiments, at least 75%, such as at least 80% or at least 85%, of the subjects are amyloid negative, as determined by visual reads of amyloid PET images, after 12 months of administration of the composition comprising a therapeutically effective amount of at least one anti-Aβ protofibril antibody and the composition comprising a therapeutically effective amount of at least one therapeutic agent. In some embodiments, at least 75%, such as at least 80%, at least 85%, at least 90%, or at least 95%, of the subjects are amyloid negative, as determined by visual reads of amyloid PET images, after 18 months of administration of the composition comprising a therapeutically effective amount of at least one anti-Aβ protofibril antibody and the composition comprising a therapeutically effective amount of at least one therapeutic agent. In some embodiments, the composition comprises 10 mg/kg of at least one anti-Aβ protofibril antibody and is administered once every two weeks or once every month. In some embodiments, the at least one anti-Aβ protofibril antibody is BAN2401.

In some embodiments, the method results in a reduced brain amyloid level after administration relative to the brain amyloid level prior to the administration. In some embodiments, the brain amyloid level is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the brain amyloid level prior to said administration. In some embodiments, the above-recited reductions in brain amyloid levels are determined by visual reads of amyloid PET images and are expressed as PET standard uptake value ratios (SUVr values).

In some embodiments, the adjusted mean change in a subject's PET SUVr value is reduced by at least −0.10, at least −0.15, at least −0.20, at least −0.25, at least −0.30, at least −0.35, at least −0.40, at least −0.45, at least −0.50, at least −0.55, at least −0.60, at least −0.65, at least −0.70, at least −0.75, at least −0.80, at least −0.85, at least −0.90, or at least −0.95 relative to the brain amyloid level prior to administration of the composition comprising at least one anti-Aβ protofibril antibody and the composition comprising a therapeutically effective amount of at least one therapeutic agent. In some embodiments, the adjusted mean change in a subject's PET SUVr value from the brain amyloid level prior to the administration of the composition comprising at least one anti-Aβ protofibril antibody and the composition comprising a therapeutically effective amount of at least one therapeutic agent is reduced by −0.20 to −0.30.

In some embodiments, comparing global cortical average versus whole cerebellum reference, the adjusted mean change from the brain amyloid level prior to the administration of the composition comprising at least one anti-Aβ protofibril antibody and the composition comprising a therapeutically effective amount of at least one therapeutic agent in a subject's PET SUVr value is reduced by at least −0.20, such as at least −0.25, after 12 months of administration of the composition comprising a therapeutically effective amount of at least one anti-Aβ protofibril antibody and the composition comprising a therapeutically effective amount of at least one therapeutic agent. In some embodiments, the adjusted mean change in a subject's PET SUVr value is reduced by at least −0.25, such as at least −0.30, relative to the subject's PET SUVr value prior to the administration, after 18 months of administration of the composition comprising a therapeutically effective amount of at least one anti-Aβ protofibril antibody and the composition comprising a therapeutically effective amount of at least one therapeutic agent.

In some embodiments, the reduction of amyloid in the brain is determined by imaging using binding of radiotracers for brain Aβ amyloid and visualized with PET. In some embodiments, the reduction in the adjusted mean change from the subject's level prior to the administration of the composition comprising a therapeutically effective amount of at least one anti-Aβ protofibril antibody and the composition comprising a therapeutically effective amount of at least one therapeutic agent is at least −50, such as at least −55 or at least −59 centiloid after 12 months of administration of the composition comprising a therapeutically effective amount of at least one anti-Aβ protofibril antibody and the composition comprising a therapeutically effective amount of at least one therapeutic agent. In some embodiments, the reduction in the adjusted mean change from the subject's level prior to the administration of the composition comprising a therapeutically effective amount of at least one anti-Aβ protofibril antibody is at least −60, such as at least −65 or at least −70 centiloid after 18 months of administration of the composition comprising a therapeutically effective amount of at least one anti-Aβ protofibril antibody and the composition comprising a therapeutically effective amount of at least one therapeutic agent.

In some embodiments, the method results in a reduced cerebrospinal fluid Aβ₁₋₄₂ level relative to the cerebrospinal fluid Aβ₁₋₄₂ level prior to the administration. In some embodiments, the method results in a reduction of cerebrospinal fluid Aβ₁₋₄₂ level of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% relative to the cerebrospinal fluid Aβ₁₋₄₂ level prior to the administration.

In some embodiments, administration of the composition comprising at least one anti-Aβ protofibril antibody and the composition comprising a therapeutically effective amount of at least one therapeutic agent results in a brain amyloid level reduction of −0.20 to −0.45, such as from −0.25 to −0.35 as determined by visual reads of amyloid PET images, relative to the brain amyloid level prior to administration of the composition comprising at least one anti-Aβ protofibril antibody and the composition comprising a therapeutically effective amount of at least one therapeutic agent. In some embodiments, administration of the composition comprising at least one anti-Aβ protofibril antibody and the composition comprising a therapeutically effective amount of at least one therapeutic agent results in a brain amyloid level reduction of at least −0.25, as determined by visual reads of amyloid PET images, relative to the brain amyloid level prior to administration of the composition comprising at least one anti-Aβ protofibril antibody. In some embodiments, administration of the composition comprising at least one anti-Aβ protofibril antibody and the composition comprising a therapeutically effective amount of at least one therapeutic agent results in a brain amyloid level reduction of at least 0.30, as determined by visual reads of amyloid PET images, relative to the brain amyloid level prior to administration of the composition comprising at least one anti-Aβ protofibril antibody and the composition comprising a therapeutically effective amount of at least one therapeutic agent.

### Adverse Events

In some embodiments, the methods provided herein do not result in the occurrence of one or more serious adverse events. In some embodiments, the methods provided herein results in one or more serious adverse events that are less severe than Grade 5, less severe than Grade 4, less severe than Grade 3, less severe than Grade 2, and/or less severe than Grade 1. In some embodiments, the subject does not report the one or more adverse events because it is less than Grade 1 severity.

In some embodiments, the methods described herein result in less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, or less than 20% of subjects experiencing a serious adverse event.

In some embodiments, the methods described herein result in less than 1%, less than 2%, less than 3%, less than 4%, less than 5%, less than 6%, less than 7%, less than 8%, less than 9%, or less than 10% of subjects experiencing vasogenic edema.

In some embodiments, the methods described herein result in less than 1%, less than 2%, less than 3%, less than 4%, less than 5%, less than 6%, less than 7%, less than 8%, less than 9%, less than 10%, less than 12%, less than 13%, less than 14%, or less than 15% of ApoE4-positive subjects experiencing vasogenic edema.

In some embodiments, the methods described herein result in less than 15% of ApoE4-positive subjects experiencing vasogenic edema.

In some embodiments, the methods described herein result in less than 1%, less than 2%, less than 3%, less than 4%, less than 5%, less than 6%, less than 7%, less than 8%, less than 9%, or less than 10% of ApoE4-negative subjects experiencing vasogenic edema.

In some embodiments, the methods described herein result in less than 10% of ApoE4-negative subjects experiencing vasogenic edema.

The contents of articles and patent documents referenced herein are incorporated by reference herein in their entirety.

### EXAMPLES

### Example 1

### Treatment of Subjects Haying Early Alzheimer's Disease

Subjects, both male and female with ages ranging from 50 to 90 years old, inclusive, having mild cognitive impairment due to Alzheimer's disease—intermediate likelihood and/or mild Alzheimer's disease dementia were screened to be eligible for treatment. 854 subjects were randomized for treatment.

The study consisted of a prerandomization phase and a randomization phase.

### Prerandomization Phase

This phase lasted up to 60 days and consisted of a screening period and a baseline period. During the screening period, subjects were assessed based on eligibility criteria. If a subject was deemed to be eligible, the subject proceeded to the baseline period.

During the baseline period, a number of analyses were performed, including clinical tests (e.g., blood tests (e.g., for determining ApoE4 status), etc.), safety magnetic resonance imaging (MRI) analyses, amyloid PET assessments (e.g., visual reads of amyloid PET images), and cerebrospinal fluid (for biomarker analysis) if the subject consented. Additional clinical testing of subjects included MMSE, CDR, ADAS-Cog, and FAQ.

### Randomization Phase

The randomization phase consisted of an 18-month treatment period and a 3-month follow-up period. Subjects were randomized to one of the following regimens:
placebo;
composition comprising 2.5 mg/kg BAN2401, once every 2 weeks;
composition comprising 5 mg/kg BAN2401, once every 2 weeks;
composition comprising 10 mg/kg BAN2401, once every 2 weeks;
composition comprising 5 mg/kg BAN2401, once a month; or
composition comprising 10 mg/kg BAN2401, once a month.

All subjects were administered an approximately 60-minute intravenous infusion every 2 weeks. BAN2401 was administered in normal saline as 60-minute IV infusions using an infusion system containing a terminal 0.22 μm in-line filter. All subjects received biweekly infusions, and subjects who had monthly dosing of BAN2401 had placebo infusion alternating with BAN2401.

All subjects were assessed based on cognitive function, safety, pharmacokinetic parameters, safety MRI, volumetric MRI and cerebrospinal fluid analysis, if the subject consented to such analysis. Additional clinical assessment included MMSE, CDR, ADAS-cog, and FAQ.

Treatment was ended at the later of 18 months, request by the subject, request of the attending physician and/or data safety monitoring board, and/or the emergence of one or more adverse events warranting the discontinuation of treatment.

After the end of treatment, subjects were evaluated as during the baseline period and randomization phase.

### Formulation

BAN2401 was supplied as a sterile, clear solution for injection containing 10 mg/mL, in a single use 10 mL vial (total 100 mg/vial). The drug product was formulated in 25 mM sodium citrate, 125 mM sodium chloride, 0.02% (w/v) polysorbate 80, and the pH was determined to be 5.7.

### Study Endpoints

The primary study objectives included (1) evaluating the efficacy of BAN2401 compared to placebo by establishing the ED₉₀ (as defined in the protocol) for BAN2401 on the Alzheimer's Disease Composite Score (ADCOMS) at 12 months of treatment in subjects with Early Alzheimer's Disease (EAD), defined as mild cognitive impairment (MCI) due to Alzheimer's disease—intermediate likelihood or mild Alzheimer's disease dementia; and (2) assess the safety and tolerability of 3 doses and 2 dose regimens of BAN2401 in subjects with early Alzheimer's disease.

Key secondary objectives included:
evaluating the effects of BAN2401 compared to placebo on brain amyloid pathophysiology at 18 months of treatment in subjects with early Alzheimer's disease as measured by PET;
evaluating the efficacy of BAN2401 compared to placebo on the ADCOMS at 18 months of treatment in subjects with early Alzheimer's disease;
evaluating the efficacy of BAN2401 compared to placebo on the Clinical Dementia Rating—Sum of Boxes (CDR-SB) at 18 months of treatment in subjects with early Alzheimer's disease;
evaluating the efficacy of BAN2401 compared to placebo on Alzheimer Disease Assessment Scale—Cognitive Subscale (ADAS-cog) in subjects with EAD at 18 months;
evaluating the effects of BAN2401 compared to placebo at 18 months on clinical status separately within subjects with MCI and mild AD dementia for the following assessments: ADCOMS, CDR-SB, and ADAS-cog;
evaluating the effects of BAN2401 compared to placebo on cerebrospinal fluid biomarkers (Aβ₁₋₄₂, t-tau, and p-tau) at 18 months of treatment in subjects with early Alzheimer's disease; and
evaluating the effects of BAN2401 compared to placebo on total hippocampal volume using volumetric magnetic resonance imaging (vMRI) at 18 months of treatment in subjects with early Alzheimer's disease.

TABLE 1
Treatment regimen enrollment.

| Duration of | | 2.5 mg/kg | 5 mg/kg | 5 mg/kg | 10 mg/kg | 10 mg/kg | |
|---|---|---|---|---|---|---|---|
| exposure | Placebo | bi-Weekly | Monthly | bi-Weekly | Monthly | bi-Weekly | Total |
| (months) | (N = 245) | (N = 52) | (N = 51) | (N = 92) | (N = 253) | (N = 161) | (N = 609) |
| n | 245 | 52 | 51 | 92 | 253 | 161 | 609 |
| Mean | 15.55 | 15.01 | 16.44 | 15.21 | 13.95 | 12.03 | 13.93 |
| Number of | 3810.0 | 780.5 | 838.6 | 1399.4 | 3529.6 | 1936.2 | 8484.2 |
| subject- | | | | | | | |
| months | | | | | | | |

TABLE 2
Overall Baseline Demographics and Characteristics.

| | | BAN2401 | Placebo |
|---|---|---|---|
| Characteristic | | (N = 587) | (N = 238) |
| Year of age | | 71.4 ± 7.91 | 71.1 ± 8.89 |
| (mean ± S.D.) | | | |
| Male (n(%)) | | 315 (53.7) | 101 (42.4) |
| Clinical Stage (n(%)) | MCl | 375 (63.9) | 154 (64.7) |
| | Mild AD | 212 (36.1) | 84 (35.3) |
| ApoE4 (n(%)) | Carriers | 420 (71.6) | 169 (71.0) |
| | Non- | 167 (28.4) | 69 (29.0) |
| | carriers | | |
| Ongoing | No | 268 (45.7) | 110 (46.2) |
| acetycholinesterase | | | |
| inhibitor/memantine | | | |
| treatment | | | |
| at baseline | | | |
| | Yes | 319 (54.3) | 128 (53.8) |
| Global CDR (n(%)) | 0.5 | 504 (85.9) | 200 (84.0) |
| | 1 | 83 (14.1) | 38 (16.0) |
| ADCOMS | | 0.378 ± 0.158 | 0.370 ± 0.166 |
| (mean ± S.D.) | | | |
| ADAS-cog^{#} | | 22.2 ± 7.4 | 22.6 ± 7.7 |
| (mean ± S.D.) | | | |
| CDR-SB | | 2.95 ± 1.37 | 2.89 ± 1.45 |
| (mean ± S.D.) | | | |
| MMSE | | 25.6 ± 2.4 | 26.0 ± 2.3 |
| (mean ± S.D.) | | | |
| PET SUVr | | 1.41 ± 0.16 | 1.40 ± 0.16 |
| (mean ± S.D.) | | | |

^{#}N = 586 in BAN2401, N = 237 in Placebo
*PET sub study; N = 214 in BAN2401, N = 98 in Placebo

TABLE 3
Baseline demographics of subjects having mild cognitive
impairment due to Alzheimer's disease-moderate likelihood.

| | | BAN2401 | Placebo |
|---|---|---|---|
| Characteristic | | (N = 375) | (N = 375) |
| Year of age | | 71.3 ± 7.47 | 71.3 ± 8.69 |
| (mean ± S.D.) | | | |
| Male (n(%)) | | 203 (54.1) | 70 (45.5) |
| ApoE4 (n(%)) | Carriers | 272 (72.5) | 110 (71.4) |
| | Non- | 103 (27.5) | 44 (28.6) |
| | carriers | | |
| Ongoing | No | 203 (54.1) | 85 (55.2) |
| acetylcholinesterase | | | |
| inhibitor/memantine | | | |
| treatment at | | | |
| baseline | | | |
| | Yes | 172 (45.9) | 69 (44.8) |
| Global CDR (n(%)) | 0.5 | 373 (99.5) | 153 (99.4) |
| | 1 | 2 (0.5) | 1(0.6) |

TABLE 4
Baseline demographics of subjects having mild Alzheimer's
disease dementia.

| | | BAN2401 | Placebo |
|---|---|---|---|
| Characteristic | | ( N = 212) | (N = 84) |
| Year of age | | 71.5 ± 8.64 | 70.7 ± 9.29 |
| (mean ± S.D.) | | | |
| Male (n(%)) | | 112 (52.8) | 31 (36.9) |
| ApoE4 (n(%)) | Carriers | 148 (69.8) | 59 (70.2) |
| | Non- | 64 (30.2) | 25 (29.8) |
| | carriers | | |
| Ongoing | No | 65 (30.7) | 25 (29.8) |
| acetylcholinesterase | | | |
| inhibitor/memantine | | | |
| treatment at baseline | | | |
| | Yes | 147 (69.3) | 59 (70.2) |
| Global CDR (n(%)) | 0.5 | 131 (61.8) | 47 (56.0) |
| | 1 | 81 (38.2) | 37 (44.0) |

### Endpoints

A primary endpoint used ADCOMS to measure clinical outcome assessment using longitudinal data through 12 months with Bayesian analysis.

Secondary endpoints (12 month and 18 month time points) included change from baseline in PET SUVr (amyloid load); conversion from amyloid positive to negative (visual read); change from baseline in ADCOMS; change from baseline in ADAS-cog; change from baseline in CDR-SB; change from baseline in CSF measures (Aβ₁₋₄₂, total-Tau, etc.).

### Results

### Cognition Results

In the overall subject population, dose-dependent, clinically meaningful, and statistically significant slower decline on clinical outcome measures of cognition and function were observed after 18 months on ADCOMS with 30% less decline on top dose and ADAS-cog with 47% less decline on top dose; and dose-dependent, clinically meaningful slower decline at 18 months on CDR-SB with 26% less decline on top dose.

Tables 5 and 6 provide the change from baseline after 12 months of administration of BAN2401, as determined by ADCOMS.

TABLE 5
Change from baseline for 2.5 mg/kg bi-weekly, 5 mg/kg monthly,
5 mg/kg bi-weekly, 10 mg/kg monthly, and 10 mg/kg bi-weekly
doses of BAN2401, as determined by ADCOMS, after 12 months.

| | | 2.5 mg/kg | 5 mg/kg | 5 mg/kg | 10 mg/kg | 10 mg/kg |
|---|---|---|---|---|---|---|
| | Placebo | bi-weekly | monthly | bi-weekly | monthly | bi-weekly |
| N | 187 | 38 | 42 | 67 | 165 | 93 |
| Obs. Mean | 0.102 | 0.149 | 0.106 | 0.098 | 0.079 | 0.076 |
| CFB (SD) | (0.155) | (0.201) | (0.169) | (0.141) | (0.163) | (0.144) |
| Obs. diff. | — | 0.047 | 0.004 | −0.004 | −0.023 | −0.026 |
| LS Mean | 0.131 | 0.158 | 0.149 | 0.139 | 0.102 | 0.085 |
| (SE) | (0.013) | (0.027) | (0.027) | (0.021) | (0.014) | (0.017) |
| LSM | — | 0.028 | 0.019 | 0.008 | −0.029 | −0.046 |
| difference | | | | | | |
| % less | — | 21.4 | 14.5 | 6.1 | −22.1 | −35.1 |
| decline | | | | | | |
| P-value | — | 0.336 | 0.514 | 0.731 | 0.101 | 0.027 |

TABLE 6
Change from baseline for the combined 10 mg/kg (once every two
weeks and once a month) doses, as determined by ADCOMS, after 12
months.

| | | Combined |
|---|---|---|
| | | 10 mg/kg |
| | Placebo | groups |
| N | 187 | 258 |
| Obs. Mean | 0.102 (0.155) | 0.078 (0.156) |
| CFB (SD) | | |
| Obs. diff. | — | −0.024 |
| LS Mean (SE) | 0.128 (0.013) | 0.093 (0.012) |
| LSM diff | — | −0.035 |
| % less decline | — | −27.3 |
| P-value | — | 0.019 |

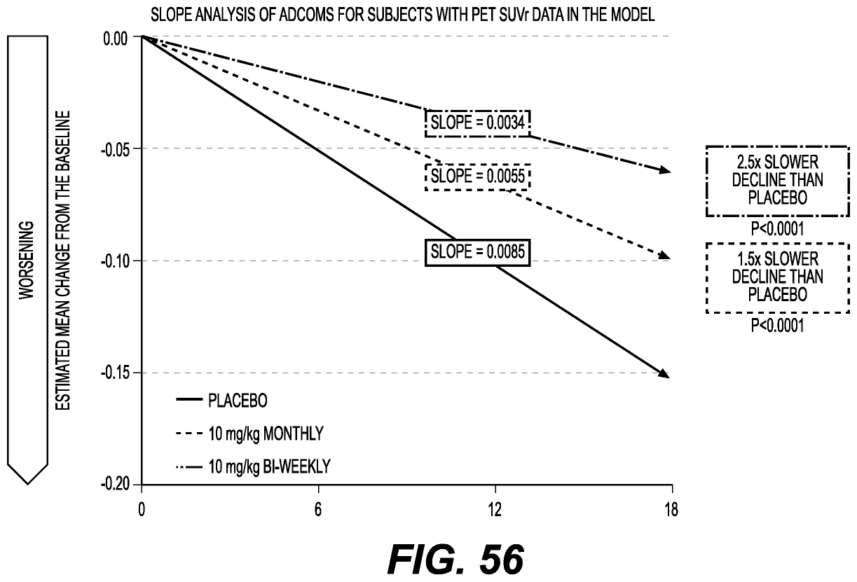

Similarly, Tables 7 and 8 provide the change from baseline after 18 months of administration of various doses of BAN2401, as determined by ADCOMS. See also FIG. 56.

TABLE 7
Change from baseline for 2.5 mg/kg bi-weekly, 5 mg/kg monthly,
5 mg/kg bi-weekly, 10 mg/kg monthly, and 10 mg/kg bi-weekly
doses of BAN2401, as determined by ADCOMS, after 18 months.

| | | 2.5 mg/kg | 5 mg/kg | 5 mg/kg | 10 mg/kg | 10 mg/kg |
|---|---|---|---|---|---|---|
| | Placebo | bi-weekly | monthly | bi-weekly | monthly | bi-weekly |
| N* | 160 | 33 | 35 | 61 | 146 | 79 |
| Obs. Mean | 0.164 | 0.167 | 0.121 | 0.166 | 0.138 | 0.122 |
| CFB (SD) | (0.197) | (0.234) | (0.194) | (0.205) | (0.215) | (0.180) |
| Obs. diff. | — | 0.003 | −0.043 | 0.002 | −0.026 | −0.042 |
| LS Mean | 0.193 | 0.173 | 0.192 | 0.199 | 0.166 | 0.136 |
| (SE) | (0.017) | (0.035) | (0.035) | (0.026) | (0.018) | (0.022) |
| LSM diff. | — | −0.020 | −0.001 | 0.006 | −0.028 | −0.057 |
| % less | — | −10.4 | −0.5 | 3.1 | −14.5 | −29.5 |
| decline | | | | | | |
| P-value | — | 0.592 | 0.971 | 0.855 | 0.228 | 0.034 |

TABLE 8
Change from baseline for the combined 10 mg/kg (once every two
weeks and once a month) doses, as determined by ADCOMS, after 18
months.

| | | Combined 10 |
|---|---|---|
| | Placebo | mg/kg groups |
| N* | 160 | 225 |
| Obs. Mean | 0.164 (0.197) | 0.132 (0.204) |
| CFB (SD) | | |
| Obs. diff. | — | −0.032 |
| LS Mean (SE) | 0.190 (0.017) | 0.152 (0.014) |
| LSM diff. | — | −0.039 |
| % less decline | — | −20.5 |
| P-value | — | 0.053 |

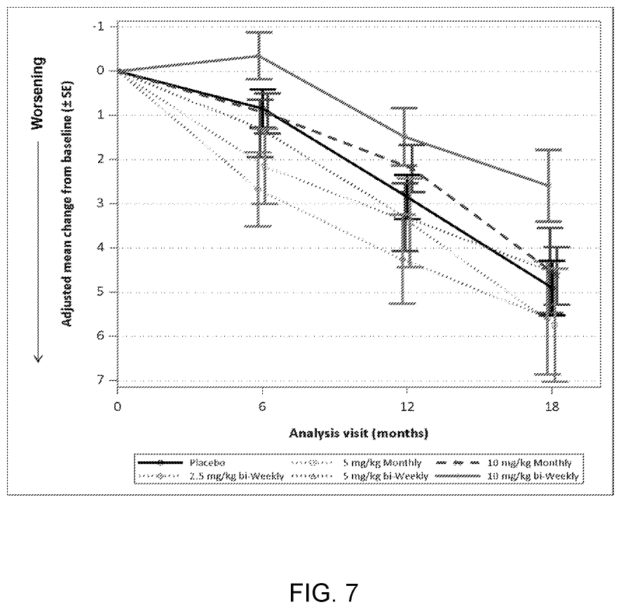

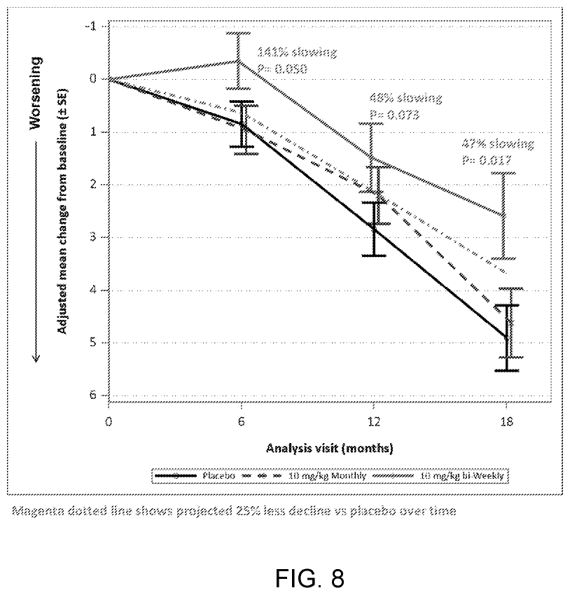

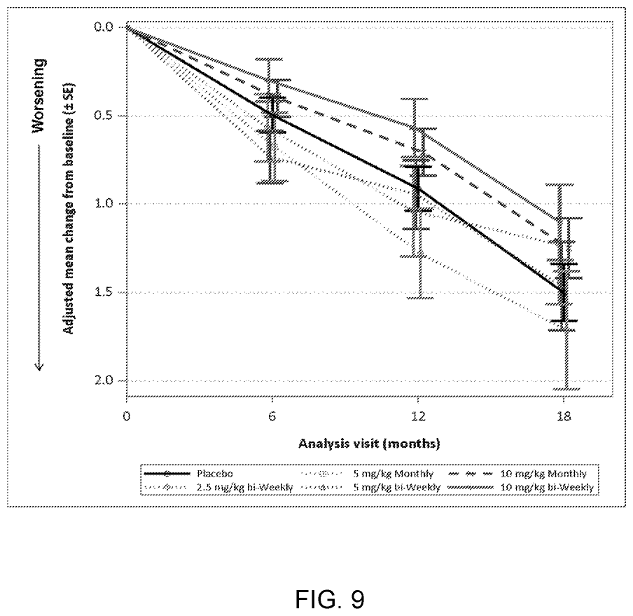

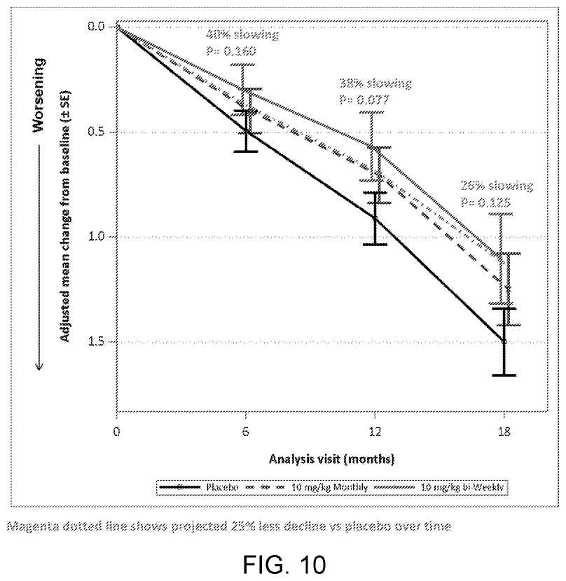

Tables 9 and 10 provide the change from baseline after 12 months of administration of various doses of BAN2401, as determined by CDR-SB. As shown in FIGS. 9 and 10, a dose of 10 mg/kg monthly and a dose of 10 mg/kg bi-weekly resulted in a slowing of cognitive decline relative to placebo, as determined by CDR-SB, after 12 and 18 months of treatment with a composition comprising a therapeutically effective amount of BAN2401. A similar trend is observed when using ADAS-cog as the statistical analysis method. See FIGS. 7 and 8.

TABLE 9
Change from baseline for 2.5 mg/kg bi-weekly, 5 mg/kg monthly,
5 mg/kg bi-weekly, 10 mg/kg monthly, and 10 mg/kg bi-weekly
doses of BAN2401, as determined by CDR-SB, after 12 months.

| | | 2.5 mg/kg | 5 mg/kg | 5 mg/kg | 10 mg/kg | 10 mg/kg |
|---|---|---|---|---|---|---|
| | Placebo | bi-weekly | monthly | bi-weekly | monthly | bi-weekly |
| N* | 188 | 38 | 42 | 70 | 166 | 94 |
| Obs. | 0.73 | 1.18 | 0.99 | 0.75 | 0.59 | 0.46 |
| Mean | (1.402) | (1.906) | (1.371) | (1.451) | (1.526) | (1.382) |
| CFB (SD) | | | | | | |
| Obs. diff. | — | 0.45 | 0.26 | 0.02 | −0.14 | −0.27 |
| LS Mean | 0.911 | 1.038 | 1.277 | 0.945 | 0.705 | 0.568 |
| (SE) | (0.124) | (0.257) | (0.253) | (0.194) | (0.133) | (0.163) |
| LSM diff. | — | 0.127 | 0.366 | 0.034 | −0.207 | −0.344 |
| % less | — | 13.9 | 40.2 | 3.7 | −22.7 | −37.8 |
| decline | | | | | | |
| P-value | — | 0.644 | 0.179 | 0.878 | 0.210 | 0.077 |

TABLE 10
Change from baseline for the combined 10 mg/kg (once every two
weeks and once a month) doses, as determined by ADCOMS, after 12
months.

| | | Combined 10 |
|---|---|---|
| | Placebo | mg/kg groups |
| N* | 188 | 260 |
| Obs. Mean | 0.73 (1.402) | 0.54 (1.476) |
| CFB (SD) | | |
| Obs. diff. | | −0.19 |
| LS Mean (SE) | 0.894 (0.123) | 0.642 (0.108) |
| LSM diff. | | −0.253 |
| % less decline | | −28.3 |
| P-value | | 0.079 |

Tables 11 and 12 provide the change from baseline after 18 months of administration of various doses of BAN2401, as determined by CDR-SB.

TABLE 11
Change from baseline for 2.5 mg/kg bi-weekly, 5 mg/kg monthly,
5 mg/kg bi-weekly, 10 mg/kg monthly, and 10 mg/kg bi-weekly
doses of BAN2401, as determined by CDR-SB, after 18 months.

| | | 2.5 mg/kg | 5 mg/kg | 5 mg/kg | 10 mg/kg | 10 mg/kg |
|---|---|---|---|---|---|---|
| | Placebo | bi-weekly | monthly | bi-weekly | monthly | bi-weekly |
| N* | 161 | 34 | 36 | 67 | 149 | 84 |
| Obs. | 1.27 | 1.37 | 1.23 | 1.26 | 1.10 | 1.04 |
| Mean CFB | (1.778) | (2.174) | (1.776) | (2.014) | (2.033) | (1.978) |
| (SD) | | | | | | |
| Obs. diff. | — | 0.10 | −0.04 | −0.01 | −0.17 | −0.23 |
| LS Mean | 1.499 | 1.227 | 1.713 | 1.463 | 1.248 | 1.102 |
| (SE) | (0.160) | (0.338) | (0.334) | (0.250) | (0.169) | (0.213) |
| LSM diff. | — | −0.271 | 0.214 | −0.036 | −0.250 | −0.396 |
| % less | — | −18.1 | 14.3 | −2.4 | −16.7 | −26.4 |
| decline | | | | | | |
| P-value | — | 0.459 | 0.555 | 0.901 | 0.255 | 0.125 |

TABLE 12
Change from baseline for the combined 10 mg/kg (once every two
weeks and once a month) doses, as determined by CDR-SB, after 18
months.

| | | Combined |
|---|---|---|
| | | 10 mg/kg |
| | Placebo | groups |
| N* | 161 | 233 |
| Obs. Mean | 1.27 | 1.08 (2.010) |
| CFB (SD) | (1.778) | |
| Obs. diff. | — | −0.19 |
| LS Mean (SE) | 1.473 (0.158) | 1.171 (0.136) |
| LSM diff. | — | −0.302 |
| % less decline | — | −20.5 |
| P-value | — | 0.119 |

Results in ApoE4-positive Subjects

As is reported below, unpredictably beneficial results were achieved when the subjects were ApoE4 positive as compared to subjects who were non-carriers of ApoE4. This is surprising at least in view of previously reported results of studies performed with other anti-Aβ antibodies.

For example, in a Phase lb trial using aducanumab, another anti-Aβ antibody outside the scope of the present disclosure, reductions in amyloid PET SUVR composite score in aducanumab-treated patients were reported to be similar in ApoE4 carriers and non-carriers (Sevigny, J. et al., “The antibody aducanumab reduces Aβ plaques in Alzheimer's disease,” Nature 537:50-56, 50-51 (Sep. 1, 2016), citing Extended Data FIG. 2b.) The results in Extended Data FIG. 2b, however, reflect better results for treatment of ApoE4 non-carriers as compared to ApoE4 carriers.

In a Phase II trial of bapineuzumab, an anti-Aβ antibody outside the scope of the present disclosure, in treatment of mild-to-moderate Alzheimer's disease, results indicated possible differences on some clinical measures by ApoE4 carrier status, with potential treatment differences favoring noncarriers. (See Salloway et al., “A phase 2 multiple ascending dose trial of bapineuzumab in mild to moderate Alzheimer disease,” Neurology 73:2061-2070, 2067 (Dec. 15, 2009).) Accordingly, two different Phase III clinical trials of bapineuzumab were conducted—one in ApoE4-positive carriers and one in ApoE4 non-carriers having mild-to-moderate Alzheimer's disease. Despite the Phase II indications, the Phase III results not only failed to distinguish between the two ApoE4 genotypes but failed to show a benefit of bapineuzumab with respect to clinical outcomes. (Salloway et al., “Two Phase 3 Trials of Bapineuzumab in Mild-to-Moderate Alzheimer's Disease,” N. Engl. J. Med. 370:322-33 (2014).) Similarly, two Phase III clinical trials with solanezumab, another anti-Aβ antibody outside the scope of the present disclosure, also did not show benefit with respect to the primary clinical outcomes in patients with mild-to-moderate Alzheimer's disease. (Salloway et al., N. Engl. J. Med. 370:322-33, 332 (2014); Doody, R.S. et al., “Phase 3 trials of solanezumab for mild-to-moderate Alzheimer's disease,” N. Engl. J. Med. 370:311-21 (2014).)

Based on results such as those discussed below, the statistically significant results reported herein with ApoE4 positive subjects as compared to non-carriers of ApoE4 were not only unexpected but unpredictable.

Unpredictable and Unexpected Results with ApoE4-Positive Subjects

Generally, ApoE4 positive subjects responded better to BAN2401 treatment than did non-carriers of ApoE4. In some embodiments, those differences were clinically meaningful and statistically significant.

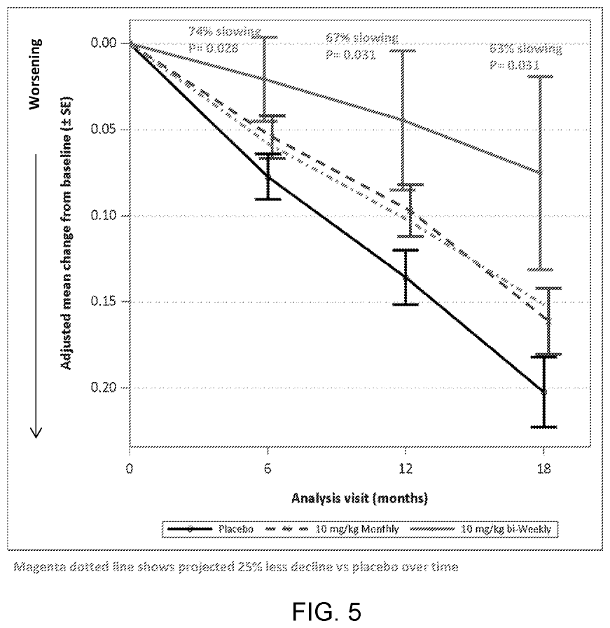

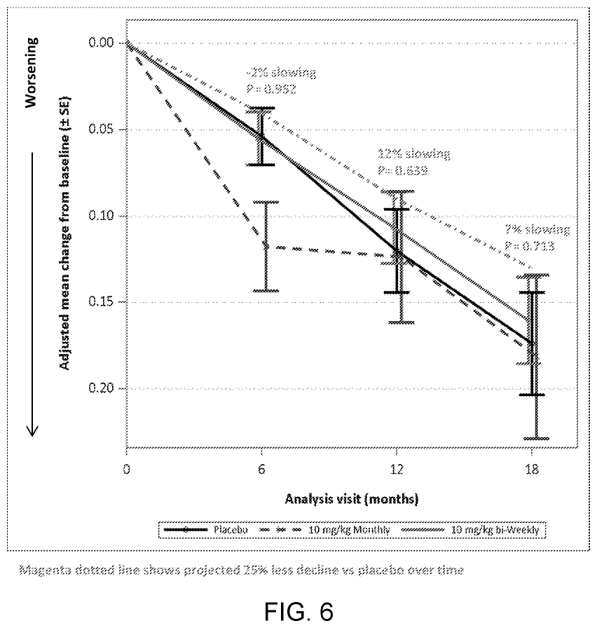

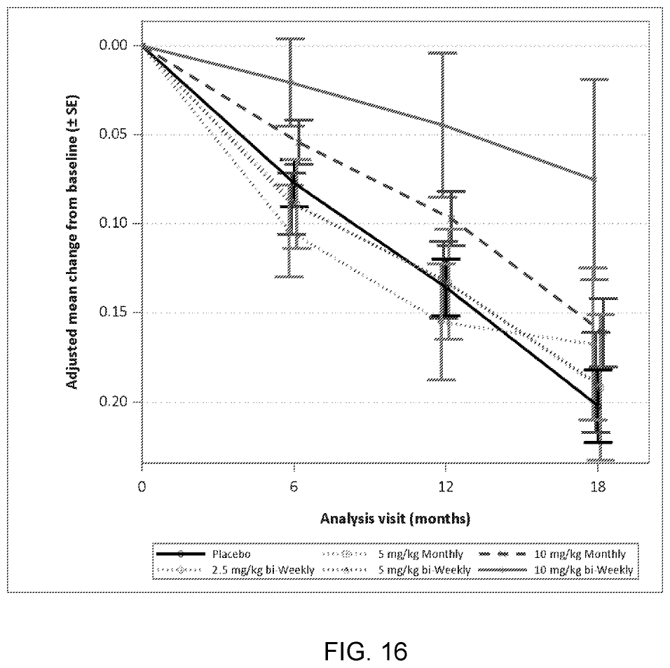

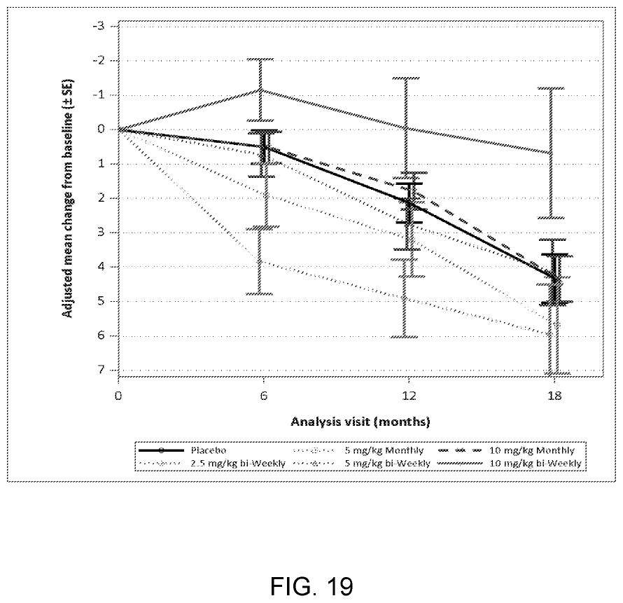

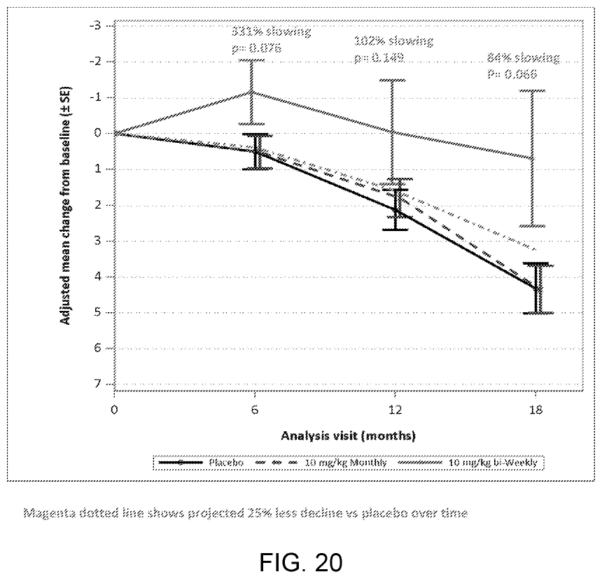

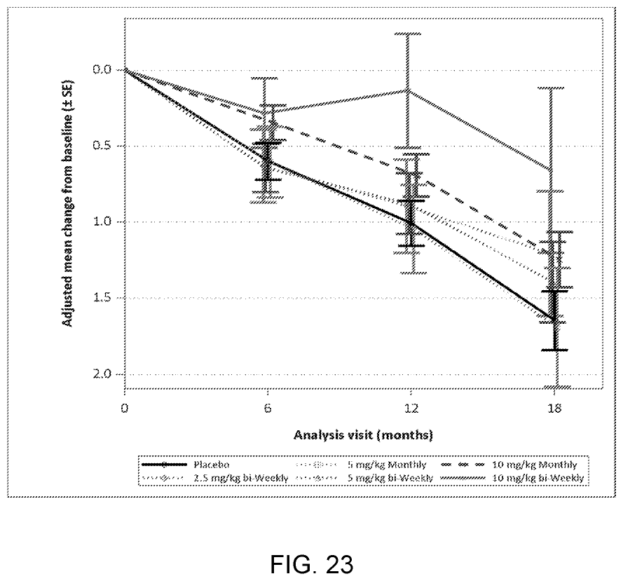

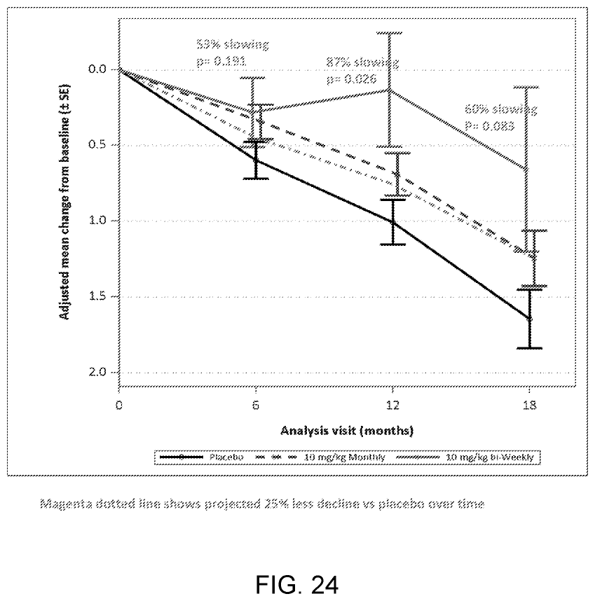

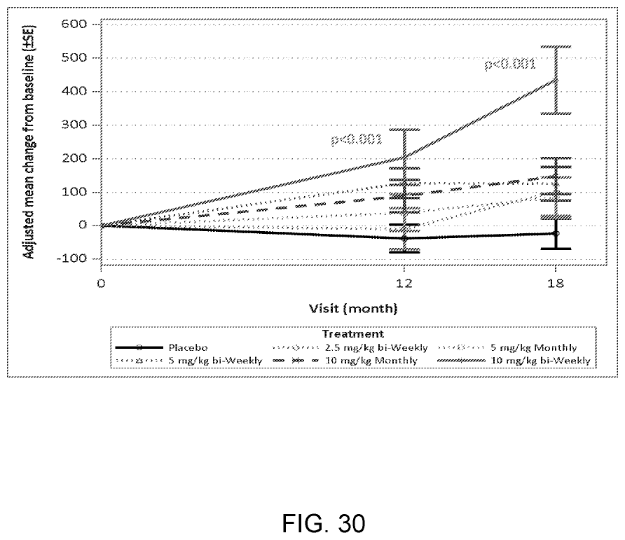

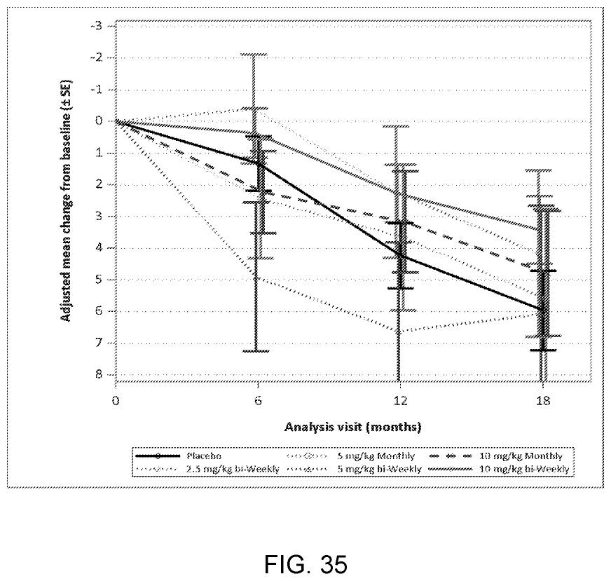

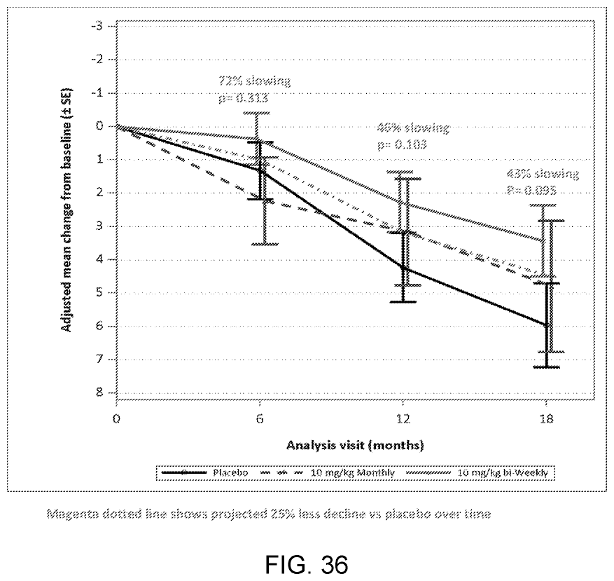

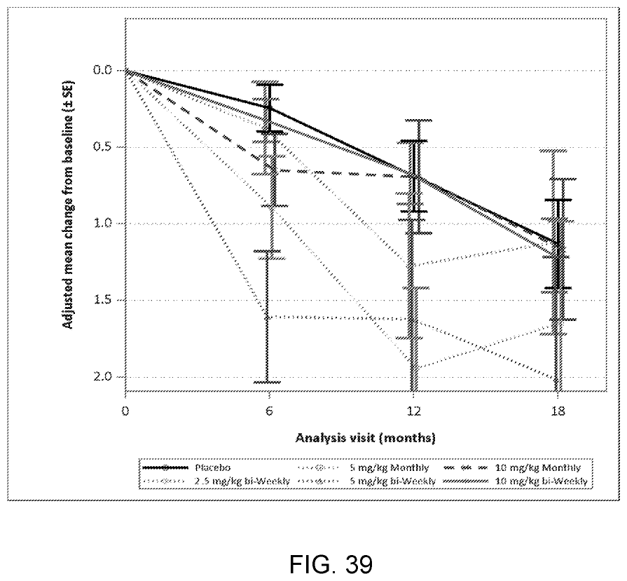

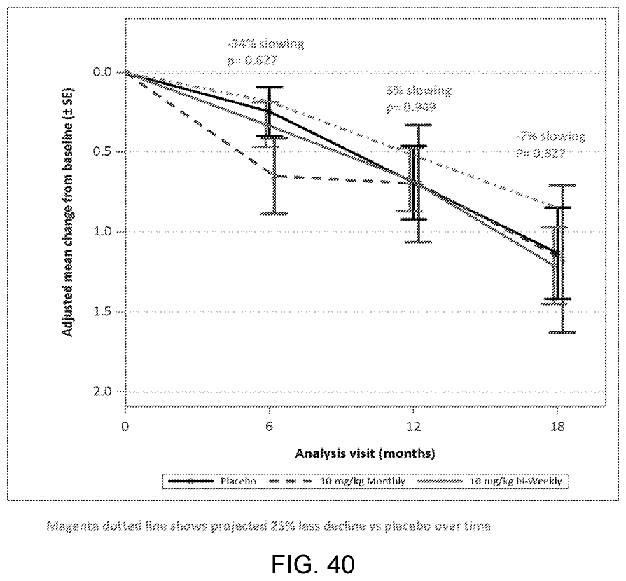

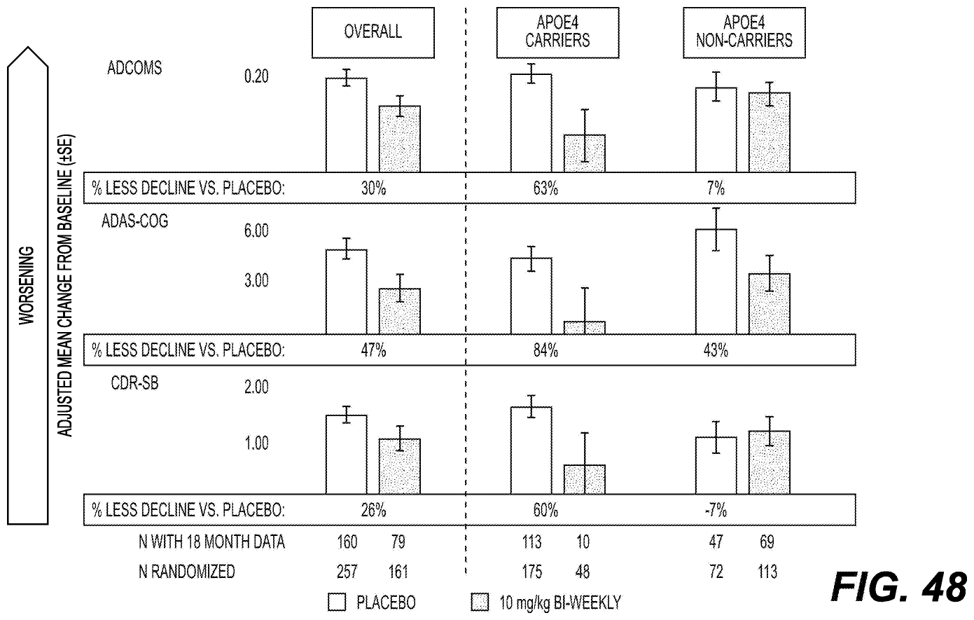

FIGS. 5 and 16 show a dose dependent slowing in cognitive decline as determined by ADCOMS in ApoE4-positive subjects who were administered various doses of BAN2401, starting at 6 months, compared to subjects who were administered placebo. See also FIG. 48. FIGS. 6 and 30 show the more modest cognitive decline after administration of to ApoE4-negative subjects. See also FIG. 48. The difference in response between ApoE4-subjects and ApoE4-negative subjects was observed regardless of whether cognitive decline was determined by ADCOMS (ApoE4-positive: FIGS. 5 and 16; ApoE4-negative: FIGS. 6 and 30), ADAS-cog (ApoE4-positive: FIGS. 19 and 20; ApoE4-negative: FIGS. 35 and 36), or CDR-SB (ApoE4-positive: FIGS. 23 and 24; ApoE4-negative: FIGS. 39 and 40).

For example, a 7% reduction in clinical decline was observed in ApoE4-negative subjects as determined by ADCOMS after 18 months of bi-weekly administration of 10 mg/kg BAN2401, whereas a 63% reduction in clinical decline was observed in ApoE4-positive subjects as determined by ADCOMS after 18 months of bi-weekly administration of 10 mg/kg BAN2401.

For example, a 43% reduction in clinical decline was observed in ApoE4-negative subjects as determined by ADCOMS after 18 months of bi-weekly administration of 10 mg/kg BAN2401, whereas an 84% reduction in clinical decline was observed in ApoE4-positive subjects as determined by ADAS-cog after 18 months of bi-weekly administration of 10 mg/kg BAN2401.

For example, a −7% reduction in clinical decline was observed in ApoE4-negative subjects as determined by CDR-SB after 18 months of bi-weekly administration of 10 mg/kg BAN2401, whereas a 60% reduction in clinical decline was observed in ApoE4-positive subjects as determined by CDR-SB after 18 months of bi-weekly administration of 10 mg/kg BAN2401.

Subjects having Mild Cognitive Impairment Due to Alzheimer's Disease—Intermediate Likelihood and of Subjects having Mild Alzheimer's Disease Dementia

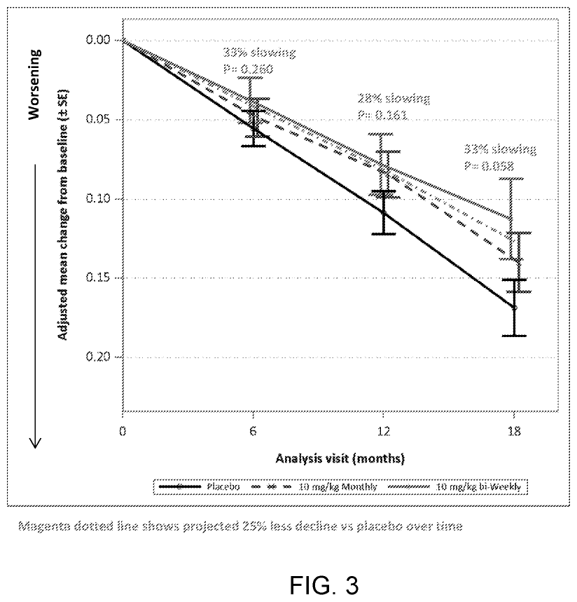

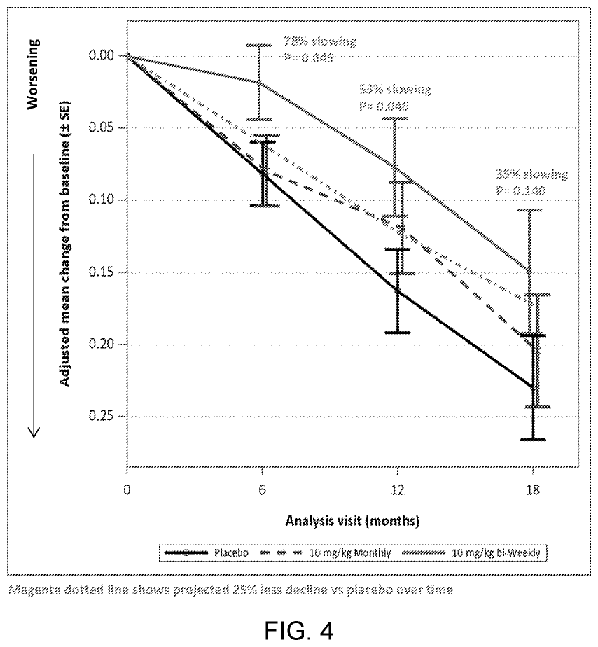

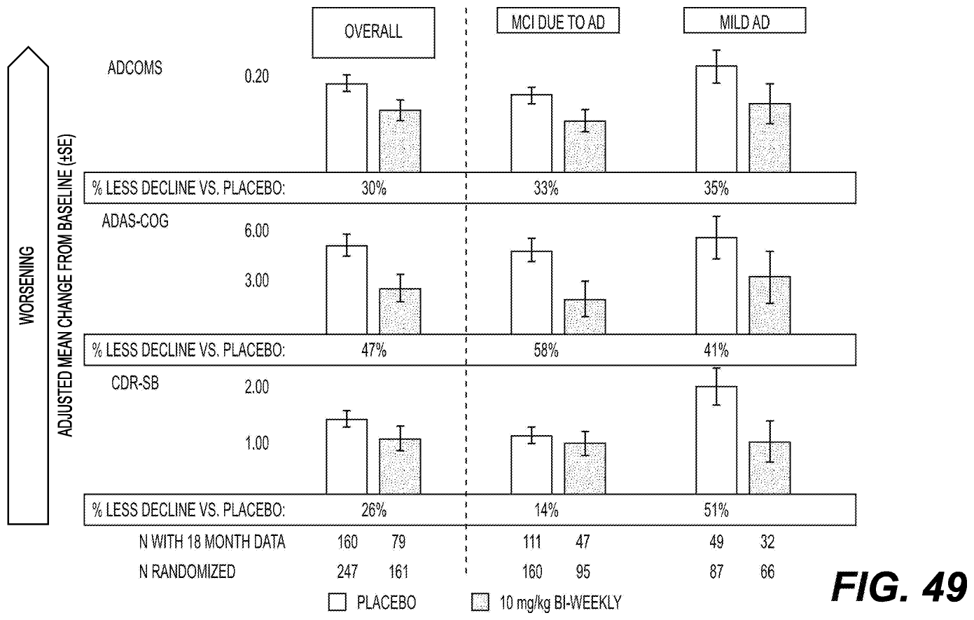

Responsiveness of subjects having mild cognitive impairment due to Alzheimer's disease—intermediate likelihood and of subjects having mild Alzheimer's disease dementia was also compared. Overall, subjects having mild cognitive impairment due to Alzheimer's disease—intermediate likelihood and subjects having mild Alzheimer's disease dementia responded well to treatment with compositions comprising BAN2401. (See, e.g., FIGS. 3, 4, and 49.)

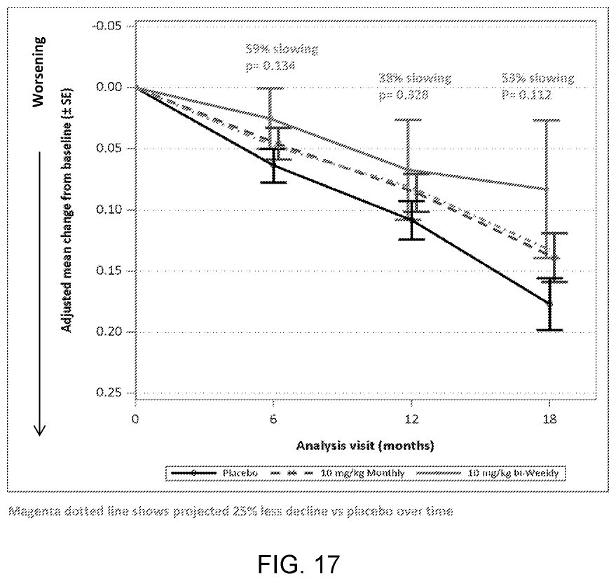

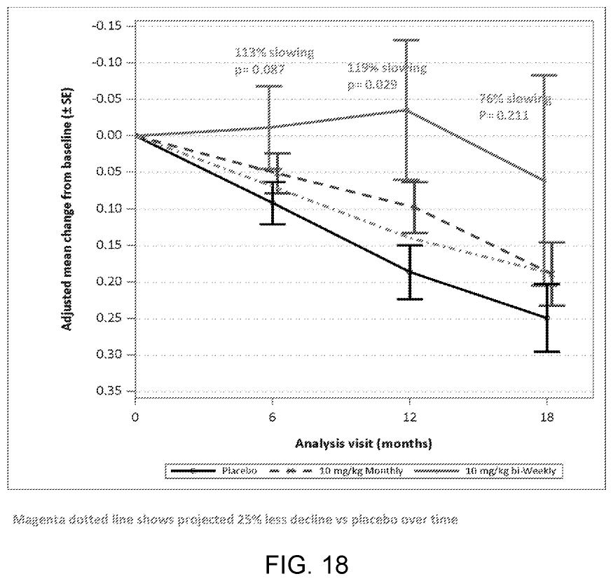

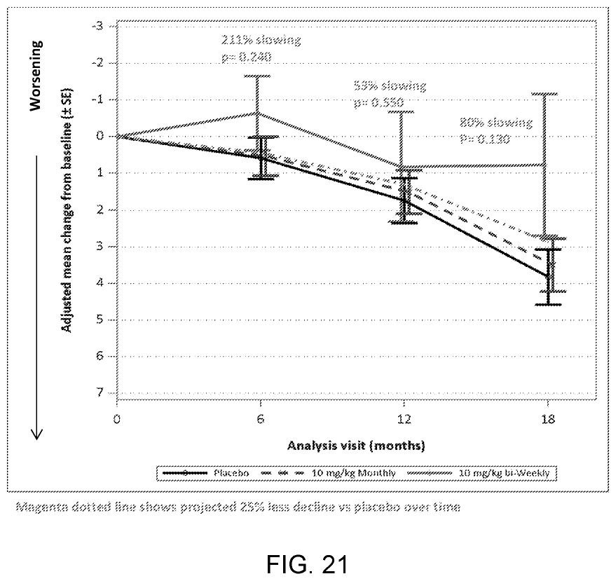

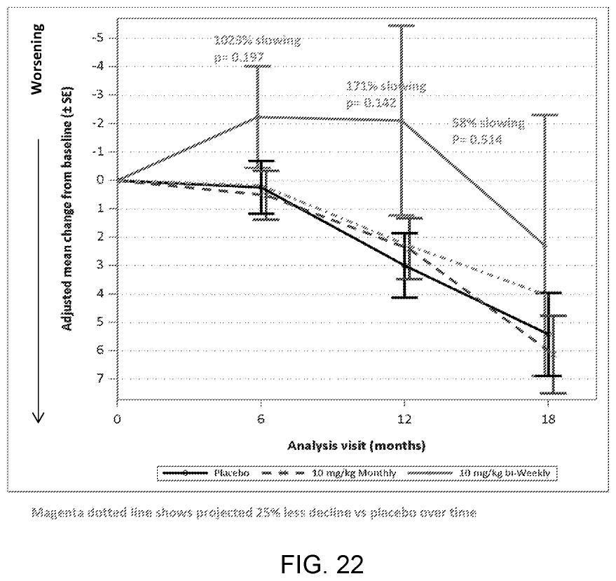

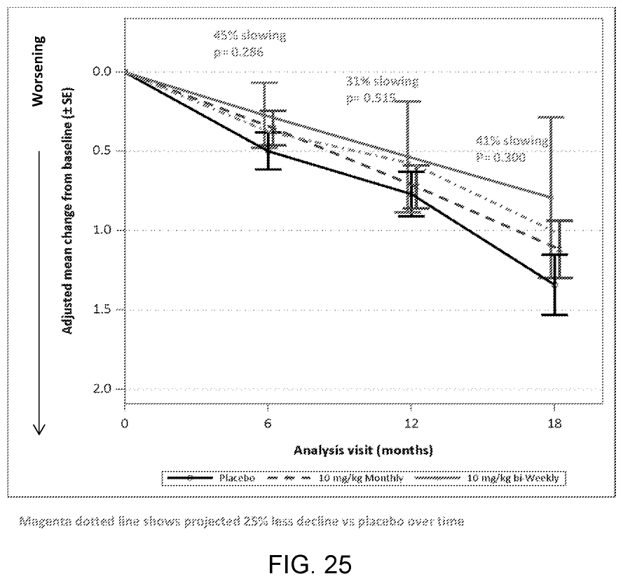

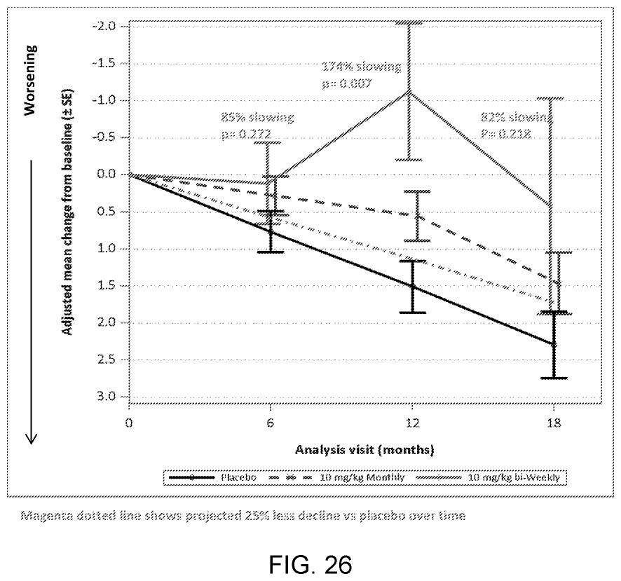

However, ApoE4-positive subjects having mild Alzheimer's disease dementia responded to treatment with BAN2401 better than subjects having mild cognitive impairment due to Alzheimer's disease dementia, as determined by ADCOMS (cf. FIGS. 17 and 18), by ADAS-cog (cf. FIGS. 21 and 22), and by CDR-SB (cf. FIGS. 25 and 26). ApoE4-positive subjects having mild Alzheimer's disease dementia responded better to treatment with a composition comprising BAN2401 than ApoE4-positive subjects having mild cognitive impairment due to Alzheimer's disease—intermediate likelihood.

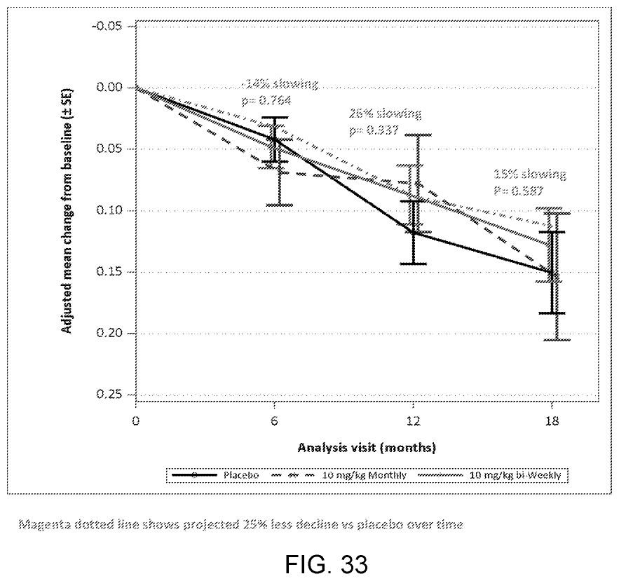

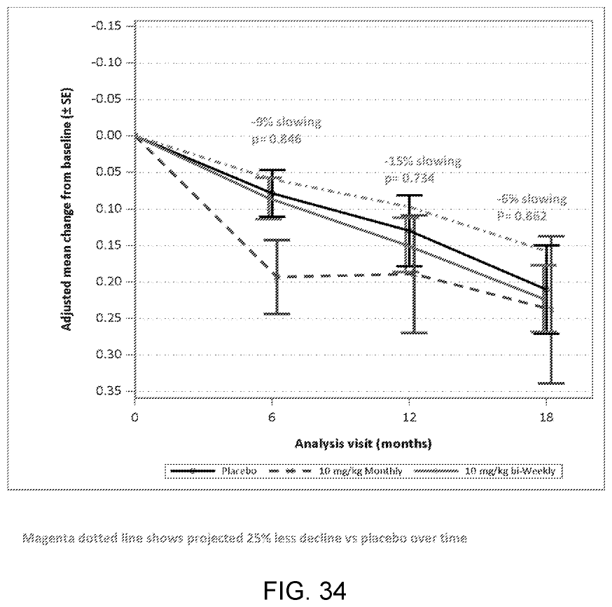

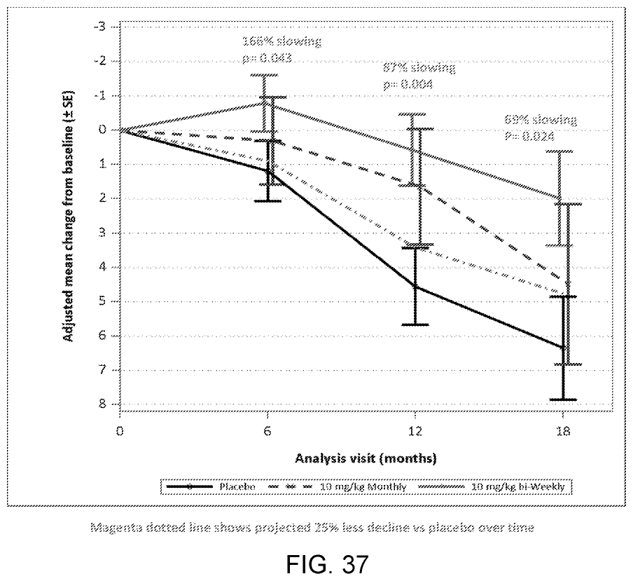

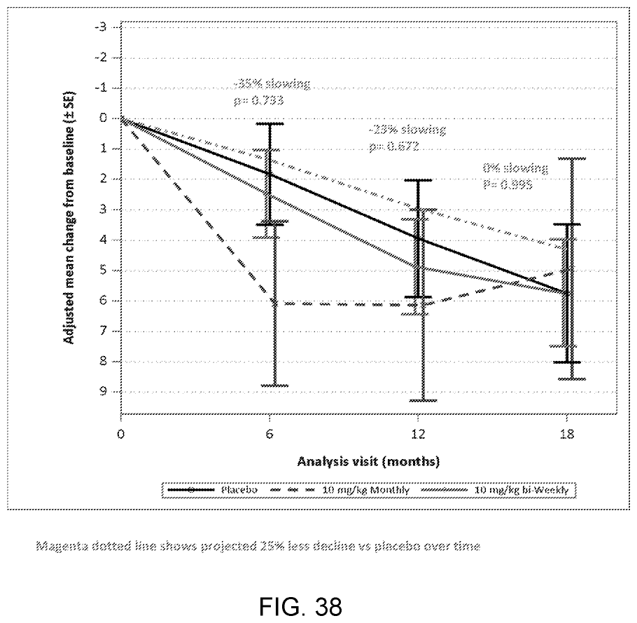

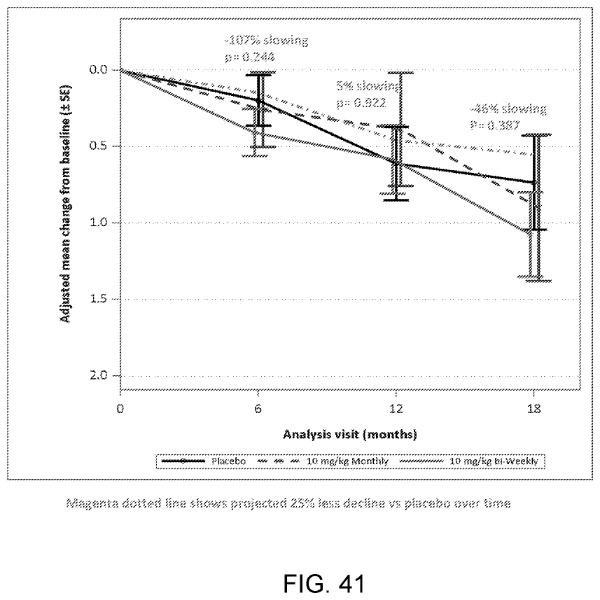

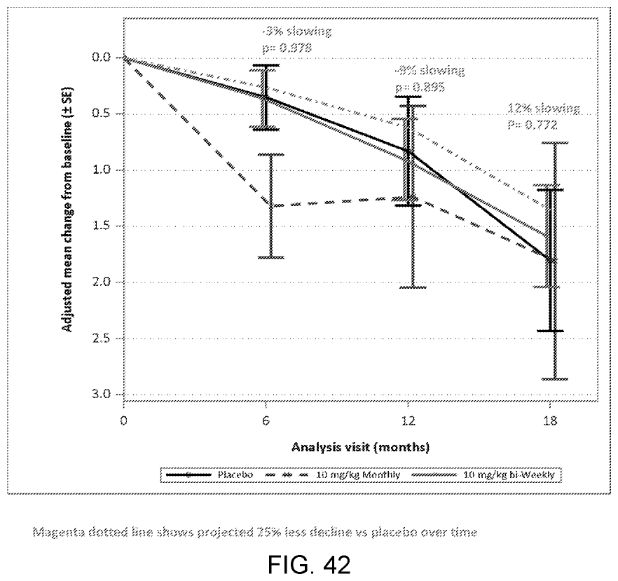

Within ApoE4-negative subjects, subjects having mild cognitive impairment due to Alzheimer's disease—intermediate likelihood and subjects having mild Alzheimer's disease dementia responded to treatment. (See FIGS. 33 and 34 (ADCOMS); FIGS. 37 and 38 (ADAS-cog); and FIGS. 41 and 42 (CDR-SB).)

### Brain Amyloid Level Results

Tables 13 and 14 provide the change from baseline of brain amyloid level in all subjects after 12 months of administration of various doses of BAN2401, as determined by PET (standard uptake value ratio (SUVR)), where the reference region is the whole cerebellum mask.

TABLE 13
Change from baseline of brain amyloid level for 2.5 mg/kg bi-weekly,
5 mg/kg monthly, 5 mg/kg bi-weekly, 10 mg/kg monthly, and 10
mg/kg bi-weekly doses of BAN2401, as determined by PET
standard uptake value ratio, after 12 months.

| | | 2.5 mg/kg | 5 mg/kg | 5 mg/kg | 10 mg/kg | 10 mg/kg |
|---|---|---|---|---|---|---|
| | Placebo | bi-weekly | monthly | bi-weekly | monthly | bi-weekly |
| N* | 96 | 27 | 28 | 25 | 89 | 43 |
| Baseline | 1.40 | 1.41 | 1.42 | 1.40 | 1.42 | 1.36 |
| SUVR | (0.16) | (0.11) | (0.17) | (0.12) | (0.18) | (0.18) |
| mean (SD) | | | | | | |
| Obs. | −0.07 | −4.11 | −5.05 | −11.02 | −11.96 | −17.09 |
| Mean % | (6.05) | (5.76) | (6.91) | (6.11) | (7.27) | (14.67) |
| CFB (SD) | | | | | | |
| Obs. % | — | −4.04 | −4.98 | −10.95 | −11.89 | −17.02 |
| CFB diff. | | | | | | |
| LS Mean | −0.26 | −4.16 | −4.75 | −11.35 | −11.78 | −17.41 |
| % CFB | (0.84) | (1.48) | (1.49) | (1.55) | (0.92) | (1.20) |
| (SE) | | | | | | |
| LSM % | — | −3.90 | −4.49 | −11.09 | −11.52 | −17.15 |
| CFB diff. | | | | | | |
| P-value | — | 0.018 | 0.007 | <0.001 | <0.001 | <0.001 |

TABLE 14
Change from baseline of brain amyloid level for the combined 10
mg/kg (once every two weeks and once a month) doses, as determined
by PET standard uptake value ratio, after 12 months.

| | | Combined |
|---|---|---|
| | Placebo | 10 mg/kg |
| N* | 96 | 132 |
| Baseline SUVR | 1.40 (0.158) | 1.40 (0.178) |
| mean (SD) | | |
| Obs. Mean % | −0.07 (6.046) | −13.64 (10.525) |
| CFB (SD) | | |
| Obs. % CFB diff. | | −13.57 |
| LS Mean % | −0.64 (0.90) | −13.84 (0.76) |
| CFB (SE) | | |
| LSM % CFB diff. | | −13.20 |
| P-value | | <0.001 |

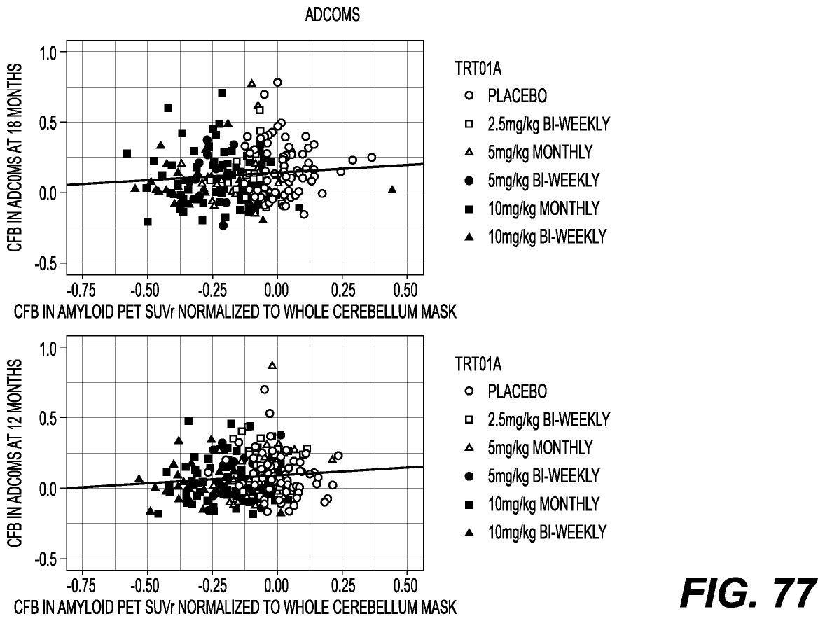

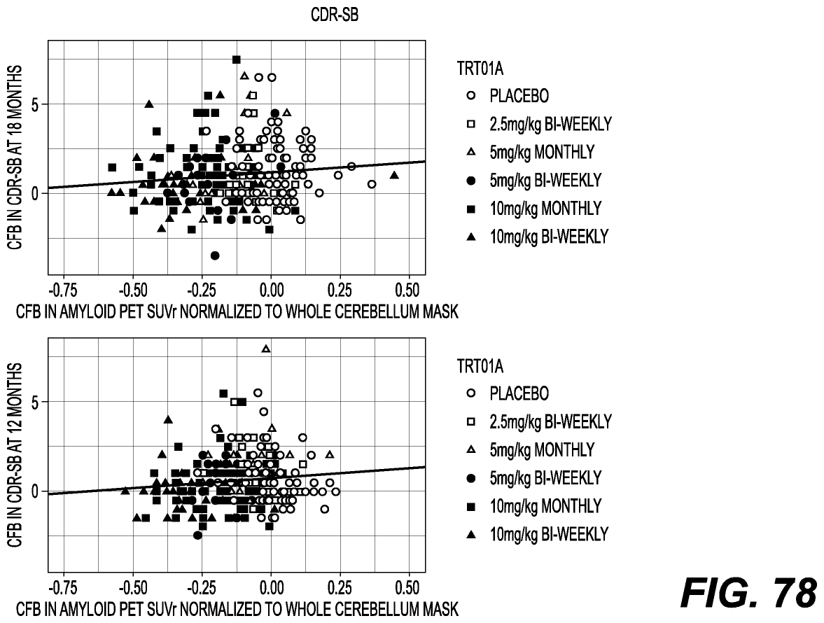

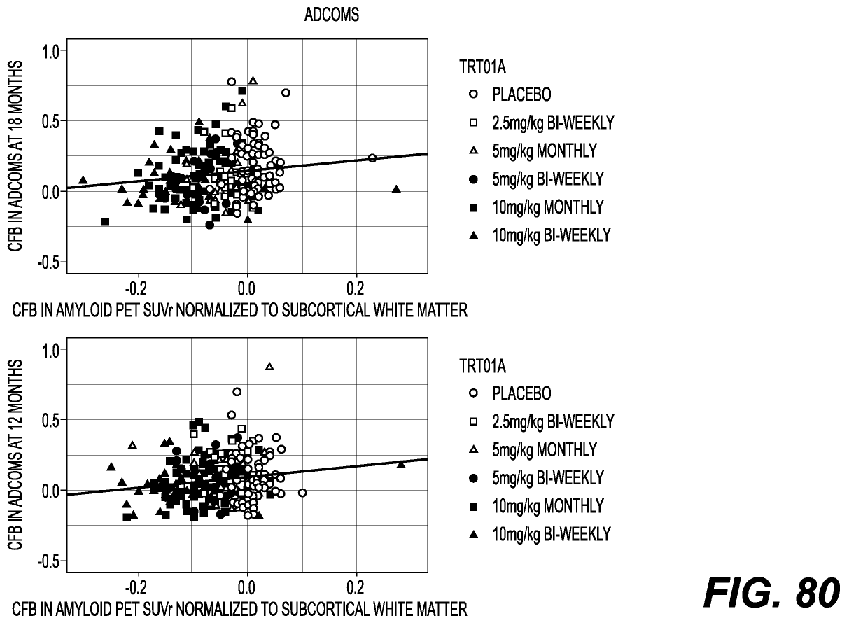

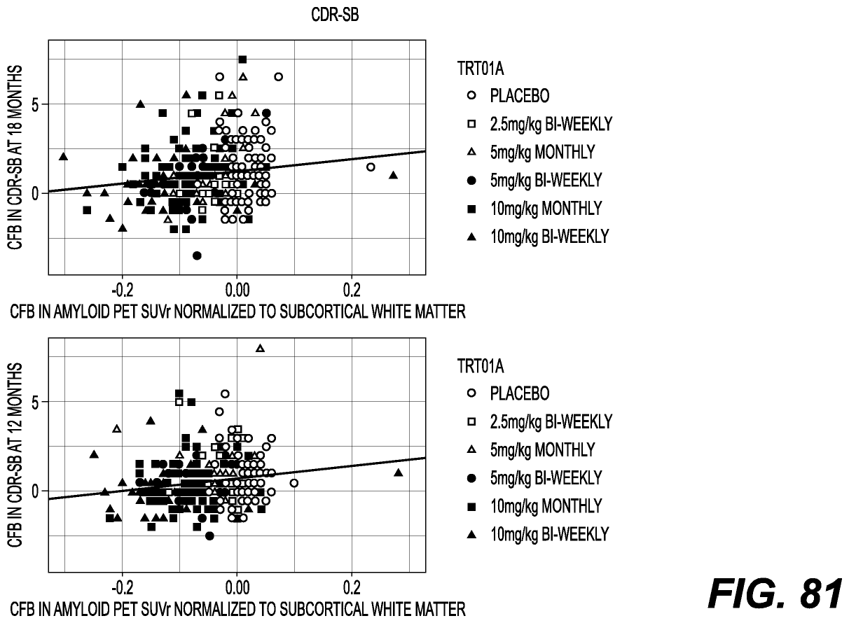

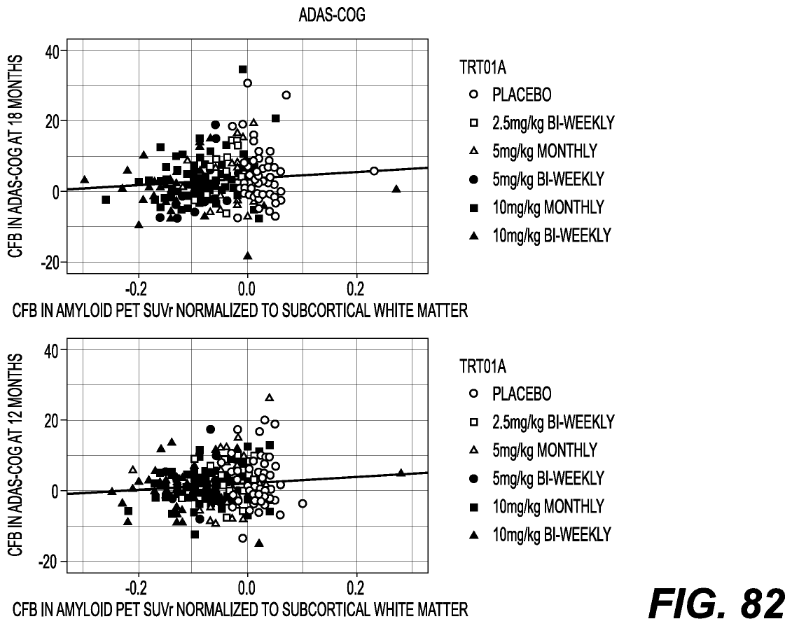

Tables 15 and 16 provide the change from baseline of brain amyloid level after 12 months of administration of various doses of BAN2401, as determined by PET (visual reads of amyloid PET images; standard uptake value ratio (SUVR)), where the reference region is the whole cerebellum mask. Change from baseline, as determined by ADCOMS and CDR-SB, positively correlated, in a statistically significant fashion, with amyloid clearance, as determined by PET (SUVR), where the reference region is the whole cerebellum mask. See FIGS. 77 and 78. Change from baseline, as determined by ADCOMS, CDR-SB, and ADAS-Cog, positively correlated, in a statistically significant fashion, with amyloid clearance, as determined by PET (SUVR), where the reference region is the subcortical white matter. See FIGS. 80-82.

TABLE 15
Change from baseline of brain amyloid level for 2.5 mg/kg bi-weekly,
5 mg/kg monthly, 5 mg/kg bi-weekly, 10 mg/kg monthly, and 10
mg/kg bi-weekly doses of BAN2401, as determined by PET
standard uptake value ratio, after 18 months.

| | | 2.5 mg/kg | 5 mg/kg | 5 mg/kg | 10 mg/kg | 10 mg/kg |
|---|---|---|---|---|---|---|
| | Placebo | bi-weekly | monthly | bi-weekly | monthly | bi-weekly |
| N* | 88 | 23 | 23 | 24 | 83 | 37 |
| Baseline SUVR | 1.40 | 1.41 | 1.42 | 1.40 | 1.42 | 1.36 |
| mean (SD) | (0.16) | (0.11) | (0.17) | (0.12) | (0.18) | (0.18) |
| Obs. Mean % | 0.78 | −5.83 | −8.99 | −13.51 | −15.12 | −19.29 |
| CFB (SD) | (7.03) | (5.15) | (6.95) | (7.85) | (7.81) | (14.91) |
| Obs. % CFB diff. | — | −6.61 | −9.77 | −14.29 | −15.90 | −20.07 |
| LS Mean % | 0.77 | −6.51 | −8.93 | −13.88 | −15.15 | −20.37 |
| CFB (SE) | (0.87) | (1.56) | (1.57) | (1.59) | (0.95) | (1.26) |
| LSM % CFB diff. | — | −7.28 | −9.70 | −14.65 | −15.92 | −21.14 |
| P-value | — | <0.001 | <0.001 | <0.001 | <0.001 | <0.001 |

TABLE 16
Change from baseline of brain amyloid level for the combined 10
mg/kg (once every two weeks and once a month) doses, as determined by
PET standard uptake value ratio, after 18 months.

| | | Combined |
|---|---|---|
| | Placebo | 10 mg/kg |
| N* | 88 | 120 |
| Baseline | 1.40 (0.158) | 1.40 (0.178) |
| SUVR mean | | |
| (SD) | | |
| Obs. Mean | 0.78 (7.03) | −16.42 (10.65) |
| % CFB (SD) | | |
| Obs. % CFB diff. | — | −15.64 |
| LS Mean % | 0.39 (0.91) | −7.07 (0.77) |
| CFB (SE) | | |
| LSM % CFB diff. | — | −17.46 |
| P-value | — | <0.001 |

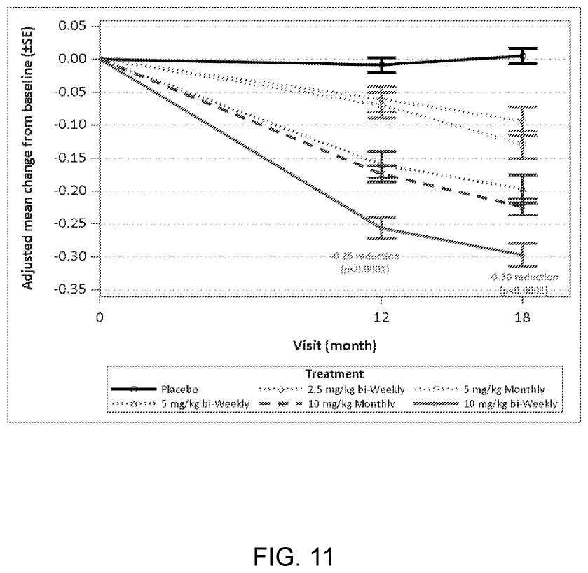

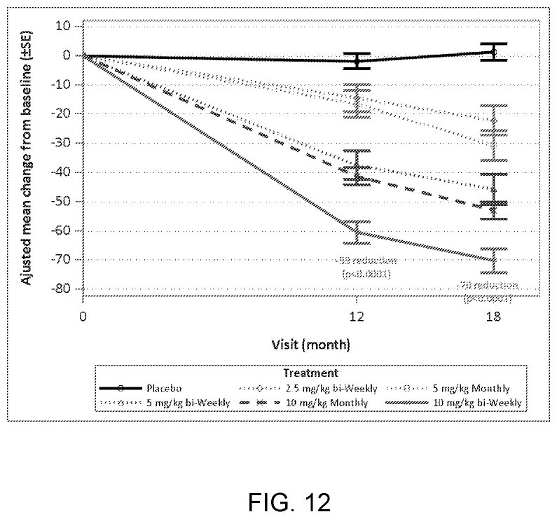

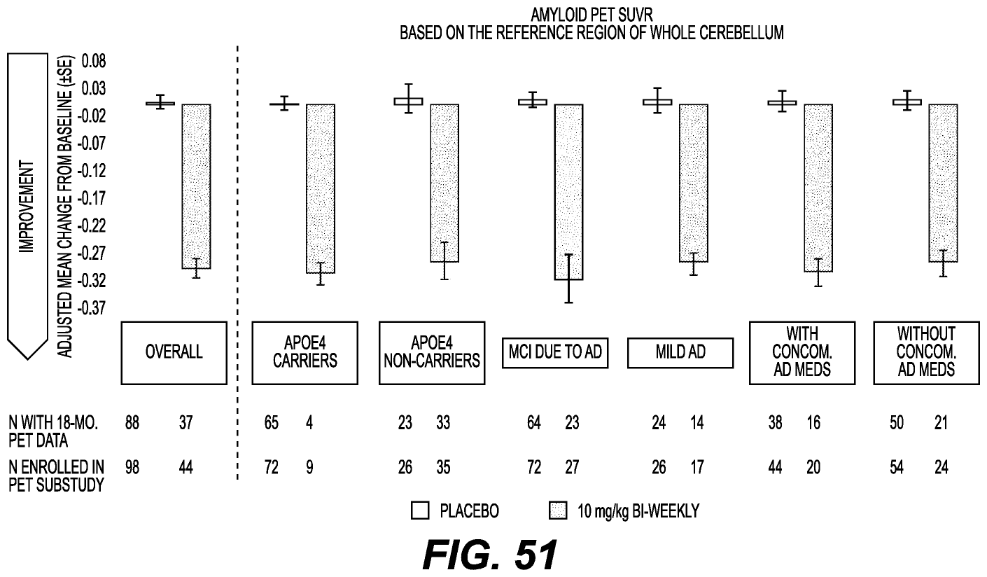

FIGS. 11 and 12 show dose-dependent reductions in brain amyloid level after administration of 2.5 mg/kg of BAN2401 bi-weekly, 5 mg/kg of BAN2401 monthly, 5 mg/kg of BAN2401 bi-weekly, 10 mg/kg of BAN2401 monthly, or 10 mg/kg of BAN2401 bi-weekly after 12 and 18 months. See also FIG. 51.

As can be seen, statistically significant reductions in brain amyloid levels were observed in subjects having early Alzheimer's disease due to administration of a composition comprising a therapeutically effective amount of at least one anti-Aβ protofibril. For example, 18 months of administration of 10 mg/kg of BAN2401 bi-weekly resulted in a -70 reduction (p <0.0001). (See, e.g., FIG. 12 (centiloids).)

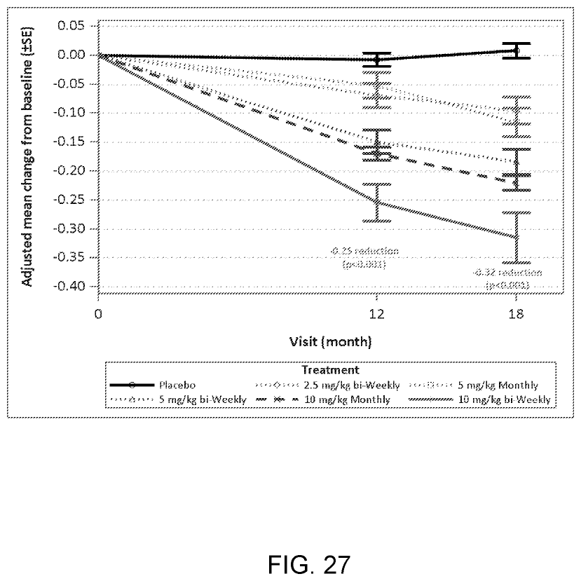

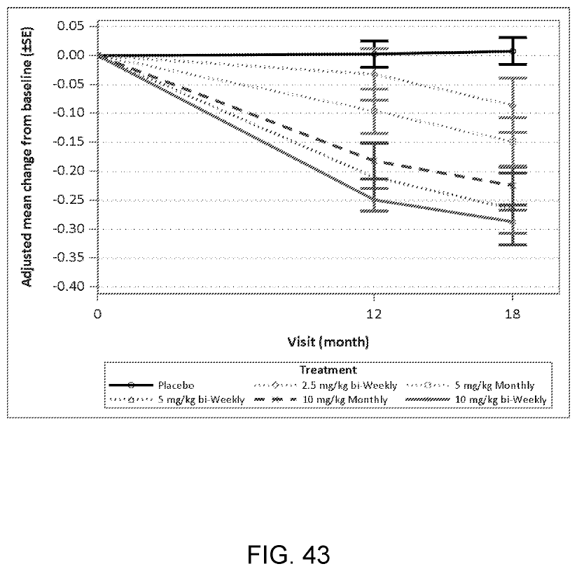

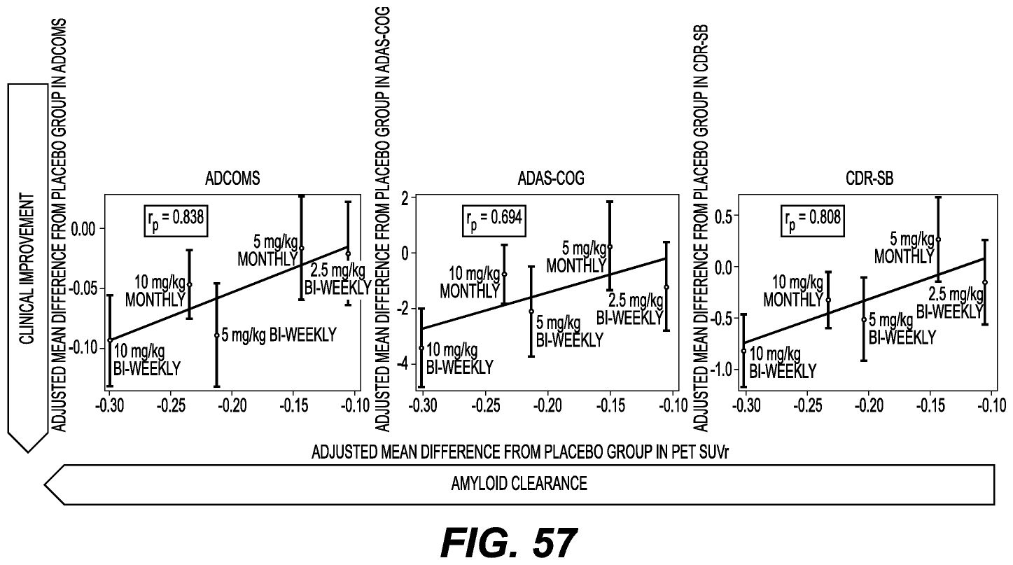

Similarly, administration of various doses of BAN2401 to ApoE4-positive subjects (FIGS. 27 (-0.32 reduction for 10 mg/kg biweekly dose after 18 months) and 28 (-76 reduction for 10 mg/kg biweekly dose after 18 months)) and ApoE4-negative subjects (FIGS. 43 (-0.29 reduction for 10 mg/kg biweekly dose after 18 months) and 44 (-68 reduction for 10 mg/kg biweekly dose after 18 months)) resulted in reduction of brain amyloid levels in those subjects. It was also found that reduction of brain amyloid level correlated with a slowing of cognitive decline. See FIG. 57.

### Conversion of Amyloid Positive to Amyloid Negative Results

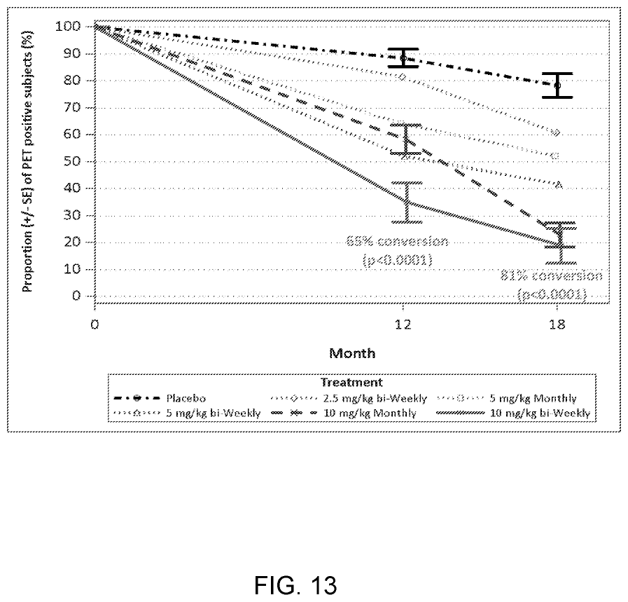

Tables 17 and 18 show the subjects who became amyloid negative after various doses of BAN2401 were administered. FIG. 13 shows that the proportion of PET positive subjects following administration of BAN2401 2.5 mg/kg of BAN2401 bi-weekly, 5 mg/kg of BAN2401 monthly, 5 mg/kg of BAN2401 bi-weekly, 10 mg/kg of BAN2401 monthly, or 10 mg/kg of BAN2401 bi-weekly after 12 and 18 months, as determined by florbetapir tracer visual read of PET.

As can be seen, significant proportions of amyloid positive subjects having early Alzheimer's disease were converted to amyloid negative due to administration of a composition comprising a therapeutically effective amount of at least one anti-Aβ protofibril. For example, after 18 months of administration of 10 mg/kg of BAN2401 bi-weekly, 81%, (p<0.0001) of amyloid positive subjects were converted to amyloid negative. (See, e.g., Table 17 and FIG. 13.)

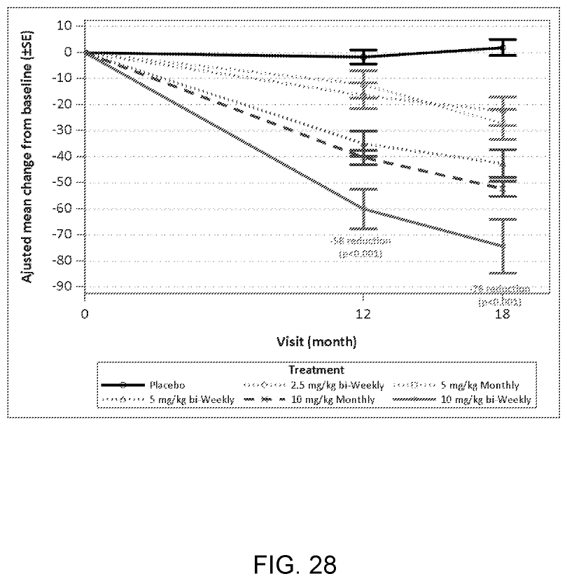

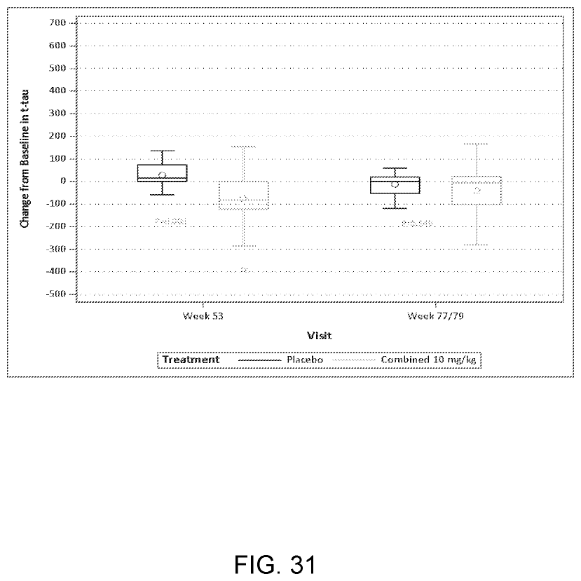

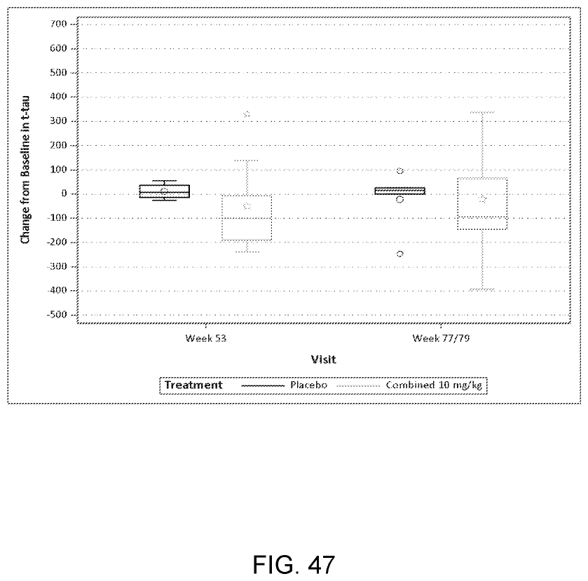

Significant proportions of ApoE4-positive (FIG. 28) and ApoE4-negative (FIG. 42) amyloid positive subjects having early Alzheimer's disease were converted to amyloid negative due to administration of a composition comprising a therapeutically effective amount of at least one anti-Aβ protofibril. However, a higher proportion of ApoE4-positive subjects were converted to amyloid negative than were ApoE4-negative subjects. For example, after 18 months of administration of 10 mg/kg of BAN2401 monthly, 79% (p<0.0001) of ApoE4-negative amyloid positive subjects were converted to amyloid negative (see, e.g., FIG. 31) whereas 100% (p<0.0001) of ApoE4-positive amyloid positive subjects were converted to amyloid negative. (See, e.g., FIG. 47.)

TABLE 17
Proportion of subjects who became amyloid negative during study across
2.5 mg/kg bi-weekly, 5 mg/kg monthly, 5 mg/kg bi-weekly, 10 mg/kg
monthly, and 10 mg/kg bi-weekly doses of BAN2401.

| | | | | | |
|---|---|---|---|---|---|
| | 2.5 mg/kg | 5 mg/kg | 5 mg/kg | 10 mg/kg | 10 mg/kg |
| Placebo | bi-weekly | monthly | bi-weekly | monthly | bi-weekly |

Analysis after 12 months

| | | | | | | |
|---|---|---|---|---|---|---|
| n/m* | 11/96 | 5/27 | 10/28 | 12/25 | 37/89 | 28/43 |
| % of neg. | 11.5 | 18.5 | 35.7 | 48.0 | 41.6 | 65.1 |
| subj. | | | | | | |
| 90% Cl for | — | (7.6, | (20.8, | (30.5, | (32.7, | (51.5, |
| proportions | | 35.1) | 53.0) | 65.9) | 50.8) | 77.1) |
| (%) | | | | | | |
| p-value | — | 0.1504 | 0.0039 | 0.0001 | <0.0001 | <0.0001 |

Analysis after 18 months

| | | | | | | |
|---|---|---|---|---|---|---|
| n/m* | 19/88 | 9/23 | 11/23 | 14/24 | 64/83 | 30/37 |
| % of neg. | 21.6 | 39.1 | 47.8 | 58.3 | 77.1 | 81.1 |
| subj. | | | | | | |
| 90% Cl for | — | (22.2, | (29.6, | (39.7, | (68.3, | (67.4, |
| proportions | | 58.3) | 66.5) | 75.4) | 84.5) | 90.8) |
| (%) | | | | | | |
| p-value | — | 0.0497 | 0.0105 | 0.0007 | <0.0001 | <0.0001 |

TABLE 18
Proportion of subjects who became amyloid negative at the
combined 10 mg/kg doses (once every two weeks and once a month).

| | |
|---|---|
| | Combined |
| | 10 mg/kg |
| Placebo | groups |

Analysis after 12 months

| | | |
|---|---|---|
| n/m * | 11/96 | 65/132 |
| % of neg. subj. | 11.5 | 49.2 |
| 90% Cl for | — | (41.8, 56.7) |
| proportions (%) | | |
| p-value | — | <0.0001 |

Analysis after 18 months

| | | |
|---|---|---|
| n/m * | 19/88 | 94/120 |
| % of neg. subj. | 21.6 | 78.3 |
| 90% Cl for | — | (71.2, 84.4) |
| proportions (%) | | |
| p-value | — | <0.0001 |

As discussed hereinabove, biomarkers were also measured. For example, cerebrospinal fluid level of Aβ₁₋₄₂ and cerebrospinal fluid level of total tau were measured. Additional biomarkers that were measured include cerebrospinal fluid levels of neurogranin and neurofilament light chain.

Dose dependent, statistically significant effects on cerebrospinal fluid level of Aβ₁₋₄₂ and statistically significant longitudinal reduction in cerebrospinal fluid level of total tau were observed.

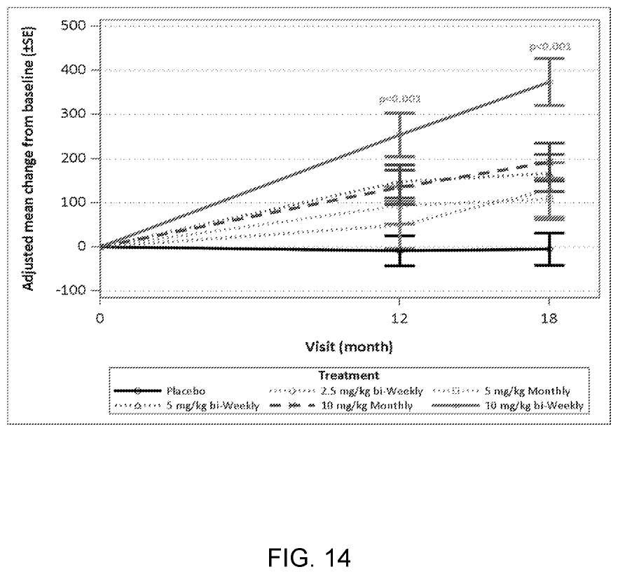

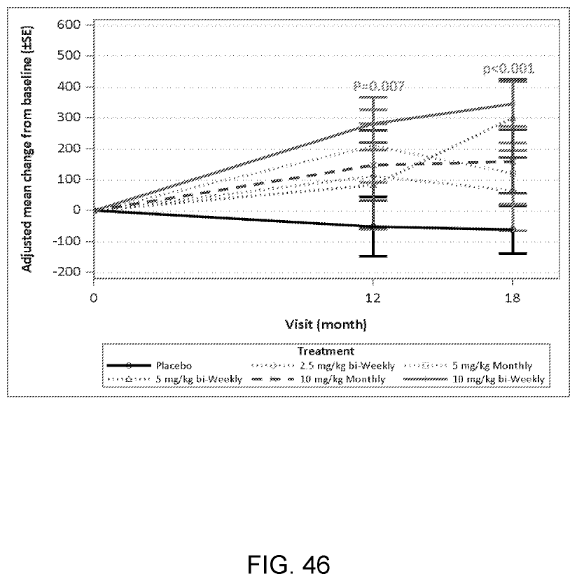

Overall (FIG. 14), and in each subgroup (FIG. 30—ApoE4-positive subjects; FIG. 46—ApoE4-negative subjects), administration of a composition comprising BAN2401 led to an increase in cerebrospinal fluid level of Aβ₁₋₄₂. Without being bound by any theory, this is speculated to be because of the normalization of cerebrospinal fluid level of Aβ₁₋₄₂, which reflects less sequestration of soluble Aβ₁₋₄₂ in the cerebrospinal fluid to Aβ plaques because of plaque removal. These results demonstrate that BAN2401 is interacting with its target.

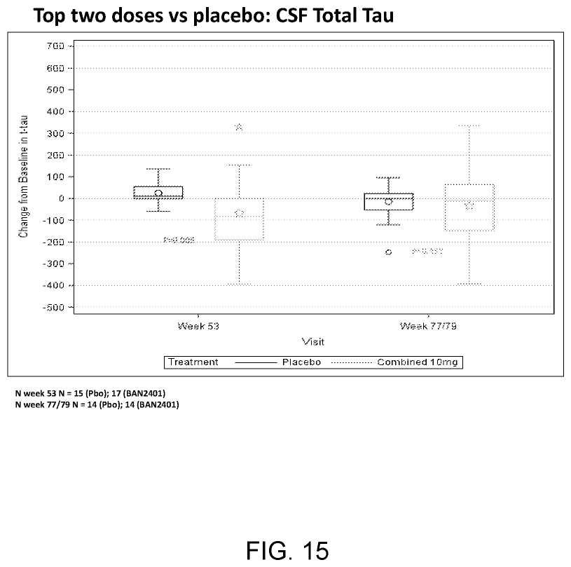

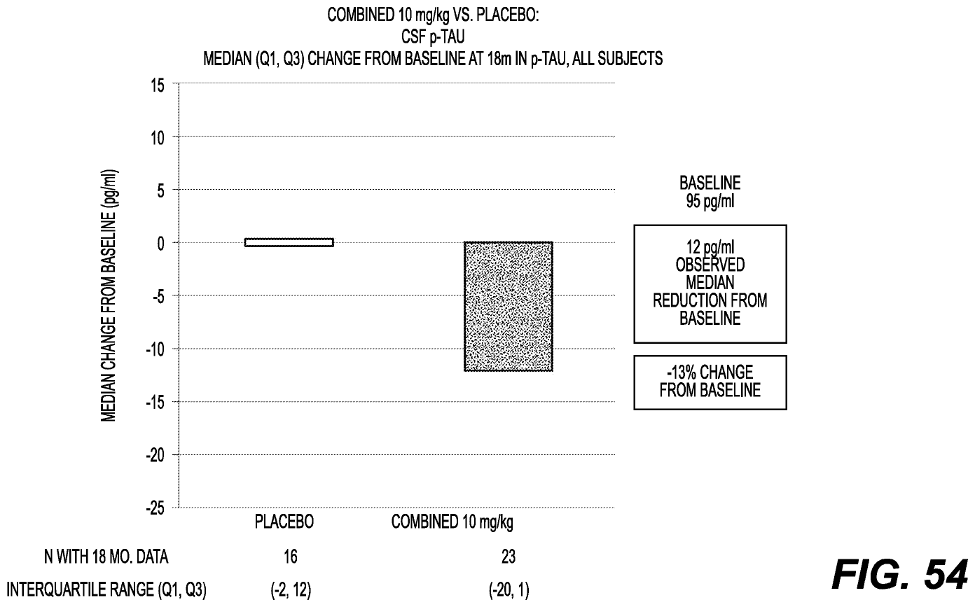

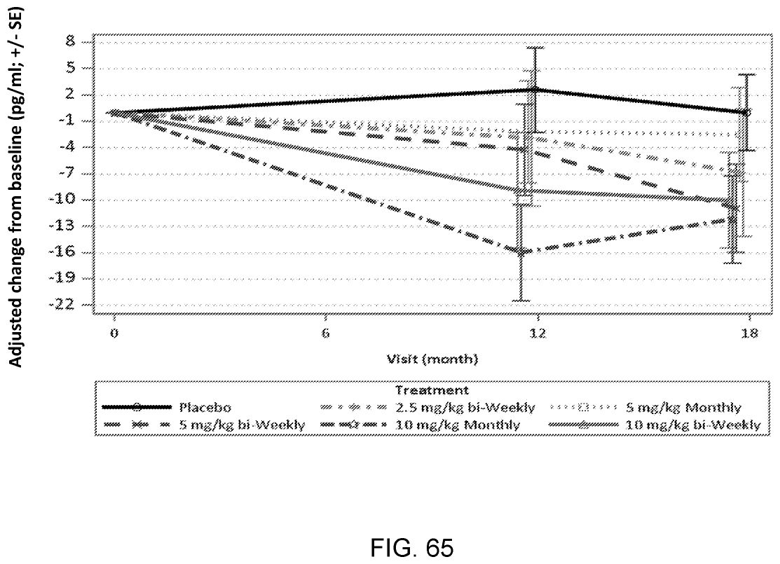

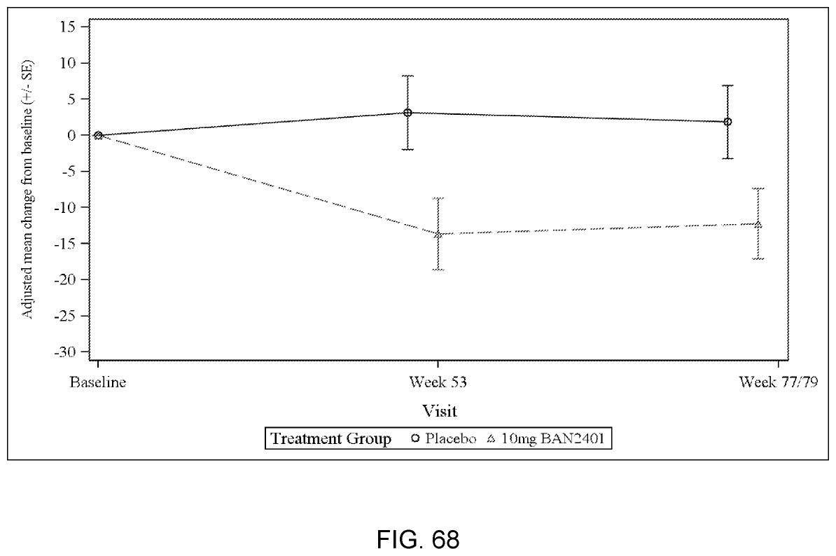

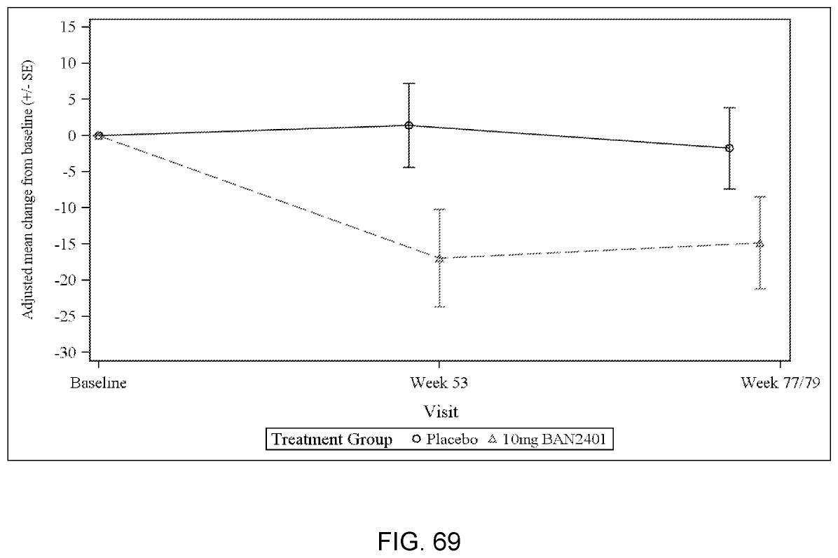

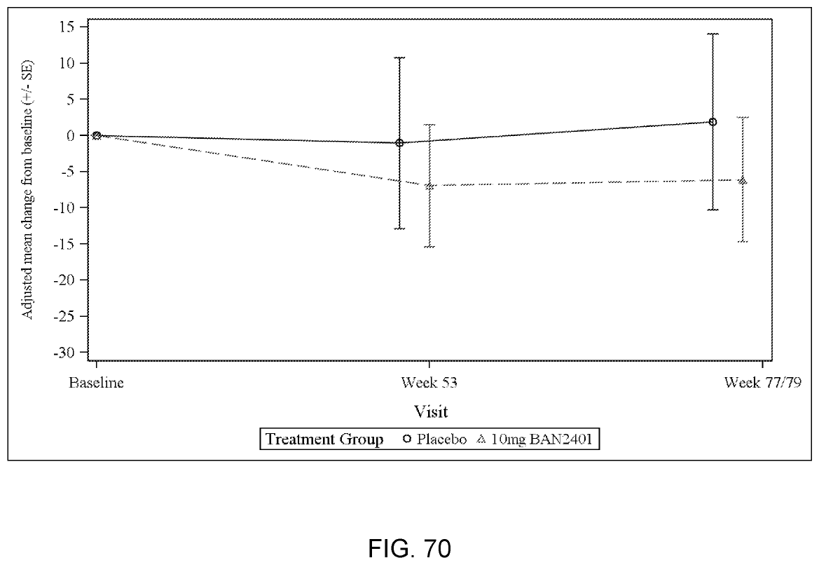

Similarly, all analytical groups of patients experienced a reduction in cerebrospinal fluid total tau. (See FIGS. 15, 31, 47, 54, 65, and 68-70.)

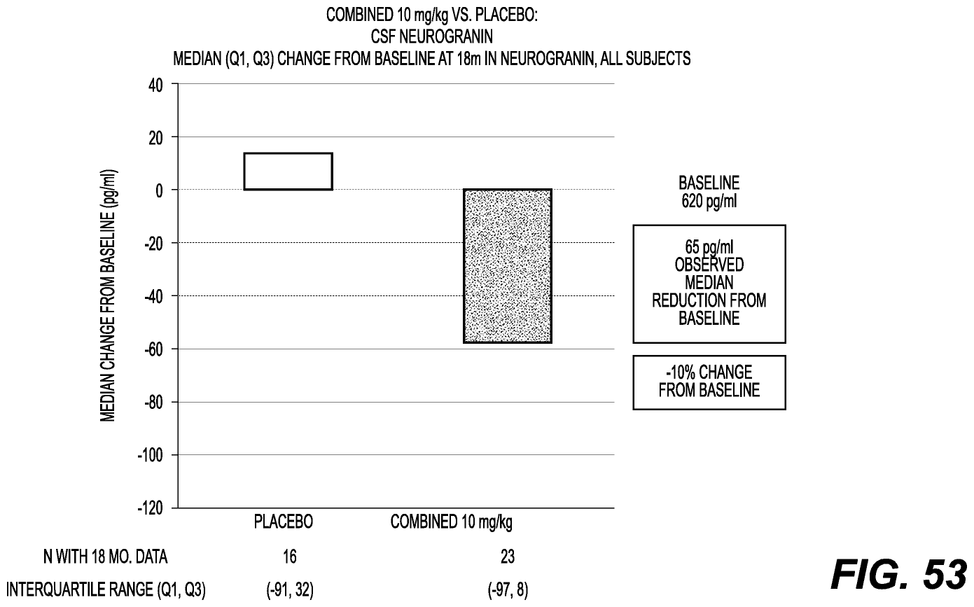

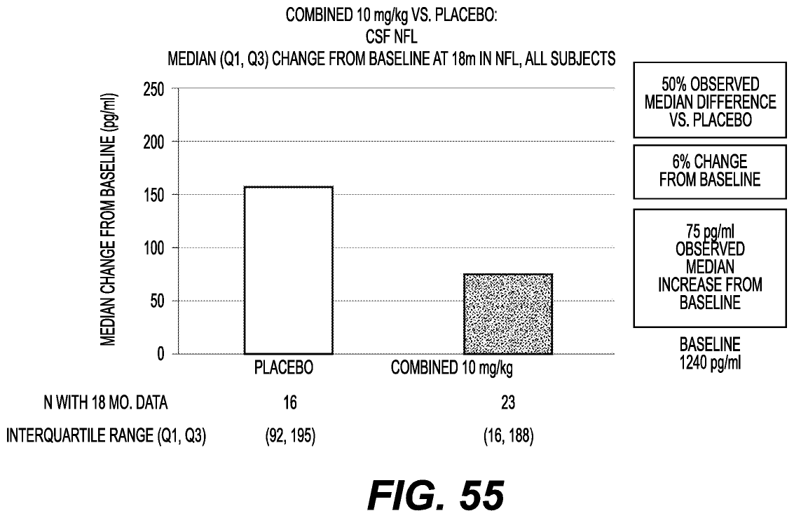

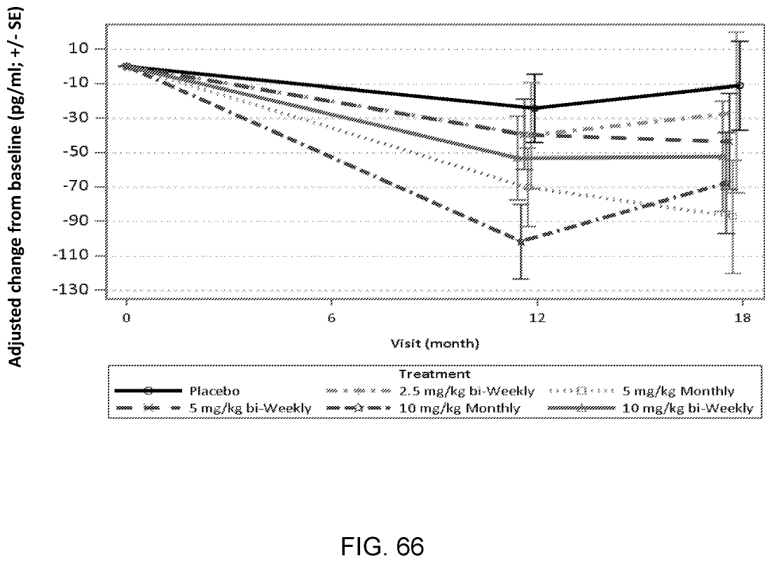

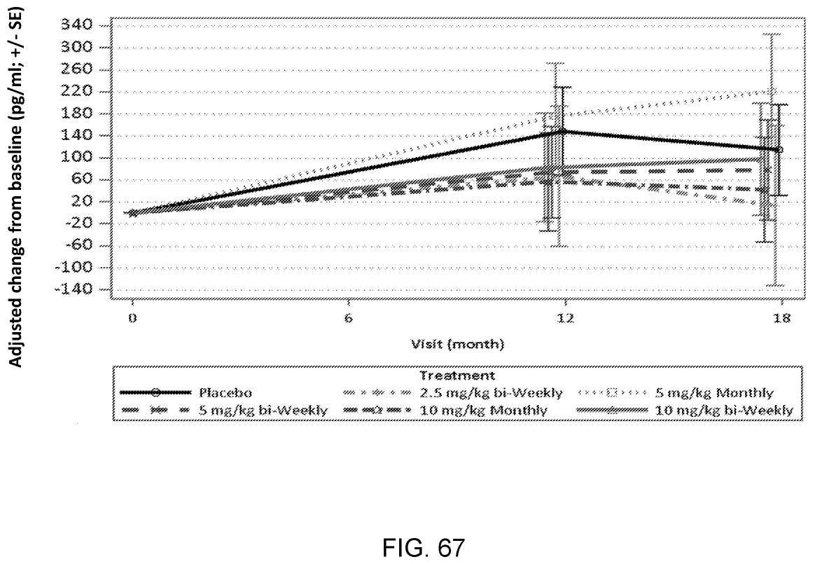

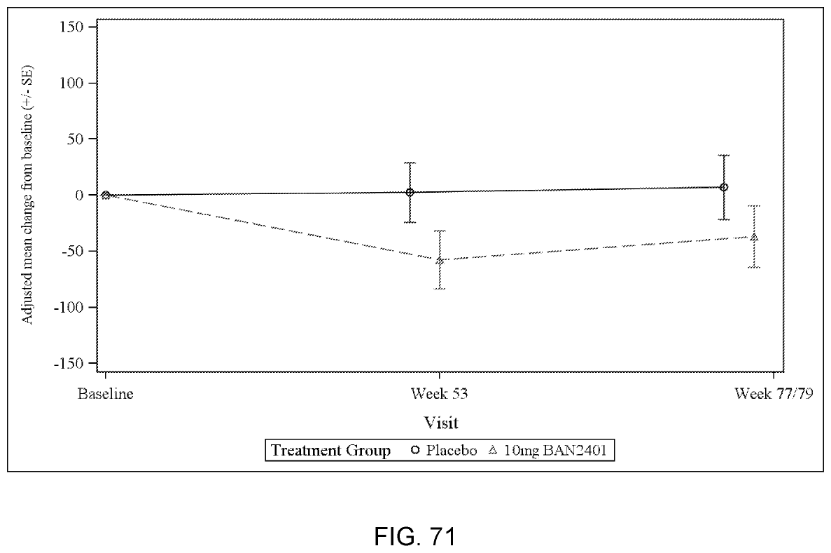

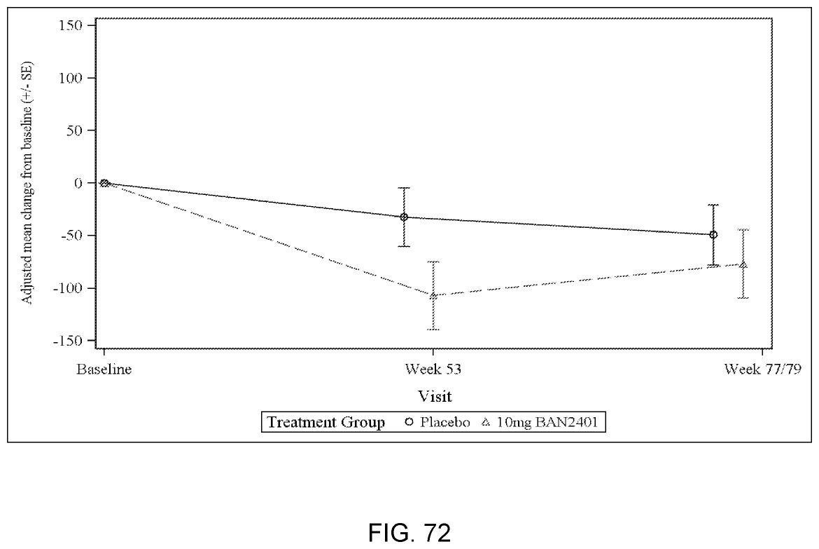

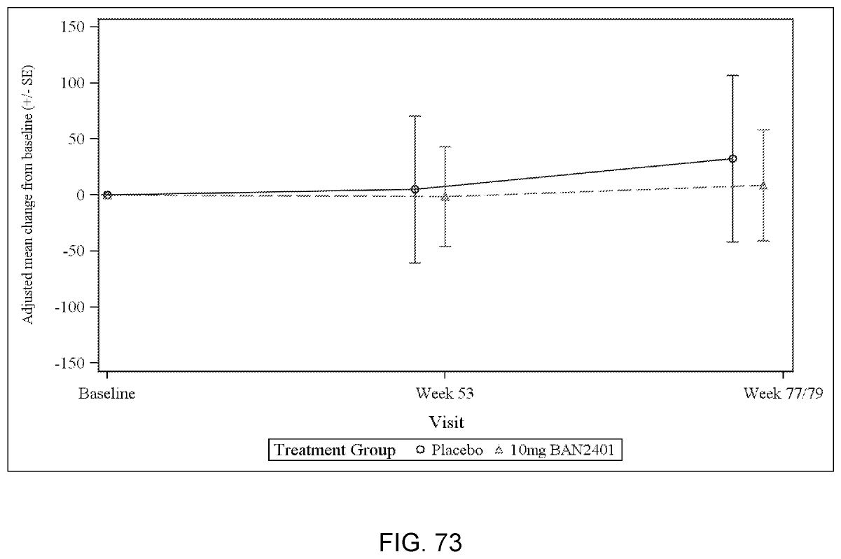

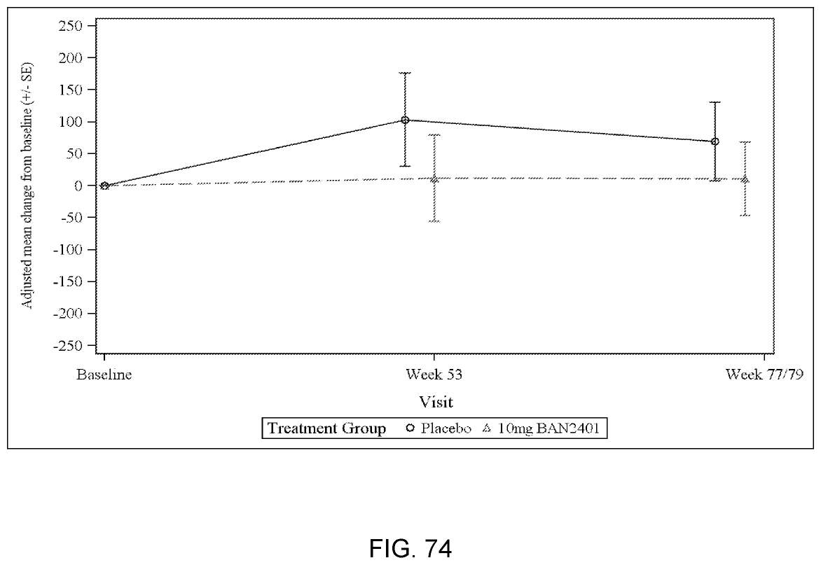

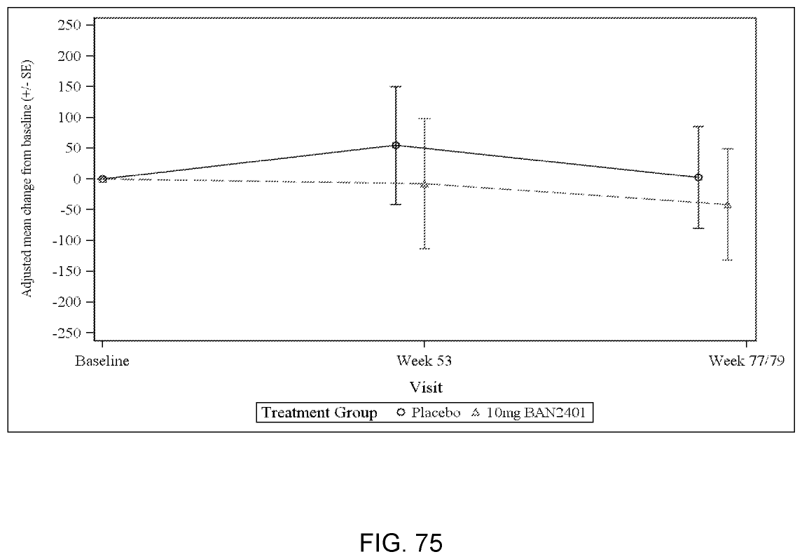

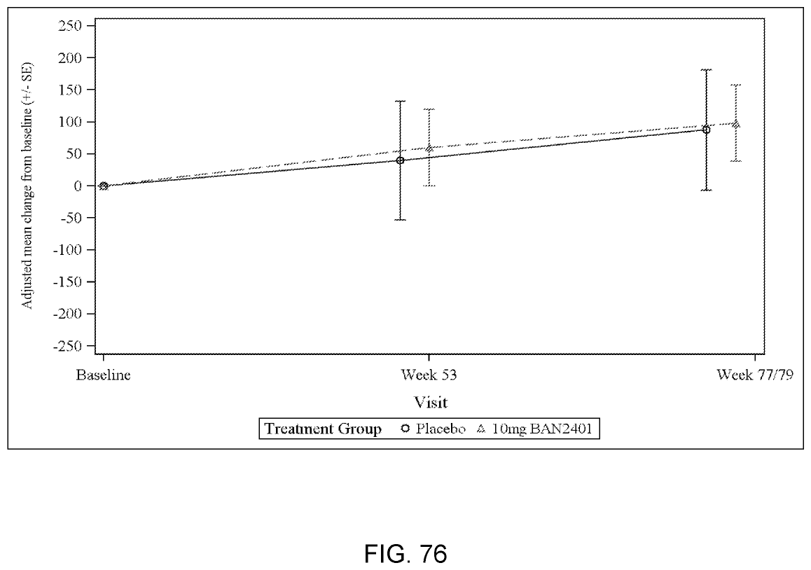

A reduction in cerebrospinal fluid level of neurogranin and a lesser amount of neurofilament light chain, relative to placebo, was observed after administration of BAN2401. (See FIGS. 53, 55, 66, 67, and 71-76.) Administration of 10 mg/kg BAN2401 bi-weekly or monthly resulted in a 10% reduction (versus baseline) of neurogranin (see FIGS. 53) and 50% less production of neurofilament light chain relative to placebo (see FIG. 55).

### Safety Results

Table 19 provides a summary of the treatment-related adverse events.

TABLE 19
Treatment Emergent Adverse Events (“TEAE”).

| | | Treatment Regimen | | | | |
|---|---|---|---|---|---|---|
| | | 2.5 mg/kg | 5 mg/kg | 5 mg/kg | 10 mg/kg | 10 mg/kg |
| | Placebo | bi-weekly | monthly | bi-weekly | monthly | bi-weekly |
| | (N = 245) | (N = 52) | (N = 51) | (N = 92) | (N = 253) | (N = 161) |
| Category | n (%) | n (%) | n (%) | n (%) | n (%) | n (%) |
| Any TEAE | 216 | 46 | 48 | 81 | 238 | 139 |
| | (88.2) | (88.5) | (94.1) | (88.0) | (94.1) | (86.3) |
| Treatment- | 65 | 23 | 25 | 31 | 135 | 76 |
| Related TEAE | (26.5) | (44.2) | (49.0) | (33.7) | (53.4) | (47.2) |
| Serious | 43 | 10 | 4 | 16 | 31 | 25 |
| Adverse | (17.6) | (19.2) | (7.8) | (17.4) | (12.3) | (15.5) |
| Event | | | | | | |
| ARIA-E | 2 | 1 | 1 | 3 | 25 | 16 |
| | (0.8) | (1.9) | (2.0) | (3.3) | (9.9) | (9.9) |
| Deaths | 2 | 2 | 0 | 1 | 2 | 0 |
| | (0.8) | (3.8) | | (1.1) | (0.8) | |
| AE leading to | 15 | 7 | 4 | 10 | 47 | 24 |
| discontinuation | (6.1) | (13.5) | (7.8) | (10.9) | (18.6) | (14.9) |
| from study | | | | | | |
| treatment | | | | | | |

Table 20 provides a summary of the most-frequent treatment-related adverse events.

TABLE 20
Most-Frequent Treatment-Related Adverse Events.

| | | Treatment Regiment | | | | |
|---|---|---|---|---|---|---|
| | | 2.5 mg | 5 mg | 5 mg | 10 mg | 10 mg |
| | Placebo | bi-weekly | monthly | bi-weekly | monthly | bi-weekly |
| | (N = 245) | (N = 52) | (N = 51) | (N = 92) | (N = 253) | (N = 161) |
| Category | n (%) | n (%) | n (%) | n (%) | n (%) | n (%) |
| Any Treatment- | 65 | 23 | 25 | 31 | 135 | 76 |
| Related TEAE | (26.5) | (44.2) | (49) | (33.7) | (53.4) | (47.2) |
| Infusion Related | 8 | 3 | 4 | 11 | 59 | 32 |
| Reaction | (3.3) | (5.8) | (7.8) | (12) | (23.3) | (19.9) |
| ARIA-E | 2 | 1 | 1 | 3 | 25 | 16 |
| | (0.8) | (1.9) | (2.0) | (3.3) | (9.9) | (9.9) |
| Cerebral | 9 | 2 | 6 | 7 | 20 | 10 |
| Microhaemorrhage | (3.7) | (3.8) | (11.8) | (7.6) | (7.9) | (6.2) |
| Headache | 10 | 3 | 1 | 1 | 25 | 8 |
| | (4.1) | (5.8) | (2.0) | (1.1) | (9.9) | (5.0) |
| Fatigue | 6 | 4 | 0 | 1 | 15 | 5 |
| | (2.4) | (7.7) | | (1.1) | (5.9) | (3.1) |

Table 21 provides a summary of the incidence of vasogenic edema (ARIA-E).

TABLE 21
Summary of Incidence of ARIA-E.

| | | Treatment Regimen | | | | |
|---|---|---|---|---|---|---|
| | | 2.5 mg/kg | 5 mg/kg | 5 mg/kg | 10 mg/kg | 10 mg/kg |
| | Placebo | bi-weekly | monthly | bi-weekly | monthly | bi-weekly |
| | (N = 245) | (N = 52) | (N = 51) | (N = 92) | (N = 253) | (N = 161) |
| Category | n (%) | n (%) | n (%) | n (%) | n (%) | n (%) |
| ARIA-E | 2 | 1 | 1 | 3 | 25 | 16 |
| | (0.8) | (1.9) | (2.0) | (3.3) | (9.9) | (9.9) |
| ApoE4+ | 2/173 | 1/38 | 1/40 | 3/84 | 23/225 | 7/48 |
| | (1.2%) | (2.6%) | (2.5%) | (3.6%) | (10.2%) | (14.6%) |
| ApoE4− | 0/72 | 0/14 | 0/11 | 0/8 | 2/28 | 9/113 |
| | | | | | (7.1%) | (8%) |

### Results in ApoE4-Positive Subjects

As discussed above, unpredictably beneficial results were achieved when the subjects are ApoE4 positive as compared to subjects who were non-carriers of ApoE4. This is surprising at least in view of previously reported results of studies performed with other anti-Aβ antibodies.

For example, in a Phase lb trial using aducanumab, another anti-Aβ antibody, reductions in amyloid PET SUVr composite score in aducanumab-treated patients were reported to be similar in ApoE4 carriers and non-carriers (Sevigny, J. et al., “The antibody aducanumab reduces Aβ plaques in Alzheimer's disease,” Nature 537:50-56, 50-51 (Sep. 1, 2016), citing Extended Data FIG. 2b.) The results in Extended Data FIG. 2b, however, reflect better results for treatment of ApoE4 non-carriers as compared to ApoE4 carriers.

In a Phase II trial of bapineuzumab, an anti-Aβ antibody outside the scope of the present disclosure, in treatment of mild-to-moderate Alzheimer's disease, results indicated possible differences on some clinical measures by ApoE4 carrier status, with potential treatment differences favoring noncarriers. (See Salloway et al., “A phase 2 multiple ascending dose trial of bapineuzumab in mild to moderate Alzheimer disease,” Neurology 73:2061-2070, 2067 (Dec. 15, 2009).) Accordingly, two different Phase III clinical trials of bapineuzumab were conducted—one in ApoE4-positive carriers and one in ApoE4 non-carriers with mild-to-moderate Alzheimer's disease. Despite the Phase II indications, the Phase III results not only failed to distinguish between the two ApoE4 genotypes but failed to show a benefit of bapineuzumab with respect to clinical outcomes. (Salloway et al., “Two Phase 3 Trials of Bapineuzumab in Mild-to-Moderate Alzheimer's Disease,” N. Engl. J. Med. 370:322-33 (2014).) Similarly, two Phase III clinical trials with solanezumab, another anti-Aβ antibody outside the scope of the present disclosure, also did not show benefit with respect to the primary clinical outcomes in patients with mild-to-moderate Alzheimer' s disease. (Salloway et al., N. Engl. J. Med. 370:322-33, 332 (2014); Doody R. S. et al., “Phase 3 trials of solanezumab for mild-to-moderate Alzheimer's disease,” N. Engl. J. Med. 370:311-21 (2014).)

Based on results such as those discussed above, the statistically significant results reported herein with ApoE4-positive subjects as compared to non-carriers of ApoE4 were not only unexpected but unpredictable.

### Pharmacokinetic Profile

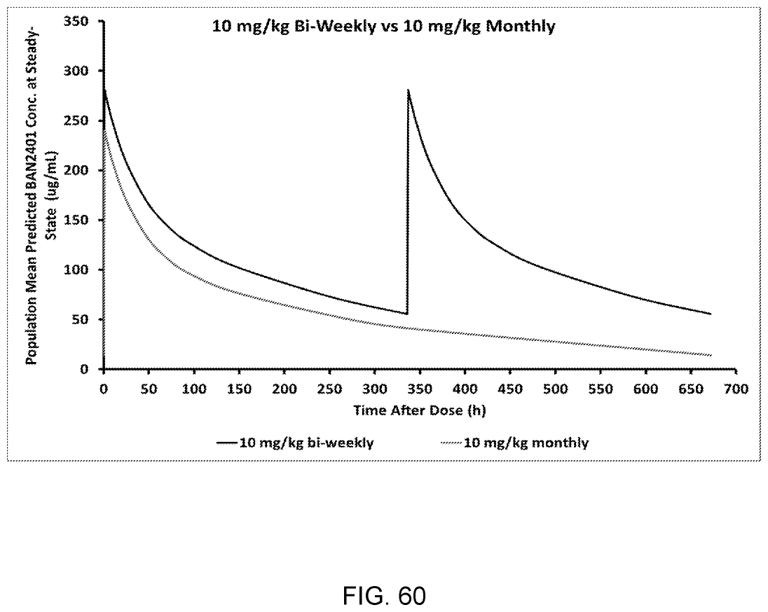

Analysis of subjects' pharmacokinetic profile showed that administration of a composition comprising BAN2401 results in a linear dose response. See, e.g., FIG. 60.

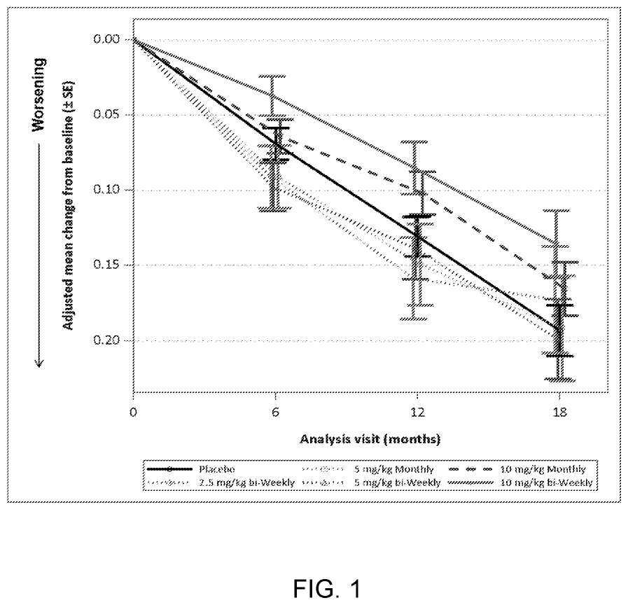

The average steady state plasma concentration for each dose is shown in Table 22. It was found that slowing of disease progression rate, as determined by ADCOMS, ADAS-cog, and CDR-SB, positively correlated with the average steady state plasma concentration of BAN2401. See, e.g., FIGS. 1, 7, and 9 (dose amount positively correlates with slowing of disease progression) and Table 22 (dose amount positively correlates with average steady state plasma concentration).

TABLE 22
Average steady state plasma concentration.

| Dose of BAN2401 | C_{ss,av} (μg/mL) |
|---|---|
| 2.5 mg/kg, | 28.7 |
| bi-weekly | |
| 5 mg/kg, | 28.7 |
| monthly | |
| 5 mg/kg, | 57.3 |
| bi-weekly | |
| 10 mg/kg, | 57.3 |
| monthly | |
| 10 mg/kg, | 114.7 |
| bi-weekly | |

Table 23 shows the population pharmacokinetic parameters after administration of BAN2401.

TABLE 23
Population pharmacokinetic parameters for BAN2401.

| | | | |
|---|---|---|---|
| | Point | | |
| Parameter | Estimate | % RSE | 95% Cl |

Clearance: CL

| | | | |
|---|---|---|---|
| Basal CL (L/h) | 0.0191 | 2.84 | 0.0180-0.0202 |
| Effect of sex on CL (ratio) | 0.786 | 3.97 | 0.725-0.847 |
| Effect of body weight on CL | 0.384 | 18.5 | 0.245-0.523 |
| (exponent) | | | |
| Effect of albumin on CL | −0.267 | 22.5 | −0.385-−0.149 |
| (exponent) | | | |

Central volume of distribution: V1

| | | | |
|---|---|---|---|
| Basal V1 (L) | 3.18 | 1.21 | 3.10-3.26 |
| Effect of body weight on V1 | 0.621 | 7.94 | 0.524-0.718 |
| (exponent) | | | |
| Effect of sex on V1 (ratio) | 0.904 | 1.85 | 0.871-0.937 |

Inter-compartment Clearance: Q

| | | | |
|---|---|---|---|
| Basal Q (L/h) | 0.0350 | 8.00 | 0.0295-0.0405 |

Peripheral volume of distribution: V2

| | | | |
|---|---|---|---|
| Basal V2 (L) | 2.24 | 8.12 | 1.88-2.60 |
| Effect of Japanese race on V2 | 0.423 | 31.2 | 0.164-0.682 |
| (ratio) | | | |

Inter-individual variability (% CV)

| | | | |
|---|---|---|---|
| CL | 41.0 | 6.49 | — |
| Covariance CL_V1 | 0.104 | 59.9 | — |
| V1 | 13.6 | 8.76 | — |
| Q | NE | — | — |
| V2 | 97.1 | 8.36 | — |

Residual variability

| | | | |
|---|---|---|---|
| Proportional Study 1 (% CV) | 13.9 | 3.51 | — |
| Proportional Study 2 (% CV) | 18.8 | 3.94 | — |
| Proportional Study 3 (% CV) | 27.5 | 0.938 | — |

Abbreviations: NE = not estimated; % RSE = percent relative standard error of the estimate = SE/parameter estimate * 100; CL = clearance; V1 = central volume of distribution; Q = inter-compartment clearance; V2 = peripheral volume of distribution; L = liter; h = hour; Cl = confidence interval; % CV = square root of variance * 100.

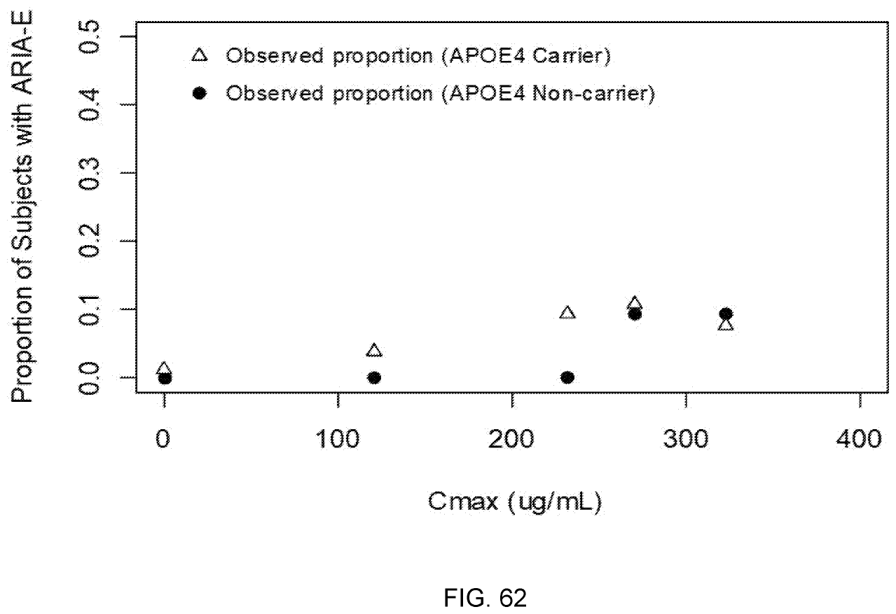

It was also found that the proportion of ApoE4-positive subjects who experienced an ARIA-E event positively correlated with maximum plasma concentration of BAN2401 at all studied doses. See FIG. 62. In contrast, the proportion of ApoE4-negative subjects who experienced an ARIA-E event and who were administered BAN2401 at a dose resulting in a maximum plasma concentration of about 230 pg/mL or less was approximately comparable to the proportion of ApoE4-negative subjects who were administered placebo. See id.

### Concomitant Administration of At Least One Alzheimer's Disease Medication Other Than BAN2401

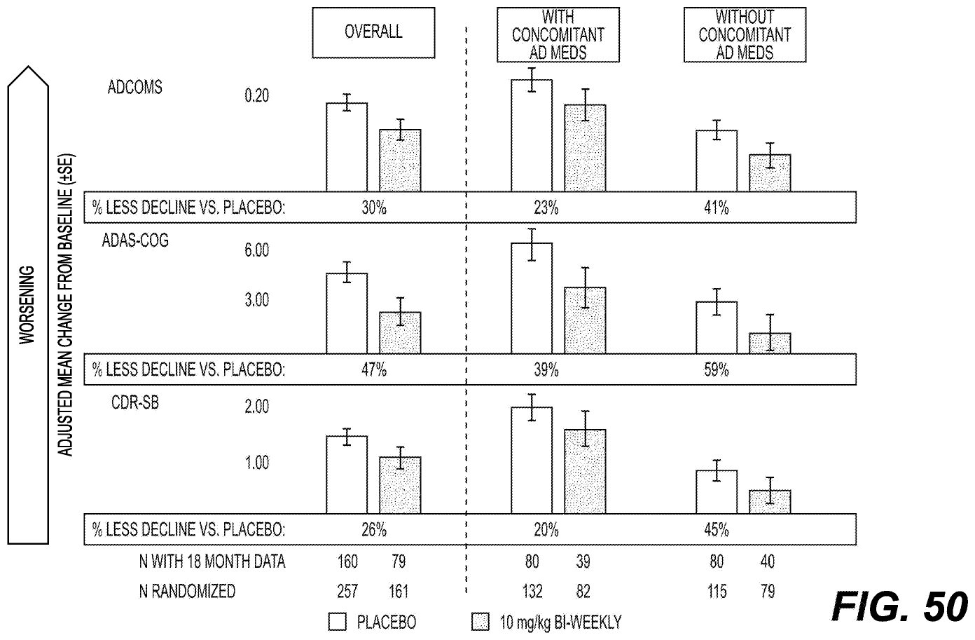

Subjects, regardless of whether they were concomitantly administered at least one Alzheimer's disease medication other than BAN2401, were administered BAN2401 as described hereinabove. It was surprisingly discovered that subjects who were not concomitantly administered at least one Alzheimer's disease medication other than BAN2401 showed a greater slowing of cognitive decline, relative to baseline, than subjects who were concomitantly administered BAN2401 and at least one Alzheimer's disease medication other than BAN2401. These findings were consistent across analyses: ADCOMS (23% slowing of cognitive decline for subjects with concomitant administration of at least one Alzheimer's disease medication other than BAN2401 vs. 41% without); ADAS-cog (39% slowing of cognitive decline for subjects with concomitant administration of at least one Alzheimer's disease medication other than BAN2401 vs. 59% without); and CDR-SB (20% slowing of cognitive decline for subjects with concomitant administration of at least one Alzheimer's disease medication other than BAN2401 vs. 45% without). See FIG. 50.

### Risk Factors for Disease Progression

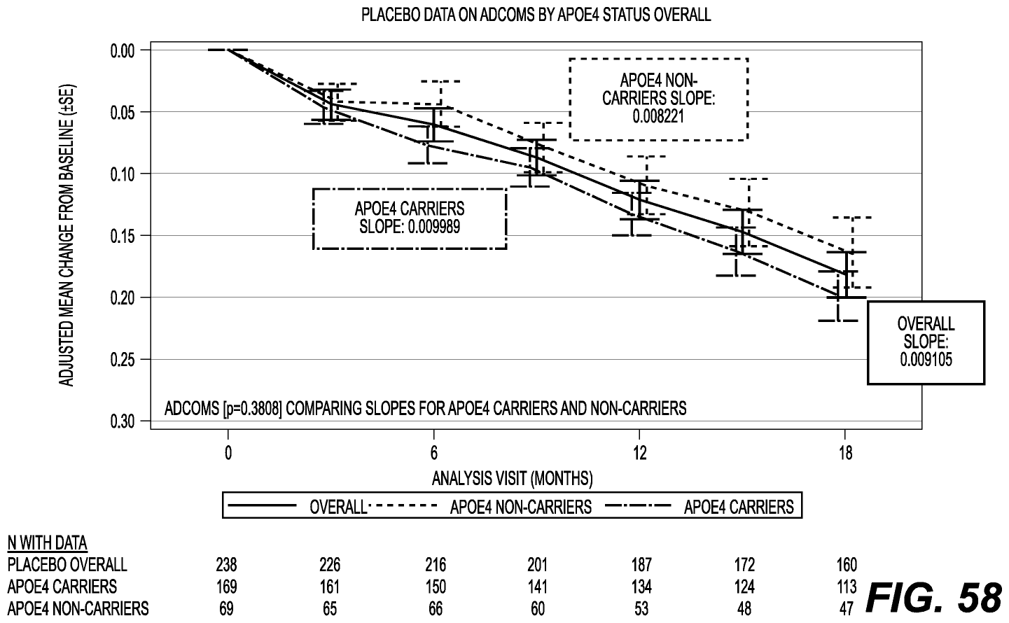

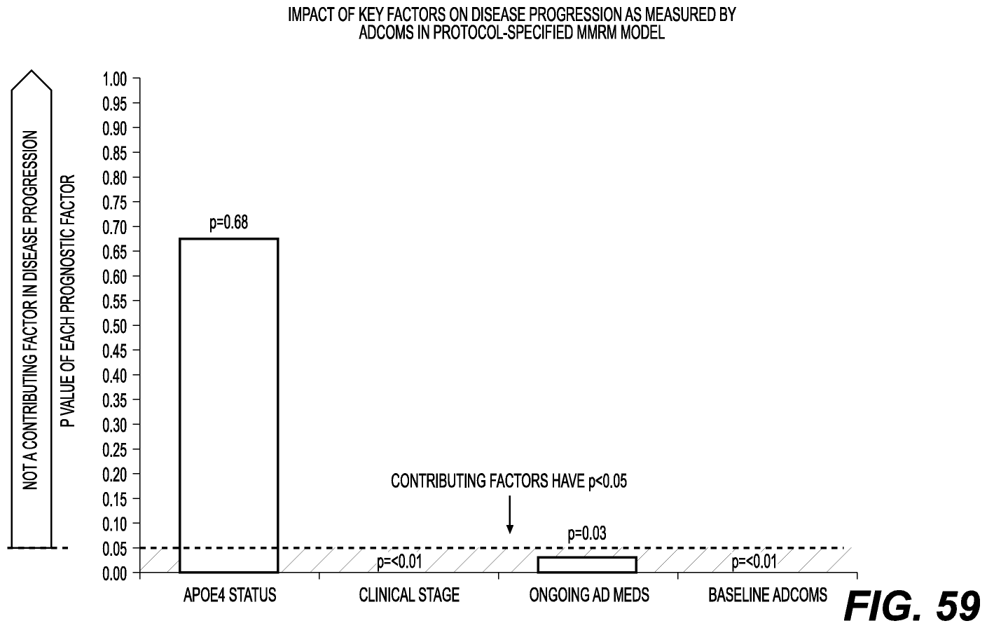

A subject's ApoE4 status was found to not be a statistically significant risk factor in determining disease progression. However, other factors, e.g., clinical stage of disease, concomitantly administration of at least one Alzheimer's disease medication other than BAN2401, and baseline ADCOMS score, were found to be statistically significant risk factors. See FIGS. 58 and 59.

### Prophetic Example A: Prevention of Alzheimer's Disease in ApoE4-Positive Subjects

Subjects regardless of gender are screened for ApoE4 carrier status. Subjects may be selected for screening based on age, e.g., 50 years and older, risk of Alzheimer's disease, and/or other criteria.

If screening determines that a subject is ApoE4-positive, the subject's brain amyloid level is measured, for example by visual reads of amyloid PET images as discussed above, or another technique known to one of ordinary skill in the art. The subject's cerebrospinal fluid Aβ₁₋₄₂ level and/or cerebrospinal fluid total tau level may also be measured.

If the subject's brain amyloid level is above a predetermined level, such as a PET SUVr value above 1.1, such as above 1.2, above 1.3, above 1.4, above 1.5, or above 1.6, a composition comprising a therapeutically effective amount of at least one anti-Aβ protofibril antibody is administered to the subject. In some embodiments, if the subject's brain amyloid level is above a predetermined level, such as PET SUVr value above 1.3, such as above 1.4, above 1.5, above 1.6, above 1.7, above 1.8, or above 1.9, a composition comprising a therapeutically effective amount of at least one anti-Aβ protofibril antibody is administered to the subject. The composition may be administered in an amount and according to a dosing regimen and route as described herein, such as those disclosed in Example 1.

If the subject's brain amyloid level is below the above-referenced predetermined level, the subject can be monitored and/or have brain amyloid levels determined at a later date, e.g., six months later.

After administration of the composition comprising a therapeutically effective amount of at least one anti-Aβ protofibril antibody to the subject, the subject's brain amyloid level is measured again. If the subject's brain amyloid level is above a predetermined level, administration of the composition is repeated. If the subject's brain amyloid level is below a predetermined level, the subject can be monitored and/or have brain amyloid levels determined at a later date, e.g., within six months.

During the later determination, if the brain amyloid level of the subject is above a predetermined level, a composition comprising a therapeutically effective amount of at least one anti-Aβ protofibril antibody is administered to the subject.

If, while the subject is monitored, the subject's brain amyloid level rises above a predetermined level, according to, e.g., PET or cerebrospinal fluid measures, administration of the composition comprising a therapeutically effective amount of at least one anti-Aβ protofibril antibody would resume. The brain amyloid level would again be measured after administration of the composition comprising a therapeutically effective amount of at least one anti-Aβ protofibril antibody.

After administration of the composition, at least one maintenance therapy agent, such as a BACE inhibitor, is optionally administered to the subject. In some embodiments, the BACE inhibitor may be elenbecestat.

In addition to measurement and consideration of brain amyloid level, cerebrospinal fluid Aβ₁₋₄₂ level, and/or cerebrospinal fluid total tau level, other measures of the subject's health are measured and/or monitored, e.g., MMSE, ADAS-cog, CDR, and FAQ.

### Prophetic Example B: Treatment of Subjects Haying Early Alzheimer's Disease

Subjects, both male and female with ages ranging from 50 to 90 years old, inclusive, having mild cognitive impairment due to Alzheimer's disease—intermediate likelihood and/or mild Alzheimer's disease dementia are screened to be eligible for treatment. A total of 990 subjects are randomized across two treatment groups, as described below, in a ratio of 1 subject administered placebo for every 2 subjects administered BAN2401.

The study consists of a prerandomization phase and a randomization phase.

### Prerandomization Phase

This phase lasts up to 60 days and consists of a screening period and a baseline period. During the screening period, subjects are assessed based on eligibility criteria. If a subject is deemed to be eligible, the subject then proceeds to the baseline period.

During the baseline period, a number of analyses are performed, including clinical tests (e.g., blood tests (e.g., for determining ApoE4 status), etc.), safety magnetic resonance imaging (MRI) analyses, amyloid PET assessments (e.g., visual reads of amyloid PET images), and cerebrospinal fluid (for biomarker analysis) if the subject consented. Additional clinical testing of subjects includes MMSE, CDR, ADAS-Cog, and FAQ.

### Randomization Phase

The randomization phase consists of an 18-month treatment period and a 3-month follow-up period. Subjects are randomized and administered either placebo or a composition comprising 10 mg/kg BAN2401, once every 2 weeks.

All subjects are administered an approximately 60-minute intravenous infusion every 2 weeks. BAN2401 was administered in normal saline as 60-minute IV infusions.

All subjects are assessed based on cognitive function, safety, pharmacokinetic parameters, safety MRI, PET imaging, volumetric MRI and cerebrospinal fluid analysis, if the subject consented to such analysis. Additional clinical assessments include MMSE, CDR, ADAS-cog, FAQ, and amyloid measurements. These measurements are taken at regular intervals, e.g., during the baseline period, 3 months, 6 months, 9 months, 12 months, 15 months, and/or 18 months after commencement of treatment.

Treatment is ended at the later of 18 months, request by the subject, request of the attending physician and/or data safety monitoring board, and/or the emergence of one or more adverse events warranting the discontinuation of treatment.

After the end of treatment, subjects are evaluated as during the baseline period and randomization phase. Subjects not participating in the Extension Phase (described below) attend a 3-month follow-up visit where parameters similar to those measured during the Randomization Phase are measured/assessed.

### Extension Phase

A subject who completes the Randomization Phase has the option to participate in the Extension Phase. During this phase, the subject remains on the same treatment as during the Randomization Phase. Similarly, monitoring of symptoms and other parameters, as described for the Randomization Phase, will also be measured during the Extension Phase. The Extension Phase will continue until termination by request of the subject, request of the attending physician and/or data safety monitoring board, emergence of one or more adverse events warranting the discontinuation of treatment, commercial availability of BAN2401 and/or study results fail to demonstrate a positive risk-benefit relationship. A 3-month follow-up visit takes place after the last dose of study drug where parameters similar to those measured during the Randomization Phase are measured/assessed.

### Formulation

BAN2401 is supplied as a sterile, clear solution for injection containing 10 mg/mL, in a single use 10 mL vial (total 100 mg/vial). The drug product was formulated in 25 mM sodium citrate, 125 mM sodium chloride, 0.02% (w/v) polysorbate 80, and the pH is determined to be 5.7. BAN2401 is administered via an infusion system containing a terminal 0.22 μM in-line filter.

### Study Endpoints

The primary study endpoint is to determine the change from baseline in the Alzheimer's disease composite score (ADCOMS) at 18 months. The key secondary endpoints include measuring change from baseline in amyloid PET SUVr composite at 18 months for brain amyloid levels and change from baseline in the ADAS-cog score at 18 months.

Secondary endpoints include changes from baseline in the CDR-SB score at 18 months and time to worsening of the CDR-SB score at 18 months.

Biomarker endpoints include change from baseline in amyloid PET SUVr composite at 6 and 12 months for brain amyloid levels; change from baseline in cerebrospinal fluid neurogranin, neurofilament light chain, Aβ₁₋₄₂, and total Tau at 12 and 18 months; and change from baseline in morphometric MRI measurements (including hippocampal volume) at 6, 12, and 18 months using vMRI.

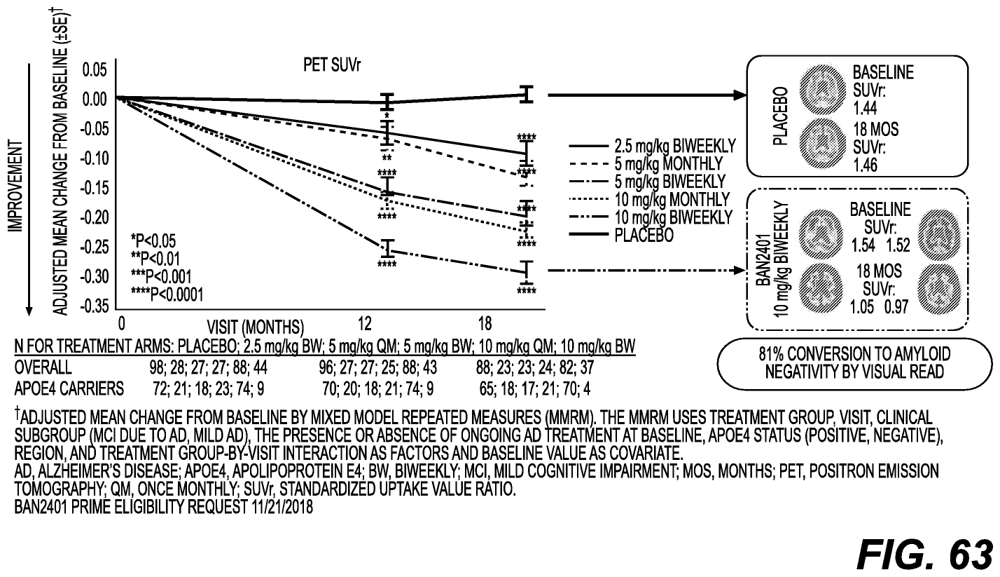

Eligible subjects for prophetic OLE Example C: Subjects who are eligible for treatment in prophetic Example C, below, are those with early AD (MCI due to AD and mild AD dementia subjects) who were treated in a multinational, multicenter, double-blind, placebo-controlled, parallel-group, 18 month study that assessed clinical safety and efficacy, and explored dose response of BAN2401 at 12 and 18 months of treatment with the Alzheimer's Disease Composite Score (ADCOMS) in early AD. In this study, a Bayesian design with response adaptive randomization across placebo or 5 active arms of BAN2401 was used in subjects with early AD on ADCOMS at 12 months. Slower decline in early AD subjects was observed at both 12 and 18-months with BAN2401 10 mg/kg biweekly dose for ADCOMS (30%-less decline), ADAS-cog (47%-less), MMSE (19%-less), and CDR-SB (26%-less). Differences were observed as early as 6-months on these clinical measures. The clinical results were accompanied by robust and dose dependent reduction in brain amyloid across all doses as measured by quantitative amyloid PET. See, e.g., FIG. 63.

### Prophetic Example C: Open Label Extension Study of BAN2401: Treatment of Subjects Haying Early Alzheimer's Disease

Eligible subjects as discussed above are treated in an open-label extension study (OLE) for up to 24 months of with BAN2401 at a dose of 10 mg/kg biweekly. In the OLE, the long-term safety and tolerability of BAN2401 are evaluated and the effects of BAN2401 on brain amyloid in subjects who participated in the previous study as summarized below are evaluated.

Approximately 200 to 250 eligible subjects as discussed above, both male and female, regardless of ApoE status, may be admitted to the OLE study based on the inclusion and exclusion criteria set forth below.

Inclusion criteria for the OLE include:
prior treatment in the previous study for at least 79 weeks or for less than 79 weeks due to:
   i. experiencing an ARIA-E event;
   ii. experiencing an ARIA-H event (e.g., superficial siderosis, microhemorrhage, and/or symptomatic microhemorrhage);
   iii. taking a medication that was prohibited during the earlier BAN2401 treatment, but which is no longer prohibited; and/or
   iv. any reason not related to prohibited medication, including any adverse event that was not related to BAN2401 treatment and not deemed to be severe or life-threatening;
having a caregiver/informant who can provide treatment-related details about the subject;
ability to provide informed consent; and
ability to attend clinic visits.

Exclusion criteria for the OLE include:
prior treatment in the previous study discontinued after less than 79 weeks for reasons other than:
   i. experiencing an ARIA-E incident;
   ii. experiencing an ARIA-H incident (e.g., superficial siderosis, microhemorrhage, and/or symptomatic microhemorrhage);
   iii. taking a medication that was prohibited during the earlier BAN2401 treatment, but which is no longer prohibited; and/or
   iv. being ApoE4-positive and receiving treatment with BAN2401 at a dose of 10 mg/kg bi-weekly; and/or
   v. experiencing an adverse event that was deemed not related to study drug (placebo or BAN2401) and that was not severe or life-threatening;
breastfeeding females or females of childbearing potential who may be or are risk of becoming pregnant; and/or
development of a condition after treatment in the previous study that would interfere with or pose a safety concern for administration of BAN2401.

Prior to administration of BAN2401 in the present OLE, participating subjects are assessed to determine baseline characteristics, such as cognitive function, safety parameters, pharmacokinetic parameters, safety MRI, PET imaging, and volumetric MRI. Additional clinical assessments may include MMSE, CDR, ADAS-cog, FAQ, and amyloid measurements.

Treatment in the present prophetic OLE example includes administration to eligible subjects a 10 mg/kg bi-weekly dose of BAN2401 for up to 24 months.

BAN2401 is supplied as any formulation suitable for administration to a human subject, for example, as a sterile, clear solution for injection containing 10 mg/mL, in a single-use 10 mL vial (total 100 mg/vial) comprising 25 mM sodium citrate, 125 mM sodium chloride, 0.02% (w/v) polysorbate 80, and having pH of 5.7. BAN2401 may be administered via an infusion system containing a terminal 0.22 μM in-line filter.

During treatment in the OLE, at various time intervals (3 months, 6 months, 9 months, 12 months, 18 months, and/or 24 months), and upon completion of treatment in the OLE, subjects are assessed by one or more assessment techniques, such baseline and/or clinical assessment techniques. Suitable assessments may include cognitive function, safety parameters, pharmacokinetic parameters, safety MRI, PET imaging, volumetric MRI, MMSE, CDR, ADAS-cog, FAQ, and amyloid measurements.

During treatment in the OLE, the subject continues to be treated with BAN2401 without interruption even if a subject develops asymptomatic ARIA-H cerebral microhemorrhage, multiple asymptomatic cerebral microhemorrhages, asymptomatic superficial siderosis, a single asymptomatic microhemorrhage, and/or asymptomatic or radiographically mild or moderate ARIA-E. If a subject develops symptomatic ARIA-H and/or asymptomatic or radiographically moderate to severe ARIA-E, BAN2401 administration is suspended until the ARIA-H or ARIA-E has radiographically stabilized or resolved and clinical features have resolved, after which BAN2401 administration may resume at the same dose and regimen. If treatment needs to be resumed more than twice in the OLE, the subject is removed from the study.

### OLE Study Assessments and Endpoints

The long-term safety and tolerability of treating subjects having early Alzheimer's disease with BAN2401 bi-weekly at 10 mg/kg is assessed in the OLE. Assessment includes incidence and severity of ARIA-H, ARIA-E, and non-ARIA-H/non-ARIA-E adverse events.

Brain amyloid level (for example, as determined by PET imaging) is determined prior to and/or at the end of the previous study, prior to and optionally during and after the present prophetic OLE treatment example with BAN2401 to assess removal and/or maintenance of absence of amyloid in the brain due to treatment with BAN2401. Brain amyloid level (e.g., by longitudinal PET imaging) is evaluated after 3 months, 6 months, 12 months, 18 months, and/or 24 months of the present prophetic OLE treatment with BAN2401, for example in terms of change from baseline in brain amyloids after the previous study as discussed above. The proportion of amyloid positive subjects, e.g., as determined by visual reads of amyloid PET images, is also assessed over time in the OLE.

Cognitive assessment of subjects during this OLE study as determined by, e.g., ADCOMS, CDR-SB, ADAS-Cog, and MMSE, may provide insight about the long-term cognitive effects of treatment with BAN2401. The assessments may provide insight about the long-term disease progression and persistence of any slowing in the rate of cognitive decline that was observed during the prior study.

Because parameters such as pharmacokinetic, hippocampal volume, and other biomarkers will be measured, this OLE study may provide insight about long-term effects of treatment with BAN2401.

Information concerning management of ARIA-E and ARIA-H incidents may be provided from this OLE study since, unlike the previous protocol, the present prophetic OLE protocol continues treatment of subjects with BAN2401 even if ARIA-E and/or ARIA-H incidents are experienced, unless the severity mandates treatment discontinuation.

Additional insight on the persistence of amyloid reduction following drug holiday (e.g., how long the reduction lasts after BAN2401 treatment is discontinued), the time course of amyloid level increase and/or deposition, and/or the time course of amyloid removal (how quickly amyloid is removed by BAN2401 treatment) may also be obtained from the present prophetic OLE study.

### Initial Results

Enrollment in the OLE study is ongoing. Baseline amyloid PET data is available from 56 subjects enrolled in the OLE study, which includes data from 39 subjects who were treated with BAN2401 in the previous study with a mean duration off of study drug of 21 months (ranging from 9 months to 52 months off of study drug). 85% (33/39) of the subjects who were previously amyloid positive and who were treated with BAN2401 (10 mg/kg either monthly or bi-weekly for 18 months) were determined to still be amyloid negative after 9 months to 52 months off of study drug. All subjects entering the OLE study who were amyloid negative in the previous study after 18 months were also amyloid negative at the baseline measurement of the OLE study, regardless of the treatment group in the prior study. Thus, these initial results unexpectedly show that BAN2401-mediated return of subjects to amyloid negativity, as determined by PET, persisted from the end of the previous study to the baseline measurement of the OLE study, despite subjects being off treatment from 9 to 52 months.

In some embodiments, the methods disclosed herein result in a reduction in amyloid. In some embodiments, the reduction in amyloid is determined by PET. In some embodiments, the reduction in amyloid, as determined by PET, persists from 9 months to 52 months after administration of BAN2401.

In some embodiments, the reduction in amyloid, as determined by PET, persists for at least 1 month after administration of BAN2401. In some embodiments, the reduction in amyloid, as determined by PET, persists for at least 2 months after administration of BAN2401. In some embodiments, the reduction in amyloid, as determined by PET, persists for at least 3 months after administration of BAN2401. In some embodiments, the reduction in amyloid, as determined by PET, persists for at least 4 months after administration of BAN2401. In some embodiments, the reduction in amyloid, as determined by PET, persists for at least 5 months after administration of BAN2401. In some embodiments, the reduction in amyloid, as determined by PET, persists for at least 6 months after administration of BAN2401. In some embodiments, the reduction in amyloid, as determined by PET, persists for at least 7 months after administration of BAN2401. In some embodiments, the reduction in amyloid, as determined by PET, persists for at least 8 months after administration of BAN2401. In some embodiments, the reduction in amyloid, as determined by PET, persists for at least 9 months after administration of BAN2401. In some embodiments, the reduction in amyloid, as determined by PET, persists for at least 10 months after administration of BAN2401.

In some embodiments, the reduction in amyloid, as determined by PET, persists for at least 15 months after administration of BAN2401. In some embodiments, the reduction in amyloid, as determined by PET, persists for at least 20 months after administration of BAN2401. In some embodiments, the reduction in amyloid, as determined by PET, persists for at least 25 months after administration of BAN2401. In some embodiments, the reduction in amyloid, as determined by PET, persists for at least 30 months after administration of BAN2401. In some embodiments, the reduction in amyloid, as determined by PET, persists for at least 35 months after administration of BAN2401. In some embodiments, the reduction in amyloid, as determined by PET, persists for at least 40 months after administration of BAN2401. In some embodiments, the reduction in amyloid, as determined by PET, persists for at least 45 months after administration of BAN2401. In some embodiments, the reduction in amyloid, as determined by PET, persists for at least 50 months after administration of BAN2401.

In some embodiments, the reduction in amyloid, as determined by PET, persists for 9 months after administration of BAN2401. In some embodiments, the reduction in amyloid, as determined by PET, persists for 10 months after administration of BAN2401. In some embodiments, the reduction in amyloid, as determined by PET, persists for 11 months after administration of BAN2401. In some embodiments, the reduction in amyloid, as determined by PET, persists for 12 months after administration of BAN2401. In some embodiments, the reduction in amyloid, as determined by PET, persists for 13 months after administration of BAN2401. In some embodiments, the reduction in amyloid, as determined by PET, persists for 14 months after administration of BAN2401. In some embodiments, the reduction in amyloid, as determined by PET, persists for 15 months after administration of BAN2401. In some embodiments, the reduction in amyloid, as determined by PET, persists for 16 months after administration of BAN2401. In some embodiments, the reduction in amyloid, as determined by PET, persists for 17 months after administration of BAN2401. In some embodiments, the reduction in amyloid, as determined by PET, persists for 18 months after administration of BAN2401. In some embodiments, the reduction in amyloid, as determined by PET, persists for 19 months after administration of BAN2401.

In some embodiments, the reduction in amyloid, as determined by PET, persists for 20 months after administration of BAN2401. In some embodiments, the reduction in amyloid, as determined by PET, persists for 21 months after administration of BAN2401. In some embodiments, the reduction in amyloid, as determined by PET, persists for 22 months after administration of BAN2401. In some embodiments, the reduction in amyloid, as determined by PET, persists for 23 months after administration of BAN2401. In some embodiments, the reduction in amyloid, as determined by PET, persists for 24 months after administration of BAN2401. In some embodiments, the reduction in amyloid, as determined by PET, persists for 25 months after administration of BAN2401. In some embodiments, the reduction in amyloid, as determined by PET, persists for 26 months after administration of BAN2401. In some embodiments, the reduction in amyloid, as determined by PET, persists for 27 months after administration of BAN2401. In some embodiments, the reduction in amyloid, as determined by PET, persists for 28 months after administration of BAN2401. In some embodiments, the reduction in amyloid, as determined by PET, persists for 29 months after administration of BAN2401.

In some embodiments, the reduction in amyloid, as determined by PET, persists for 30 months after administration of BAN2401. In some embodiments, the reduction in amyloid, as determined by PET, persists for 31 months after administration of BAN2401. In some embodiments, the reduction in amyloid, as determined by PET, persists for 32 months after administration of BAN2401. In some embodiments, the reduction in amyloid, as determined by PET, persists for 33 months after administration of BAN2401. In some embodiments, the reduction in amyloid, as determined by PET, persists for 34 months after administration of BAN2401. In some embodiments, the reduction in amyloid, as determined by PET, persists for 35 months after administration of BAN2401. In some embodiments, the reduction in amyloid, as determined by PET, persists for 36 months after administration of BAN2401. In some embodiments, the reduction in amyloid, as determined by PET, persists for 37 months after administration of BAN2401. In some embodiments, the reduction in amyloid, as determined by PET, persists for 38 months after administration of BAN2401. In some embodiments, the reduction in amyloid, as determined by PET, persists for 39 months after administration of BAN2401.

In some embodiments, the reduction in amyloid, as determined by PET, persists for 40 months after administration of BAN2401. In some embodiments, the reduction in amyloid, as determined by PET, persists for 41 months after administration of BAN2401. In some embodiments, the reduction in amyloid, as determined by PET, persists for 42 months after administration of BAN2401. In some embodiments, the reduction in amyloid, as determined by PET, persists for 43 months after administration of BAN2401. In some embodiments, the reduction in amyloid, as determined by PET, persists for 44 months after administration of BAN2401. In some embodiments, the reduction in amyloid, as determined by PET, persists for 45 months after administration of BAN2401. In some embodiments, the reduction in amyloid, as determined by PET, persists for 46 months after administration of BAN2401. In some embodiments, the reduction in amyloid, as determined by PET, persists for 47 months after administration of BAN2401. In some embodiments, the reduction in amyloid, as determined by PET, persists for 48 months after administration of BAN2401. In some embodiments, the reduction in amyloid, as determined by PET, persists for 49 months after administration of BAN2401.

In some embodiments, the reduction in amyloid, as determined by PET, persists for 50 months after administration of BAN2401. In some embodiments, the reduction in amyloid, as determined by PET, persists for 51 months after administration of BAN2401. In some embodiments, the reduction in amyloid, as determined by PET, persists for 52 months after administration of BAN2401. Prophetic Example D: Prevention of Alzheimer's disease

Subjects may be selected for screening based on age, e.g., 50 years and older, risk of Alzheimer's disease, and/or other criteria. A subject's ApoE4 carrier status is determined, however, it does not affect the subject's eligibility to participate in the study.

The subject's brain amyloid level is measured, for example by visual reads of amyloid PET images as discussed above, or another technique known to one of ordinary skill in the art. The subject's cerebrospinal fluid Aβ₁₋₄₂ level and/or cerebrospinal fluid total tau level may also be measured.

If the subject's brain amyloid level is above a predetermined level, such as a PET SUVr value above 1.1, such as above 1.2, above 1.3, above 1.4, above 1.5, or above 1.6, a composition comprising a therapeutically effective amount of at least one anti-Aβ protofibril antibody is administered to the subject. In some embodiments, if the subject's brain amyloid level is above a predetermined level, such as PET SUVr value above 1.3, such as above 1.4, above 1.5, above 1.6, above 1.7, above 1.8, or above 1.9, a composition comprising a therapeutically effective amount of at least one anti-Aβ protofibril antibody is administered to the subject. The composition may be administered in an amount and according to a dosing regimen and route as described herein, such as those disclosed in Example 1.

If the subject's brain amyloid level is below the above-referenced predetermined level, the subject can be monitored and/or have brain amyloid levels determined at a later date, e.g., six months later.

After administration of the composition comprising a therapeutically effective amount of at least one anti-Aβ protofibril antibody to the subject, the subject's brain amyloid level is measured again. If the subject's brain amyloid level is above a predetermined level, administration of the composition is repeated. If the subject's brain amyloid level is below a predetermined level, the subject can be monitored and/or have brain amyloid levels determined at a later date, e.g., within six months.

During the later determination, if the brain amyloid level of the subject is above a predetermined level, a composition comprising a therapeutically effective amount of at least one anti-Aβ protofibril antibody is administered to the subject.

If, while the subject is monitored, the subject's brain amyloid level rises above a predetermined level, according to, e.g., PET or cerebrospinal fluid measures, administration of the composition comprising a therapeutically effective amount of at least one anti-Aβ protofibril antibody would resume. The brain amyloid level would again be measured after administration of the composition comprising a therapeutically effective amount of at least one anti-Aβ protofibril antibody.

After administration of the composition, at least one maintenance therapy agent, such as a BACE inhibitor, is optionally administered to the subject. In some embodiments, the BACE inhibitor may be elenbecestat.

In addition to measurement and consideration of brain amyloid level, cerebrospinal fluid Aβ₁₋₄₂ level, and/or cerebrospinal fluid total tau level, other measures of the subject's health are measured and/or monitored, e.g., MMSE,

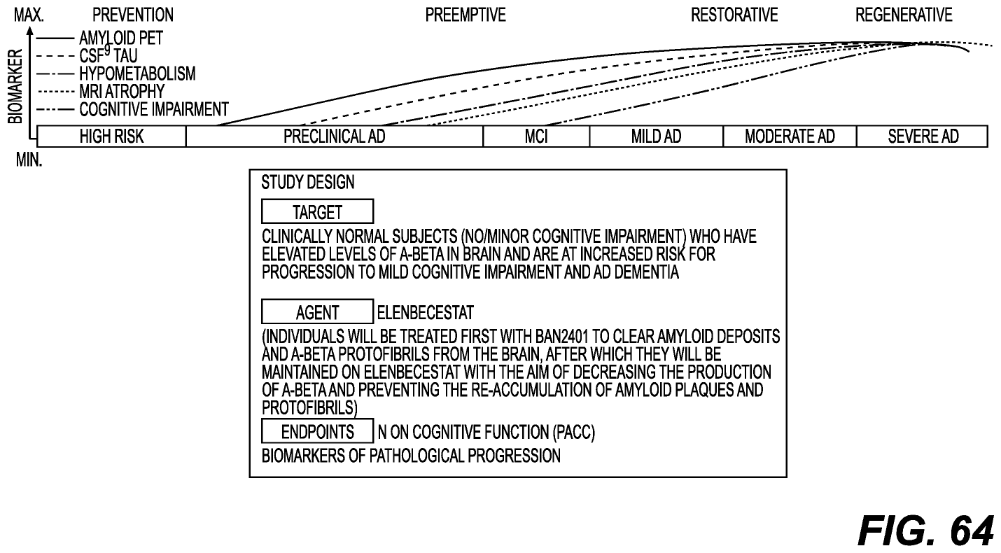

ADAS-cog, CDR, and FAQ. See, e.g., FIG. 64.

### SEQUENCE LISTING

TABLE 24
Amino acid sequences of
mAb variable regions

| | | SEQ | |
|---|---|---|---|
| | IgG | ID | |
| mAb | chain | NO | Amino acid sequence |
| BAN2401 | Heavy | 1 | EVQLVESGGGLVQPGGSLRLSCSASG |
| | chain | | FTFSSFGMHWVRQAPGKGLEWVAYIS |
| | | | SGSSTIYYGDTVKGRFTISRDNAKNS |
| | | | LFLQMSSLRAEDTAVYYCAREGGYYY |
| | | | GRSYYTMDYWGQGTTVTVSS |
| BAN2401 | Light | 2 | DVVMTQSPLSLPVTPGAPASISCRSS |
| | chain | | QSIVHSNGNTYLEWYLQKPGQSPKLL |
| | | | IYKVSNRFSGVPDRFSGSGSGTDFTL |
| | | | RISRVEAEDVGIYYCFQGSHVPPTFG |
| | | | PGTKLEIK |

TABLE 25
Amino acid sequences of
mAb constant regions

| | | | SEQ | |
|---|---|---|---|---|
| | IgG | | ID | |
| mAb | chain | Class | NO | Amino acid sequence |
| BAN2401 | Heavy | IgG1 | 3 | ASTKGPSVFPLAPSSKSTSG |
| | chain | | | GTAALGCLVKDYFPEPVTVS |
| | | | | WNSGALTSGVHTFPAVLQSS |
| | | | | GLYSLSSVVTVPSSSLGTQT |
| | | | | YICNVNHKPSNTKVDKRVEP |
| | | | | KSCDKTHTCPPCPAPELLGG |
| | | | | PSVFLFPPKPKDTLMISRTP |
| | | | | EVTCVVVDVSHEDPEVKFNW |
| | | | | YVDGVEVHNAKTKPREEQYN |
| | | | | STYRVVSVLTVLHQDWLNGK |
| | | | | EYKCKVSNKALPAPIEKTIS |
| | | | | KAKGQPREPQVYTLPPSREE |
| | | | | MTKNQVSLTCLVKGFYPSDI |
| | | | | AVEWESNGQPENNYKTTPPV |
| | | | | LDSDGSFFLYSKLTVDKSRW |
| | | | | QQGNVFSCSVMHEALHNHYT |
| | | | | QKSLSLSPGK |
| BAN2401 | Light | kappa | 4 | RTVAAPSVFIFPPSDEQLKS |
| | chain | | | GTASVVCLLNNFYPREAKVQ |
| | | | | WKVDNALQSGNSQESVTEQD |
| | | | | SKDSTYSLSSTLTLSKADYE |
| | | | | KHKVYACEVTHQGLSSPVTK |
| | | | | SFNRGEC |

TABLE 26
Amino acid sequences of mAb CDRs

| | IgG | SEQ ID | Amino acid |
|---|---|---|---|
| mAb | chain | NO | sequence |
| BAN2401 | HCDR1 | 5 | SFGMH |
| | HCDR2 | 6 | YISSGSSTIYYGDTVKG |
| | HCDR3 | 7 | EGGYYYGRSYYTMDY |
| BAN2401 | LCDR1 | 8 | RSSQSIVHSNGNTYLE |
| | LCDR2 | 9 | KVSNRFS |
| | LCDR3 | 10 | FQGSHVPPT |

SEQ ID NO: 11
heavy chain:
evqlvesggglvqpggslrlscsasgftfssfgmhwvrqapgkglewvay
issgsstiyygdtvkgrftisrdnaknslflqmsslraedtavyycareg
gyyygrsyytmdywgqgttvtvssastkgpsvfplapsskstsggtaalg
clvkdyfpepvtvswnsgaltsgvhtfpavlqssglyslssvvtvpsssl
gtqtyicnvnhkpsntkvdkrvepkscdkthtcppcpapellggpsvflf
ppkpkdtlmisrtpevtcvvvdvshedpevkfnwyvdgvevhnaktkpre
eqynstyrvvsvltvlhqdwlngkeykckvsnkalpapiektiskakgqp
repqvytlppsreemtknqvsltclvkgfypsdiavewesngqpennykt
tppvldsdgsfflyskltvdksrwqqgnvfscsvmhealhnhytqkslsl
spgk
light chain:
dvvmtqsplslpvtpgapasiscrssqsivhsngntylewylqkpgqspk
lliykvsnrfsgvpdrfsgsgsgtdftlrisrveaedvgiyycfqgshvp
ptfgpgtkleikrtvaapsvfifppsdeqlksgtasvvcllnnfypreak
vqwkvdnalqsgnsqesvteqdskdstyslsstltlskadyekhkvyace
vthqglsspvtksfnrgec

What is claimed:

1. A method of reducing clinical decline in a subject having early Alzheimer's disease comprising administering a composition comprising a therapeutically effective amount of at least one anti-Aβ protofibril antibody.

2. The method according to claim 1, wherein the at least one anti-Aβ protofibril antibody comprises three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3);
and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

3. The method according to claim 1, wherein the subject having early Alzheimer's disease has been diagnosed as having mild cognitive impairment due to Alzheimer's disease—intermediate likelihood and/or has been diagnosed as having mild Alzheimer's disease dementia.

4. The method according to any one of claims 1 to 3, wherein the subject is ApoE4-positive.

5. The method according to any one of claims 1 to 4, wherein the at least one anti-Aβ protofibril antibody is BAN2401.

6. The method according to claim 5, wherein the composition comprises 5 mg/kg to 10 mg/kg BAN2401 relative to the weight of the subject.

7. The method according to claim 5, wherein the composition comprises 10 mg/kg BAN2401 relative to the weight of the subject.

8. The method according to any one of claims 1 to 7, wherein the composition is administered once every 2 weeks or once every month.

9. The method according to any one of claims 1 to 8, wherein the clinical decline is reduced by at least 45% relative to placebo as determined by ADCOMS after 6 months of administration of the composition.

10. The method according to any one of claims 1 to 9, wherein the clinical decline is reduced by at least 35% relative to placebo as determined by ADCOMS after 12 months of administration of the composition.

11. The method according to any one of claims 1 to 10, wherein the clinical decline is reduced by at least 30% relative to placebo as determined by ADCOMS after 18 months of administration of the composition.

12. The method according to any one of claims 1 to 11, wherein the clinical decline is reduced by at least 47% relative to placebo as determined by ADAS-Cog after 18 months of administration of the composition.

13. The method according to any one of claims 1 to 12, wherein the clinical decline is reduced by at least 26% relative to placebo as determined by CDR-SB after 18 months of administration of the composition.

14. The method according to claim 11, wherein the subject having early Alzheimer's disease has been diagnosed as having mild cognitive impairment due to Alzheimer's disease—intermediate likelihood, and wherein the clinical decline is reduced by at least 33% relative to placebo as determined by ADCOMS after 18 months of administration of the composition.

15. The method according to claim 11, wherein the subject having early Alzheimer's disease has been diagnosed as having mild Alzheimer's disease dementia, and wherein the clinical decline is reduced by at least 35% relative to placebo as determined by ADCOMS after 18 months of administration of the composition.

16. The method according to any one of claims 9 to 15, wherein the composition comprises 10 mg/kg of at least one anti-Aβ protofibril antibody and is administered once every two weeks or once every month.

17. The method according to claim 16, wherein the at least one anti-Aβ protofibril antibody is BAN2401.

18. The method according to any one of claims 9 to 17, wherein the subject is ApoE4-positive.

19. The method according to claim 18, wherein the clinical decline is reduced by at least 63% relative to placebo as determined by ADCOMS after 18 months of administration of the composition.

20. The method according to claim 18, wherein the clinical decline is reduced by at least 84% relative to placebo as determined by ADAS-Cog after 18 months of administration of the composition.

21. The method according to claim 18, wherein the clinical decline is reduced by at least 60% relative to placebo as determined by CDR-SB after 18 months of administration of the composition.

22. The method according to any one of claims 1 to 17, wherein the subject is ApoE4-negative.

23. The method according to any one of claims 1 to 22, wherein said administration results in a reduction of cerebrospinal fluid level of Aβ₁₋₄₂, total tau, phospho-tau, and/or neurogranin.

24. The method according to any one of claims 1 to 23, wherein said administration results in a slowing of increase in cerebrospinal fluid level of neurofilament light chain.

25. The method according to any one of claims 1 to 24, wherein the subject is concomitantly administered at least one Alzheimer's disease medication other than BAN2401.

26. The method according to any one of claims 1 to 24, wherein the subject is not concomitantly administered at least one Alzheimer's disease medication other than BAN2401.

27. A method of converting an amyloid positive subject having early Alzheimer's disease to amyloid negative comprising administering a composition comprising a therapeutically effective amount of at least one anti-Aβ protofibril antibody.

28. The method according to claim 27, wherein the at least one anti-Aβ protofibril antibody comprises three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3);
and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

29. The method according to claim 27, wherein the subject having early Alzheimer's disease has been diagnosed as having mild cognitive impairment due to Alzheimer's disease—intermediate likelihood and/or has been diagnosed as having mild Alzheimer's disease dementia.

30. The method according to any one of claims 27 to 29, wherein the subject is ApoE4-positive.

31. The method according to any one of claims 27 to 30, wherein the at least one anti-Aβ protofibril antibody is BAN2401.

32. The method according to any one of claims 27 to 31, wherein the composition comprises 5 mg/kg to 10 mg/kg BAN2401 relative to the weight of the subject.

33. The method according to claim 32, wherein the composition comprises 10 mg/kg BAN2401 relative to the weight of the subject.

34. The method according to any one of claims 27 to 33, wherein the composition is administered once every 2 weeks or once every month.

35. The method according to any one of claims 27 to 31, wherein administration of the composition results in 50% to 100% of subjects being amyloid negative as determined by visual reads of amyloid PET images after 6 months of administration of the composition.

36. The method according to any one of claims 27 to 31, wherein administration of composition results in at least 55% of the subjects being amyloid negative, as determined by visual reads of amyloid PET images, after 12 months of administration of the composition.

37. The method according to any one of claims 27 to 31 wherein administration of the composition results in at least 70% of the subjects being amyloid negative, as determined by visual reads of amyloid PET images, after 18 months of administration of the composition.

38. The method according to any one of claims 35 to 37, wherein the composition comprises 10 mg/kg of at least one anti-Aβ protofibril antibody and is administered once every two weeks or once every month.

39. The method according to any one of claims 35 to 38, wherein the at least one anti-Aβ protofibril antibody is BAN2401.

40. The method according to any one of claims 35 to 39, wherein the subject is ApoE4-positive.

41. The method according to any one of claims 35 to 39, wherein the subject is ApoE4-negative.

42. The method according to any one of claims 35 to 41, wherein said administration results in a reduction of cerebrospinal fluid level of Aβ₁₋₄₂, total tau, phospho-tau, and/or neurogranin.

43. The method according to any one of claims 35 to 42, wherein said administration results in a slowing of increase in cerebrospinal fluid level of neurofilament light chain.

44. A method of reducing brain amyloid level in a subject in need thereof comprising administering a composition comprising a therapeutically effective amount of at least one anti-Aβ protofibril antibody.

45. The method according to claim 44, wherein the antibody comprises three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3); and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

46. The method according to claim 44 or 45, wherein the subject has Alzheimer's disease, Down's Syndrome, chronic traumatic encephalopathy, cerebral amyloid angiopathy, Lewy Body Dementia, or another brain disease or conditions with Aβ peptide-containing soluble and/or insoluble Aβ aggregates.

47. The method according to claim 46, wherein the Alzheimer's disease is early Alzheimer's disease.

48. The method according to claim 47, wherein the subject having early Alzheimer's disease has been diagnosed as having mild cognitive impairment due to Alzheimer's disease—intermediate likelihood and/or has been diagnosed as having mild Alzheimer's disease dementia.

49. The method according to any one of claims 44 to 48, wherein the subject is ApoE4-positive.

50. The method according to any one of claims 44 to 49, wherein the at least one anti-Aβ protofibril antibody is BAN2401.

51. The method according to claim 50, wherein the composition comprises 2.5 mg/kg to 10 mg/kg BAN2401 relative to the weight of the subject.

52. The method according to claim 51, wherein the composition comprises 10 mg/kg BAN2401 relative to the weight of the subject.

53. The method according to any one of claims 44 to 52, wherein the composition is administered once every 2 weeks or once every month.

54. The method according to any one of claims 44 to 53, wherein the adjusted mean change from baseline in a subject's PET SUVr value is reduced by at least −0.20 after 12 months of administration of the composition.

55. The method according to any one of claims 44 to 53, wherein the adjusted mean from baseline in a subject's PET SUVr value is reduced by at least −0.25 after 18 months of administration of the composition.

56. The method according to any one of claims 44 to 55, wherein the composition comprises 10 mg/kg of at least one anti-Aβ protofibril antibody and is administered once every two weeks or once every month.

57. The method according to any one of claims 44 to 55, wherein the at least one anti-Aβ protofibril antibody is BAN2401.

58. The method according to any one of claims 44 to 57, wherein the subject is ApoE4-positive.

59. The method according to any one of claims 44 to 53, wherein the subject is ApoE4-negative.

60. The method according to any one of claims 44 to 59, wherein said administration results in a reduction of cerebrospinal fluid level of Aβ₁₋₄₂, total tau, phospho-tau, and/or neurogranin.

61. The method according to any one of claims 44 to 60, wherein said administration results in a slowing of increase in cerebrospinal fluid level of neurofilament light chain.

62. A method of preventing Alzheimer's disease in a subject comprising:
measuring the brain amyloid level of the subject;
if the brain amyloid level of the subject is above a first predetermined level, administering a composition comprising a therapeutically effective amount of at least one anti-Aβ protofibril antibody,
measuring the post-administration brain amyloid level of the subject,
administering the composition if the post-administration brain amyloid level is above a second predetermined level,
monitoring the brain amyloid level of the subject after administration until the brain amyloid level of the subject is below a first predetermined level,
optionally administering at least one additional therapeutic agent suitable for maintenance of the predetermine brain amyloid level chosen from BACE inhibitors, gamma secretase inhibitors, gamma secretase modulators, Aβ peptide generation inhibitors other than said at least one anti-Aβ protofibril antibody, agents that lower Aβ peptide levels other than said at least one anti-Aβ protofibril antibody, and a combination thereof.

63. The method according to claim 62, wherein the pre- and/or post-administration brain amyloid level determination further comprises determining a pre-administration cerebrospinal fluid level of Afti-42, total tau, phospho-tau, neurogranin, and/or neurofilament light chain.

64. The method according to claim 62 or 63, further comprising:
administering the composition if the post-administration cerebrospinal fluid level of Aβ₁₋₄₂, total tau, phospho-tau, neurogranin, and/or neurofilament light chain is above a predetermined level.

65. The method according to any one of claims 62 to 64, wherein the at least one anti-Aβ protofibril antibody is BAN2401.

66. The method according to claim 65, wherein the composition comprises 2.5 mg/kg to 10 mg/kg BAN2401 relative to the weight of the subject.

67. The method according to claim 66, wherein the composition comprises 10 mg/kg BAN2401 relative to the weight of the subject.

68. The method according to any one of claims 62 to 67, wherein the composition is administered once every 2 weeks or once every month.

69. The method according to any one of claims 62 to 68, wherein the brain amyloid level is reduced after said administration relative to the brain amyloid level prior to said administration.

70. The method according to any one of claims 62 to 69, wherein said administration is discontinued if the brain amyloid level is reduced by at least 10%, at least 20%, at least 30%, at least 40%, at least 50%, at least 60%, at least 70%, at least 80%, at least 90%, or 100% relative to the brain amyloid level prior to said administration.

71. The method according to any one of claims 62 to 70, wherein the brain amyloid level is evaluated by visual reads of amyloid PET images.

72. The method according to according to any one of claims 62 to 71, wherein the cerebrospinal fluid level of Aβ₁₋₄₂ is reduced after said administration relative to the cerebrospinal fluid level of Aβ₁₋₄₂ prior to said administration.

73. The method according to any one of claims 62 to 72, wherein the cerebrospinal fluid level of total tau is reduced after said administration relative to the cerebrospinal fluid level of total tau prior to said administration.

74. The method according to any one of claims 62 to 73, wherein the cerebrospinal fluid level of phospho-tau is reduced after said administration relative to the cerebrospinal fluid level of phospho-tau prior to said administration.

75. The method according to any one of claims 62 to 74, wherein the cerebrospinal fluid level of neurogranin is reduced after said administration relative to the cerebrospinal fluid level of neurogranin prior to said administration.

76. The method according to any one of claims 62 to 75, wherein the rate of cerebrospinal fluid level increase of neurofilament light chain is less after said administration relative to the rate of cerebrospinal fluid level increase prior to said administration.

77. The method according to any one of claims 62 to 76, wherein the optionally administered at least one additional therapeutic agent suitable for maintenance of the predetermined brain amyloid level is a BACE inhibitor.

78. The method according to claim 77, wherein the BACE inhibitor is elenbecestat.

79. The method according to any one of claims 62 to 78, wherein the subject is ApoE4-positive.

80. A method of preventing Alzheimer's disease in a subject comprising:
measuring brain amyloid level of the subject;
if the brain amyloid level of the subject is above a first predetermined level, administering a composition comprising a therapeutically effective amount of at least one anti-Aβ protofibril antibody and a composition comprising a therapeutically effective amount of at least one therapeutic agent chosen from BACE inhibitors, gamma secretase inhibitors, gamma secretase modulators, Aβ peptide generation inhibitors other than said at least one anti-Aβ protofibril antibody, and agents that lower the levels of Aβ peptide other than said at least one anti-Aβ protofibril antibody,
measuring the post-administration brain amyloid level of the subject,
administering a composition comprising a therapeutically effective amount of at least one anti-Aβ protofibril antibody and a composition comprising a therapeutically effective amount of at least one therapeutic agent chosen from BACE inhibitors, gamma secretase inhibitors, gamma secretase modulators, Aβ peptide generation inhibitors other than said at least one anti-Aβ protofibril antibody, and agents that lower the levels of Aβ peptide other than said at least one anti-Aβ protofibril antibody if the post-administration brain amyloid level is above a second predetermined level,
monitoring the brain amyloid level of the subject after administration until the brain amyloid level of the subject is below a first predetermined level,
administering at least one additional therapeutic agent suitable for maintenance of the predetermine brain amyloid level chosen from BACE inhibitors, gamma secretase inhibitors, gamma secretase modulators, Aβ peptide generation inhibitors other than said at least one anti-Aβ protofibril antibody, and agents that lower the levels of Aβ peptide other than said at least one anti-Aβ protofibril antibody.

81. The method according to claim 80, wherein the pre- and/or post-administration brain amyloid level determination further comprises determining a pre-administration cerebrospinal fluid level of Aβ₁₋₄₂, total tau, phospho-tau, neurogranin, and/or neurofilament light chain.

82. The method according to claim 80 or 81, further comprising:
administering the composition if the post-administration cerebrospinal fluid level of Aβ₁₋₄₂, total tau, phospho-tau, neurogranin, and/or neurofilament light chain is above a predetermined level.

83. The method according to any one of claims 80 to 82, wherein the at least one anti-Aβ protofibril antibody is BAN2401.

84. The method according to any one of claims 80 to 83, wherein the at least one therapeutic agent is a BACE inhibitor.

85. The method according to claim 84, wherein the BACE inhibitor is elenbecestat.

86. The method according to claim 80, wherein the composition comprises 2.5 mg/kg to 10 mg/kg BAN2401 relative to the weight of the subject.

87. The method according to claim 86, wherein the composition comprises 10 mg/kg BAN2401 relative to the weight of the subject.

88. The method according to any one of claims 80 to 87, wherein the composition is administered once every 2 weeks or once every month.

89. The method according to any one of claims 80 to 88, wherein the brain amyloid level is reduced after said administration relative to the brain amyloid level prior to said administration.

90. The method according to any one of claims 80 to 89, wherein said administration is discontinued if the brain amyloid level is reduced by at least 10%, at least 20%, at least 30%, at least 40%, at least 50%, at least 60%, at least 70%, at least 80%, at least 90%, or 100% relative to the brain amyloid level prior to said administration.

91. The method according to any one of claims 80 to 90, wherein the brain amyloid level is evaluated by visual reads of amyloid PET images.

92. The method according to according to any one of claims 80 to 91, wherein the cerebrospinal fluid level of Aβ₁₋₄₂ is reduced after said administration relative to the cerebrospinal fluid level of Aβ₁₋₄₂ prior to said administration.

93. The method according to any one of claims 80 to 92, wherein the cerebrospinal fluid level of total tau is reduced after said administration relative to the cerebrospinal fluid level of total tau prior to said administration.

94. The method according to any one of claims 80 to 93, wherein the cerebrospinal fluid level of phospho-tau is reduced after said administration relative to the cerebrospinal fluid level of phospho-tau prior to said administration.

95. The method according to any one of claims 80 to 94, wherein the cerebrospinal fluid level of neurogranin is reduced after said administration relative to the cerebrospinal fluid level of neurogranin prior to said administration.

96. The method according to any one of claims 80 to 95, wherein the rate of cerebrospinal fluid level increase of neurofilament light chain is less after said administration relative to the rate of cerebrospinal fluid level increase prior to said administration.

97. The method according to any one of claims 80 to 96, wherein the optionally administered at least one additional therapeutic agent suitable for maintenance of the predetermined brain amyloid level is a BACE inhibitor.

98. The method according to claim 97, wherein the BACE inhibitor is elenbecestat.

99. A method of treating a subject having early Alzheimer's disease comprising administering a composition comprising a therapeutically effective amount of at least one anti-Aβ protofibril antibody, wherein clinical decline of the subject is reduced by at least 35% relative to placebo as determined by ADCOMS after 6 months of administration of the composition, by at least 30% relative to placebo as determined by ADCOMS after 12 months of administration of the composition, and/or by at least 25% relative to placebo as determined by ADCOMS after 18 months of administration of the composition.

100. The method according to claim 99, wherein the at least one anti-Aβ protofibril antibody comprises three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3);
and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

101. The method according to claim 99 or 100, wherein the subject having early Alzheimer's disease has been diagnosed as having mild cognitive impairment due to Alzheimer's disease—intermediate likelihood and/or has been diagnosed as having mild Alzheimer's disease dementia.

102. The method according to any one of claims 99 to 101, wherein the subject is ApoE4-positive.

103. The method according to any one of claims 99 to 102, wherein the at least one anti-Aβ protofibril antibody is BAN2401.

104. The method according to claim 103, wherein the composition comprises 5 mg/kg to 10 mg/kg BAN2401 relative to the weight of the subject.

105. The method according to claim 103, wherein the composition comprises 10 mg/kg BAN2401 relative to the weight of the subject.

106. The method according to any one of claims 99 to 105, wherein the composition is administered once every 2 weeks or once every month.

107. The method according to any one of claims 99 to 106, wherein the clinical decline is reduced by at least 45% relative to placebo as determined by ADCOMS after 6 months of administration of the composition.

108. The method according to any one of claims 99 to 106, wherein the clinical decline is reduced by at least 35% relative to placebo as determined by ADCOMS after 12 months of administration of the composition.

109. The method according to any one of claims 99 to 106, wherein the clinical decline is reduced by at least 30% relative to placebo as determined by ADCOMS after 18 months of administration of the composition.

110. The method according to any one of claims 99 to 106, wherein the clinical decline is reduced by at least 47% relative to placebo as determined by ADAS-COG after 18 months of administration of the composition.

111. The method according to any one of claims 99 to 106, wherein the clinical decline is reduced by at least 26% relative to placebo as determined by CDR-SB after 18 months of administration of the composition.

112. The method according to claim 109, wherein the subject having early Alzheimer's disease has been diagnosed as having mild cognitive impairment due to Alzheimer's disease—intermediate likelihood, and wherein the clinical decline is reduced by at least 33% relative to placebo as determined by ADCOMS after 18 months of administration of the composition.

113. The method according to claim 109, wherein the subject having early Alzheimer's disease has been diagnosed as having mild Alzheimer's disease dementia, and wherein the clinical decline is reduced by at least 35% relative to placebo as determined by ADCOMS after 18 months of administration of the composition.

114. The method according to any one of claims 107 to 109, wherein the composition comprises 10 mg/kg of at least one anti-Aβ protofibril antibody and is administered once every two weeks or once every month.

115. The method according to claim 114, wherein the at least one anti-Aβ protofibril antibody is BAN2401.

116. The method according to any one of claims 99 to 115, wherein the subject is ApoE4-positive.

117. The method according to claim 116, wherein the clinical decline is reduced by at least 63% relative to placebo as determined by ADCOMS after 18 months of administration of the composition.

118. The method according to claim 116, wherein the clinical decline is reduced by at least 84% relative to placebo as determined by ADAS-Cog after 18 months of administration of the composition.

119. The method according to claim 116, wherein the clinical decline is reduced by at least 60% relative to placebo as determined by CDR-SB after 18 months of administration of the composition.

120. The method according to any one of claims 99 to 114, wherein the subject is ApoE4-negative.

121. The method according to any one of claims 99 to 120, wherein said administration results in a reduction of cerebrospinal fluid level of Aβ₁₋₄₂, total tau, phospho-tau, and/or neurogranin.

122. The method according to any one of claims 99 to 121, wherein said administration results in a slowing of increase in cerebrospinal fluid level of neurofilament light chain.

123. The method according to any one of claims 99 to 122, wherein the subject is concomitantly administered at least one Alzheimer's disease medication other than BAN2401.

124. The method according to any one of claims 99 to 122, wherein the subject is not concomitantly administered at least one Alzheimer's disease medication other than BAN2401.

125. A method of treating a subject having early Alzheimer's disease comprising administering a composition comprising a therapeutically effective amount of at least one anti-Aβ protofibril antibody,
wherein the severity of at least one symptom associated with Alzheimer's disease is reduced by at least 10%, 20%, 30%, 40%, 50%, 60%, 70%, 80%, 90%, or 95% relative to the severity of the same symptom in the same subject prior to treatment.

126. The method according to claim 125, wherein the at least one anti-Aβ protofibril antibody comprises three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3);
and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

127. The method according to claim 125 or 126, wherein the subject having early Alzheimer's disease has been diagnosed as having mild cognitive impairment due to Alzheimer's disease—intermediate likelihood and/or has been diagnosed as having mild Alzheimer's disease dementia.

128. The method according to any one of claims 125 to 127, wherein the subject is ApoE4-positive.

129. The method according to any one of claims 125 to 128, wherein the at least one anti-Aβ protofibril antibody is BAN2401.

130. The method according to claim 129, wherein the composition comprises 5 mg/kg to 10 mg/kg BAN2401 relative to the weight of the subject.

131. The method according to claim 129, wherein the composition comprises 10 mg/kg BAN2401 relative to the weight of the subject.

132. The method according to any one of claims 125 to 131, wherein the composition is administered once every 2 weeks or once every month.

133. The method according to any one of claims 125 to 132, wherein the at least one symptom associated with Alzheimer's disease is clinical decline, and wherein the clinical decline is reduced by at least 45% relative to placebo as determined by ADCOMS after 6 months of administration of the composition.

134. The method according to any one of claims 125 to 132, wherein the at least one symptom associated with Alzheimer's disease is clinical decline, and wherein the clinical decline is reduced by at least 35% relative to placebo as determined by ADCOMS after 12 months of administration of the composition.

135. The method according to any one of claims 125 to 132, wherein the at least one symptom associated with Alzheimer's disease is clinical decline, and wherein the clinical decline is reduced by at least 30% relative to placebo as determined by ADCOMS after 18 months of administration of the composition.

136. The method according to any one of claims 125 to 132, wherein the clinical decline is reduced by at least 47% relative to placebo as determined by ADAS-Cog after 18 months of administration of the composition.

137. The method according to any one of claims 125 to 132, wherein the clinical decline is reduced by at least 26% relative to placebo as determined by CDR-SB after 18 months of administration of the composition.

138. The method according to claim 135, wherein the subject having early Alzheimer's disease has been diagnosed as having mild cognitive impairment due to Alzheimer's disease—intermediate likelihood, and wherein the clinical decline is reduced by at least 33% relative to placebo as determined by ADCOMS after 18 months of administration of the composition.

139. The method according to claim 135, wherein the subject having early Alzheimer's disease has been diagnosed as having mild Alzheimer's disease dementia, and wherein the clinical decline is reduced by at least 35% relative to placebo as determined by ADCOMS after 18 months of administration of the composition.

140. The method according to any one of claims 125 to 139, wherein the composition comprises 10 mg/kg of at least one anti-Aβ protofibril antibody and is administered once every two weeks or once every month.

141. The method according to claim 140, wherein the at least one anti-Aβ protofibril antibody is BAN2401.

142. The method according to any one of claims 125 to 141, wherein the subject is ApoE4-positive.

143. The method according to claim 142, wherein the clinical decline is reduced by at least 63% relative to placebo as determined by ADCOMS after 18 months of administration of the composition.

144. The method according to claim 142, wherein the clinical decline is reduced by at least 84% relative to placebo as determined by ADAS-Cog after 18 months of administration of the composition.

145. The method according to claim 142, wherein the clinical decline is reduced by at least 60% relative to placebo as determined by CDR-SB after 18 months of administration of the composition.

146. The method according to any one of claims 125 to 145, wherein the at least one symptom associated with Alzheimer's disease is chosen from clinical decline and brain amyloid level.

147. The method according to any one of claims 125 to 146, wherein said administration results in a reduction of cerebrospinal fluid level of Aβ₁₋₄₂, total tau, phospho-tau, and/or neurogranin.

148. The method according to any one of claims 125 to 147, wherein said administration results in a slowing of increase in cerebrospinal fluid level of neurofilament light chain.

149. The method according to any one of claims 125 to 148, wherein the subject is concomitantly administered at least one Alzheimer's disease medication other than BAN2401.

150. The method according to any one of claims 125 to 148, wherein the subject is not concomitantly administered at least one Alzheimer's disease medication other than BAN2401.

151. A method of treating a subject having early Alzheimer's disease comprising administering a composition comprising a therapeutically effective amount of at least one anti-Aβ protofibril antibody,
wherein the severity of at least one symptom associated with Alzheimer's disease is reduced by at least 10%, 20%, 30%, 40%, 50%, 60%, 70%, 80%, 90%, or 95% relative to the severity of the same symptom in subjects that received placebo.

152. The method of claim 151, wherein the severity of at least one symptom associated with Alzheimer's disease is determined by ADCOMS, PET, MMSE, CDR-SB, and/or ADAS-Cog.

153. The method according to claim 151, wherein the at least one anti-Aβ protofibril antibody comprises three heavy chain complementarity determining regions (HCDR1, HCDR2, and HCDR3) comprising amino acid sequences of SEQ ID NO: 5 (HCDR1), SEQ ID NO: 6 (HCDR2), and SEQ ID NO: 7 (HCDR3);
and three light chain complementarity determining regions (LCDR1, LCDR2, and LCDR3) comprising amino acid sequences of SEQ ID NO: 8 (LCDR1), SEQ ID NO: 9 (LCDR2), and SEQ ID NO: 10 (LCDR3).

154. The method according to claim 151, wherein the subject having early Alzheimer's disease has been diagnosed as having mild cognitive impairment due to Alzheimer's disease—intermediate likelihood and/or has been diagnosed as having mild Alzheimer's disease dementia.

155. The method according to any one of claims 151 to 154, wherein the subject is ApoE4-positive.

156. The method according to any one of claims 151 to 155, wherein the at least one anti-Aβ protofibril antibody is BAN2401.

157. The method according to claim 156, wherein the composition comprises 5 mg/kg to 10 mg/kg BAN2401 relative to the weight of the subject.

158. The method according to claim 156, wherein the composition comprises 10 mg/kg BAN2401 relative to the weight of the subject.

159. The method according to any one of claims 151 to 158, wherein the composition is administered once every 2 weeks or once every month.

160. The method according to any one of claims 151 to 159, wherein the clinical decline is reduced by at least 45% relative to placebo as determined by ADCOMS after 6 months of administration of the composition.

161. The method according to any one of claims 151 to 159, wherein the at least one symptom associated with Alzheimer's disease is clinical decline, and wherein clinical decline is reduced by at least 35% relative to placebo as determined by ADCOMS after 12 months of administration of the composition.

162. The method according to any one of claims 151 to 159, wherein the at least one symptom associated with Alzheimer's disease is clinical decline, and wherein the clinical decline is reduced by at least 30% relative to placebo as determined by ADCOMS after 18 months of administration of the composition.

163. The method according to any one of claims 151 to 159, wherein the clinical decline is reduced by at least 47% relative to placebo as determined by ADAS-Cog after 18 months of administration of the composition.

164. The method according to any one of claims 151 to 159, wherein the clinical decline is reduced by at least 26% relative to placebo as determined by CDR-SB after 18 months of administration of the composition.

165. The method according to claim 162, wherein the subject having early Alzheimer's disease has been diagnosed as having mild cognitive impairment due to Alzheimer's disease—intermediate likelihood, and wherein the clinical decline is reduced by at least 33% relative to placebo as determined by ADCOMS after 18 months of administration of the composition.

166. The method according to claim 162, wherein the subject having early Alzheimer's disease has been diagnosed as having mild Alzheimer's disease dementia, and wherein the clinical decline is reduced by at least 35% relative to placebo as determined by ADCOMS after 18 months of administration of the composition.

167. The method according to any one of claims 160 to 166, wherein the composition comprises 10 mg/kg of at least one anti-Aβ protofibril antibody and is administered once every two weeks or once every month.

168. The method according to claim 167, wherein the at least one anti-Aβ protofibril antibody is BAN2401.

169. The method according to any one of claims 160 to 167, wherein the subject is ApoE4-positive.

170. The method according to claim 169, wherein the clinical decline is reduced by at least 63% relative to placebo as determined by ADCOMS after 18 months of administration of the composition.

171. The method according to claim 169, wherein the clinical decline is reduced by at least 84% relative to placebo as determined by ADAS-Cog after 18 months of administration of the composition.

172. The method according to claim 169, wherein the clinical decline is reduced by at least 60% relative to placebo as determined by CDR-SB after 18 months of administration of the composition.

173. The method according to any one of claims 151 to 169, wherein the at least one symptom associated with Alzheimer's disease is chosen from clinical decline and brain amyloid level.

174. The method according to any one of claims 151 to 173, wherein said administration results in a reduction of cerebrospinal fluid level of Aβ₁₋₄₂, total tau, phospho-tau, and/or neurogranin.

175. The method according to any one of claims 151 to 173, wherein said administration results in a slowing of increase in cerebrospinal fluid level of neurofilament light chain.

176. The method according to any one of claims 151 to 173, wherein the subject is concomitantly administered at least one Alzheimer's disease medication other than BAN2401.

177. The method according to any one of claims 151 to 173, wherein the subject is not concomitantly administered at least one Alzheimer's disease medication other than BAN2401.

178. The method according to any one of claims 80 to 99, wherein the subject is ApoE4-positive.

179. The method according to any one of claim 27, 62 to 78, 80 to 98, 125, or 151, wherein the subject is ApoE4-negative.
